(19) United States
(12) Patent Application Publication
(10) Pub. No.: US 2021/0040452 A1
(43) Pub. Date: Feb. 11, 2021

(54) HEMATOPOIETIC CELLS AND METHODS OF USING AND GENERATING THE SAME

(71) Applicant: The Regents of the University of California, Oakland, CA (US)

(72) Inventor: Ann C. Zovein, San Francisco, CA (US)

(73) Assignee: The Regents of the University of California, Oakland, CA (US)

(21) Appl. No.: 15/735,115

(22) PCT Filed: Jun. 9, 2016
(86) PCT No.: PCT/US16/36747

§ 371 Date: Dec. 8, 2017

Related U.S. Application Data
(60) Provisional application No. 62/173,352, filed on Jun. 9, 2015.

(51) Int. Cl. C12N 5/0789; A61K 35/28

(52) U.S. Cl. CPC C12N 5/0647; C12N 2501/15; C12N 2501/60; A61K 35/28

(57) ABSTRACT

The disclosure relates to compositions comprising hematopoietic cells and methods of using the same. The disclosure also relates to methods of reprogramming endothelial cells into hematopoietic cells by exposing the endothelial cells to at least one hematopoietic effector.

### RELATED APPLICATIONS

This application is a National Stage application filed under 35 U.S.C. § 371 of International Application No. PCT/US2016/036747, filed on Jun. 9, 2016, which claims priority to U.S. Provisional Ser. No. 62/173,352, filed Jun. 9, 2015, each of which is incorporated by reference in its entirety.

### STATEMENT REGARDING FEDERALLY SPONSORED RESEARCH

This invention was made with government support under Grant No. HL117743, awarded by the National Institutes of Health. The government has certain rights in the invention.

### SUBMISSION OF SEQUENCE LISTING

The Sequence Listing associated with this application is filed in electronic format via EFS-Web and hereby incorporated by reference into the specification in its entirety. The name of the text file containing the Sequence Listing is Sequence Listing_37944_0003U2. The size of the text file is 92 KB and the text file was created on Nov. 12, 2019.

### FIELD OF THE DISCLOSURE

The disclosure relates generally to compositions comprising endothelial cells comprising one or a plurality of hematopoietic activators and/or silencers. The disclosure also relates to method of making and using hematopoietic stem cells and progenitor cells from treatment of the endothelial cells for treating disorders.

### BACKGROUND

Endothelial to hematopoietic transition (EHT) during embryogenesis provides the first long term hematopoietic stem and progenitor cells (HSPC) for the organism. The generation of hematopoietic cells from the endothelium occurs during a narrow window in development (embryonic day (E) 10-12 in mouse (de Bruijn et al., 2000), and ˜4-6 weeks in the human (Tavian et al., 1996)). The most well studied site for HSPC emergence is the developing aorta located in the embryonic aortagonad-mesonephros (AGM) region (de Bruijn et al., 2000; North et al., 1999). Intra-aortic hematopoietic clusters appear transiently in the AGM region, and then are thought to migrate to the fetal liver, and ultimately the bone marrow for long-term adult hematopoiesis. Previous studies have demonstrated a requirement of the transcription factor Runx1 for the transition of endothelial cells to a hematopoietic fate (Chen et al., 2009; North et al., 1999). Runx1 expression is noted within a subset of endothelial cells in hemogenic vascular beds but is then localized to hematopoietic cells as intra-aortic clusters emerge (Tober et al., 2013). The transcription factor Sox17 has also been shown to be important for the generation of hemogenic endothelium (Clarke et al., 2013b), as well as playing a role in HSC survival (Kim et al., 2007). However, while SOX17 promotes hemogenic endothelial specification, continued or overexpression has been noted to inhibit the direct transition to hematopoietic fate (Clarke et al., 2013a; Nobuhisa et al., 2014).

### SUMMARY OF EMBODIMENTS

The present disclosure encompasses the recognition that it is possible to convert certain types of endothelial cells, specifically endothelium, into long term hematopoietic stem cells and progenitor cells (HSPCs). The present disclosure generally relates to methods of differentiating endothelial cells into HSPCs by acquiring and culturing the endothelial cells, then exposing them to a combination of hematopoietic effectors for a period of time sufficient to use them for further studies or treatment of disease. In some embodiments, the relative protein levels of the transcription factors Runx1 and Sox17 are manipulated by the one or more hematopoietic effectors to initiate hematopoiesis on a single-cell level.

The present disclosure relates to a method of differentiating an endothelial cell into a stem cell comprising: exposing the endothelial cell to an effective amount of at least one hematopoietic effector for a time period sufficient to induce expression or activation of a hematopoietic pathway; and exposing the endothelial cell to an effective amount of an hematopoietic effector for a time period sufficient to inhibit the hematopoietic pathway.

In some embodiments, the step of exposing endothelial cell to an effective amount of an hematopoietic effector for a time period sufficient to induce expression of a hematopoietic pathway is preceded by a step of isolating one or a plurality of endothelial cells. In some embodiments, the step of isolating one or a plurality of endothelial cells comprises isolating endothelial cell from an umbilical cord or from umbilical cord tissue. In some embodiments, the step of exposing endothelial cell to an effective amount of an hematopoietic effector for a time period sufficient to induce expression of a hematopoietic pathway is preceded by a step of culturing one or a plurality of endothelial cells.

In some embodiments, the time period sufficient to induce expression or activation of a hematopoietic pathway is from about 1 day to about 6 days. In some embodiments, the time period sufficient to inhibit or deactivate the hematopoietic pathway is from about 1 days to about 3 days.

In some embodiments, the step of exposing the endothelial cell to an effective amount of an hematopoietic effector comprises culturing the endothelial cell in the presence of a nucleic acid sequence encoding a hematopoietic activator or a functional fragment thereof. In some embodiments, the step of exposing the endothelial cell to an effective amount of an hematopoietic effector comprises culturing the endothelial cell in the presence of a nucleic acid sequence encoding a hematopoietic silencer or a functional fragment thereof.

The present disclosure relates to a nucleic acid sequence encoding the hematopoietic activator, wherein the nucleic acid is an episome or plasmid. In some embodiments, the nucleic acid sequence encoding the hematopoietic silencer is an episome or plasmid.

In some embodiments, the steps of exposing the endothelial cell to a pharmacologically effective amount of an hematopoietic effector comprises transfecting a nucleic acid encoding a hematopoietic activator or a functional fragment thereof into the endothelial cell. In some embodiments, the steps of exposing the endothelial cell to a pharmacologically effective amount of an hematopoietic effector comprises transfecting a nucleic acid encoding a hematopoietic silencer or a functional fragment thereof into the endothelial cell.

The present disclosure relates to any of the disclosed methods herein, wherein the step of exposing the endothelial cell to a pharmacologically effective amount of a hematopoietic effector comprises exposing the endothelial cell with one or a plurality of small chemical compounds at a pharmacologically effective concentration and for a time period sufficient to silence or to activate the hematopoietic pathway. In some embodiments, the hematopoietic effector is Sox17 or a functional fragment thereof. In some embodiments, the hematopoietic effector is Runx1 or a functional fragment thereof. In some embodiments, the hematopoietic activator is Sox17 or a functional fragment thereof. In some embodiments, the hematopoietic silencer is Runx1 or a functional fragment thereof. In some embodiments, the cell is exposed to a nucleic acid encoding 1, 2, 3, 4, or more hematopoietic effectors. In some embodiments, the cell is exposed to 1, 2, 3, 4, or more hematopoietic effectors, or functional variant or functional fragment thereof in any of the disclosed methods. In some of the embodiments, differentiation of an endothelial cell into a HSPC is achieved by exposure of the endothelial cell to no more than 2 hematopoietic effectors. In some of the embodiments, differentiation of an endothelial cell into a HSPC is achieved by exposure of the endothelial cell to no more than 1 hematopoietic activator and no more than 1 hematopoietic activator.

The disclosure further relates to any of the disclosed methods further comprising exposing the endothelial cell to one or a plurality of cellular transcription factors chosen from one or a combination of: OCT4, SOX2, KLF4, cMYC, LIN28, NANOG, or any functional fragment thereof. In some embodiments, the method further comprises culturing the endothelial cell for a period of time and under conditions sufficient to cause expression of CD41 and/or c-kit.

The present disclosure also relates to a method of producing a hematopoietic stem cell comprising dedifferentiating an endothelial cell. In some embodiments, the step of dedifferentiating the endothelial cell comprises: exposing a endothelial cell to an effective amount of an hematopoietic effector for a time period sufficient to induce expression or of a hematopoietic pathway; and exposing the endothelial cell to a to an effective amount of an hematopoietic effector for a time period sufficient to inhibit the hematopoietic pathway. In some embodiments, the step of exposing endothelial cell to a to an effective amount of an hematopoietic activator for a time period sufficient to induce expression of a hematopoietic pathway is preceded by a step of isolating one or a plurality of endothelial cells. In some embodiments, the step of isolating one or a plurality of endothelial cells comprises isolating endothelial cell from an umbilical cord or from umbilical cord tissue. In some embodiments, the step of exposing endothelial cell to a to an effective amount of an hematopoietic effector for a time period sufficient to induce expression of a hematopoietic pathway is preceded by a step of culturing one or a plurality of endothelial cells.

In some embodiments, the time period sufficient to induce expression of a hematopoietic pathway is from about 1 day to about 6 days. In some embodiments, the time period sufficient to inhibit the hematopoietic pathway is from about 1 days to about 3 days.

In some embodiments, the step of exposing the endothelial cell to an effective amount of an hematopoietic effector comprises culturing the endothelial cell in the presence of a nucleic acid sequence encoding a hematopoietic activator or a functional fragment thereof. In some embodiments, the step of exposing the endothelial cell to an effective amount of an hematopoietic effector comprises culturing the endothelial cell in the presence of a nucleic acid sequence encoding a hematopoietic silencer or a functional fragment thereof.

In some embodiments, the nucleic acid sequence encoding the hematopoietic activator is an episome or plasmid. In some embodiments, the nucleic acid sequence encoding the hematopoietic silencer is an episome or plasmid.

In some embodiments, the steps of exposing the endothelial cell to an effective amount of an hematopoietic effector comprises transfecting a nucleic acid encoding a hematopoietic activator or a functional fragment thereof into the endothelial cell. In some embodiments, the steps of exposing the endothelial cell to an effective amount of an hematopoietic effector comprises transfecting a nucleic acid encoding a hematopoietic silencer or a functional fragment thereof into the endothelial cell.

In some embodiments, the step of exposing the endothelial cell to an effective amount of an hematopoietic effector comprises exposing the endothelial cell with one or a plurality of small chemical compounds at a pharmacologically effective concentration and for a time period sufficient to silence the hematopoietic pathway. In some embodiments, the hematopoietic effector is Sox17 or a functional fragment thereof. In some embodiments, the hematopoietic effector is Runx1 or a functional fragment thereof.

In some embodiments, the method further comprises exposing the endothelial cell to one or a plurality of cellular transcription factors chosen from one or a combination of: OCT4, SOX2, KLF4, cMYC, LIN28, NANOG, or any functional fragment thereof. In some embodiments, the method further comprises culturing the endothelial cell for a period of time and under conditions sufficient to cause expression of CD41 and/or c-kit.

The present disclosure also relates to a method of preparing an in vitro culture of stem cells comprising: (a) exposing an endothelial cell to an effective amount of an hematopoietic effector for a time period sufficient to induce expression of a hematopoietic pathway; and (b) exposing an endothelial cell to an effective amount of an hematopoietic effector for a time period sufficient to inhibit the hematopoietic pathway, such that sequential exposure to both steps of (a) and (b) causes dedifferentiation of the endothelial cell to a stem cell.

In some embodiments, any of the methods disclosed herein further comprise analyzing the cells for expression of one or more genes or functional fragments thereof that is indicative of the endothelial cell acquiring a hematopoietic lineage.

The present disclosure also relates to a method of generating a library of hematopoietic cells comprising: exposing an endothelial cell to an effective amount of an hematopoietic effector for a time period sufficient to induce expression of a hematopoietic pathway; and exposing the endothelial cell to an effective amount of an hematopoietic effector for a time period sufficient to inhibit the hematopoietic pathway.

In some embodiments, the method further comprises isolating an endothelial cell from a subject with a predetermined genetic background before exposing the endothelial cell to an effective amount of an hematopoietic effector for a time period sufficient to induce expression of a hematopoietic pathway. In some embodiments, the method further comprises culturing the endothelial cell in growth media for no less than 4 days. In some embodiment, the method further comprises analyzing an endothelial cell to identify a predetermined genetic background of the endothelial cell before exposing the endothelial cell to an effective amount of an hematopoietic effector for a time period sufficient to induce expression of a hematopoietic pathway. In some embodiments, the method further comprises storing the endothelial cell at or below −80 degrees Celsius.

Any embodiments of the methods disclosed herein may further comprise cataloguing the genetic background of the endothelial cell before, contemporaneously with, or after storing the endothelial cell such that one creates a library of information relative to the phenotype of the cells in the library.

The present disclosure relates, in some embodiments, the steps of (a) exposing an endothelial cell to an effective amount of an hematopoietic effector for a time period sufficient to induce expression of a hematopoietic pathway; and (b) exposing the endothelial cell to an effective amount of an hematopoietic effector for a time period sufficient to inhibit the hematopoietic pathway are repeated in respect to a plurality of endothelial cells; and wherein each endothelial cell exposed to a hematopoietic effector is stored at or below −80 degrees Celsius.

In some embodiments, the step of exposing endothelial cell to a to an effective amount of an hematopoietic effector for a time period sufficient to induce expression of a hematopoietic pathway is preceded by a step of isolating one or a plurality of endothelial cells. In some embodiments, the step of isolating one or a plurality of endothelial cells comprises isolating endothelial cell from an umbilical cord or from umbilical cord tissue. In some embodiments, the step of exposing endothelial cell to a to an effective amount of an hematopoietic effector for a time period sufficient to induce expression of a hematopoietic pathway is preceded by a step of culturing one or a plurality of endothelial cells.

In some embodiments, the time period sufficient to induce expression of a hematopoietic pathway is from about 1 day to about 6 days. In some embodiments, the time period sufficient to inhibit the hematopoietic pathway is from about 1 days to about 3 days.

In some embodiments, the step of exposing the endothelial cell to an effective amount of an hematopoietic effector comprises culturing the endothelial cell in the presence of a nucleic acid sequence encoding a hematopoietic activator or a functional fragment thereof. In some embodiments, the step of exposing the endothelial cell to an effective amount of an hematopoietic effector comprises culturing the endothelial cell in the presence of a nucleic acid sequence encoding a hematopoietic silencer or a functional fragment thereof.

In some embodiments, the nucleic acid sequence encoding the hematopoietic activator is an episome or plasmid. In some embodiments, the nucleic acid sequence encoding the hematopoietic silencer is an episome or plasmid.

In some embodiments, the steps of exposing the endothelial cell to an effective amount of an hematopoietic effector comprises transfecting a nucleic acid encoding the hematopoietic activator or a functional fragment thereof into the endothelial cell. In some embodiments, the steps of exposing the endothelial cell to an effective amount of an hematopoietic effector comprises transfecting a nucleic acid encoding the hematopoietic silencer or a functional fragment thereof into the endothelial cell.

In some embodiments, the step of exposing the endothelial cell to an effective amount of an hematopoietic effector comprises exposing the endothelial cell with one or a plurality of small chemical compounds at a pharmacologically effective concentration and for a time period sufficient to silence the hematopoietic pathway.

In some embodiments, the hematopoietic effector is Sox17 or a functional fragment thereof. In some embodiments, the hematopoietic effector is Runx1 or a functional fragment thereof.

In some embodiments, the method further comprises exposing the endothelial cell to one or a plurality of cellular transcription factors chosen from one or a combination of: OCT4, SOX2, KLF4, cMYC, LIN28, NANOG, or any functional fragment thereof. In some embodiments, the method further comprises culturing the endothelial cell for a period of time and under conditions sufficient to cause expression of CD41 and/or c-kit.

The present disclosure also relates to a method of decreasing rejection of transplanted hematopoietic cells in a subject comprising transplanting one or a plurality of hematopoietic cells derived from an endothelial cell known to contain a Human Leukocyte Antigen (HLA) class I, HLC class II, and/or endothelial cell antigens that are compatible with the subject.

In some embodiments, prior to transplanting one or a plurality of hematopoietic cells derived from an endothelial lineage, the method comprises: exposing one or a plurality of endothelial cells to an effective amount of a hematopoietic effector for a time period sufficient to induce expression of a hematopoietic pathway; and exposing the one or a plurality endothelial cells to an effective amount of a hematopoietic effector for a time period sufficient to inhibit the hematopoietic pathway.

In some embodiments, the method further comprises identifying the a HLA class I, HLA class II, and/or endothelial cell antigen compatibility of the endothelial cell. In some embodiments, the method further comprises identifying the a HLA class I, HLA class II, and/or endothelial cell antigen compatibility of the subject. In some embodiments, the method further comprises matching the a HLA class I, HLA class II, and/or endothelial cell antigen compatibility of the endothelial cell with the subject prior to transplanting one or a plurality of hematopoietic cells derived from an endothelial cell.

The present disclosure also relates to a cell comprising a nucleic acid sequence encoding one or a plurality of hematopoietic silencers. In some embodiments, the cell further comprises a nucleic acid sequence encoding one or a plurality of hematopoietic activators. In some embodiments, the nucleic acid sequence encoding one or a plurality of hematopoietic silencers comprises a nucleic acid sequence with at least 70%, 75%, 80%, 85%, 90%, 95%, 96%, 97%, 98%, 99% or 100% sequence identity to SEQ ID NO:2.

The present disclosure also relates to a cell comprising a nucleic acid sequence encoding one or a plurality of hematopoietic activators. In some embodiments, the a nucleic acid sequence encoding one or a plurality of hematopoietic activators comprises a nucleic acid sequence with at least 70%, 75%, 80%, 85%, 90%, 95%, 96%, 97%, 98%, 99% or 100% sequence identity to SEQ ID NO:1.

The present disclosure also relates to a method of treating or preventing cancer of the blood in a subject in need thereof comprising: administering to the subject one or a plurality of hematopoietic stem cells derived from one or a plurality of endothelial cells.

In some embodiments, the method further comprises steps: (a) exposing the one or a plurality of endothelial cells to an effective amount of a hematopoietic effector for a time period sufficient to induce activation or expression of a hematopoietic pathway; and (b) exposing the one or a plurality endothelial cells to an effective amount of a hematopoietic effector for a time period sufficient to inhibit the hematopoietic pathway, prior to administering the one or plurality of hematopoietic stem cells, such that sequential exposure of the one or plurality of endothelial cells to at least one hematopoietic activator and at least one hematopoietic silencer cause the one or plurality of endothelial cells to dedifferentiate into one or a plurality of hematopoietic stem cells. In some embodiments, steps (a) and (b) are performed ex vivo.

The present disclosure also relates to a method of performing a cellular transplant in a subject in need of a bone marrow cells comprising: administering to the subject one or a plurality of hematopoietic stem cells derived from one or a plurality of endothelial cells.

In some embodiments, the method further comprises steps: (a) exposing the one or a plurality of endothelial cells to an effective amount of a hematopoietic effector for a time period sufficient to induce expression of a hematopoietic pathway; and (b) exposing the one or a plurality endothelial cells to an effective amount of a hematopoietic effector for a time period sufficient to inhibit the hematopoietic pathway, prior to administering the one or plurality of hematopoietic stem cells, such that sequential exposure of the one or plurality of endothelial cells to at least one hematopoietic activator and at least one hematopoietic silencer cause the one or plurality of endothelial cells to dedifferentiate into one or a plurality of hematopoietic stem cells. In some embodiments, the steps (a) and (b) are performed ex vivo.

The present disclosure also relates to a library of cells comprising any one or plurality of cells disclosed herein. The present disclosure also relates to a library of cells comprising one or a plurality of hematopoietic stem cells derived from endothelial cells disclosed herein or any of the methods disclosed herein.

In certain embodiments, the methods described above further comprise exposing the endothelial cell to a pharmacologically effective amount of transforming growth factor β1 (TGFβ1) or a functional fragment thereof, or a pharmacologically effective amount of a nucleic acid sequence encoding the TGFβ1 or a functional fragment thereof. In certain embodiments, the methods described above further comprise culturing the endothelial cell in the presence of a nucleic acid sequence encoding a TGFβ1 or a functional fragment thereof.

In certain aspects, the invention also relate to a cell comprising a heterologous nucleic acid sequence encoding one or a plurality of hematopoietic silencers. In certain embodiments, the cell further comprises a heterologous nucleic acid sequence encoding one or a plurality of hematopoietic activators. In certain embodiments, the nucleic acid sequence encoding one or a plurality of hematopoietic silencers comprises a nucleic acid sequence with at least 70%, 75%, 80%, 85%, 90%, 95%, 96%, 97%, 98%, 99% or 100% sequence identity to SEQ ID NO:2.

In certain aspects, the invention also relates to a cell comprising a heterologous nucleic acid sequence encoding one or a plurality of hematopoietic activators. In certain embodiments, the nucleic acid sequence encoding one or a plurality of hematopoietic activators comprises a nucleic acid sequence with at least 70%, 75%, 80%, 85%, 90%, 95%, 96%, 97%, 98%, 99% or 100% sequence identity to SEQ ID NO:1. In certain embodiments, the cell comprises a plasmid or episome comprising the heterologous nucleic acid sequence. In certain embodiments, the cell is a hematopoietic stem cell.

In certain embodiments, the invention relates to a pharmaceutical composition comprising any of the cells described above.

### BRIEF DESCRIPTION OF THE DRAWINGS

FIG. 1 depicts a paradigms of hematopoietic origins. While there is evidence of a bipotential hemangioblast in the early yolk sac, it does not account for all yolk sac endothelium and hematopoietic cells. Therefore, mesodermal precursors may separately contribute to the endothelium and blood. Hemogenic endothelium, in contrast, requires an endothelial intermediate for the production of blood cells, and may account for the majority of definitive blood cells and a subpopulation of the endothelium.

FIGS. 2A, 2B, and 2C depict inducible VE-cadherin Cre allows tracing of endothelial-derived hematopoiesis. FIG. 2A depicts a schema of inducible Cre excision after tamoxifen administration. The VE-cadherin promoter drives a Cre recombinase (VEC-Cre) with a mutated estrogen receptor ERT2. When crossed to a Cre reporter line with LacZ or EYFP in frame after a “floxed” stop codon (flanking loxP sites: triangles), the Cre is sequestered in the cytoplasm until tamoxifen is administered, and its metabolite 4-hydroxytamoxifen (4OHT) binds to the ERT, allowing Cre access to the nucleus to excise the DNA flanked by loxP sites. In this case, the STOP cassette is removed allowing constitutive expression of the reporter in that cell and all progeny. FIG. 2B depicts a schema demonstrating deletion and/or reporter activity (blue) after tamoxifen induced Cre expression. The inducible Cre allows labeling a particular population (VE-cadherin+) within a particular time frame (e.g. the tamoxifen window). FIG. 2C depicts a demonstration that when hemogenic endothelial vascular beds (E10-11) are dissected and induced with 4-OHT in vitro (24 hrs) we can detect labeled endothelium (LacZ reporter) and hematopoietic cells (YFP+CD45+). The hematopoietic cells are endothelial derived as induction of circulating blood cells from the same embryos do not demonstrate any labeling.

FIG. 2D depicts bone marrow transplantation. When constitutive VE-cadherin Cre adult bone marrow (crossed to a LacZ R26R Cre reporter line) was transplanted into lethally irradiated adult mice, reconstitution persisted at 8 months post-transplant. The percent of LacZ positive cells mirrored that of the transplanted population (by FACSgal47%) suggesting a robust hematopoietic stem compartment within the VE-cadherin (endothelial) progeny.

FIG. 2E depicts vitelline artery HSC capacity. (TOP) The vitelline arteries from VE-cadherin Cre/R26R LacZ embryos at E10.0 were plated for 7-10 days in hematopoietic methylcellulose colony forming assays. Various colonies formed including: definitive erythroid (BFU-e), macrophage (CFUM) and granulocyte/macrophage (CFU-GM). (BOTTOM) When analyzed for AGM hematopoietic stem markers CD34 and c-kit (by FACS), it became apparent that the stem compartment was highly enriched within the VEcadherin lineage traced population (i.e. hemogenic endothelium).

FIG. 2F depicts EYFP imaging. Still images from a VE-cadherin Cre/R26R EYFP E10.0 embryo culture. Left: YFP+ yolk sac vessels with circulating YFP+ circulating cells (arrows). Right: A cardiac silhouette from a video of the beating heart (arrows).

FIGS. 3A, 3B, and 3C depict β1 integrin constructs and hypomorphic protein expression. FIG. 3A depicts the VE-cadherin Cre contruct and three different β1 integrin contructs. The original “floxed” used demonstrates loxP sites flanking the entire gene. The β1 null construct depicts disruption of exon 2 of the gene. The last floxed construct has loxP sites flanking just exon 3 of the gene. FIG. 3B depicts the survival curve depicts a shift to the left when an original β1 floxed construct is bred over a null background β1f/n;Cre+, suggesting some inefficiency of Cre exision. FIG. 3C depicts β1 integrin protein analysis by FACS depicts a late loss of protein within homozygous β1f/f;Cre+ endothelial cells over time (as compared to heterozygous β1f/+;Cre+).

FIGS. 3D and 3E depict β1 integrin ablation results in abnormal endothelial polarity. FIG. 3D depicts when β1 integrin is ablated in endothelium, the deleted cells lose their typical squamous morphology (arrowheads) and become cuboidal (arrows). Heterozygous control on left. FIG. 3E depicts the resultant effect of β1 integrin deletion on the endothelium is an increase in adhesion and loss of polarity, as evidenced by the decrease in Par-3.

FIG. 3F depicts asymmetric β1 integrin and Par-3 expression. Within the AGM at E11 (left) β1 integrin is localized to the endothelium and the inner core of the hematopoietic cluster, while Par-3 (arrows) is localized to the outer layer of the cluster. In the myocardium (right) at E12.5, a cell undergoing division also demonstrates asymmetric Par-3 expression with respect to β1 integrin.

FIGS. 4A-4J depict immunofluorescence of human hemogenic endothelium. FIG. 4A depicts GA week 6. DAPI identifies overall morphology of transverse section of aorta (boxed area). FIG. 4B depicts higher magnification of the boxed area in (FIG. 4A) an intra-aortic cluster is labeled by RUNX1 and PECAM1. Arrowheads depict single RUNX1+ cells associated with the endothelium. FIG. 4C depicts higher magnification of boxed area in (FIG. 4B) with DAPI and 3D volume rendered RUNX1. FIG. 4D depicts GA week 7; ACE/CD143 labels endothelium and one RUNX1+ cell (arrowhead) is noted. FIG. 4E depicts GA week 8; VE-Cadherin (VEC) labels endothelium, and RUNX1 cells are embedded in the vessel wall, while a circulating RUNX1+ is noted in the lumen (arrowheads). FIG. 4F depicts GA week 6; SOX17 and RUNX1 reveal SOX17 endothelial cells. Ventral RUNX1+ cells are embedded in the endothelium (arrowheads). FIG. 4G depicts higher magnification of boxed area in (FIG. 4F); RUNX1+ cells (arrowheads) demonstrate low levels of SOX17. FIGS. 4H and 4 depict Mean Fluorescence Intensity (MFI) of SOX17 and RUNX1 in selected cells (n=12). Arrowheads correspond to values measured in the three cells identified with arrowheads in (FIG. 4G). FIG. 4J depicts ratio of RUNX1 MFI to SOX17 MFI of cells in (FIG. 4H), and corresponding high (>1.0) intermediate (>0.1 to <1.0) and low (<0.1) ratios. The cells indicated with arrowheads in (FIG. 4F) and (FIG. 4G) correspond to the high ratios (>1.0).

FIGS. 5A-5J depict surface marker and gene expression of hematopoietic cell clusters. FIG. 5A depicts the endothelial layer and attached hematopoietic cell clusters are CD31+, while CD117+ identifies cells in the HSPC clusters (arrowhead). FIG. 5B CD41 marker expression notable in endothelial associated hematopoietic cell clusters (arrowhead) identified by CD31+ staining. FIG. 5C depicts CD45+ is noted in mature HCs and few cluster associated cells (arrowheads). SOX17 is localized to the underlying endothelium identified by CD31+ staining. FIG. 5D Sox17 strongly marks nuclei of underlying CD31+ endothelial cells (arrow) compared to the largely dim, punctate staining apparent in HSPC clusters (arrowhead). FIG. 5E depicts E10.5 DA was co-stained with CD31, Sox17 and fluorescent conjugated Lectin HPA (HPA-488), a protein with a strong affinity for the golgi apparatus membranes. Puncate Sox17 staining in the HSPC cluster (arrowhead) co-localizes with the golgi marker (Co-localization). FIG. 5F depicts HSPC cluster from FIG. 5E volume-rendered to highlight Sox17 and HPA-488 co-localization. FIG. 5G depicts DA of E10.0 Mlc2a-deficient mice lacking normal blood flow were evaluated for HSPC cluster protein expression patterns using IF. While mutants appeared to have disrupted CD31+ Sox17+ aortic endothelial architecture, emerging HSPC clusters appear phenotypically normal in the absence of shear stress. Runx1+ marks HSPC clusters. FIG. 5H depicts gating strategy for E10.5 wildtype embryonic cell isolation, FACS sorted using CD31, CD117, and CD45 conjugated antibodies prior to RT-PCR. FIG. 5I depicts gating strategy as in FIG. 5H using markers CD31, CD41, and CD45. FIG. 5J depicts E10.5 wildtype embryo cells sorted using CD41+ as a marker of HSPC clusters. Enriched populations of endothelial cells (CD31+ CD41− CD45−), HSPC cluster cells (CD31+ CD41+ CD45), maturing HSPC cluster/HSC cells (CD31+ CD41+ CD45+), and mature hematopoietic cells (CD31− CD45+) were evaluated from gene expression via Real Time RT-PCR (bar graphs). RT-PCR demonstrates increased Runx1 and Gata2 transcripts in population transitioning from endothelial cells to hematopoietic clusters cells, and decreased Sox17, Notch1, and Cdh5 transcripts. Differing letters represent significance between groups where a versus b is significant to a p value <0.05. Error bars indicated standard error of the mean.

FIGS. 6A-6O depict RUNX1 and SOX17 in murine hemogenic endothelium. FIG. 6A depicts RUNX1 and SOX17 immunofluorescence in a sagittal section of an E9.5 aorta. FIG. 6B depicts higher magnification of region denoted by the boxed area in (FIG. 6A) with single panels of VEC, RUNX1 and SOX17 reveals heterogeneous populations of RUNX1+ cells among SOX17+ cells. FIG. 6C depicts MFI of RUNX1 and SOX17 per cell pictured in (FIG. 6B). FIG. 6D. Ratios of RUNX1/SOX17 of individual cells depicted in (FIG. 6B). FIG. 6E depicts VEC, RUNX1 and SOX17 immunofluorescence in a transverse section of an E9.5 embryo (aorta and vitelline artery as indicated). Single channels in black and white. FIGS. 6F and 6G depict ratios of RUNX1/SOX17 per single cell in the dorsal aorta (FIG. 6F) and vitelline artery (FIG. 6G) depicted in (FIG. 6E) reveal ratios >1.0 in a few cells per anatomical site. FIG. 6 depicts transverse section of an E10.5 aorta with CD41, RUNX1, and SOX17. CD41+ marks intra-aortic clusters. SOX17 is noted in the endothelium and RUNX1 in intra-aortic clusters. FIG. 6J depicts a visualization of intra-aortic clusters at E10.5 with c-kit and RUNX1. SOX17 is noted in the endothelium, while RUNX1 is in intra-aortic clusters with membrane expression of c-kit. FIG. 6K depicts MFI levels of SOX17 in relation to CD41+ and CD41− cell populations (left) and ckit+ and c-kit− cell populations (right) in the E10.5 aorta. CD41+ cells, and separately c-kit+ cells, exhibit low levels of SOX17. FIG. 6L depicts MFI levels of RUNX1 in relation to CD41+ and CD41− cell populations (left) and ckit+ and c-kit− cell populations (right) in the E10.5 aorta. CD41+ cells, and separately c-kit+ cells, exhibit high levels of RUNX1. FIG. 6M depicts correlation plot of RUNX1 and SOX17 MFI's of 112 single cells corresponding to image analysis in (FIGS. 6I-6L). Correlation coefficient r of −0.78 indicates a strong negative correlation between RUNX1 and SOX17 MFI levels. p value and n as shown. FIG. 6N depicts a large intra-aortic cluster contains rounded cells with high levels of RUNX1, and non-nuclear localization of SOX17, while endothelial cells with flat appearance exhibit high nuclear levels of SOX17. FIG. 6O depicts RUNX1/SOX17 ratios of cells calculated from MFIs in cluster cells and endothelium reveal high >1.0 ratios in intra-aortic clusters and low ratios (<0.1) in endothelium.

FIGS. 7A-7F depict the temporal endothelial loss of Sox17. FIG. 7A depicts the recombination levels of Sox17^{f/f} explants as measured by tdTomato (Td+) detection by FACS. Cells from homozygous embryos that express detectable tdTomato are presumed to have recombined at least one R26R allele. Among the compartments analyzed, no significant differences in recombination were found between f/+ and f/f cells. Left most graph depicts total number of cells that were traced (Td+), middle graphs total % of cells within EC (CD31+) and HC (CD45+) compartments (traced and untraced), and rightmost graph is the percent of ECs that were traced (Td+). Error bars indicated SEM. Significance was determined by Student's t-test. FIG. 7B depicts scanning electron micro graphs of wildtype and in vivo Cre induced (tamoxifen induction at E9.5) Sox17^{f/f} dorsal aortic sections from E11 embryos. Arrowheads indicate endothelial-associated clusters with hematopoietic morphology. There are no appreciable differences in the endothelial layer or associated clusters after Sox17 ablation. FIG. 7C depicts gating strategy for populations evaluated in the calculation of the HE ratio. FIG. 7D depicts evaluation of proliferation and cell death after Sox17 loss of function. Top: BrdU incorporation after 2 hours of incubation in AGM explants of Sox17 floxed embryos after tamoxifen Cre induction demonstrates no significant differences in BrdU incorporation of ECs (Td+CD31+45−) or HCs (Td+CD45+CD31−). Bottom: Cell death analysis, as measured by AnnexinV+ staining, in the same context shows no significant differences in either EC or HC compartments. FIG. 7E depicts percentages of traced (Td+) maturing HSPC populations (CD31− CD117+ Ly6A+ 45+) are significantly increased in homozygous explants. FIG. 7F depicts E9.5 AGMs were explanted and induced, followed by FACS analysis for determination of the HE ratio 24 hours later. Sox17 homozygous mutant explants trend toward a higher HE ratio compared to heterozygotes.

FIGS. 8A-8F depict correlative microscopy of aortic endothelium. FIG. 8A depicts sagittal section of E10.5 embryonic aorta. RUNX1 identifies a large cell cluster (arrowhead), SOX17. FIG. 8B depicts scanning Electron Micrograph (SEM) of aorta in (FIG. 8A) reveals the overall topography of the aorta and intra-aortic clusters. FIG. 8C depicts higher magnification of intra-aortic clusters of boxed area in (FIG. 8B) marked by PECAM1, RUNX1 and SOX17. FIG. 8D depicts scanning EM of the same intra-aortic cluster in boxed region in (FIG. 8B) and immunofluorescence in (FIG. 8C) exhibits the heterogeneous membrane morphology of cells comprising the intra-aortic cluster. FIG. 8E depicts endothelium proximal to the large cluster (from FIGS. 8B, 8C and 8D) identified by white dashed lines. Each single cell is designated by a letter (A-O), with immunofluorescence of SOX17 in green and RUNX1 in magenta, and corresponding scanning EM with overlay. FIG. 8F depicts another region of the E10.5 aorta with a small group of cells of various cell morphology as depicted by RUNX1 and SOX17 immunostaining and corresponding scanning EM with overlay.

FIGS. 9A and 9B depict conservation of SOX17 regulatory sites in hematopoietic genes across species and cell type. FIG. 9A depicts SOX17 ChIP was performed in human cell lines (HUAECs) and putative binding sites of RUNX1 and GATA2 were evaluated. Error bars indicate SEM. FIG. 9B depicts EMSA validated SOX17 binding sites for Runx1 and Gata2 demonstrate evolutionary conservation. Sequences shown in FIG. 9B are RUNX1_A1 Mus musculus (SEQ ID NO: 192); RUNX1_A1 Homo sapiens (SEQ ID NO: 193); RUNX1_A1 Pan troglodytes (SEQ ID NO: 194); RUNX1_A1 Consensus (SEQ ID NO: 195); RUNX1_A2 Mus musculus (SEQ ID NO: 196); RUNX1_A2 Homo sapiens (SEQ ID NO: 197); RUNX1_A2 Pan troglodytes (SEQ ID NO: 198); and RUNX1_A2 Consensus (SEQ ID NO: 199).

FIG. 10A-10M depict single cell analysis of endothelial-to-hematopoietic transition. FIG. 10A. Region of aortic endothelium at E11.5 with RUNX1 and SOX17 immunofluorescence. Boxed area identifies a large intra-aortic cluster. RUNX1+ smaller clusters identified by arrowheads, and single cells by asterisks. FIG. 10B depicts RUNX1 and SOX17 MFI levels corresponding to cells in the cluster in boxed area in FIG. 10A. FIG. 10C depicts measured RUNX1/SOX17 ratios per cell in boxed area in (FIG. 10A). Arrows signify the ratio of two separate individual cells. FIG. 10D depicts SEM image of boxed area in (FIG. 10A) and corresponding MFIs in (FIG. 10B) and ratios in (FIG. 10C). The arrows identify cells with corresponding high ratios in (FIG. 10C). FIG. 10E depicts SEM image of four round cells attached to the endothelium with corresponding high RUNX1, low SOX17 MFI levels. Each cell is numbered signifying a high RUNX1/SOX17 ratio. FIG. 10F depicts SEM image of two cells with high RUNX1 and low-intermediate SOX17 MFI levels, resulting in a high ratio, as evidenced by cell numbers (#8 and #9). Corresponding MFIs are depicted with arrowheads. FIG. 10G depicts SEM image of two cells with moderate RUNX1 and low-intermediate SOX17 MFI levels, resulting in an intermediate ratio, as evidenced by cell numbers #13 and #17. Corresponding MFIs are depicted with arrowheads. Also note that while surface morphology suggest cell #13 and #14 may be one cell, there are two distinct nuclei by immunofluorescence analysis in which one nuclei has an intermediate ratio (number #13), the other has a low ratio #14. FIG. 10H depicts bar graph of RUNX1/SOX17 ratios of all cells depicted in FIGS. 10E, 10F, and 10G. FIG. 10I depicts correlation of RUNX1/SOX17 ratios to number of protrusions per μm2 surface, for cells depicted in FIGS. 10E, 10F, and 10G. Correlation coefficient r suggests a direct correlation of high ratios to more protrusions per cell surface area. FIG. 10J depicts percentage of cells with protrusions at E10.5 and E11.5 in RUNX1+ and SOX17+ subpopulations of aortic endothelium. Protrusions can be found primarily in RUNX1+ cells. FIG. 10K depicts left: An aortic cell with an intermediate SOX17+RUNX1+ ratio surrounded by cells with low ratios. Middle: A rounder cell with a high RUNX1/SOX17 ratio and surrounding cells with intermediate and low ratios. Right: Very rounded RUNX1/SOX17 high cell, and other cells with high, intermediate and low ratios. FIG. 10L depicts rounded cell with a high ratio, and ultra high-resolution image (boxed area) of protrusions on the surface the cell. FIG. 10M depicts schema of the endothelial to hematopoietic transition.

FIGS. 11A-11K depict Notch pathway targets and the impact of decreased Notch signaling on EHT. FIG. 11A depicts SOX17 ChIP was performed in human cell lines (HUAECs) and putative binding sites of NOTCH1, DLL4, and COUPTF-II were evaluated. FIG. 11B depicts EMSA evaluation of the murine Notch1 ChIP site A, which exhibited the highest enrichment, did not demonstrate in vitro binding, suggesting the likelihood of required co-binding partners for this particular ChP region. FIG. 11C depicts evolutionary conservation of EMDSA validated SOX17 binding sites for Notch1, D114, and CoupTFII. Sequences shown in FIG. 11C are DLL4_C3 Mus Musculus (SEQ ID NO: 200); DLL4_C3 Homo sapiens (SEQ ID NO: 201); DLL4_C3 Pan troglodytes (SEQ ID NO: 202); DLL4_C3 Consensus (SEQ ID NO: 203); NOTCH1_B Mus Musculus (SEQ ID NO: 204); NOTCH1_B Homo sapiens (SEQ ID NO: 205); NOTCH1_B Pan troglodytes (SEQ ID NO: 206); NOTCH1_B Consensus (SEQ ID NO: 207); COUPTFII_B Mus Musculus (SEQ ID NO: 208); COUPTFII_B Homo sapiens (SEQ ID NO: 209); COUPTFII_B Pan troglodytes (SEW ID NO: 210); and COUPTFII_B Consensus (SEQ ID NO: 211). FIG. 11D depicts Notch1^{f/f} explants do not demonstrate any differences in maturing HSPC populations (CD31−CD117+Ly6A+45+Td+) from Notch1^{f/+}. FIG. 11E depicts wild type E11 whole AGM explants were treated with DAPT, a γ-secretase inhibitor, for 24 hours at the indicated molar concentration. Control explants were treated with DMSO. A significant increase in the HE ratio is visible with 50-100 μM DAPT in comparison to control. Error bars indicate SEM. FIGS. 11F and 11G depict annexin V+ staining in the CD31+CD45− Td+ traced endothelial (EC) and the hematopoietic CD45+CD31−Td+ (HC) compartments from Notch1^{f/f} AGM explants demonstrate no appreciable differences in cell death when compared to Notch1^{f/+}. FIG. 11H depicts recombination levels of Notch1^{f/f} AGM explants as measured by tdTomato (Td+) detection by FACS. No significant differences in recombination were found between f/+ and f/f cells. Left most graph depicts total number of cells that were traced (Td+), middle graph total % of cells within EC (CD31+) and HC (CD45+) compartments (traced and untraced), and rightmost graph is the percent of ECs that were traced (Td+). Error bars indicate SEM. FIG. 11I depicts scanning electron microscopy of in vivo Cre induced Notch1^{f/f} dorsal aortic sections at E11 (tamoxifen induction at E9.5) demonstrate EC-associated projections (arrows). Hematopoietic clusters appeared to exhibit relative normal morphology (arrowhead). FIG. 11J depicts Runx1 binding site consensus sequence. FIG. 11K depicts sequence and evolutionary conservation of Runz1 ChIP-enriched site A within the Sox17 promoter. Sequences shown in FIG. 11K are SOX17_CHIP_SITE_A Homo sapiens (SEQ ID NO: 212); SOX17_CHIP_SITE_A Pan troglydes (SEQ ID NO: 213); SOX17_CHIP_SITE_A Bos taurus (SEQ ID NO: 214); and SOX17_CHIP_SITE_A Mus Musculus (SEQ ID NO: 215).

FIGS. 12A-12F depict hematopoietic cell clusters down-regulate arterial gene expression. FIGS. 12A-12E have single channels in black and white, scale bars as shown. E10.5 wildtype dorsal aorta (DA). FIG. 12A depicts hematopoietic cell clusters of the AGM at E10.5. The endothelial layer and attached hematopoietic cell clusters are CD31+ (grey). RUNX1 (grey) is notable in cells comprising the hematopoietic cluster (arrowhead). SOX17 (light grey) expression is localized to the endothelial layer (arrow). DAPI in dark grey. FIG. 12B depicts GATA2 (light grey) is notable in the hematopoietic cell cluster (arrowhead). CD31 (grey), and DAPI (dark grey). FIG. 12C depicts SOX17 (light grey) immunofluorescence is noted in the cell nuclei of the endothelial layer, as compared to the associated cell cluster. CD31 in grey, and DAPI in dark grey. FIG. 12D depicts Notch pathway activation (grey) as measured in the TP-1 Venus mouse line is notable in the endothelial layer (arrow) but less so in the associated hematopoietic cell cluster, CD31 in dark grey. DAPI in grey. FIG. 12E depicts CD144 (red) labels the endothelium and hematopoietic cluster cells (arrowhead), Sox17 in grey, and Runx1 in dark grey. FIG. 12F depicts embryos at E10.5 were sorted based on cell surface markers to isolate endothelial cells (CD31+CD117−CD45−), hematopoietic cluster cells (CD31+CD117+CD45−), maturing cluster cells and pre-HSCs (CD31+CD117+CD45+), and mature hematopoietic cells (CD31−CD45+). Bar graphs depict transcript expression (RT-PCR) in each subgroup for Runx1, Gata2, Sox17, Notch1, and Cdh5 (CD144). Differing letters represent significance between groups where a versus b, or b versus c, or a versus c, is significant to a p value <0.01 or less, n=3 litters, 24 embryos

FIGS. 13A-13J depict endothelial to hematopoietic conversion is increased after Sox17 loss. FIG. 13A depicts schema and bar graph of qRT-PCR analyses of sorted endothelial cells from E11 embryos after in vivo Sox17 ablation at E9.5. Error bars indicate standard error of the mean (n=3 litters, embryos pooled by genotype). LOF, loss of function. FIG. 13B depicts immunofluorescence of Sox17 heterozygous and homozygous embryos at E10.5 after in vivo Cre induction (tamoxifen induction at E9.5). Hematopoietic clusters are labeled by CD117 (grey), Cre traced endothelial and cluster cells in grey (Td+). SOX17 (light grey) is absent in homozygous mutant endothelium. DAPI in dark grey. DA, dorsal aorta. Scale bar denotes 10 μm. Single channels in black and white. FIG. 13C depicts a schematic of AGM explant analysis depicts in vitro Cre lineage tracing and calculation of hemogenic output (HE ratio); the ratio between percent labeled (Td+) hematopoietic cells (CD45+CD31−) to percent labeled (Td+) endothelial cells (CD31+CD45−). 4OHT, 4-hydroxytamoxifen. In FIGS. 13D-13F, each data point represents a separate embryo/AGM explant, littermates are depicted by the same data point color and shape. Bar indicates group mean. p values calculated on student's t-test between groups, significance also validated by two-way ANOVA, (supplementary table 1). FIG. 13D depicts the HE ratio of Sox17 homozygous (f/f) and heterozygous (f/+) mutant explants. f/+ n=45, f/f n=38, 15 litters. FIG. 13E depicts the percentage of traced Td+ hemogenic endothelial and cluster cells, designated as CD31+CD41+. f/+ n=37, f/f n=26, 9 litters. FIG. 13F depicts the percentage of traced (Td+) pre-HSCs (identified as CD31+CD117+Ly6A+CD45+, f/+ n=14, f/f n=27, 7 litters. FIG. 13G depicts a schema showing overexpression analyses in wildtype AGM explants at E11.0. FIG. 13H depicts immunofluorescence of E11.0 AGM explant after human adenoviral Sox17-GFP exposure. GFP in light grey, SOX17 in grey, and DAPI in dark grey. Scale bar as indicated. FIG. 13I depicts a cell sorting strategy for endothelial cells (CD31+) after exposure to AdhSox17-GFP (GFP), where GFP+ and GFP− populations were gated. FIG. 13J depicts a bar graph of qRT-PCR analyses of sorted E11.0 AGM CD31+ cells after AdhSox17-GFP exposure. Error bars indicate standard error of the mean. GFP− CD31+ population served as a control, set to one for comparisons of fold change, n=3 litters, embryos pooled, p values as indicated. In FIGS. 13A-13J, p values reflect student's t-test.

FIGS. 14A-14G depict SOX17 directly binds Runx1 and Gata2 for repression of hematopoietic fate. FIG. 14A depicts SOX17 chromatin immunoprecipitation (ChP) qRT-PCR of E11.0 sorted endothelial cells. Letters denote regions with SOX17 binding site consensus sequences upstream of Runx1 and Gata2 promoters, and Lef1 as a positive control. Error bars indicate standard error of the mean (SEM). IgG control set to one for comparisons of fold change, n=3 litters, embryos pooled, p values as indicated. FIG. 14B depicts electrophoretic mobility shift assay (EMSA) of putative SOX17 binding sites within ChIP sequences designated by letters in FIG. 14A. Each lane represents biotin-labeled duplexed oligonucleotides containing the Lef1 promoter SOX17 binding site (Lef1_Biot). Addition of rSox17-Flag produces a specific shift, indicating protein-DNA complex (lane 2), which is competed away by unlabeled Lef1 (Lef1_s1), while mutant probe does not compete (Lef1_Δ_s1). Similar designations are used for putative binding sites (and mutants) in Runx1 and Gata2 sequences. Asterisks denote competitive binding. FIG. 14C depicts bar graph depicts luciferase activity of Gata2 and Runx1 promoters after Sox17 siRNA versus control (scramble). p values as indicated. Error bars represent SEM. FIG. 14D depicts immunofluorescence of hematopoietic cell clusters (arrowhead) in E10.5 dorsal aorta (DA) of Sox17f/f and Sox17f/+ mutants (after tamoxifen mediated Cre induction at E9.5). Traced cells labeled in dark grey (Td+), SOX17 in light grey, and RUNX1 in grey. DAPI in darkest grey. Scale bar 10 μm. Single channels in black and white. FIG. 14E depicts GATA2 (light grey) and SOX17 (grey) immunofluorescence of hematopoietic cell clusters in E10.5 dorsal aorta (DA) of Sox17f/+ and Sox17f/f (arrowhead) mutants (iCre induction at E9.5). Traced cells in dark grey (Td+), SOX17 in grey, and RUNX1 in light grey. DAPI in darkest grey. Scale bar denotes 10 μm. Single channels in black and white. FIG. 14F depicts SOX17 siRNA knockdown in HUAECs and qRT-PCR analysis. Control represents treatment with lipofectin alone, SOX17 siRNA compared to scrambled (n=3 experiments, error bars indicate SEM). p values as indicated. FIG. 14G depicts adenoviral-mediated overexpression of hSOX17 in HUAECs and qRT-PCR analyses, p values calculated with respect to Adeno-GFP infected cells, control represents uninfected cells (n=3 experiments, error bars indicate SEM).

FIGS. 15A-15H depict the role of the Notch pathway in endothelial to hematopoietic fate decisions. FIG. 15A depicts SOX17 (ChIP) qRT-PCR of E11.0 sorted endothelial cells. Letters denote regions upstream of Notch1, D114, and CoupTFII promoters, and Lef1 as a positive control. Error bars indicate standard error of the mean. IgG control set to one for comparisons of fold change, n=3 litters, embryos pooled, p values as indicated. FIG. 15B depicts EMSA of putative SOX17 binding sites within ChP sequences (designated by letters in FIG. 15A). Each lane represents biotin-labeled duplexed oligonucleotides spanning the Lef1 promoter SOX17 binding site (Lef1_Biot). Addition of rSox17-Flag produces a specific shift, indicating protein-DNA complex (lane 2), which is competed away by unlabeled Lef1 (Lef1_s1), while mutant probe does not compete (Lef1_Δ_s1). Similar designations are used for putative binding sites (and mutants) in Notch1, D114, and CoupTFII sequences. Asterisks denote competition. FIG. 15C depicts Schematic of AGM explant analysis depicts in vitro Cre lineage tracing and calculation of hemogenic output (HE ratio); the ratio between percent labeled (Td+) hematopoietic cells (CD45+CD31−) to percent labeled (Td+) endothelial cells (CD31+CD45−). In FIGS. 15D-15G, each data point represents a separate embryo/AGM explant, littermates are depicted by the same data point color and shape. Bar indicates group mean. p-values calculated on student's t-test between groups, significance also validated by two-way ANOVA, supplementary table 2. FIG. 15D depicts the HE ratio of Notch1 homozygous (f/f) and heterozygous (f/+) mutant explants. f/+ n=18, f/f n=21, 6 litters. LOF, loss of function. FIG. 15E depicts a percentage of traced Td+ hemogenic endothelial and cluster cells, designated as CD31+CD41+. f/+ n=12, f/f n=13, 4 litters. FIG. 15F depicts a percentage of traced (Td+) pre-HSCs (identified as CD31+CD117+Ly6A+CD45+) f/+ n=10, f/f n=6, 3 litters. FIG. 15G depicts BrdU+ cells measured after 2 hour incubation with BrdU in traced ECs (left) and traced HCs (right) demonstrates a significant increase in HC proliferation, f/+ n=6, f/f n=10, 3 litters. FIG. 15H depicts Schema and bar graph of qRT-PCR analyses of sorted endothelial cells from E11 embryos after in vivo Notch1 ablation at E9.5. Error bars indicate standard error of the mean (n=3 litters, embryos pooled by genotype). LOF, loss of function.

FIGS. 16A-16F depict parsing endothelial and hematopoietic fates during EHT. FIG. 16A depicts a schematic depicting Sox17 or Notch1 loss of function (LOF) and strategy for evaluating Notch overexpression (mNICD-GFP) in Sox17 mutants. NICD, Notch1 intracellular domain. FIG. 16B depicts HE ratios of E11 AGM explants in Sox17 mutants with and without Notch overexpression (+N1). Center-lines represent median values, box represents 25th-75th percentiles, bars represent minimum and maximum values. f/+(−N1) n=7, f/+(+N1) n=3, f/f (−N1) n=5, f/f (+N1) n=8, 3 litters. p values calculated on student's t-test between groups, significance also validated by two-way ANOVA, (Supplementary Table 1). FIG. 16C depicts immunofluorescence of a representative hematopoietic cluster in a E10.5 Sox17f/f(+N1) AGM after in vivo induction of Cre and NICD at E9.5. SOX17 in grey, traced ECs (Td+) in dark grey and RUNX1+ in light grey. DAPI in darkest grey. Scale bar denotes 10 μm. FIG. 16D depicts RUNX1 chromatin immunoprecipitation (ChP) PCR of E11.0 sorted endothelial cells. Letters denote evaluated regions containing RUNX1 binding site consensus sequences upstream of the Sox17 promoter. Error bars indicate standard error of the mean. IgG control set to one for comparisons of fold change, n=3 litters, embryos pooled, p values as indicated. FIG. 16E depicts adenoviral-mediated overexpression of hRUNX1 in HUAECs and qRT-PCR analyses, p values calculated with respect to Adeno-GFP infected cells, control represents uninfected cells (n=3 experiments, error bars indicate SEM). FIG. 16F depicts a schematic depicting the cell fate switch from endothelial to hematopoietic fate, and the governing regulatory pathways of EHT. Sox17 inhibition of Runx1 and Gata2 maintains endothelial fate. Loss of Sox17 inhibition in the context of decreased Notch activity promotes hematopoietic fate conversion.

FIG. 17A depicts the strategy for directed reprogramming of endothelium to hemogenic endothelium using Sox17 and Runx1 episomals in addition to DAPT.

FIG. 17B depicts hematopoietic like cells emerging from mature endothelial populations (right). HUAEC=Human umbilical arterial cell line; HUVEC=venous endothelial cell line. Runx1 episomal plasmid has an E2Crimson tracer which can track cells still retaining the vector.

FIG. 18A depicts cells emerging from culture that are round in morphology and express hematopoietic markers Runx1 and CD45 after losing Runx1-Crimson labeled episomal and Sox17, although early budding of hematopoietic cells appear to retain Runx1 episomals (CD31/CD45 right panel) initially.

FIG. 18B depicts FACS analysis of cultures after reprogramming. The FACS analysis demonstrates new populations of CD45+ and CD34+CD45+ hematopoietic cells.

FIG. 18C depicts Giemsa stain of sorted hematopoietic cell subsets that emerged from the endothelium.

FIG. 19A depicts episomals engineered to deliver direct reprogramming factors Sox17 and Runx1.

FIG. 19B depicts varying Sox17 protein levels after the first step of the protocol. Cell subsets exhibited Sox17 protein levels of high, mid and low. When stained for endogenous Sox17 protein after introduction of Sox17 episomal (right) versus endogenous expression in passage 5 (p5) human umbilical venous endothelial cells (left), there is an increase in endogenous Sox17 levels but in a very heterogeneous pattern. Thus future iterations of the protocol will be to sort Sox17high, Sox17mid and Sox17low cells to test which may be best for reprogramming.

FIG. 20 depicts FACS analysis of cultures after reprogramming. The FACS analysis demonstrates that CD45+ CD34− hematopoietic cells are of smaller size (FSC) (gated in left panel), which may be indicative of their hematopoietic fate/potential.

FIG. 21 depicts FACS analysis of hematopoietic output. The FACS analysis suggests a replacement of CD34+ only endothelial cells (lower left plot) to CD45+ only hematopoietic cells (lower right plot). Unstained and IgG controls are on the top left and right, respectively.

FIG. 22 depicts Giemsa stains of sorted hematopoietic cell subsets (from different replicates) and cells that are maintained in the culture dish. These stains suggest that there are various hematopoietic morphology types that are not captured by sorting on current markers.

FIG. 23 depicts gene expression in hematopoietic stem/progenitor cells (HSPCs) during cell maturation. Tgfβ1 and cyclin genes increase as hematopoietic stem and progenitor cells (HSPCs) mature in the mouse, and become transplantable in adults. Preliminary mouse studies suggest that adding Tgfβ1 to newly formed murine HSPCs may accelerate their maturation. Hence we will incorporate adding Tgfβ1 to the final stages of our human reprogramming cultures. Tgfβ1 addition to protocol may enhance “transplantability” of cells generated from reprogramming.

FIG. 24A depicts hematopoietic cell clusters. The endothelial layer and attached hematopoietic cell clusters are CD31+, while CD117+ identifies cells in the HSPC clusters (arrowhead).

FIG. 24B depicts CD41 marker expression in endothelial associated hemtopoietic cells. CD41 marker expression is notable in endothelial associated hematopoietic cell clusters (arrowhead) identified by CD31+staining.

FIG. 24C depicts SOX17 localization in hematopoietic cells (HCs). CD45+ (grey) is noted in mature HCs and few cluster associated cells (arrowheads). SOX17 is localized to the underlying endothelium identified by CD31+staining.

FIG. 24D depicts Sox17 localization in endothelial cells. Sox17 strongly marks nuclei of underlying CD31+endothelial cells (arrow) compared to the largely dim, punctate staining apparent in HSPC clusters (arrowhead).

FIG. 24E depicts Sox 17 localization in HSPC clusters. E10.5 DA was costained with CD31 (red), Sox17 (purple) and fluorescent conjugated Lectin HPA (HPA-488), a protein with a strong affinity for the golgi apparatus membranes. Punctate Sox17 staining in the HSPC cluster (arrowhead) co-localizes with the golgi marker (Co-localization).

FIG. 24F depicts Sox17 and HPA-488 co-localization in HSPC clusters. The HSPC cluster from FIG. 24E was volume-rendered to highlight Sox17 and HPA-488 co-localization. In FIG. 24A-24F, DA=dorsal aorta, DAPI=nuclear stain, scale bar as stated in μm.

FIG. 24G depicts dorsal aorta (DA) of Mlc2a-deficient mice lacking normal blood flow. DA of E10.0 Mlc2a-deficient mice lacking normal blood flow were evaluated for HSPC cluster protein expression patterns using IF. While mutants appeared to have disrupted CD31+Sox17+ aortic endothelial architecture, emerging HSPC clusters appear phenotypically normal in the absence of shear stress. Runx1+marks HSPC clusters

FIG. 24H depicts the gating strategy for E10.5 wildtype embryonic cell isolation, FACS sorted using CD31, CD117, and CD45 conjugated antibodies prior to RT PCR.

FIG. 24I depicts the gating strategy as in FIG. 24H using markers CD31, CD41, and CD45.

FIG. 24J depicts E 10.5 wildtype embryo cells sorted using CD41+as a marker of HSPC clusters. Enriched populations of endothelial cells (CD31+CD4rCD45−), HSPC cluster cells (CD31+CD41+CD45), maturing HSPC cluster/HSC cells (CD31+CD41+CD45+), and mature hematopoietic cells (CD31′CD45+) were evaluated for gene expression via Real Time RTPCR (bar graphs). Real Time RT-PCR demonstrates increased Runx1 and Gata2 transcripts in populations transitioning from endothelial cells to hematopoietic cluster cells, and decreased Sox17, Notch 1, and Cdh5 transcripts. Differing letters represent significance between groups where a versus b is significant to a p value <0.05. Error bars indicate standard error of the mean (SEM). N=3 litters

FIG. 25A depicts recombination levels of Sox1 ill explants as measured by tdTomato (Td+) detection by FACS. Cells from homozygous embryos that express detectable tdTomato are presumed to have recombined at least one R26R allele. Among the compartments analyzed, no significant differences in recombination were found between f/+ and f/f cells. Left most graph depicts total number of cells that were traced (Td+), middle graphs total % of cells within EC (CD31+) and HC (CD45+) compartments (traced and untraced), and rightmost graph is the percent of ECs that were traced (Td+). Error bars indicate SEM. Significance was determined by Student's t-test. ns=not significant.

FIG. 25B depicts scanning electron micrographs of wildtype and in vivo Cre induced (tamoxifen induction at E9.5) Sox17^{f/f} dorsal aortic sections from E11 embryos. Arrowheads indicate endothelial associated clusters with hematopoietic morphology. There are no appreciable differences in the endothelial layer or associated clusters after Sox17 ablation.

FIG. 25C depicts the gating strategy for populations evaluated in the calculation of the HE ratio.

FIG. 25D depicts evaluation of proliferation and cell death after Sox17 loss of function. Top: BrdU incorporation after 2 hours of incubation in AGM explants of Sox17 floxed embryos after tamoxifen Cre induction demonstrates no significant differences in BrdU incorporation of ECs (Td⁺CD31⁺45⁻) or HCs (Td⁺CD45⁺CD31⁻). (f/+ n=14, f/f n=15). Bottom: Cell death analysis, as measured by AnnexinV+ staining, in the same context shows no significant differences in either EC or HC compartments (f/+ n=18, f/f n=13).

FIG. 25E depicts percentages of traced (Td+) maturing HSPC populations (CD31⁻ CD117⁺Ly6A⁺45⁺) are significantly increased in homozygous explants.

FIG. 25F depicts E9.5 AGMs explanted and induced as in FIG. 25B, followed by FACS analysis for determination of the HE ratio 24 h later (f/+ n=12, f/f n=10). Sox17 homozygous mutant explants trend toward a higher HE ratio compared to heterozygotes. For FIG. 25D-25F, each data point represents a separate embryo/AGM explant, littermates are depicted by the same data point color and shape. Bar indicates group mean. P-values calculated on student's t-test between groups. ns=not significant.

FIG. 26A depicts SOX17 ChiP performed in human cell lines (HUAECs) and evaluation of putative binding sites of RUNX1 and GATA2. Error bars indicate SEM. Inset left: SOX17 binding site consensus sequence.

FIG. 26B depicts validated SOX17 binding sites for Runx1 and Gata2 demonstrating evolutionary conservation. Sequences shown in FIG. 26B are RUNX1_A1 Mus musculus (SEQ ID NO: 216); RUNX1_A1 Homo sapiens (SEQ ID NO: 217); RUNX1_A1 Pan troglodytes (SEQ ID NO: 218); RUNX1_A1 Consensus (SEQ ID NO: 219); RUNX1_A2 Mus musculus (SEQ ID NO: 220); RUNX1_A2 Homo sapiens (SEQ ID NO: 221); RUNX1_A2 Pan troglodytes (SEQ ID NO: 222); RUNX1_A2 Consensus (SEQ ID NO: 223); GATA2_B1 Mus musculus (SEQ ID NO: 224); GATA2_B1 Homo sapiens (SEQ ID NO: 225); GATA2_B1 Pan troglodytes (SEQ ID NO: 226); GATA2_B1 Consensus (SEQ ID NO: 227); GATA2_B2 Mus musculus (SEQ ID NO: 228); GATA2_B2 Homo sapiens (SEQ ID NO: 229); GATA2_B2 Pan troglodytes (SEQ ID NO: 230); and GATA2_B2 Consensus (SEQ ID NO: 231).

FIG. 27A depicts SOX17 ChiP performed in human cell lines (HUAECs). Putative binding sites of NOTCH1, DLL4, (n=1) and COUPTF-II (n=3) were evaluated.

FIG. 27B depicts EMSA evaluation of the murine Notch1 ChP site A (FIG. 27A), which exhibited the highest enrichment, did not demonstrate in vitro binding, suggesting the likelihood of required co-binding partners for this particular ChiP region.

FIG. 27C depicts evolutionary conservation of EMSA validated SOX17 binding sites for Notch1, DLL4 and CoupTFII. Sequences shown in FIG. 27C are DII4_C3 Mus Musculus (SEQ ID NO: 232); DII4_C3 Homo sapiens (SEQ ID NO: 233); DII4_C3 Pan troglodytes (SEQ ID NO: 234); DII4_C3 Consensus (SEQ ID NO: 235); NOTCH1_B Mus Musculus (SEQ ID NO: 236); NOTCH1_B Homo sapiens (SEQ ID NO: 237); NOTCH1_B Pan troglodytes (SEQ ID NO: 238; NOTCH1_B Consensus (SEQ ID NO: 239); COUPTFII_B Mus Musculus (SEQ ID NO: 240); COUTPFII_B Homo sapiens (SEQ ID NO: 241); COUPTFII_B Pan troglodytes (SEQ ID NO: 242); and COUPTFII_B Consensus (SEQ ID NO: 243).

FIG. 27D depicts Notch1^{f/f} explants in maturing HSPC populations The explants do not demonstrate any differences in maturing HSPC populations CD31⁻CD117⁺Ly6A⁺45⁺Td⁺) from Notch1^{f/+}.

FIG. 27E depicts wild type E11 whole AGM explants treated with DAPT, a y-secretase inhibitor, for 24 hours at the indicated molar concentration. Control explants were treated with DMSO (vehicle). A significant increase in the HE ratio is visible with 50-100 μM DAPT in comparison to control. Error bars indicate SEM. p-values based on one-way ANOVA. Control n=7, 25 μM n=4, 50 μM n=9, 100 μM n=8, 200 μM n=3.

FIG. 27F and FIG. 27G depict Annexin V+ staining in the CD31⁺CD45⁻Td⁺ traced endothelial (EC) and the hematopoietic CD45⁺CD31⁻Td⁺ (HC) compartments from Notch1f/f AGM explants. The staining demonstrates no appreciable differences in cell death when compared to Notch1f/⁺. f/+ n=7, f/f n=6. For FIG. 27D-27G, each data point represents a separate embryo/AGM explant, littermates are depicted by the same data point color and shape.

Bar indicates group mean. P-values calculated on student's t-test between groups.

FIG. 27H depicts recombination levels of Notch1^{f/f} AGM explants as measured by tdTomato (Td⁺) detection by FACS. No significant differences in recombination were found between f/+ and f/f cells. Left most graph depicts total number of cells that were traced (Td⁺), middle graph total % of cells within EC (CD31⁺) and HC (CD45⁺) compartments (traced and untraced), and rightmost graph is the percent of ECs that were traced (Td⁺). Error bars indicate SEM. Significance was determined by student's t-test. n.s.=not significant.

FIG. 27I depicts scanning electron microscopy of in vivo Cre induced Notch1^{f/f} dorsal aortic sections at E11 (tamoxifen induction at E9.5) demonstrating EC-associated projections (arrows). Hematopoietic clusters appeared to exhibit relatively normal morphology (arrowhead).

FIG. 27J depicts a Runx1 binding site consensus sequence.

FIG. 27K depicts sequence and evolutionary conservation of Runx1 ChIP-enriched site A within the Sox17 promoter. Lighter type signifies the putative Runx1 binding site. Sequences shown in FIG. 27K are SOX17_CHIP_SITE_A Homo sapiens (SEQ ID NO: 244); SOX17_CHIP_SITE_A Pan troglydes (SEQ ID NO: 245); SOX17_CHIP_SITE_A Bos taurus (SEQ ID NO: 246); and SOX17_CHIP_SITE_A Mus Musculus (SEQ ID NO: 247).

FIG. 28A depicts traced hematopoietic populations (Td⁺CD117⁺CD45⁺) from in vivo induced Sox17^{f/f} (tamoxifen induction at E9.5) were FACS-sorted and plated in methylcellulose CFU assays. Colony forming units (CFUs) were scored after 7 days as granulocyte, erythrocyte, monocyte, and megakaryocyte (CFU-GEMM), granulocyte and monocyte (CFU-GM), and burst-forming units with erythrocytes (BFU-E). There exists a significant decrease in CFU capacity of Sox17 homozygous animals. Error bars represent SEM. P-value reflects a student's t-test on total number of colonies. (n=20 embryos from 3 litters).

FIG. 28B depicts CFUs of colonies that were picked and genotyped. All genotyped CFUs are from Cre positive flf embryos and represented two excised alleles (Δ/Δ) one excised allele (f/Δ), or no excision of floxed exons (f/f). Percentage of CFUs by genotype. Double excision occurs at similar percentages as to known inducible endothelial Cre recombination measured by Td⁺ (n=75 CFUs).

FIG. 28C depicts the same populations co-cultured on OP9-DL1 stromal cells in a lymphoid differentiation assay. After 5 week OP9-DL1 co-culture, cells were evaluated for T lymphoid subtypes (CD45⁺TCRb⁺ and CD8 and CD4 single and double positivity). Sox17 homozygous animals exhibited decreased percentages of all subtypes, but the differences were not significant. Error bars indicate SEM. (n=19 embryos).

FIG. 28D depicts CFU assays as described above for Notch1^{f/f} embryos. No significant decrease in Notch1^{f/f} CFU ability is observed (n=25 embryos from 3 litters).

FIG. 28E depicts the number of fully excised colonies. There is a relatively low number of fully excised colonies (Δ/Δ), suggesting a loss of this cell population (n=233 CFUs).

FIG. 28F depicts OP9-DL1 co-culture of cells from Notch1^{f/f} embryos demonstrating significantly decreased lymphoid differentiation capacity (n=13 embryos).

FIG. 28G depicts excision of Sox17 alleles detected by PCR after isolated colony selection. CFUs were found to be non-excised (f/f), excised at one allele (F/Δ), or excised at both alleles (Δ/Δ).

FIG. 28H depicts excision of Notch 1 alleles detected by PCR after isolated CFU colony selection.

### DETAILED DESCRIPTION OF ILLUSTRATIVE EMBODIMENTS

The terminology used herein is for the purpose of describing particular embodiments only and is not intended to be limiting. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the disclosure described herein are capable of operation in other sequences than described or illustrated herein.

The following terms or definitions are provided solely to aid in the understanding of the disclosure. Unless specifically defined herein, all terms used herein have the same meaning as they would to one skilled in the art of the present disclosure. Practitioners are particularly directed to Sambrook et al., Molecular Cloning: A Laboratory Manual, 2nd ed., Cold Spring Harbor Press, Plainsview, N.Y. (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

As used in the specification and the appended claims, the singular forms “a,” “an” and “the” include plural referents unless the context clearly dictates otherwise.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods. For recitation of numeric ranges herein, each intervening number therebetween with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6, 9, and 7.0 are explicitly contemplated.

“Coding sequence” or “encoding nucleic acid” as used herein may mean refers to the nucleic acid (RNA, DNA, or RNA/DNA hybrid molecule) that comprises a nucleotide sequence which encodes a protein. The coding sequence may further include initiation and termination signals operably linked to regulatory elements including a promoter and polyadenylation signal capable of directing expression in the cells of an individual or mammal to whom the nucleic acid is administered.

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

As used herein, the term “functional fragment” means any portion of a polypeptide that is of a sufficient length to retain at least partial biological function that is similar to or substantially similar to the wild-type polypeptide upon which the fragment is based. In some embodiments, a functional fragment of a polypeptide is a polypeptide that comprises or possesses 80, 85, 90, 95, 96, 97, 98, or 99% sequence identity to any polypeptides disclosed in Table 1 and has sufficient length to retain at least partial binding affinity to one or a plurality of ligands that bind to the polypeptides in Table 1. In some embodiments, a functional fragment of a nucleic acid is a nucleic acid that comprises or possesses 70, 75, 80, 85, 90, 95, 96, 97, 98, or 99% sequence identity to any nucleic acid to which it is being compared and has sufficient length to retain at least partial function related to the nucleic acid to which it is being compared. In some embodiments, the fragment is a fragment of any polypeptide disclosed in Table 1 and has a length of at least about 10, about 20, about 30, about 40, about 50, about 60, about 70, about 80, about 90, or about 100 contiguous amino acids. In some embodiments, the fragment is a fragment of any polypeptide disclosed in Table 1 and has a length of at least about 50 amino acids. In some embodiments, the fragment is a fragment of any polypeptide disclosed in Table 1 and has a length of at least about 100 amino acids. In some embodiments, the fragment is a fragment of any polypeptide disclosed in Table 1 and has a length of at least about 150 amino acids. In some embodiments, the fragment is a fragment of any polypeptide disclosed in Table 1 and has a length of at least about 200 amino acids. In some embodiments, the fragment is a fragment of any polypeptide disclosed in Table 1 and has a length of at least about 250 amino acids. In some embodiments, the fragment is a fragment of any polypeptide disclosed in Table 1 and has a length of at least about 300 amino acids. In some embodiments, the fragment is a fragment of any polypeptide disclosed in Table 1 and has a length of at least about 350 amino acids. In some embodiments, the fragment is a fragment of any polypeptide disclosed in Table 1 and has a length of at least about 400 amino acids. In some embodiments, the fragment is a fragment of any polypeptide disclosed in Table 1 and has a length of at least about 450 amino acids. In some embodiments, the fragment is a fragment of any polypeptide disclosed in Table 1 and has a length of at least about 500 amino acids. In some embodiments, the fragment is a fragment of any polypeptide disclosed in Table 1 and has a length of at least about 550 amino acids. In some embodiments, the fragment is a fragment of any polypeptide disclosed in Table 1 and has a length of at least about 600 amino acids. In some embodiments, the fragment is a fragment of any polypeptide disclosed in Table 1 and has a length of at least about 650 amino acids. In some embodiments, the fragment is a fragment of any polypeptide disclosed in Table 1 and has a length of at least about 700 amino acids. In some embodiments, the fragment is a fragment of any polypeptide disclosed in Table 1 and has a length of at least about 750 amino acids. In some embodiments, the fragment is a fragment of any polypeptide disclosed in Table 1 and has a length of at least about 800 amino acids. In some embodiments, the fragment is a fragment of any polypeptide disclosed in Table 1 and has a length of at least about 850 amino acids. In some embodiments, the fragment is a fragment of any polypeptide disclosed in Table 1 and has a length of at least about 900 amino acids. In some embodiments, the fragment is a fragment of any polypeptide disclosed in Table 1 and has a length of at least about 950 amino acids. In some embodiments, the fragment is a fragment of any polypeptide disclosed in Table 1 and has a length of at least about 1000 amino acids. In some embodiments, the fragment is a fragment of any polypeptide disclosed in Table 1 and has a length of at least about 1050 amino acids. In some embodiments, the fragment is a fragment of any polypeptide disclosed in Table 1 and has a length of at least about 1250 amino acids. In some embodiments, the fragment is a fragment of any polypeptide disclosed in Table 1 and has a length of at least about 1500 amino acids. In some embodiments, the fragment is a fragment of any polypeptide disclosed in Table 1 and has a length of at least about 1750 amino acids. In some embodiments, the fragment is a fragment of any polypeptide disclosed in Table 1 and has a length of at least about 2000 amino acids. In some embodiments, the fragment is a fragment of any polypeptide disclosed in Table 1 and has a length of at least about 2250 amino acids. In some embodiments, the fragment is a fragment of any polypeptide disclosed in Table 1 and has a length of at least about 2500 amino acids. In some embodiments, the fragment is a fragment of any polypeptide disclosed in Table 1 and has a length of at least about 2750 amino acids. In some embodiments, the fragment is a fragment of any polypeptide disclosed in Table 1 and has a length of at least about 3000 amino acids.

In some embodiments, the fragment is a fragment of any polypeptide disclosed in Table 1 and has a length of no more than about 10, about 20, about 30, about 40, about 50, about 60, about 70, about 80, about 90, or about 100 contiguous amino acids. In some embodiments, the fragment is a fragment of any polypeptide disclosed in Table 1 and has a length of no more than about 50 amino acids. In some embodiments, the fragment is a fragment of any polypeptide disclosed in Table 1 and has a length of no more than about 100 amino acids. In some embodiments, the fragment is a fragment of any polypeptide disclosed in Table 1 and has a length of no more than about 150 amino acids. In some embodiments, the fragment is a fragment of any polypeptide disclosed in Table 1 and has a length of no more than about 200 amino acids. In some embodiments, the fragment is a fragment of any polypeptide disclosed in Table 1 and has a length of no more than about 250 amino acids. In some embodiments, the fragment is a fragment of any polypeptide disclosed in Table 1 and has a length of no more than about 300 amino acids. In some embodiments, the fragment is a fragment of any polypeptide disclosed in Table 1 and has a length of no more than about 350 amino acids. In some embodiments, the fragment is a fragment of any polypeptide disclosed in Table 1 and has a length of no more than about 400 amino acids. In some embodiments, the fragment is a fragment of any polypeptide disclosed in Table 1 and has a length of no more than about 450 amino acids. In some embodiments, the fragment is a fragment of any polypeptide disclosed in Table 1 and has a length of no more than about 500 amino acids. In some embodiments, the fragment is a fragment of any polypeptide disclosed in Table 1 and has a length of no more than about 550 amino acids. In some embodiments, the fragment is a fragment of any polypeptide disclosed in Table 1 and has a length of no more than about 600 amino acids. In some embodiments, the fragment is a fragment of any polypeptide disclosed in Table 1 and has a length of no more than about 650 amino acids. In some embodiments, the fragment is a fragment of any polypeptide disclosed in Table 1 and has a length of no more than about 700 amino acids. In some embodiments, the fragment is a fragment of any polypeptide disclosed in Table 1 and has a length of no more than about 750 amino acids. In some embodiments, the fragment is a fragment of any polypeptide disclosed in Table 1 and has a length of no more than about 800 amino acids. In some embodiments, the fragment is a fragment of any polypeptide disclosed in Table 1 and has a length of no more than about 850 amino acids. In some embodiments, the fragment is a fragment of any polypeptide disclosed in Table 1 and has a length of no more than about 900 amino acids. In some embodiments, the fragment is a fragment of any polypeptide disclosed in Table 1 and has a length of no more than about 950 amino acids. In some embodiments, the fragment is a fragment of any polypeptide disclosed in Table 1 and has a length of no more than about 1000 amino acids. In some embodiments, the fragment is a fragment of any polypeptide disclosed in Table 1 and has a length of no more than about 1050 amino acids. In some embodiments, the fragment is a fragment of any polypeptide disclosed in Table 1 and has a length of no more than about 1250 amino acids. In some embodiments, the fragment is a fragment of any polypeptide disclosed in Table 1 and has a length of no more than about 1500 amino acids. In some embodiments, the fragment is a fragment of any polypeptide disclosed in Table 1 and has a length of no more than about 1750 amino acids. In some embodiments, the fragment is a fragment of any polypeptide disclosed in Table 1 and has a length of no more than about 2000 amino acids. In some embodiments, the fragment is a fragment of any polypeptide disclosed in Table 1 and has a length of no more than about 2250 amino acids. In some embodiments, the fragment is a fragment of any polypeptide disclosed in Table 1 and has a length of no more than about 2500 amino acids. In some embodiments, the fragment is a fragment of any polypeptide disclosed in Table 1 and has a length of no more than about 2750 amino acids. In some embodiments, the fragment is a fragment of any polypeptide disclosed in Table 1 and has a length of no more than about 3000 amino acids.

In some embodiments, the fragment is a fragment of any polypeptide disclosed in Table 1 and has a length from about 10 to about 3000 amino acids. In some embodiments, the fragment is a fragment of any polypeptide disclosed in Table 1 and has a length from about 10 to about 3000 amino acids. In some embodiments, the fragment is a fragment of any polypeptide disclosed in Table 1 and has a length from about 10 to about 2750 amino acids. In some embodiments, the fragment is a fragment of any polypeptide disclosed in Table 1 and has a length from about 10 to about 2500 amino acids. In some embodiments, the fragment is a fragment of any polypeptide disclosed in Table 1 and has a length from about 10 to about 2250 amino acids. In some embodiments, the fragment is a fragment of any polypeptide disclosed in Table 1 and has a length from about 10 to about 2000 amino acids. In some embodiments, the fragment is a fragment of any polypeptide disclosed in Table 1 and has a length from about 10 to about 1750 amino acids. In some embodiments, the fragment is a fragment of any polypeptide disclosed in Table 1 and has a length from about 10 to about 1500 amino acids. In some embodiments, the fragment is a fragment of any polypeptide disclosed in Table 1 and has a length from about 10 to about 1250 amino acids. In some embodiments, the fragment is a fragment of any polypeptide disclosed in Table 1 and has a length from about 10 to about 1000 amino acids. In some embodiments, the fragment is a fragment of any polypeptide disclosed in Table 1 and has a length from about 10 to about 950 amino acids. In some embodiments, the fragment is a fragment of any polypeptide disclosed in Table 1 and has a length from about 10 to about 850 amino acids. In some embodiments, the fragment is a fragment of any polypeptide disclosed in Table 1 and has a length from about 10 to about 800 amino acids. In some embodiments, the fragment is a fragment of any polypeptide disclosed in Table 1 and has a length from about 10 to about 750 amino acids. In some embodiments, the fragment is a fragment of any polypeptide disclosed in Table 1 and has a length from about 10 to about 700 amino acids. In some embodiments, the fragment is a fragment of any polypeptide disclosed in Table 1 and has a length from about 10 to about 650 amino acids. In some embodiments, the fragment is a fragment of any polypeptide disclosed in Table 1 and has a length from about 10 to about 600 amino acids. In some embodiments, the fragment is a fragment of any polypeptide disclosed in Table 1 and has a length from about 10 to about 550 amino acids. In some embodiments, the fragment is a fragment of any polypeptide disclosed in Table 1 and has a length from about 10 to about 500 amino acids. In some embodiments, the fragment is a fragment of any polypeptide disclosed in Table 1 and has a length from about 10 to about 450 amino acids. In some embodiments, the fragment is a fragment of any polypeptide disclosed in Table 1 and has a length from about 10 to about 400 amino acids. In some embodiments, the fragment is a fragment of any polypeptide disclosed in Table 1 and has a length from about 10 to about 350 amino acids. In some embodiments, the fragment is a fragment of any polypeptide disclosed in Table 1 and has a length from about 10 to about 300 amino acids. In some embodiments, the fragment is a fragment of any polypeptide disclosed in Table 1 and has a length from about 10 to about 250 amino acids. In some embodiments, the fragment is a fragment of any polypeptide disclosed in Table 1 and has a length from about 10 to about 200 amino acids. In some embodiments, the fragment is a fragment of any polypeptide disclosed in Table 1 and has a length from about 10 to about 150 amino acids. In some embodiments, the fragment is a fragment of any polypeptide disclosed in Table 1 and has a length from about 10 to about 100 amino acids. In some embodiments, the fragment is a fragment of any polypeptide disclosed in Table 1 and has a length from about 10 to about 90 amino acids. In some embodiments, the fragment is a fragment of any polypeptide disclosed in Table 1 and has a length from about 10 to about 80 amino acids. In some embodiments, the fragment is a fragment of any polypeptide disclosed in Table 1 and has a length from about 10 to about 70 amino acids. In some embodiments, the fragment is a fragment of any polypeptide disclosed in Table 1 and has a length from about 10 to about 60 amino acids. In some embodiments, the fragment is a fragment of any polypeptide disclosed in Table 1 and has a length from about 10 to about 50 amino acids. In some embodiments, the fragment is a fragment of any polypeptide disclosed in Table 1 and has a length from about 10 to about 40 amino acids. In some embodiments, the fragment is a fragment of any polypeptide disclosed in Table 1 and has a length from about 10 to about 30 amino acids. In some embodiments, the fragment is a fragment of any polypeptide disclosed in Table 1 and has a length from about 10 to about 20 amino acids.

In some embodiments, the fragment is a fragment of any polypeptide disclosed in Table 1 and has a length from about 10 to about 3000 amino acids. In some embodiments, the fragment is a fragment of any polypeptide disclosed in Table 1 and has a length from about 20 to about 3000 amino acids. In some embodiments, the fragment is a fragment of any polypeptide disclosed in Table 1 and has a length from about 30 to about 3000 amino acids. In some embodiments, the fragment is a fragment of any polypeptide disclosed in Table 1 and has a length from about 40 to about 3000 amino acids. In some embodiments, the fragment is a fragment of any polypeptide disclosed in Table 1 and has a length from about 50 to about 3000 amino acids. In some embodiments, the fragment is a fragment of any polypeptide disclosed in Table 1 and has a length from about 60 to about 3000 amino acids. In some embodiments, the fragment is a fragment of any polypeptide disclosed in Table 1 and has a length from about 70 to about 3000 amino acids. In some embodiments, the fragment is a fragment of any polypeptide disclosed in Table 1 and has a length from about 80 to about 3000 amino acids. In some embodiments, the fragment is a fragment of any polypeptide disclosed in Table 1 and has a length from about 90 to about 3000 amino acids. In some embodiments, the fragment is a fragment of any polypeptide disclosed in Table 1 and has a length from about 100 to about 3000 amino acids. In some embodiments, the fragment is a fragment of any polypeptide disclosed in Table 1 and has a length from about 150 to about 3000 amino acids. In some embodiments, the fragment is a fragment of any polypeptide disclosed in Table 1 and has a length from about 200 to about 3000 amino acids. In some embodiments, the fragment is a fragment of any polypeptide disclosed in Table 1 and has a length from about 250 to about 3000 amino acids. In some embodiments, the fragment is a fragment of any polypeptide disclosed in Table 1 and has a length from about 300 to about 3000 amino acids. In some embodiments, the fragment is a fragment of any polypeptide disclosed in Table 1 and has a length from about 350 to about 3000 amino acids. In some embodiments, the fragment is a fragment of any polypeptide disclosed in Table 1 and has a length from about 400 to about 3000 amino acids. In some embodiments, the fragment is a fragment of any polypeptide disclosed in Table 1 and has a length from about 450 to about 3000 amino acids. In some embodiments, the fragment is a fragment of any polypeptide disclosed in Table 1 and has a length from about 500 to about 3000 amino acids. In some embodiments, the fragment is a fragment of any polypeptide disclosed in Table 1 and has a length from about 550 to about 3000 amino acids. In some embodiments, the fragment is a fragment of any polypeptide disclosed in Table 1 and has a length from about 600 to about 3000 amino acids. In some embodiments, the fragment is a fragment of any polypeptide disclosed in Table 1 and has a length from about 650 to about 3000 amino acids. In some embodiments, the fragment is a fragment of any polypeptide disclosed in Table 1 and has a length from about 700 to about 3000 amino acids. In some embodiments, the fragment is a fragment of any polypeptide disclosed in Table 1 and has a length from about 750 to about 3000 amino acids. In some embodiments, the fragment is a fragment of any polypeptide disclosed in Table 1 and has a length from about 800 to about 3000 amino acids. In some embodiments, the fragment is a fragment of any polypeptide disclosed in Table 1 and has a length from about 850 to about 3000 amino acids. In some embodiments, the fragment is a fragment of any polypeptide disclosed in Table 1 and has a length from about 900 to about 3000 amino acids. In some embodiments, the fragment is a fragment of any polypeptide disclosed in Table 1 and has a length from about 950 to about 3000 amino acids. In some embodiments, the fragment is a fragment of any polypeptide disclosed in Table 1 and has a length from about 1000 to about 3000 amino acids. In some embodiments, the fragment is a fragment of any polypeptide disclosed in Table 1 and has a length from about 1050 to about 3000 amino acids. In some embodiments, the fragment is a fragment of any polypeptide disclosed in Table 1 and has a length from about 1250 to about 3000 amino acids. In some embodiments, the fragment is a fragment of any polypeptide disclosed in Table 1 and has a length from about 1500 to about 3000 amino acids. In some embodiments, the fragment is a fragment of any polypeptide disclosed in Table 1 and has a length from about 1750 to about 3000 amino acids. In some embodiments, the fragment is a fragment of any polypeptide disclosed in Table 1 and has a length from about 2000 to about 3000 amino acids. In some embodiments, the fragment is a fragment of any polypeptide disclosed in Table 1 and has a length from about 2250 to about 3000 amino acids. In some embodiments, the fragment is a fragment of any polypeptide disclosed in Table 1 and has a length from about 2500 to about 3000 amino acids. In some embodiments, the fragment is a fragment of any polypeptide disclosed in Table 1 and has a length from about 2750 to about 3000 amino acids.

As used herein, the term “hematopoietic pathway” refers to the genetic or developmental pathway in an animal responsible for a cell in a differentiated state to revert or begin to revert to a cell morphologically and functionally equivalent to a hematopoietic stem cell or hematopoietic progenitor cell. In some embodiments, the hematopoietic pathway is triggered by activation or stimulation of Notch1 in endothelial cells. In some embodiments, the hematopoietic pathway is triggered by inhibition or repression of Notch1 in hemogenic endothelial cells.

As used herein, the term “hematopoietic effector” refers to those compounds (small chemical compounds, nucleic acids, amino acid sequences, or hybrids thereof), that change or alter the activation state of the hematopoietic pathway in a cell. In some embodiments, the hematopoietic effector is a “hematopoietic activator” that activates or promotes activation of the hematopoietic pathway in a cell. In some embodiments, a hematopoietic activator is any of the activators listed on Table 1 or functional fragments thereof. In some embodiments, the hematopoietic effector is a “hematopoietic silencer” that inhibits or represses the function of one or more hematopoietic activators or the hematopoietic pathway in a cell. In some embodiments, the presence of a hematopoietic silencer stimulates activation of the hematopoietic pathway in a cell. In some embodiments, a hematopoietic silencer is any of the silencers listed on Table 1 or functional fragments thereof. It is possible that, if the cell has been exposed to a hematopoietic activator for a period of time sufficient to alter its phenotype, a second exposure of the same effector to the cell can lead to the hematopoietic pathway being inhibited or repressed, such that the same hematopoietic effector is both an activator and silencer.

As used herein the term “heterologous” refers to a nucleic acid sequence that is operably linked to another nucleic acid sequence to which it is not operably linked in nature, or to which it is operably linked at a different location in nature. For example, a protein-coding nucleic acid sequence operably linked to a promoter which is not the native promoter of this protein-coding sequence is considered to be heterologous to the promoter. In some embodiments, the heterologous sequence comprises a plasmid or episome.

As used herein, “sequence identity” is determined by using the stand-alone executable BLAST engine program for blasting two sequences (bl2seq), which can be retrieved from the National Center for Biotechnology Information (NCBI) ftp site, using the default parameters (Tatusova and Madden, FEMS Microbiol Lett., 1999, 174, 247-250; which is incorporated herein by reference in its entirety).

The term “subject” is used throughout the specification to describe an animal from which a cell sample is taken or an animal to which a disclosed cell or nucleic acid sequences have been administered. In some embodiment, the animal is a human. For diagnosis of those conditions which are specific for a specific subject, such as a human being, the term “patient” may be interchangeably used. In some instances in the description of the present disclosure, the term “patient” will refer to human patients suffering from a particular disease or disorder. In some embodiments, the subject may be a human suspected of having or being identified as at risk to develop cancer of the blood. In some embodiments, the subject may be diagnosed as having cancer of the blood or being identified as at risk to develop cancer of the blood. In some embodiments, the subject is suspected of having or has been diagnosed with requiring a bone marrow transplant. In some embodiments, the subject may be a human suspected of having or being identified as at risk to develop bone marrow transplants. In some embodiments, the subject may be a mammal which functions as a source of the endothelial cell sample. In some embodiments, the subject may be a non-human animal from which an endothelial cell sample is isolated or provided. The term “mammal” encompasses both humans and non-humans and includes but is not limited to humans, non-human primates, canines, felines, murines, bovines, equines, caprines, and porcines.

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions. Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. In some embodiments, the nucleic acid is isolated from an organism.

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

“Pharmacologically effective amount” or “pharmacologically effective concentration” as used herein means an amount or concentration, respectively, of a compound (relative to what the term modifies) that is sufficient to alter the condition of a cell exposed to that compound as compared to the cell unexposed to the same compound. In the case of some embodiments, the pharmacologically effective amount” or “pharmacologically effective concentration refers to the amount of a compound sufficient to alter the hematopoietic pathway of a cell as compared to the hematopoietic pathway of the same cell unexposed to the compound.

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5-10° C. lower than the thermal melting point (Tₘ) for the specific sequence at a defined ionic strength pH. The Tₘ may be the temperature (under defined ionic strength, pH, and nucleic concentration) at which 50% of the probes complementary to the target hybridize to the target sequence at equilibrium (as the target sequences are present in excess, at Tₘ, 50%> of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50%> formamide, 5×SSC, and 1% SDS, incubating at 42° C., or, 5×SSC, 1% SDS, incubating at 65° C., with wash in 0.2×SSC, and 0.1% SDS at 65° C.

“Substantially complementary” as used herein may mean that a first sequence is at least 60%, 65%, 70%, 75%, 80%, 85%, 90%, 95%, 96%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

“Substantially identical” as used herein may mean that, in respect to a first and a second sequence, a first and second sequence are at least 60%, 65%, 70%, 75%, 80%, 85%, 90%, 95%, 96%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 1000 or more nucleotides or amino acids, or with respect to nucleic acids, if the first sequence is substantially complementary to the complement of the second sequence.

In some embodiments, any of the nucleic acids disclosed herein can encode variants of any of the polypeptides disclosed herein. “Variant” used herein with respect to a nucleic acid means (i) a portion or fragment of a referenced nucleotide sequence; (ii) the complement of a referenced nucleotide sequence or portion thereof, (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto. “Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157: 105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within 2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

Nucleic acid molecules or nucleic acid sequences of the disclosure include those coding sequences comprising one or more of: any of the amino acid sequences identified in Table 1 and functional fragments thereof that possess no less than 65, 70, 75, 80, 85, 90, 95, 96, 97, 98, or 99% sequence identity with the coding sequences of the amino acid sequences disclosed herein.

“Vector” used herein means, in respect to a nucleic acid sequence, a nucleic acid sequence comprising a regulatory nucleic acid sequence that controls the replication or expression of an expressible gene. A vector may be either a self-replicating, extrachromosomal vector or a vector which integrates into a host genome. Alternatively, a vector may also be a vehicle comprising the aforementioned nucleic acid sequence. A vector may be a plasmid, bacteriophage, viral particle (isolated, attenuated, recombinant, etc.). A vector may comprise a double-stranded or single-stranded DNA, RNA, or hybrid DNA/RNA sequence comprising double-stranded and/or single-stranded nucleotides. In some embodiments, the vector is a viral vector that comprises a nucleic acid sequence that is a viral packaging sequence responsible for packaging one or plurality of nucleic acid sequence that encode one or a plurality of polypeptides. In some embodiments, the vector comprises a viral particle comprising a nucleic acid sequence operably linked to a regulatory sequence, wherein the nucleic acid sequence encodes a fusion protein comprising one or a plurality of structural viral polypeptides or fragments thereof. The disclosure relates to any vector comprising a nucleic acid sequence comprising SEQ ID NO:3, SEQ ID NO:4, and/or any functional fragment or variant thereof comprising 75%, 80%, 85%, 90%, 91%, 92%, 93%, 94%, 95%, 96%, 97%, 98%, or 99% sequence identity to SEQ ID NO:3 or SEQ ID NO:4. In some embodiments, the disclosure relates to the vectors comprising, consisting of or consisting essentially of SEQ ID NO:1 and/or SEQ ID NO:2. In some embodiments, the disclosure relates to the vectors comprising variants or functional fragments of SEQ ID NO:1 and/or SEQ ID NO:2. In some embodiments, the disclosure relates to the vectors comprising variants or functional fragments of SEQ ID NO:1 and/or SEQ ID NO:2 that comprises 75%, 80%, 85%, 90%, 91%, 92%, 93%, 94%, 95%, 96%, 97%, 98%, or 99% sequence identity to SEQ ID NO:1 or SEQ ID NO:2.

“Viral vector” as disclosed herein means, in respect to a vehicle, any virus, virus-like particle, virion, viral particle, or pseudotyped virus that comprises a nucleic acid sequence that directs packaging of a nucleic acid sequence in the virus, virus-like particle, virion, viral particle, or pseudotyped virus. In some embodiments, the virus, virus-like particle, virion, viral particle, or pseudotyped virus is capable of transferring a vector (such as a nucleic acid vector) into and/or between host cells. In some embodiments, the virus, virus-like particle, virion, viral particle, or pseudotyped virus is capable of transferring a vector (such as a nucleic acid vector) into and/or between target cells, such as a endothelial cell or hematopoietic cell in culture.

The disclosure relates to a method of differentiating an endothelial cell into a hematopoietic cell. In some embodiments, the hematopoietic cell is a hematopoietic stem cell or a hematopoietic progenitor cell. In some embodiments, methods disclosed herein comprise a two-step process of differentiating an endothelial cell into a hemogenic endothelial cell and then subsequently differentiating the hemogenic endothelial cell into a hematopoietic stem cell or progenitor cell. For purposes of this disclosure, the two-step process of differentiation may require stimulation and repression or inhibition of the hematopoietic pathway. In some embodiments, the method of differentiating the endothelial cells disclosed herein comprises exposing the endothelial cell to at least one hematopoietic activator of the hematopoietic pathway, such as but not limited to SOX17, at a concentration and for a time period sufficient to cause overexpression of the Notch1 and a change in character from a endothelial cell to a hemogenic endothelial cell. In some embodiments, the method of differentiating the endothelial cells disclosed herein comprises exposing the endothelial cell to at least one hematopoietic silencer of the hematopoietic pathway, such as but not limited to RUNX1, at a concentration and for a time period sufficient to inhibit expression Notch1 at levels associated with a hemogenic cell unexposed to at least one hematopoietic silencer and to change in character from a hemogenic endothelial cell to hematopoietic stem cell or hematopoietic progenitor cell. In some embodiments, the method of differentiating the endothelial cells disclosed herein comprises exposing the endothelial cell to at least two hematopoietic silencers of the hematopoietic pathway, such as but not limited to RUNX1 and a γ-secretase inhibitor, at a concentration and for a time period sufficient to inhibit expression of Notch1 and SOX17 at levels associated with a hemogenic cell unexposed to at least one hematopoietic silencer, such that the hemogenic endothelial cell changes in character from a hemogenic endothelial cell to hematopoietic stem cell or hematopoietic progenitor cell. In some embodiments, the methods disclosed herein require sequential exposure of the endothelial cell to: (a) at least one hematopoietic activator at a concentration and for time period sufficient to alter the endothelial cell to a hemogenic endothelial cell; and (b) at least two hematopoietic silencers at a concentration and for time period sufficient to alter the hemogenic endothelial cell to a hematopoietic cell. In some embodiments, the hematopoietic silencer is DAPT. In some embodiments, the endothelial cell is exposed to a nucleic acid sequence encoding RUNX1 and to a γ-secretase inhibitor. In some embodiments, the endothelial cell is exposed to a nucleic acid sequence encoding RUNX1 and is simultaneously exposed to DAPT.

The disclosure generally relates to altering the expression of Notch1 and SOX17 by introduction of or exposure of the endothelial cell to compounds that modulate the hematopoietic pathway, such that the SOX17 expression is increased in the endothelial cell as compared to an endothelial cell unexposed to the compound or compounds, and, after a time sufficient to revert the character of the endothelial cell to a hemogenic endothelial cell. The disclosure generally relates to altering the expression of Notch1 and SOX17 by introduction of or exposure of the endothelial cell to compounds that modulate the hematopoietic pathway, such that the Notch1 expression is decreased in the endothelial cell as compared to an endothelial cell unexposed to the compound or compounds, and, after a time sufficient to revert the character of the endothelial cell to a hematopoietic stem cell or progenitor cell. The disclosure generally relates to altering the expression of Notch1 and SOX17 by introduction of or exposure of the endothelial cell to compounds that modulate the hematopoietic pathway, such that the endothelial cell becomes a hematopoietic stem cell or progenitor cell. In some embodiments, the disclosure relates to methods of altering the expression of Notch1 and SOX17 in an endothelial cell by first exposing the endothelial cell to a compound or compounds that activate expression of Notch1 at a concentration and for a time period sufficient to alter the character of the endothelial cell to a hemogenic endothelial cell, and second sequentially exposing the hemogenic endothelial cell to a compound or compounds at a concentration and for a time period sufficient to reduce expression of both Notch1 and Sox17 in the endothelial cell as compared to a hemogenic endothelial cell unexposed to the compound or compounds. In some embodiments, the hematopoietic activator or compound is a nucleic acid sequence encoding SOX17 or a functional fragment thereof. In some embodiments, the hematopoietic silencer or compound is a nucleic acid sequence encoding RUNX1 or a functional fragment thereof. In some embodiments, the compound is a γ-secretase inhibitor. In some embodiments, the compound is DAPT. In some embodiments, the endothelial cell is exposed to one or a combination of any of the activators listed in Table 1, at a concentration and for a time period sufficient to alter the change the cell to a hemogenic endothelial cell. In some embodiments, the endothelial cell is exposed to one or a combination of any of the silencers listed in Table 1, optionally after exposure to the one or combination of activators, at a concentration and for a time period sufficient to alter the change the endothelial cell to a hematopoietic stem cell or progenitor cell.

The disclosure relates to methods by which endothelial cells can be de-differentiated into hematopoietic stem cells. Reprogramming of the endothelial cells may be accomplished by exposing the endothelial cells to one or a plurality of hematpoietic effectors disclosed herein for a time sufficient to sequentially activate, then deactivate the hematopoietic pathway. Hematopoietic stem cells were similar to human hematopoietic stem cells (HSCs) cells in morphology, proliferation, surface antigens, gene expression, epigenetic status of pluripotent cell-specific genes. Furthermore, these cells could be transplanted into mammals and exhibit HSC function. These findings demonstrate that hematopoietic cells can be generated from endothelial cells, which were thought to be terminally differentiated.

The hematopoietic cells in the pharmaceutical compositions may be derived by a biopsy of a transplant donor (optionally frozen after differentiation and harvesting) followed by expansion in culture using standard cell culture techniques. Placental tissue or umbilical cord tissue may be biopsied from a subject. The starting material is composed of three mm punch biopsies collected using standard aseptic practices. The biopsies are collected by the treating physician, placed into a vial containing sterile phosphate buffered saline (PBS). The biopsies are shipped in a 2-8° C. refrigerated shipper back to the manufacturing facility. After arrival at the manufacturing facility, the biopsy is inspected and, upon acceptance, transferred directly to the manufacturing area.

A cell of the disclosure may be cultured in the following manner. Cells are incubated at 37±2.0° C. with 5.0±1.0% CO2 and fed every three to five days in the T-500 flask and every five to seven days in the ten layer cell stack (10 CS). Cells should not remain in the T-500 flask for more than 10 days prior to passaging. QC release testing for safety of the Bulk Drug Substance includes sterility and endotoxin testing. When cell confluence in the T-500 flask is ≥95%, cells are passaged to a 10 CS culture vessel. Alternately, two Five Layer Cell Stacks (5 CS) or a 10 Layer Cell Factory (10 CF) can be used in place of the 10 CS. Passage to the 10 CS is performed by removing the spent media, washing the cells, and treating with Trypsin-EDTA to release adherent cells in the flasks into the solution. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37±2.0° C. with 5.0±1.0% CO2 and fed with fresh Complete Growth Media every five to seven days. Cells should not remain in the 10 CS for more than 20 days prior to passaging.

Primary Harvest: When cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process Quality Control (QC) testing performed to determine total viable cell count and cell viability.

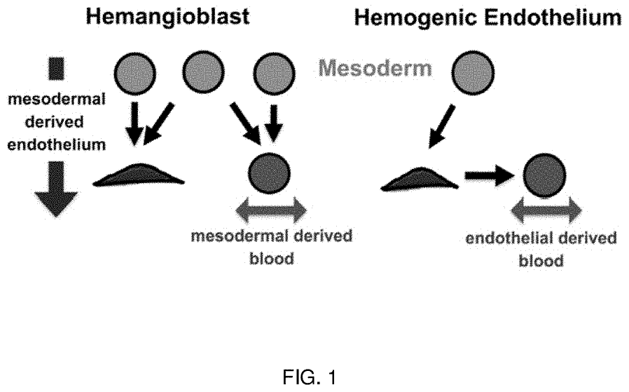

If additional cells are required after receiving cell count results from the primary 10 CS harvest, an additional passage into multiple cell stacks (up to four 10 CS) is performed (Step 5a in FIG. 1). For additional passaging, cells from the primary harvest are added to a 2 L media bottle containing fresh Complete Growth Media. Resuspended cells are added to multiple cell stacks and incubated at 37±2.0° C. with 5.0±1.0% CO2. The cell stacks are fed and harvested as described above, except cell confluence must be 80% or higher prior to cell harvest. The harvest procedure is the same as described for the primary harvest above. A mycoplasma sample from cells and spent media is collected, and cell count and viability performed as described for the primary harvest above.

Alternate Manufacturing Methods

Alternatively, cells can be passaged from either the T-175 flask (or alternatives) or the T-500 flask (or alternatives) into a spinner flask containing microcarriers as the cell growth surface. Microcarriers are small bead-like structures that are used as a growth surface for anchorage dependent cells in suspension culture. They are designed to produce large cell yields in small volumes.

In this apparatus, a volume of Complete Growth Media ranging from 50 mL-300 mL is added to a 500 mL, IL or 2 L sterile disposable spinner flask. Sterile microcarriers are added to the spinner flask. The culture is allowed to remain static or is placed on a stir plate at a low RPM (15-30 RRM) for a short period of time (1-24 hours) in a 37±2.0° C. with 5.0±1.0% CO2 incubator to allow for adherence of cells to the carriers. After the attachment period, the speed of the spin plate is increased (30-120 RPM). Cells are fed with fresh Complete Growth Media every one to five days, or when media appears spent by color change.

Cells are collected at regular intervals by sampling the microcarriers, isolating the cells and performing cell count and viability analysis. The concentration of cells per carrier is used to determine when to scale-up the culture. When enough cells are produced, cells are washed with PBS and harvested from the microcarriers using trypsin-EDTA and seeded back into the spinner flask in a larger amount of microcarriers and higher volume of Complete Growth Media (300 mL-2 L). Alternatively, additional microcarriers and Complete Growth Media can be added directly to the spinner flask containing the existing microcarrier culture, allowing for direct bead-to-bead transfer of cells without the use of trypsiziation and reseeding. Alternatively, if enough cells are produced from the initial T-175 or T-500 flask, the cells can be directly seeded into the scale-up amount of microcarriers. After the attachment period, the speed of the spin plate is increased (30-120 RPM). Cells are fed with fresh Complete Growth Media every one to five days, or when media appears spent by color change. When the concentration reaches the desired cell count for the intended indication, the cells are washed with PBS and harvested using trypsin-EDTA.

Microcarriers used within the disposable spinner flask may be made from poly blend such as BioNOC II® (Cesco Bioengineering, distributed by Bellco Biotechnology, Vineland, N.J.) and FibraCel® (New Brunswick Scientific, Edison, N.J.), gelatin, such as Cultispher-G (Percell Biolytica, Astrop, Sweden), cellulose, such as Cytopore™ (GE Healthcare, Piscataway, N.J.) or coated/uncoated polystyrene, such as 2D MicroHex™ (Nunc, Weisbaden, Germany), Cytodex® (GE Healthcare, Piscataway, N.J.) or Hy-Q Sphere™ (Thermo Scientific Hyclone, Logan, Utah).

Alternatively, cells can be processed on poly blend 2D microcarriers such as BioNOC II® and FibraCel® using an automatic bellow system, such as FibraStage™ (New Brunswick Scientific, Edison, N.J.) or BelloCell® (Cesco Bioengineering, distributed by Bellco Biotechnology, Vineland, N.J.) in place of the spinner flask apparatus. Cells from the T-175 (or alternatives) or T-500 flask (or alternatives) are passaged into a bellow bottle containing microcarriers with the appropriate amount of Complete Growth Media, and placed into the system. The system pumps media over the microcarriers to feed cells, and draws away media to allow for oxygenation in a repeating fixed cycle. Cells are monitored, fed, washed and harvested in the same sequence as described above.

Alternatively, cells can be processed using automated systems. After digestion of the biopsy tissue or after the first passage is complete (T-175 flask or alternative), cells may be seeded into an automated device. One method is an Automated Cellular Expansion (ACE) system, which is a series of commercially available or custom fabricated components linked together to form a cell growth platform in which cells can be expanded without human intervention. Cells are expanded in a cell tower, consisting of a stack of disks capable of supporting anchorage-dependent cell attachment. The system automatically circulates media and performs trypsiziation for harvest upon completion of the cell expansion stage.

Alternatively, the ACE system can be a scaled down, single lot unit version comprised of a disposable component that consists of cell growth surface, delivery tubing, media and reagents, and a permanent base that houses mechanics and computer processing capabilities for heating/cooling, media transfer and execution of the automated programming cycle.

Upon receipt, each sterile irradiated ACE disposable unit will be unwrapped from its packaging and loaded with media and reagents by hanging pre-filled bags and connecting the bags to the existing tubing via aseptic connectors. The process continues as follows: Inside a biological safety cabinet (BSC), a suspension of cells from a biopsy that has been enzymatically digested is introduced into the “pre-growth chamber” (small unit on top of the cell tower), which is already filled with Initiation Growth Media containing antibiotics. From the BSC, the disposable would be transferred to the permanent ACE unit already in place.

After approximately three days, the cells within the pre-growth chamber are trypsinized and introduced into the cell tower itself, which is pre-filled with cell culture media. Here, the “bubbling action” caused by CO2 injection force the media to circulate at such a rate that the cells spiral downward and settle on the surface of the discs in an evenly distributed manner.

The cells are allowed to multiply. At this time, confluence will be checked (method unknown at time of writing) to verify that culture is growing. Also at this time, the media disclosed herein will be replaced with fresh media disclosed herein. At the end of the culture period, the confluence is checked once more to verify that there is sufficient growth to possibly yield the desired quantity of cells for the intended treatment.

If the culture is sufficiently confluent, it is harvested. The spent media (supernatant) is drained from the vessel; PBS is pumped into the vessel (to wash the media, FBS from the cells) and drained almost immediately; trypsin-EDTA is pumped into the vessel to detach the cells from the growth surface; the trypsin/cell mixture is drained from the vessel and enter the spin separator; cryopreservative is pumped into the vessel to rinse any residual cells from the surface of the discs, and be sent to the spin separator as well; the spin separator collects the cells and then evenly resuspend the cells in the shipping/injection medium; from the spin separator, the cells will be sent through an inline automated cell counting device or a sample collected for cell count and viability testing via laboratory analyses. Once a specific number of cells has been counted and the proper cell concentration has been reached, the harvested cells are delivered to a collection vial that can be removed to aliquot the samples for cryogenic freezing.

Alternatively, automated robotic systems may be used to perform cell feeding, passaging, and harvesting for the entire length or a portion of the process. Cells can be introduced into the robotic device directly after digest and seed into the T-175 flask (or alternative). The device may have the capacity to incubate cells, perform cell count and viability analysis and perform feeds and transfers to larger culture vessels. The system may also have a computerized cataloging function to track individual lots. Existing technologies or customized systems may be used for the robotic option, such as the products obtained from The Automation Partnership (TAP).

C. Preparation of Cell Suspension

At the completion of culture expansion, the cells are harvested and washed, then formulated to contain from about 1.0 to about 2.7×10⁷ cells/mL, with a target of about 2.2×10⁷ cells/mL. Alternatively, the target can be adjusted within the formulation range to accommodate different indication doses. In some embodiments, the pharmaceutical composition consists of a population of viable, hematopoietic cells derived from endothelial lineages suspended in a cryopreservation medium consisting of Iscove's Modified Dulbecco's Medium (IMDM) and Profreeze-CDM™ (Lonza, Walkerville, Md.) plus 7.5% dimethyl sulfoxide (DMSO). Alternatively, a lower DMSO concentration may be used in place of 7.5%. Alternatively, CryoStor™ CS5 or CryoStor™ CS10 (BioLife Solutions, Bothell, Wash.) may be used in place of IMDM/Profreeze/DMSO.

After completion of the controlled rate freezing step, vials comprising any of the disclosed pharmaceutical compositions of hematopoietic cells are transferred to a cryogenic freezer for storage in the vapor phase. After cryogenic freezing, the Pharmaceutical composition is submitted for Quality Control testing. Pharmaceutical composition specifications also include cell count and cell viability testing performed prior to cryopreservation and performed again for Pharmaceutical composition—Cryovial. Viability of the cells must be at least about 65%, about 75%, 85% or higher for product release. Cell count and viability are conducted using an automated cell counting system (Guava Technologies), which utilizes a combination of permeable and impermeable fluorescent, DNA-intercalating dyes for the detection and differentiation of live and dead cells. Alternatively, a manual cell counting assay employing the trypan blue exclusion method may be used in place of the automated cell method above or other automated cell counting systems may be used to perform the cell count and viability method, including Cedex (Roche Innovatis AG, Bielefield, Germany), ViaCell™ (Beckman Coulter, Brea, Calif.), NuceloCounter™ (New Brunswick Scientific, Edison, N.J.), Countless® (Invitrogen, division of Life Technologies, Carlsbad, Calif.), or Cellometer® (Nexcelom Biosciences, Lawrence, Mass.). Pharmaceutical composition—Cryovial samples must meet a cell count specification of 1.0-2.7×10⁷ cells/mL prior to release. Sterility and endotoxin testing are also conducted during release testing.

In addition to cell count and viability, purity/identity of the pharmaceutical composition is performed and must confirm the suspension contains 98% or more hematopoietic cells. The usual cell contaminants include other cells types or those cells that did not undergo de-differentiation in accordance with the methods disclosed herein. The purity/identify assay employs fluorescent-tagged antibodies against biomarkers associated with to quantify the percent purity of a hematopoietic cell population. Cell count and viability is determined by incubating the samples with Viacount Dye Reagent and analyzing samples using the Guava PCA system. The reagent is composed of two dyes, a membrane-permeable dye which stains all nucleated cells, and a membrane-impermeable dye which stains only damaged or dying cells. The use of this dye combination enables the Guava PCA system to estimate the total number of cells present in the sample, and to determine which cells are viable, apoptotic, or dead.

Cryovial used to prepare the final dosage unit consists of hematopoietic cells or hematopoietic progenitor cells that are harvested from the final culture vessel, formulated to the desired cell concentration and cryopreserved in cryovials. Pharmaceutical composition Cryovial is stored in a cryopreservation medium consisting of IDM and Profreeze™ plus 7.5% DMSO to a target of 2.2×10⁷ cells/mL. After exposure to a controlled rate freezing cycle, the cryovialed Pharmaceutical composition is stored frozen in the vapor phase of a liquid nitrogen freezer.

Harvested cells are pooled, formulated in a cryopreservation media that includes Profreeze, DMSO and IMDM media, aliquoted into cryovials and stored frozen in liquid nitrogen as the Pharmaceutical composition—Cryovial material via controlled rate freezing.

The caps and vials are radiation sterilized and received sterile from the manufacturer. The required volume of bulk material needed for treatment is removed from frozen storage, thawed, and pooled. The cells are washed with 4× bulk volume of PBS and centrifuged at 150×g for 10 minutes (5±3° C.). This is followed by a wash with 4× bulk volume of DMEM by resuspension and centrifugation at 150×g for 10 minutes (5±3° C.). The washed cells are resuspended in DMEM without phenol red to a target concentration of 1.0-2.0×10⁷ cells/mL. Alternatively, the second 4× wash and final resuspension can be performed with Hypothermosol®-FRS (BioLife Solutions, Bothell, Wash.). The final sterile cryovial containers are then manually filled in a Biological Safety Cabinet to a volume of 1.2 mL/container. The pharmaceutical composition comprising one or plurality of hematopoietic stem cells is stored at 2-8° C. until shipment in a 2-8° C. refrigerated shipper to the administration site. Alternatively, Pharmaceutical composition vials can be removed from cryogenic storage and shipped directly to the administration site for dilution and administration. In the direct injection concept, the cells are harvested and prepared for cryopreservation at a higher cell concentration (3.0-4.0×10⁷ cell/mL as compared to the current target of 2.2×10⁷ cells/mL). When an injection is pending, the frozen vial will be shipped to the study site on dry ice or in a liquid nitrogen dewar. The administration site thaws the vial by hand or with a heat block, and performs a 1:1 ratio dilution of the frozen cells at the study site using a typical injection diluent such as bacteriostatic water, sterile water, sodium chloride, or phosphate buffered saline. Alternatively, DMEM may be used as the diluent. This concept eliminates the need to wash and prepare a fresh suspension of the injection for overnight shipment to the study site.

Alternatively, cells freshly harvested from flasks or cells stacks can be adjusted to a target concentration of 1.0-2.0×10⁷ cells/mL in DMEM, undergo all Bulk Harvest and Pharmaceutical composition—Cryovial testing described above and shipped fresh overnight to the administration site in a 2-8° C. refrigerated shipper as the final injection product. In this scenario, sterility and mycoplasma testing may be performed upstream from the harvest to allow time for results prior to shipment.

Library Generation and Banking

Bone Marrow Transplantation

In some embodiments, the methods of the disclosure relate to differentiation of an endothelial cell, which can be accomplished by any of the methods disclosed in the examples or components of those methods. The disclosure relates to a method of differentiating an endothelial cell comprising exposing the cell to a pharmacologically effective amount of a hematopoietic activator or compound and/or a hematopoietic silencer or compound for a time sufficient to differentiate the endothelial cell into a hematopoietic stem cell or hemogenic cell. In some embodiments, the cells that are differentiated are stored in freezing temperatures until thawed. In some embodiments one or a plurality of hematopoietic stem cells derived from an endothelial lineage are administered in a therapeutically effective amount to a subject in need thereof. in some embodiments, the subject has been diagnosed with or is suspected of having a hematopoietic disorder. In some embodiments, the hematopoietic disorder is cancer associated with one or more blood cells.

De-differentiating an endothelial cell may require a series of sequential steps. the disclosure relates to altering the expression of proteins in an endothelial cell by exposing the endothelial cell to one or a plurality of hematopoietic effectors either simultaneously or in sequence in pharmacologically effective amounts and for a period sufficient to alter the protein expression profiles of the endothelial cells. In some embodiments, the step of exposing the endothelial cells to one or a plurality of effectors comprises transfecting the endothelial cell with a nucleic acid sequence comprising a regulatory sequence in operable communication with one or a plurality of expressible nucleic acids sequences encoding the to one or a plurality of hematopoietic effectors. In some embodiments, the hematopoietic effectors comprise any one or combination of the effectors set forth in Table 1. In some embodiments, the hemtoapoietic effectors are chosen from one or a combination of RUNX1 or Sox17, or functional fragments thereof.

In some cases, iHeps are cultured for a period of time prior to transplantation (e.g., in HCM™ for 2 days). Cells (e.g., iHeps) can be provided to the individual (i.e., administered into the individual) alone or with a suitable substrate or matrix, e.g. to support their growth and/or organization in the tissue to which they are being transplanted (e.g., liver). In some embodiments, the matrix is a scaffold (e.g., an organ scaffold). In some embodiments, 1×103 or more cells will be administered (e.g., transplanted), for example 5×103 or more cells, 1×104 or more cells, 5×104 or more cells, 1×105 or more cells, 5×105 or more cells, 1×10s or more cells, 5×106 or more cells, 1×107 or more cells, 5×107 or more cells, 1×108 or more cells, 5×108 or more cells, 1×109 or more cells, 5×109 or more cells, or 1×1010 or more cells. In some embodiments, subject cells are administered into the individual on microcarriers (e.g., cells grown on biodegradable microcarriers).

The cells induced by the subject methods may be administered in any physiologically acceptable excipient (e.g., William's E medium), where the cells may find an appropriate site for survival and function (e.g., organ reconstitution). The cells may be introduced by any convenient method (e.g., injection, catheter, or the like).

The cells may be introduced to the subject (i.e., administered into the individual) via any of the following routes: parenteral, subcutaneous, intravenous, intracranial, intraspinal, intraocular, or into spinal fluid. The cells may be introduced by injection (e.g., direct local injection), catheter, or the like. Examples of methods for local delivery (e.g., delivery to the liver) include, e.g., by bolus injection, e.g. by a syringe, e.g. into a joint or organ; e.g., by continuous infusion, e.g. by cannulation, e.g. with convection (see e.g. US Application No. 20070254842, incorporated here by reference); or by implanting a device upon which the cells have been reversably affixed (see e.g. US Application Nos. 20080081064 and 20090196903, incorporated herein by reference).

In some cases, iHeps are administered into an individual by ultrasound-guided liver injection. In this way, cells can be placed directly into a bloodstream (e.g., in humans, or even in mice using a small animal ultrasound system). Brightness mode (B-mode) can be used to acquire two-dimensional images for an area of interest with a transducer and cells can be injected in solution (e.g., 100 μI to 300 μI, e.g., 200 μI of, for example, William's E medium) into one site or many sites (e.g., 1-30 sites) in the blood using, for example, a 30 gauge needle.

The number of administrations of treatment to a subject may vary. Introducing cells into an individual may be a one-time event; but in certain situations, such treatment may elicit improvement for a limited period of time and require an on-going series of repeated treatments. In other situations, multiple administrations of hematopoietic stem cells may be required before an effect is observed. As will be readily understood by one of ordinary skill in the art, the exact protocols depend upon the disease or condition, the stage of the disease and parameters of the individual being treated.

A “therapeutically effective dose” or “amount” or “therapeutic dose” is an amount sufficient to effect desired clinical results (i.e., achieve therapeutic efficacy). A therapeutically effective dose can be administered in one or more administrations. For purposes of this disclosure, a therapeutically effective dose of hematopoietic stem cells is an amount that is sufficient, when administered to (e.g., transplanted into) the individual, to palliate, ameliorate, stabilize, reverse, prevent, slow or delay the progression of the disease state (e.g., blood cell disorder) by, for example, providing functions normally provided by a subject with healthy blood.

In some embodiments, a therapeutically effective dose of hematopoietic stem cells is about 1×10³ or more cells (e.g., 5×10³ or more, 1×10⁴ cells, 5×10⁴ or more, 1×10⁵ or more, 5×10⁵ or more, 1×10⁶ or more, 5×10⁶ or more, 1×10⁷ cells, 5×10⁷ or more, 1×10⁸ or more, 5×10⁸ or more, 1×10⁹ or more, 5×10⁹ or more, or 1×10¹⁰ or more). In some embodiments, a therapeutically effective dose of hematopoietic stem cells is in a range of from about 1×10³ cells to about 1×10¹⁰ cells (e.g, from about 5×10³ cells to about 1×10¹⁰ cells, from about 1×10⁴ cells to about 1×10¹⁰ cells, from about 5×10⁴ cells to about 1×10¹⁰ cells, from about 1×10⁵ cells to about 1×10¹⁰ cells, from about 5×10⁵ cells to about 1×10¹⁰ cells, from about 1×10⁶ cells to about 1×10¹⁰ cells, from about 5×10⁵ cells to about 1×10¹⁰ cells, from about 1×10⁷ cells to about 1×10¹⁰ cells, from about 5×10⁷ cells to about 1×10¹⁰ cells, from about 1×10⁸ cells to about 1×10¹⁰ cells, from about 5×10⁸ cells to about 1×10¹⁰, from about 5×10³ cells to about 5×10⁹ cells, from about 1×10⁴ cells to about 5×10⁹ cells, from about 5×10⁴ cells to about 5×10⁹ cells, from about 1×10⁵ cells to about 5×10⁹ cells, from about 5×10⁵ cells to about 5×10⁹ cells, from about 1×10⁶ cells to about 5×10⁹ cells, from about 5×10⁶ cells to about 5×10⁹ cells, from about 1×10⁷ cells to about 5×10⁹ cells, from about 5×10⁷ cells to about 5×10⁹ cells, from about 1×10⁸ cells to about 5×10⁹ cells, from about 5×10⁸ cells to about 5×10⁹, from about 5×10³ cells to about 1×10⁹ cells, from about 1×10⁴ cells to about 1×10⁹ cells, from about 5×10⁴ cells to about 1×10⁹ cells, from about 1×10⁵ cells to about 1×10⁹ cells, from about 5×10⁵ cells to about 1×10⁹ cells, from about 1×10⁶ cells to about 1×10⁹ cells, from about 5×10⁶ cells to about 1×10⁹ cells, from about 1×10⁷ cells to about 1×10⁹ cells, from about 5×10⁷ cells to about 1×10⁹ cells, from about 1×108 cells to about 1×10⁹ cells, from about 5×10⁸ cells to about 1×10⁹, from about 5×10³ cells to about 5×10⁸ cells, from about 1×10⁴ cells to about 5×10⁸ cells, from about 5×10⁴ cells to about 5×10⁸ cells, from about 1×10⁵ cells to about 5×10⁸ cells, from about 5×10⁵ cells to about 5×10⁸ cells, from about 1×10⁶ cells to about 5×10⁸ cells, from about 5×10⁶ cells to about 5×10⁸ cells, from about 1×10⁷ cells to about 5×10⁸ cells, from about 5×10⁷ cells to about 5×10⁸ cells, or from about 1×10⁸ cells to about 5×10⁸ cells).

The cells of this disclosure can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types.

Cells of the subject methods may be genetically altered in order to introduce genes useful in the differentiated hepatocytes, e.g. repair of a genetic defect in an individual, selectable marker, etc. Cells may also be genetically modified to enhance survival, control proliferation, and the like. Cells may be genetically altered by transfection or transduction with a suitable vector, homologous recombination, or other appropriate technique, so that they express a gene of interest. In some embodiments, a selectable marker is introduced, to provide for greater purity of the desired differentiating cell.

The cells of this disclosure can also be genetically altered in order to enhance their ability to be involved in tissue regeneration, or to deliver a therapeutic gene to a site of administration. A vector is designed using the known encoding sequence for the desired gene, operatively linked to a promoter that is either pan-specific or specifically active in hematopoetic cells.

Many vectors useful for transferring exogenous genes into target mammalian cells are available. The vectors may be episomal, e.g. plasmids, virus derived vectors such cytomegalovirus, adenovirus, etc., or may be integrated into the target cell genome, through homologous recombination or random integration, e.g. retrovirus derived vectors such MMLV, HIV-1, ALV, etc. For modification of hematopoietic stem cells, lentiviral vectors are preferred. Lentiviral vectors such as those based on HIV or FIV gag sequences can be used to transfect non-dividing cells, such as the resting phase of human stem cells (see Uchida et al. (1998) P.N.A.S. 95(20): 1 1939-44).

Combinations of retroviruses and an appropriate packaging line may also find use, where the capsid proteins will be functional for infecting the target cells. Usually, the cells and virus will be incubated for at least about 24 hours in the culture medium. The cells are then allowed to grow in the culture medium for short intervals in some applications, e.g. 24-73 hours, or for at least two weeks, and may be allowed to grow for five weeks or more, before analysis. Commonly used retroviral vectors are “defective”, i.e. unable to produce viral proteins required for productive infection. Replication of the vector requires growth in the packaging cell line.

The host cell specificity of the retrovirus is determined by the envelope protein, env (p120). The envelope protein is provided by the packaging cell line. Envelope proteins are of at least three types, ecotropic, amphotropic and xenotropic. Retroviruses packaged with ecotropic envelope protein, e.g. MMLV, are capable of infecting most murine and rat cell types. Ecotropic packaging cell lines include BOSC23 (Pear et al. (1993) P.N.A.S. 90:8392-8396). Retroviruses bearing amphotropic envelope protein, e.g. 4070A (Danos ef al, supra.), are capable of infecting most mammalian cell types, including human, dog and mouse. Amphotropic packaging cell lines include PA12 (Miller ef al. (1985) Mol. Cell. Biol. 5:431-437); PA317 (Miller ef al. (1986) MpJ. CelL BioL 6:2895-2902) GRIP (Danos et al. (1988) PNAS 85:6460-6464). Retroviruses packaged with xenotropic envelope protein, e.g. AKR env, are capable of infecting most mammalian cell types, except murine cells.

The vectors may include genes that must later be removed, e.g. using a recombinase system such as Cre/Lox, or the cells that express them destroyed, e.g. by including genes that allow selective toxicity such as herpesvirus TK, bcl-xs, etc. Suitable inducible promoters are activated in a desired target cell type, either the transfected cell, or progeny thereof. By transcriptional activation, it is intended that transcription will be increased above basal levels in the target cell by at least about 100 fold, more usually by at least about 1000 fold.

### Sox/Runx Transfection and Culture Protocol

Culture Dish-Collagen Coating:

Make sterile filtered 0.02 M acetic acid
(11.5 μl glacial acetic acid in 10 mL sterile ddH20)
Coat dishes in 1:100 dilution of Bovine Collagen-I (Trevigen)
1 mL per 35 mm well.
37° C. for >1 hour to polymerize.
Wash three times with 1×PBS. Let air dry in hood. Use within two days.

Recovery Media:
Medium 200 (Gibco)+1×LVES (Gibco, 50×), sterile filter

General HUVEC Transfection Protocol:
Life technologies Neon 100 ul Transfection kit. Use standard protocol for adherent cells.
In brief: 5×10⁵ HUVECs (passage 5 or less)(VEC technologies) and 2 μg plasmid per each 100 ul transfection reaction, using R buffer.
Pulse Voltage: 1350v, Pulse Width: 30 ms, Pulse Number: 1
After transfection, suspend cells into 2 mL of recovery media in a collagen-coated 35 mm dish. Let recover overnight.

### Workflow:

1) Prior to experiment, passage HUVECs 1:3 every two days at 37° C., 5% CO₂.
2) Day 0. Transfect HUVECs with 2 μg pCXLE-CAG:Sox17+CMV:eGFP (SEQ ID NO:1) maxi-prepped episomal plasmid (endotoxin-free) onto collagen-coated plates with 2 mL recovery media.
3) Day 1. Switch from recovery media to MCDB-131 media (VEC technologies)
4) Day 3. Confluent cells should be trypsinized (0.25%), quenched with HEK media (DMEM+10% FBS+1% pen/strep) and passage cells 1:2 onto collagen-coated dishes.
5) Day 6 Transfect HUVECs (same protocol as above) with 2 μg pCXLE-CAG:Runx1+CMV:E2 (SEQ ID NO:2)—Crimson maxi-prepped episomal plasmid (endotoxin-free) onto collagen-coated plates in recovery media.
6) Day 7 morning: switch to MCDB-131 complete media.
7) Day 7 afternoon: Add MCDB-131 complete with DAPT (Sigma) (1×, 25 uM) from 1000× stock in DMSO.
8) Day 7 will be recovery. Budding is observed on days 8 and 9.
9) Media is replenished on day 9 for extended observation.

In some embodiments, the methods of the disclosure also relate to the reprogramming of endothelial cells into hematopoietic cells by transduction of endothelial cells with transcription factors and/or vascular niche induction. To establish vascular niche platform, endothelial cells were purified and transduced with a lentiviral vector expressing the adenoviral E4ORF1 gene (E4ECs, VeraVecs, Angiocrine Bioscience, New York, N.Y.). Purified CD45− CD133− c-Kit− CD31+ and clonal populations of CD45− CD144+ CD31+ CD62E+ full-term human umbilical vein endothelial cells (HUVECs) and adult primary human dermal microvascular endothelial cells (hDMEC) were cultured in endothelial cell growth medium. Then, HUVECs or hDMECs were transduced with lentiviral vectors expressing GFP and a combination of transcription factors: FOSB, GFI1, RUNX1 and SPI1 (FGRS). After 3 days, GFP+ FGRS-transduced endothelial cells were plated in co-culture with 30-50% subconfluent E4EC monolayers supplemented with serum-free haematopoietic media composed of Stem-Span SFEM, 10% KnockOut serum replacement, 5 ng ml-1 FGF-2, 10 ng ml-1 EGF, 20 ng ml-1 SCF, 20 ng ml-1 FLT3, 20 ng ml-1 TPO, 20 ng ml-1 IGF-1, 10 ng ml-1 IGF-2, 10 ng ml-1 TL-3 and 10 ng ml-1 TL-6. After 3-4 weeks of co-culture, outgrown GFP1 reprogrammed endothelial cells into human multipotent progenitor cells (rEC-hMPPs) formed typical grape-like haematopoietic colonies. After 4 weeks, human CD45+ rEC-hMPPs were FACS sorted for: (1) immunophenotypic analyses; (2) methylcellulose-CFC assay; (3) molecular profiling; (4) comparative genomic hybridization; and (5) transplanted retro-orbitally into primary sublethally irradiated (275 rad) 6-week-old NSG mice or sublethally irradiated (100 rad) 2-weekold mice neonates. After 3 months, sorted, bone-marrow-derived human CD45+ cells (hCD45+ cells) or whole bone marrow of the primary engrafted mice were transplanted into secondary recipients. After 3 months of primary and 6 months of the secondary transplantation, engrafted hCD45+ cells in bone marrow, spleen and peripheral blood of mice were FACS sorted and processed for: (1) multivariate immunophenotypic analyses; (2) clonal and oligo-clonal CFC assay; and (3) molecular profiling. Tissues of the engrafted mice were processed for histological examination to rule out malignant transformation.

In some embodiments, the methods of the disclosure also relate to one or more of the following methods and techniques:

A. Cultures.

Adult and neonatal dermal fibroblasts were cultured in F12-DMEM media supplemented with (1) IGFII and bFGF, or (2) IGFII, bFGF, Flt3 and SCF, on Matrigel-coated plates. Lentiviral vectors (pSIN) containing cDNAs of OCT4. NANOG, SOX2 and LIN28 were obtained from Addgene and were transfected into 293-FT cells using the virapower packaging kit (Invitrogen). Fibroblast transductions were performed at 24 h post 104 seeding on Matrigel. For derivation of CD45+ cells, fibroblasts were transduced with OCT4 expressing lentivirus and cultured in media (1) or (2), and iPSCs were derived as previously described15. Further haematopoietic differentiation was carried out using EB media supplemented with haematopoietic cytokines.

B. Functional/Phenotype Analysis.

Flow cytometry analysis of hematopoietic and pluripotency markers was performed using FACSCalibur (Beckman Coulter), and analysis was performed using the FlowJo 8.8.6 software. Cell sorting was performed using FACSAria II (Becton-Dickinson); Histological profiling of hematopoietic cells was performed using Cytospin and Giemsa-Wright staining and confirmed by the McMaster Pathology and Hematology Group; CFU formation was assayed using Methocult and Megacult kits from Stem Cell Technologies; Macrophage phagocytosis assay was performed using Fluorescein conjugated latex beads (Sigma) as particle tracers to analyse uptake bymonocytes derived from CD45+Fibs^{OCT4} cells; in vivo engraftment capacity was evaluated by intrafemoral injection of CD45^{+ve} cells into NSG mice. Ten weeks later bone marrow from injected femur, contralateral bones and spleen was analysed for the presence of human cells by flow cytometry; teratoma formation was evaluated by intratesticular injection into NOD/SCID mice. Resulting teratomas were evaluated for the presence of mesoderm, endoderm and ectoderm through histological examination.

C. Molecular Analysis.

For qPCR and microarray analysis, RNA was extracted using a total RNA purification kit (Norgen). Microarray analysis was done using Human Gene 1.0 ST arrays (Affymetrix) and dChIP software. OCT4 DNA occupancy (OCT4 ChIP) was done as previously described⁴⁵.

D. Preparation of Cells.

The autologous fibroblasts are derived by outgrowth from a tissue biopsy followed by expansion in culture using standard cell culture techniques. The starting material is composed of three 3-mm punch biopsies collected using standard aseptic practices. The biopsies are collected by the treating physician, placed into a vial containing sterile phosphate buffered saline (PBS). The biopsies are shipped in a 2-8° C. refrigerated shipper back to the manufacturing facility.

After arrival at the manufacturing facility, the biopsy is inspected and, upon acceptance, transferred directly to the manufacturing area. Upon initiation of the process, the biopsy tissue is then washed prior to enzymatic digestion. After washing, a Liberase Digestive Enzyme Solution is added without mincing, and the biopsy tissue is incubated at 37.0±2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6.

After digestion, Initiation Growth Media (IMDM, GA, 10% Fetal Bovine Serum (FBS)) is added to neutralize the enzyme, cells are pelleted by centrifugation and resuspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-75 flask.

Cells are incubated at 37±2.0° C. with 5.0±1.0% C02 and fed with fresh Complete Growth Media every three to five days. All feeds in the process are performed by removing half of the Complete Growth Media and replacing the same volume with fresh media. Alternatively, full feeds can be performed. Cells should not remain in the T-175 flask greater than 30 days prior to passaging. Confluence is monitored throughout the process to ensure adequate seeding densities during culture splitting. When cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask.

Morphology is evaluated at each passage and prior to harvest to monitor the culture purity throughout the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes appear round and irregularly shaped and, at higher confluence, they appear organized in a cobblestone formation. At lower confluence, keratinocytes are observable in small colonies.

Cells are incubated at 37±2.0° C. with 5.0±1.0% C02 and fed every three to five days in the T-500 flask and every five to seven days in the ten layer cell stack (IOCS). Cells should not remain in the T-500 flask for more than 10 days prior to passaging. Quality Control (QC) release testing for safety of the Bulk Pharmaceutical composition includes sterility and endotoxin testing. When cell confluence in the T-500 flask is >95%, cells are passaged to a 10 CS culture vessel. Alternately, two Five Layer Cell Stacks (5 CS) or a 10 Layer Cell Factory (10 CF) can be used in place of the 10 CS. IOCS.

Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete

Growth Media. The contents of the 2 L bottle are transferred into the 10 CS and seeded across all layers. Cells are then incubated at 37±2.0° C. with 5.0±1.0% C02 and fed with fresh Complete Growth Media every five to seven days. Cells should not remain in the IOCS for more than 20 days prior to passaging. The passaged fibroblasts are rendered substantially free of immunogenic proteins present in the culture medium by incubating the expanded fibroblasts for a period of time in protein free medium.

Primary Harvest: When cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

For treatment of nasolabial folds, the total cell count must be 3.4×10⁸ cells and viability 85% or higher. Alternatively, total cell yields for other indications can range from about 3.4×10⁸ to 1×10⁹ cells. Cell count and viability at harvest are critical parameters to ensure adequate quantities of viable cells for formulation of the Pharmaceutical composition. If total viable cell count is sufficient for the intended treatment, an aliquot of cells and spent media are tested for mycoplasma contamination. Mycoplasma testing is performed. Harvested cells are formulated and cryopreserved. If additional cells are required after receiving cell count results from the primary 10 CS harvest, an additional passage into multiple cell stacks (up to four 10 CS) is performed (Step 5a in FIG. 1). For additional passaging, cells from the primary harvest are added to a 2 L media bottle containing fresh Complete Growth Media. Resuspended cells are added to multiple cell stacks and incubated at 37±2.0° C. with 5.0±1.0% CO₂. The cell stacks are fed and harvested as described above, except cell confluence must be 80% or higher prior to cell harvest. The harvest procedure is the same as described for the primary harvest above. A mycoplasma sample from cells and spent media is collected, and cell count and viability performed as described for the primary harvest above.

The method decreases or eliminates immunogenic proteins by avoiding their introduction from animal-sourced reagents. To reduce process residuals, cells are cryopreserved in protein-free freeze media, then thawed and washed prior to prepping the final injection to further reduce remaining residuals.

E. Preparation of Cell Suspension.

At the completion of culture expansion, the cells are harvested and washed, then formulated to contain from about 1.0 to about 2.7×10⁷ cells/mL, with a target of 2.2×10⁷ cells/mL. Alternatively, the target can be adjusted within the formulation range to accommodate different indication doses. The pharmaceutical composition consists of a population of viable, autologous human fibroblast cells suspended in a cryopreservation medium consisting of Iscove's Modified Dulbecco's Medium (IMDM) and Profreeze-CDM™ (Lonza, Walkerville, Md.) plus 7.5% dimethyl sulfoxide (DMSO). Alternatively, a lower DMSO concentration may be used in place of 7.5% or CryoStor™ CS5 or CryoStor™ CS10 (BioLife Solutions, Bothell, Wash.) may be used in place of IMDM/Profreeze/DMSO. The freezing process consists of a control rate freezing step to the following ramp program:

STEP 1: Wait at 4.0° C.

STEP 2: 1.0° C./minC/m to −4.0° C. (sample probe)

STEP 3: 25.0° C./minC/m to −40° C. (chamber probe)

STEP 4: 10.0° C./minC/m to −12.0° C. (chamber probe)

STEP 5: 1.0° C./minC/m to −40° C. (chamber probe)

STEP 6: 10.0° C./minC/m to −90° C. (chamber probe)

STEP 7: End

After completion of the controlled rate freezing step, Bulk Pharmaceutical composition vials are transferred to a cryogenic freezer for storage in the vapor phase. After cryogenic freezing, the Pharmaceutical composition is submitted for Quality Control testing. Pharmaceutical composition specifications also include cell count and cell viability testing performed prior to cryopreservation and performed again for Pharmaceutical composition—Cryovial. Viability of the cells must be 85%> or higher for product release. Cell count and viability are conducted using an automated cell counting system (Guava Technologies), which utilizes a combination of permeable and impermeable fluorescent, DNA-intercalating dyes for the detection and differentiation of live and dead cells.

Alternatively, a manual cell counting assay employing the trypan blue exclusion method may be used in place of the automated cell method above. Alternatively, other automated cell counting systems may be used to perform the cell count and viability method, including Cedex (Roche Innovatis AG, Bielefield, Germany), ViaCell™ (Beckman Coulter, Brea, Calif.),

NuceloCounter™ (New Brunswick Scientific, Edison, N.J.), Countless® (Invitrogen, division of Life Technologies, Carlsbad, Calif.), or Cellometer® (Nexcelom Biosciences, Lawrence, Mass.). Pharmaceutical composition—Cryovial samples must meet a cell count specification of 1.0-2.7×107 cells/mL prior to release. Sterility and endotoxin testing are also conducted during release testing. In addition to cell count and viability, purity/identity of the Pharmaceutical composition is performed and must confirm the suspension contains 98% or more fibroblasts. The usual cell contaminants include keratinocytes. The purity/identify assay employs fluorescent-tagged antibodies against CD90 and CD 104 (cell surface markers for fibroblast and keratinocyte cells, respectively) to quantify the percent purity of a fibroblast cell population. CD90 (Thy-1) is a 35 kDa cell-surface glycoprotein. Antibodies against CD90 protein have been shown to exhibit high specificity to human fibroblast cells. CD 104, integrin β4 chain, is a 205 kDa transmembrane glycoprotein which associates with integrin a6 chain (CD49f) to form the α6/β4 complex. This complex has been shown to act as a molecular marker for keratinocyte cells (Adams and Watt 1991).

Antibodies to CD 104 protein bind to 100% of human keratinocyte cells. Cell count and viability is determined by incubating the samples with Viacount Dye Reagent and analyzing samples using the Guava PCA system. The reagent is composed of two dyes, a membrane—permeable dye which stains all nucleated cells, and a membrane-impermeable dye which stains only damaged or dying cells. The use of this dye combination enables the Guava PCA system to estimate the total number of cells present in the sample, and to determine which cells are viable, apoptotic, or dead.

Alternatively, cells can be passaged from either the T-175 flask (or alternatives) or the T-500 flask (or alternatives) into a spinner flask containing microcamers as the cell growth surface. Microcamers are small bead-like structures that are used as a growth surface for anchorage dependent cells in suspension culture. They are designed to produce large cell yields in small volumes.

In this apparatus, a volume of Complete Growth Media ranging from 50 mL-300 mL is added to a 500 mL, IL or 2 L sterile disposable spinner flask. Sterile microcarriers are added to the spinner flask. The culture is allowed to remain static or is placed on a stir plate at a low RPM (15-30 RRM) for a short period of time (1-24 hours) in a 37±2.0° C. with 5.0±1.0% C02 incubator to allow for adherence of cells to the carriers. After the attachment period, the speed of the spin plate is increased (30-120 RPM). Cells are fed with fresh Complete Growth Media every one to five days, or when media appears spent by color change.

Cells are collected at regular intervals by sampling the microcarriers, isolating the cells and performing cell count and viability analysis. The concentration of cells per carrier is used to determine when to scale-up the culture. When enough cells are produced, cells are washed with PBS and harvested from the microcarriers using trypsin-EDTA and seeded back into the spinner flask in a larger amount of microcarriers and higher volume of Complete Growth Media (300 mL-2 L). Alternatively, additional microcarriers and Complete Growth Media can be added directly to the spinner flask containing the existing microcarrier culture, allowing for direct bead-to-bead transfer of cells without the use of trypsinization and reseeding. Alternatively, if enough cells are produced from the initial T-175 or T-500 flask, the cells can be directly seeded into the scale-up amount of microcarriers.

After the attachment period, the speed of the spin plate is increased (30-120 RPM). Cells are fed with fresh Complete Growth Media every one to five days, or when media appears spent by color change. When the concentration reaches the desired cell count for the intended indication, the cells are washed with PBS and harvested using trypsin-EDTA. All release testing, cryopreservation and preparation of Drug Product—Injection would follow the process described in Sections C and D. Microcarriers used within the disposable spinner flask may be made from poly blend such as BioNOC II® (Cesco Bioengineering, distributed by Bellco Biotechnology, Vineland, N.J.) and FibraCel® (New Brunswick Scientific, Edison, N.J.), gelatin, such as Cultispher-G (Percell Biolytica, Astrop, Sweden), cellulose, such as Cytopore™ (GE Healthcare, Piscataway, N.J.) or coated/uncoated polystyrene, such as 2D MicroHex™ (Nunc, Weisbaden, Germany), Cytodex® (GE Healthcare, Piscataway, N.J.) or Hy-Q Sphere™ (Thermo Scientific Hyclone, Logan, Utah).

Alternatively, cells can be processed on poly blend 2D microcarriers such as BioNOC II® and FibraCel® using an automatic bellow system, such as FibraStage™ (New Brunswick Scientific, Edison, N.J.) or BelloCell® (Cesco Bioengineering, distributed by Bellco Biotechnology, Vineland, N.J.) in place of the spinner flask apparatus. Cells from the T-175 (or alternatives) or T-500 flask (or alternatives) are passaged into a bellow bottle containing microcarriers with the appropriate amount of Complete Growth Media, and placed into the system. The system pumps media over the microcarriers to feed cells, and draws away media to allow for oxygenation in a repeating fixed cycle. Cells are monitored, fed, washed and harvested in the same sequence as described above.

Alternatively, cells can be processed using automated systems. After digestion of the biopsy tissue or after the first passage is complete (T-175 flask or alternative), cells may be seeded into an automated device. One method is an Automated Cellular Expansion (ACE) system, which is a series of commercially available or custom fabricated components linked together to form a cell growth platform in which cells can be expanded without human intervention. Cells are expanded in a cell tower, consisting of a stack of disks capable of supporting anchorage-dependent cell attachment. The system automatically circulates media and performs trypsinization for harvest upon completion of the cell expansion stage.

Alternatively, the ACE system can be a scaled down, single lot unit version comprised of a disposable component that consists of cell growth surface, delivery tubing, media and reagents, and a permanent base that houses mechanics and computer processing capabilities for heating/cooling, media transfer and execution of the automated programming cycle. Upon receipt, each sterile irradiated ACE disposable unit will be unwrapped from its packaging and loaded with media and reagents by hanging pre-filled bags and connecting the bags to the existing tubing via aseptic connectors. The process continues as follows: Inside a biological safety cabinet (BSC), a suspension of cells from a biopsy that has been enzymatically digested is introduced into the “pre-growth chamber” (small unit on top of the cell tower), which is already filled with Initiation Growth Media containing antibiotics. From the BSC, the disposable would be transferred to the permanent ACE unit already in place.

After approximately three days, the cells within the pre-growth chamber are trypsinized and introduced into the cell tower itself, which is pre-filled with Complete Growth Media. Here, the “bubbling action” caused by CO₂ injection force the media to circulate at such a rate that the cells spiral downward and settle on the surface of the discs in an evenly distributed manner.

For approximately seven days, the cells are allowed to multiply. At this time, confluence will be checked (method unknown at time of writing) to verify that culture is growing. Also at this time, the Complete Growth Media will be replaced with fresh Complete Growth Media. CGM will be replaced every seven days for three to four weeks. At the end of the culture period, the confluence is checked once more to verify that there is sufficient growth to possibly yield the desired quantity of cells for the intended treatment.

If the culture is sufficiently confluent, it is harvested. The spent media (supernatant) is drained from the vessel. PBS will then is pumped into the vessel (to wash the media, FBS from the cells) and drained almost immediately. Trypsin-EDTA is pumped into the vessel to detach the cells from the growth surface. The trypsin/cell mixture is drained from the vessel and enter the spin separator. Cryopreservative is pumped into the vessel to rinse any residual cells from the surface of the discs, and be sent to the spin separator as well. The spin separator collects the cells and then evenly resuspend the cells in the shipping/injection medium. From the spin separator, the cells will be sent through an inline automated cell counting device or a sample collected for cell count and viability testing via laboratory analyses. Once a specific number of cells has been counted and the proper cell concentration has been reached, the harvested cells are delivered to a collection vial that can be removed to aliquot the samples for cryogenic freezing.

Alternatively, automated robotic systems may be used to perform cell feeding, passaging, and harvesting for the entire length or a portion of the process. Cells can be introduced into the robotic device directly after digest and seed into the T-175 flask (or alternative). The device may have the capacity to incubate cells, perform cell count and viability analysis and perform feeds and transfers to larger culture vessels. The system may also have a computerized cataloging function to track individual lots. Existing technologies or customized systems may be used for the robotic option.

In some embodiments, the invention relates to a pharmaceutical composition comprising a pharmacologically effective amount of the hematopoietic stem cells and progenitor cells describe herein and a pharmaceutically acceptable carrier. By “pharmaceutically acceptable carrier” is meant any carrier, diluent or excipient which is compatible with the biological component of a pharmaceutical composition and not deleterious to the recipient. Such carriers include, but are not limited to, saline, buffered saline, dextrose, water (e.g. water suitable for injection or sterile water), glycerol, ethanol, and combinations thereof.

SEQ ID NO:3=the coding sequence for SOX17 which appears within SEQ ID NO:1 beginning at bp #1736 and continues to between bp #2970 and bp #2980 SEQ ID NO:4=the coding sequence for RUNX1 which appears within SEQ ID NO:2 beginning between bp #1736 and bp #1750 and continuing to between bp #3180 and bp #3190.

TABLE 1
Activators

| Gene/pathway | NCBI Gene ID | Compound/drug |
|---|---|---|
| Notch | Gene ID: 4851 | Notch ligands - Dll4, Dll1 and Jag1, DAPT/gamma- |
| | | secretase |
| Sox17 | Gene ID: 64321 | TBD |
| Tgb1 | Gene ID: 21803 | Tgfb1, and inhibitors, SB-431542 . . . See |
| | | http://www.rndsystems.com/product_results.aspx?m=6456&c=199 |
| BMPs/smads | Gene ID: 652 | BMP proteins and small molecule inhibitors K02288, |
| | | DMH1, SB431542, inhibitors noggin |
| Runx1 | Gene ID: 861 | TBD |
| Gata2 | Gene ID: 2624 | TBD |
| Scl/Tal1 | Gene ID: 6886 | TBD |
| VEGF | Gene ID: 7422 | rVEGF, VEGF inhibitors - Sugen, Avastin |
| non-cell | | AGM/stromal cell lines |
| autonmous | | |
| role/multiple | | |
| pathways | | |
| PDGF | Gene IDs: 5156 | recombinant protein and inhibitors imatinib, sunitinib, |
| | and 5159 | sorafenib, pazopanib and nilotinib. |

Silencers

| Gene/pathway | Gene ID | Compound/drug |
|---|---|---|
| Notch | Gene ID: 4851 | Notch ligands - Dll4, Dll1 and Jag1, DAPT/gamma- |
| | | secretase |
| Sox17 | Gene ID: 64321 | TBD |
| Tgb1 | Gene ID: 21803 | Tgfb1, and inhibitors, SB-431542 . . . See |
| | | http://www.rndsystems.com/product_results.aspx?m=6456&c=199 |
| BMPs/smads | Gene ID: 652 | BMP proteins and small molecule inhibitors K02288, |
| | | DMH1, SB431542, inhibitors noggin |
| chromatin | Gene ID: 1105 | TBD |
| remodelers | | |
| (chdl) | | |
| VEGF | Gene ID: 7422 | rVEGF, VEGF inhibitors - Sugen, Avastin |
| PDGF | Gene IDs: 5156 | recombinant protein and inhibitors imatinib, sunitinib, |
| | and 5159 | sorafenib, pazopanib and nilotinib. |

### REFERENCES

All of the references, patent applications, or other documents listed in this application and the Examples section are herein incorporated by reference in their entireties.
Barns C. M., Christison-Lagay E. A., and Folkman J. (2007). The placenta theory and the origin of infantile hemangioma. Lymphat. Resl Biol. 5:245-55.
Bertrand, J. Y., Chi, N. C., Santoso, B., Teng, S., Stainier, D. Y. R. and Traver, D. (2010). Haematopoietic stem cells derive directly from aortic endothelium during development. Nature 464, 108-111.
Boisset, J.-C., van Cappellen, W., Andrieu-Soler, C., Galjart, N., Dzierzak, E. and Robin, C. (2010). In vivo imaging of haematopoietic cells emerging from the mouse aortic endothelium. Nature 464, 116-120.
Boye E., Yu Y., Paranya G., Mulliken J. B., Olsen B. R., and Bischoff J. (2001). Clonality and altered behavior of endothelial cells from hemangiomas. J. Clin. Invest. 107:745-52.
Burns C. E., Traver D., Mayhall E. Shepard J. L., and Zon L. I. (2005). Hematopoietic stem cell fate is established by the Notch-Runx pathway. Genes Dev. 19:2331-42.
Challen G. A., Goodell M. A. (2010). Runx1 isoforms show differential expression patterns during hematopoietic development but have similar functional effects in adult hematopoietic stem cells. Exp Hematol. 28:403-16.
Chen, M. J., Yokomizo, T., Zeigler, B. M., Dzierzak, E. and Speck, N. A. (2009). Runx1 is required for the endothelial to haematopoietic cell transition but not thereafter. Nature 457, 887-891.
Chenau J., Michelland S., de Fraipont F., et al. (2009). The cell line secretome, a suitable tool for investigating proteins released in vivo by tumors: application to the study of p53-modulated proteins secreted in lung cancer cells. J. Proteome. Res. 8:4579-91.
Chicha L., Feki A., Boni A. Irion O. Hovatta O., and Jaconi M. (2011). Human pluripotent stem cells differentiated in fully defined medium generate hematopoietic CD34− and CD34+ progenitors with distinct characteristics. PLoS One. 6:e14733.
Clarke, R. L., Yzaguirre, A. D., Yashiro-Ohtani, Y., Bondue, A., Blanpain, C., Pear, W. S., Speck, N. A. and Keller, G. (2013a). The expression of Sox17 identifies and regulates haemogenic endothelium. Nat Cell Biol.
Clarke, R. L., Yzaguirre, A. D., Yashiro-Ohtani, Y., Bondue, A., Blanpain, C., Pear, W. S., Speck, N. A. and Keller, G. (2013b). The expression of Sox17 identifies and regulates haemogenic endothelium. Nat Cell Biol.
Crampton S. P., Davis J., and Hughes C. C. (2010) Isolation of human umbilical vein endothelial cells (HUVEC). J. Vis. Exp. 183.
de Bruijn, M. F., Speck, N. A., Peeters, M. C. and Dzierzak, E. (2000). Definitive hematopoietic stem cells first develop within the major arterial regions of the mouse embryo. EMBO J. 19, 2465-2474.
Eilken, H. M., Nishikawa, S.-I. and Schroeder, T. (2009). Continuous single-cell imaging of blood generation from haemogenic endothelium. Nature 457, 896-900.
Espin-Palazón, R., Stachura, D. L., Campbell, C. A., Garcia-Moreno, D., Del Cid, N., Kim, A. D., Candel, S., Meseguer, J., Mulero, V. and Traver, D. (2014). Proinflammatory signaling regulates hematopoietic stem cell emergence. Cell 159, 1070-1085.
Green L. K., Klima M., and Burns T. R. (1988). Extramedullary hematopoiesis occurring in a hemangioma of the skin. Arch. Dermatol. 124:1720-1.
He, Q., Zhang, C., Wang, L., Zhang, P., Ma, D., Lv, J. and Liu, F. (2015). Inflammatory signaling regulates hematopoietic stem and progenitor cell emergence in vertebrates. Blood 125, 1098-1106.
Itinteang T., Tan S. T., Brasch H., and Day D. J. (2010). Haemogenic endothelium in infantile haemangioma. J. Clin. Pathol. 63:982-6.
Jinnin M., Medici D., Park L., et al. (2008). Suppressed NFAT-dependent VEGFR1 expression and constitutive VEGFR2 signaling in infantile hemangioma. Nat. Med. 14:1236-46.
Jokubaitis, V. J., Sinka, L., Driessen, R., Whitty, G., Haylock, D. N., Bertoncello, I., Smith, I., Péault, B., Tavian, M. and Simmons, P. J. (2008). Angiotensin-converting enzyme (CD143) marks hematopoietic stem cells in human embryonic, fetal, and adult hematopoietic tissues. Blood 111, 4055-4063.
Khan Z. A., Melero-Martin J. M., Wu X., et al. (2006). Endothelial progenitor cells from infantile hemangioma and umbilical cord blood display unique cellular responses to endostatin. Blood. 108:915-21.
Kim, I., Saunders, T. L. and Morrison, S. J. (2007). Sox17 Dependence Distinguishes the Transcriptional Regulation of Fetal from Adult Hematopoietic Stem Cells. Cell 130, 470-483.
Krol A., and MacArthur C. J. (2005). Congenital hemangiomas: rapidly involuting and noninvoluting congenital hemangiomas. Arch. Facial. Plast. Surg. 7:307-11.
Li, Y., Esain, V., Teng, L., Xu, J., Kwan, W., Frost, I. M., Yzaguirre, A. D., Cai, X., Cortes, M., Maijenburg, M. W., et al. (2014). Inflammatory signaling regulates embryonic hematopoietic stem and progenitor cell production. Genes & Development gad.253302.114.
Marshall C. J., Sinclair J. C., Thrasher A. J., and Kinnon C. (2007). Bone morphogenetic protein 4 modulates c-Kit expression and differentiation potential in murine embryonic aorta-gonad-mesonephros haematopoiesis in bitro. Br. J. Haematol. 139:321-30.
Nobuhisa, I., Osawa, M., Uemura, M., Kishikawa, Y., Anani, M., Harada, K., Takagi, H., Saito, K., Kanai-Azuma, M., Kanai, Y., et al. (2014). Sox17-mediated maintenance of fetal intra-aortic hematopoietic cell clusters. Mol. Cell. Biol. 34, 1976-1990.
North, T., Gu, T. L., Stacy, T., Wang, Q., Howard, L., Binder, M., Marin-Padilla, M. and Speck, N. A. (1999). Cbfa2 is required for the formation of intra-aortic hematopoietic clusters. Development 126, 2563-2575.
Ritter M. R., Reinisch J., Friedlander S. F., Frieldlander M. (2006). Myeloid cells in infantile hemangioma. Am. J. Pathol. 168:621-8.
Robin, C., Ottersbach, K., Boisset, J. C., Oziemlak, A. and Dzierzak, E. (2011). CD41 is developmentally regulated and differentially expressed on mouse hematopoietic stem cells. Blood 117, 5088-5091.
Sawamiphak, S., Kontarakis, Z. and Stainier, D. Y. R. (2014). Interferon Gamma Signaling Positively Regulates Hematopoietic Stem Cell Emergence. DEVCEL 31, 640-653.
Szabo E., Rampalli S., Risueno R. M. (2010) Direct conversion of human bibroblasts to multilineage blood progenitors. Nature. 468:521-6.
Takahashi K., Yamanaka S. (2006). Induction of pluripotent stem cells from mouse embryonic and adult fibroblasts cultures by defined factors. Cell. 126:663-76.
Tan S. T., Hasan Q., Velickovic M., Ruger B. M., Davis R. P., and Davis P. F. (2000). A novel in vitro human model of hemangioma. Mod. Pathol. 13:92-9.
Tavian M., Hallais M. F., and Peault B. (1999). Emergence of intraembryonic hematopoietic precursors in the pre-liver human embryo. Development. 126:793-803.
Tavian, M., Coulombel, L., Luton, D., Clemente, H. S., DIETERLEN-LIEVRE, F. and Peault, B. (1996). Aorta-associated CD34+ hematopoietic cells in the early human embryo. Blood 87, 67-72.
Tober, J., Yzaguirre, A. D., Piwarzyk, E. and Speck, N. A. (2013). Distinct temporal requirements for Runx1 in hematopoietic progenitors and stem cells. Development.
Wu J. K., Adepoju O., De Silva D., et al. (2010). A switch in Notch gene expression parallels stem cell to endothelial transition in infantile hemangioma. Angiogenesis, 13:15-23.
Yamamoto, S., Niida, S., Azuma, E., Yanagibashi, T., Muramatsu, M., Huang, T. T., Sagara, H., Higaki, S., Ikutani, M., Nagai, Y., et al. (2015). Inflammation-induced endothelial cellderived extracellular vesicles modulate the cellular status of pericytes. Sci. Rep. 5, 8505-10.
Yokomizo, T. and Dzierzak, E. (2010). Three-dimensional cartography of hematopoietic clusters in the vasculature of whole mouse embryos. Development 137, 3651-3661.
Yoon M. J., Koo B. K., Song R. (2008). Mind Bomb-1 is essential for intraembryonic hematopoiesis in the aortic endothelium and the subaortic patches. Mol Cell Biol. 28:4794-804.
Zang W. J., Wu L. Q., Liu H. L. et al. (2009). Abnormal blood vessels formed by human liver cavernous hemangioma endothelial cells in nude mice are suitable for druf evaluation. Microvasc. Res. 78:379-85.
Zovein, A. C., Hofmann, J. J., Lynch, M., French, W. J., Turlo, K. A., Yang, Y., Becker, M. S., Zanetta, L., Dejana, E., Gasson, J. C., et al. (2008). Fate tracing reveals the endothelial origin of hematopoietic stem cells. Cell Stem Cell 3, 625-636.

Any journal article or patent application disclosed herein is incorporated by reference in its entirety.

### EXAMPLES

### Example 1: Determine Whether Endothelial Derived Hematopoietic Cells from the AGM, Yolk Sac, and Placenta have True Stem Cell Identity and Capacity

Initial studies, using an inducible ex vivo fate tracing strategy, have demonstrated that the endothelium of the AGM, yolk sac, and placenta can separately give rise to hematopoietic cells (Zovein et al. 2008). These cells were traced in vivo to the adult bone marrow, implicating definitive hematopoietic capacity. However, the separate contribution of each vascular bed to adult hematopoiesis remains to be seen. In order to investigate whether endothelial derived blood cells from each of these sources has a stem cell identity and differentiation potential, we will conduct ex vivo organ cultures of the placenta, yolk sac and AGM. Using the previously described inducible Cre mouse line, we will induce endothelial lineage tracing to label emerging hematopoietic cells within the separate organ cultures, and analyze their cell surface markers by FACS for stem and differentiation markers, and investigate gene expression within the various sites. In addition, we will also sort the labeled hematopoietic cells from their respective cultures and evaluate their ability to long-term reconstitute adult irradiated hosts (the gold standard for HSCs).

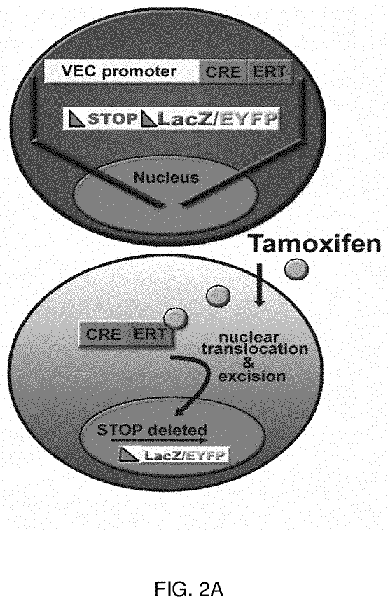

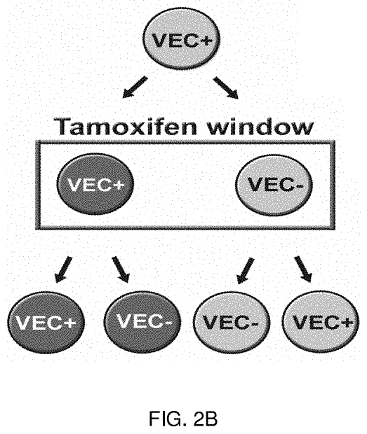

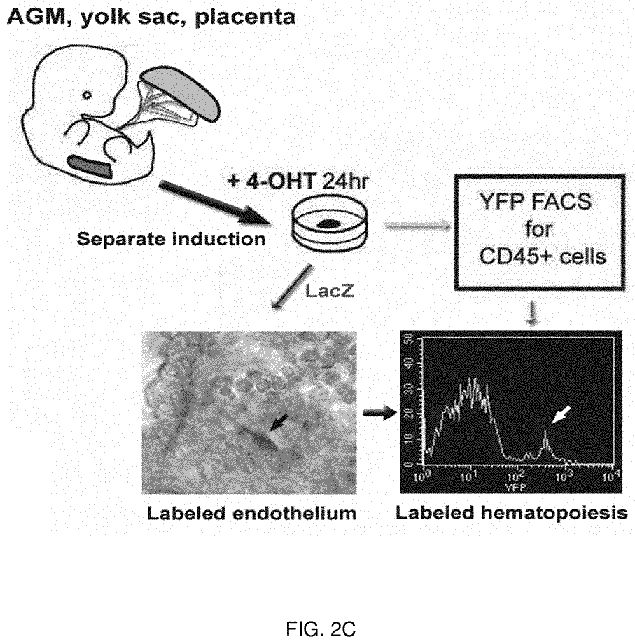

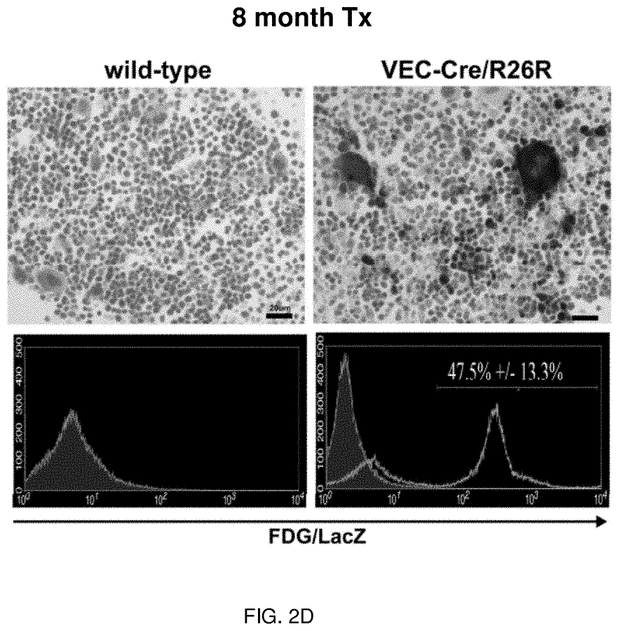

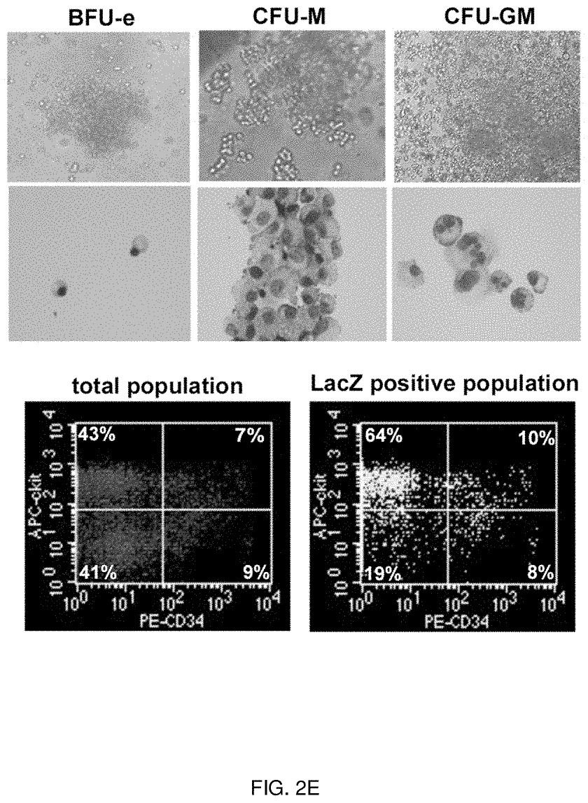

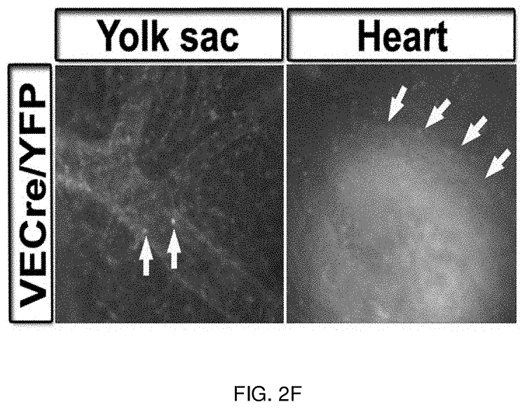

Genetic tracing using an inducible VE-cadherin Cre allowed separate in vitro induction of each hemogenic endothelial site to demonstrate endothelial-derived hematopoiesis (FIGS. 2A, 2B, 2C). Long-term lineage tracing in vivo showed that the cells traced in the mid-gestation embryo were capable of definitive hematopoiesis. However, questions remain as to whether all the sites (AGM, placenta, yolk sac) investigated in vitro contributed to the adult bone marrow in vivo.

To understand the contribution of each hemogenic vascular bed to adult hematopoiesis, in vitro induction followed by bone marrow transplantation will be necessary. In addition, it would be critical to understand whether the HSC phenotype differs among hemogenic vascular sites, which can be assessed through FACS analysis of stem markers, and methylcellulose colony assays. We have previously demonstrated that within the constitutive VE-cadherin Cre line (Alva et al., 2006), labeled bone marrow is capable of repopulating irradiated hosts with the same level of engraftment (% labeled hematopoietic cells) as the donor population (FIG. 2D; Monvoisin et al., 2006).

In addition, we have demonstrated proficiency with FACS and methylcellulose colony assays of embryonic tissues, as well as adult bone marrow. When the vitelline artery from a VE-cadherin Cre/R26R LacZ line is brought to single cell suspension and cultured in methylcellulose for 7-10 days, it gives rise to multiple types of hematopoietic colonies: definitive erythroid (BFU-e), Macrophage (CFUM), and Granulocyte/Macrophage (CFU-GM) (FIG. 2E). When analyzed for stem cell markers c-kit and CD34, there is a large enrichment of CD34+ and c-kit+ single and double positive cells within the LacZ population, suggesting that the HSC compartment is predominantly made up of VE-cadherin (endothelial) progeny. The c-kit+/CD34+ double positive population has been previously shown to define true HSCs within the AGM, and thus may identify HSCs derived from hemogenic endothelium (Delassus et al., 1999). As the FACS-gal assay used to evaluate LacZ expression can have variability and auto-fluorescence within embryonic tissues (Zovein et al., 2008), we have bred all our lines with the EYFP R26R line to improve FACS and cell sorting efficiency (Srinivas et al., 2001), see FIG. 2C.

Another benefit to breeding the VE-cadherin Cre lines into R26R EYFP reporters is the ability to image the tissue or embryos ex vivo. We have imaged live embryos at E10 of the VE-cadherin Cre/YFP line to confirm the feasibility of real time imaging. We are able to monitor blood flow of endothelial derived hematopoietic cells, and well as any cell migration. The still images are depicted in FIG. 2F.

### Example 2: Determine Whether Endothelial Derived Hematopoietic Cells from the AGM, Yolk Sac, and Placenta have True Stem Cell Identity and Capacity (Prophetic)

### Rationale

While our initial studies demonstrated that endothelial derived hematopoietic cells contribute to adult bone marrow in vivo when lineage traced, there is still some debate as to whether all hemogenic endothelial sites during early to midgestation are capable of definitive hematopoiesis. While the AGM1, placenta¹⁰², and yolk sac⁴⁷ have all been shown to repopulate irradiated hosts, the studies were complicated by established circulation between hemogenic sites, and/or the inability to fate trace the lineage responsible for HSC origin. It is also unclear whether the various hemogenic sites would differ in their hematopoietic repertoire, as there have been numerous reports regarding the various primitive and definitive hematopoietic waves within the yolk sac¹⁰³. Another possibility may be that while the stem markers expressed by endothelial derived HSCs may vary from each site, the ultimate capacity for adult repopulation may not. If indeed the true potential of HSCs is similar across the various hemogenic vascular beds, it would imply a similarity across niches, and thus an overarching program from which to coax endothelium to produce blood.

### Experimental Approach

By inducing endothelial fate tracing in each hemogenic vascular site, in vitro, and collecting the labeled blood, we can specifically analyze hematopoietic cells for phenotype and function.

a) In vitro fate tracing. We have previously demonstrated, using a tamoxifen inducible VE-cadherin Cre line crossed to a R26R EYFP reporter, that endothelial induction of specific vascular beds (AGM, placenta and yolk sac) results in hematopoietic progeny. The culture system involves dissection of each tissue with overnight culture on a 40 μm mesh (air liquid interface) in Myelocult media (Stem Cell technologies) supplemented with 10-6M hydrocortisone and 10 μM 4-hydroxyprogesterone (4-OHT) (Sigma). Organs from an entire litter can be induced separately and then pooled for analysis. One littermate is cultured in absence of 4-OHT for a negative fluorescence control. As the induction only results in a small subset of labeled endothelium (˜4%) and a larger cohort of hematopoietic cells (˜25%)11, our analysis and conclusions cannot describe total numbers of HSCs produced from each hemogenic endothelial site, but can qualitatively answer whether or not HSC capacity exists at each site. Our initial evaluation spanned from E10.5 to E12.5. As endothelial derived blood was detected at all time-points, the question remained whether there was an onset and extinction to the hemogenic ability of the endothelium. For our current analysis, we will evaluate E8.5-E13.5 AGM, placenta and yolk sac (E8.5 requires evaluation of the allantois instead of the placenta).

b) Phenotype evaluation.

FACS analysis. Multiple stem markers are expressed on HSCs during development, and include c-kit, Sca-1, AA4.1, CD34, CD41, CD45, and Mac-1. In particular, AGM HSCs have been described to be c-kit+/CD34+97. Yet CD34+ is also an endothelial marker104, and thus may not help distinguish hematopoietic populations for transplant. The definitive subset of hematopoietic cells appears to mature from a c-kit/CD34/CD41 stage to a c-kit/CD34/CD41/CD45 phenotype from the AGM to the fetal liver105. However, it is unclear whether CD41 also may label hemogenic endothelium^{13, 105}. After overnight organ culture, the tissues and cells in media will be spun down separately and brought to single-cell suspensions, and then stained with the antibodies to the markers mentioned above, and analyzed via fluorescent activated cell sorting (FACS). We have access to, and proficiency using, either a 4-color FACS-Calibur machine or 8-color LSRII Flow cytometer within the Broad Stem Cell Institute Core. As the EYFP+ induced endothelium will be likely associated with the tissue growing on the culture mesh, and the labeled hematopoietic progeny will be in the media, we can separately evaluate the labeled populations from each for phenotypic markers. And in addition, use defined endothelial markers, such as Ephrin B2, an arterial marker¹⁰⁶, to further distinguish endothelial cells from hematopoietic cells.

Genetic Analysis. The EYFP labeling of endothelial derived blood in our culture system also permits ease of fluorescent cell sorting. Through the previously mentioned core, we also have access to a FACSAria high-speed multicolor cell sorter. By sorting EYFP positive cells from both the endothelial cells (EphrinB2+) (associated with the organ culture) and the hematopoietic cells (ckit+/CD41+) in the media, we can obtain mRNA from the resulting populations and evaluate gene expression through Microarray. The UCLA Clinical Microarray core has ample experience running differential gene arrays using Affymetrix chips and data analysis on RNA isolated from as little as 10,000 cells (using the Stratagene Microprep RNA kit). By measuring the differential gene expression of labeled endothelium and hematopoietic cells between each site (AGM, placenta, and yolk sac) we can understand how extracellular markers may predict genetic expression, which in turn may predict function. As it would become laborious and expensive to compare genetic expression between all three sites at all time-points, we will focus on the E11.0-11.5 timepoint when budding is seen to occur in the AGM6, and HSC emergence is underway in the placentaio¹⁰².

c) Functional evaluation—transplant. As mentioned above, the culture system of EYFP tracing allows for cell sorting of populations. The true hallmark of a stem cell is the ability to repopulate irradiated hosts by self-renewal (long term hematopoiesis >8 months) and differentiation to all lineages. We will sort EYFP+/c-kit+/CD41+/EphrinB2− cells (endothelial derived HSCs without arterial contamination) from the different sites (AGM, yolk sac and placenta from E11.0-11.5 cultures), and transplant into lethally irradiated hosts as described65. Animals will then be monitored for engraftment by intermittent FACS analysis of peripheral blood (obtained by retro-orbital bleeding), and upon sacrifice at 8 months to 1 year, FACS analysis of the bone marrow. Both peripheral blood and bone marrow will be evaluated for EYFP+ multi-lineage reconstitution by expression of the following surface markers: Erythroid (CD71+/Ter119+), Myeloid (Gr-1+/Mac-1+), T cell (CD3+ or CD4+/CD8+), B cell (B220+), HSC (Lineage−,Sca-1+,ckit+ CD150+). The term embryo equivalent (ee), which is commonly used when transplanting embryonic tissue, was meant to represent the total number of HSCs within the organ transplanted, and is usually diluted to various amounts 3-.001ee^{60,102}. As the 4-OHT induction does not result in Cre expression in every endothelial cell, we will transplant all the EYFP+/ckit+/CD41+/EphrinB2− cells induced per litter (generally 5 embryo equivalents, as one embryo is not induced as a control). We can evaluate the EYFP+ embryo equivalent fraction of the total by measuring (by FACS) the percentage of EYFP+ and EYFP− compartments within the ckit+/CD41+/EphrinB2− population. This percentage of EYFP+ within the population multiplied by the number of embryos induced (likely 5) will give us the true embryo equivalent. As a precaution 2×104 unlabeled wild-type bone marrow cells will be co-transplanted with our target population to ensure short-term survival of the recipients, as previously described¹⁰⁷.

### Expected Results

Throughout the developmental period proposed (E8.5 to E13.5), hemogenic endothelium is likely active from E8.5 to E12.5 with a peak capacity around E10.5-11.5. During this period, all sites (AGM, yolk sac and placenta) can be predicted to have long term repopulating ability. However, it is also probable that each site will have slightly different phenotypic characteristics and have variations in lineage commitment. The yolk sac for example may have a higher erythroid contribution than lymphoid.

### Example 3: β1 Integrin Constructs for Cell Specific Deletion

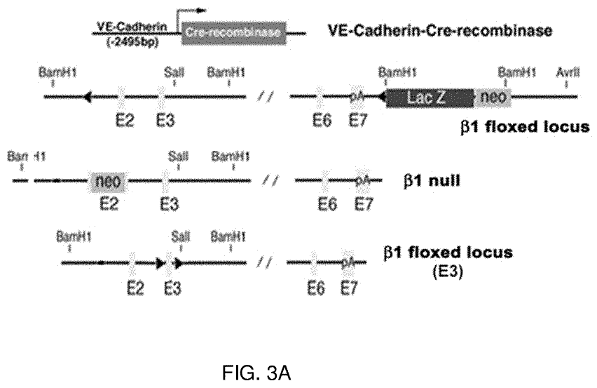

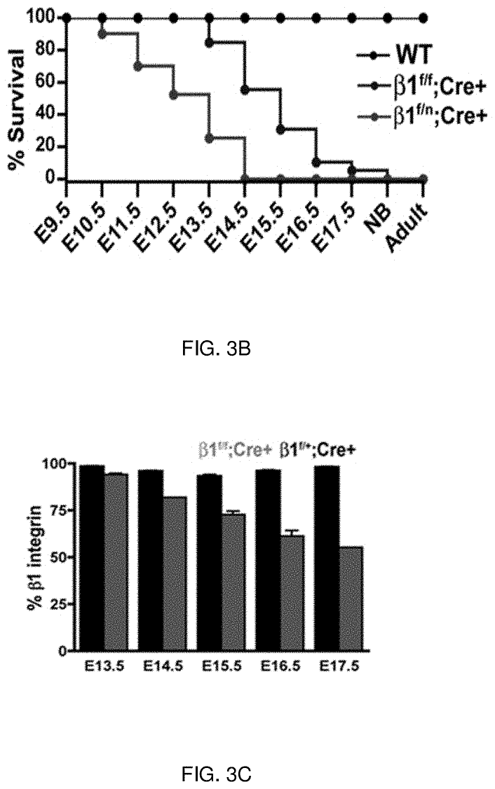

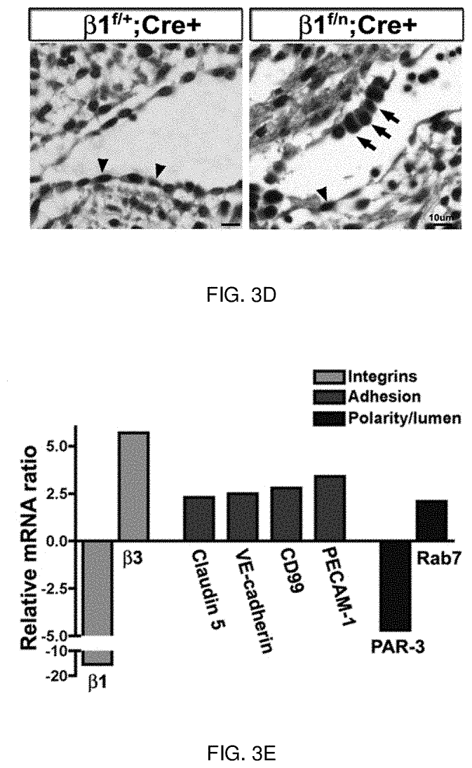

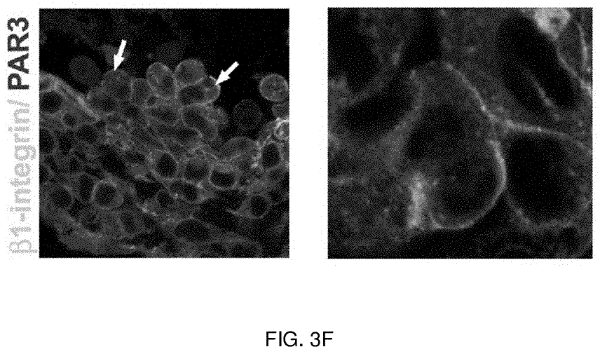

There are multiple floxed β1 integrin constructs available for tissue specific deletion. We have analyzed endothelial cell-specific deletion of β1 integrin by VE-cadherin Cre recombinase (β1^{f/f}; Cre+) using a floxed β1 integrin construct that spans a large region (20 kb; Potocnik et al., 2000), resulting in a hypomorphic phenotype with lethality from E13.5 to E17.5 (FIG. 3B). When a floxed allele is replaced by a “null” allele (β1^{f/n}; Cre+), an earlier lethality is noted (FIGS. 3A, 3B, 3C), but the lethality still occurs later than previously reported endothelial specific deletions using shorter (and possibly more efficient) floxed β1 integrin constructs (Carlson et al., 2008; Tanjore et al., 2008; Lei et al., 2008).

Using the β1^{f/n}; Cre+ line, which bypasses early vascular morphogenesis abnormalities encountered with other β1 endothelial deletions, we observed abnormal endothelial cell shape and polarity (FIG. 3D). When β1 integrin deleted endothelial cell gene expression was evaluated by microarray, it was noted that the endothelium exhibited an increase in adhesion, and loss of par-3 expression (FIG. 3E).

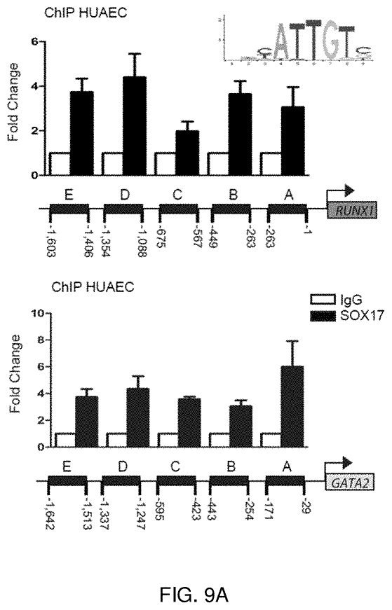

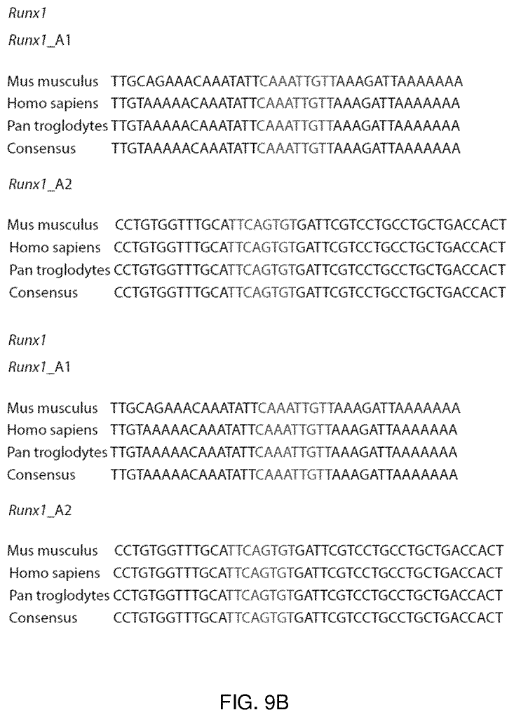

When the AGM at E11 was investigated for the polarity protein Par-3, in the context of β1 integrin, asymmetric expression was noted within hematopoietic clusters (FIG. 3F). It appears that Par-3 is expressed in cells dividing away from the endothelial niche as delineated by β1 integrin expression. This suggests that a defined polarity, set up by the opposed expression of β1 integrin and Par-3, may play a role in HSC emergence and expansion within the AGM. In addition, when mitotic arrest was induced by injecting nocodazole into mouse embryos at E12.5, 2 hrs prior to sacrifice, there were multiple cell division arrests evidenced in the myocardium, which also demonstrated asymmetric Par-3 and 1 integrin expression (FIG. 9). Unfortunately, the endothelium was not arrested in mitosis likely due to differing cell cycle kinetics or possibly the penetration of the drug. The in vitro culture system to evaluate hemogenic endothelium through fate tracing (FIG. 3C) will allow the addition of mitotic agents to the culture media for cell cycle arrest, as has been demonstrated in the epithelium (Lechler and Fuchs, 2005).

### Example 4: β1 Integrin Constructs for Cell Specific Deletion (Prophetic)

### Rationale

Many stem niches share similar principles of stem cell emergence. A parent stem cell polarizes from either contact dependent polarity cues from the niche, or alternatively due to an inherent polarity¹⁷. As a result, a specifically oriented divisional plane is established with disproportional expression of proteins allowing the parent cell to divide asymmetrically, giving rise to a differentiated daughter cell. β1 integrin has been demonstrated to be involved in niche contact dependent polarity within epithelial stem compartments in the mouse²², as well as follicle stem cells in the Drosophila ovary¹⁰⁸. We have demonstrated that β1 integrin, an important molecule in matrix guidance and mechanotransduction of endothelium¹⁰⁹, also plays a critical role in endothelial polarity¹⁸. Cell polarity in the epithelium consists of an ordered apical-basal distribution of polarity proteins, which include polarity complex members Par-3, Par-6 and atypical protein kinase C (aPKC). In endothelium, the loss of β1 integrin results in a loss of Par-3, and subsequent loss of normal endothelial squamous cell shape, which can be rescued with the re-addition of Par-3. When hemogenic endothelium is imaged in the AGM, we notice a distinct polarized expression of Par-3 in daughter (hematopoietic) cells located away from the niche and in direct opposition to the β1 integrin expressing cells in direct contact with the β1 integrin+ endothelial niche (FIG. 3F). Furthermore, when we examine genetic expression within the AGM, as compared to the circulating blood, we find increased expression of β1 integrin, Par-3 and Par-6 (FIG. 3G) suggesting that similar to the mechanism of epithelial asymmetric division, hemogenic endothelium may also divide according to a Par-3-β1 integrin directed polarity.

### Experimental Approach

To understand the role of β1 integrin in the process of hemogenic endothelium, we will induce endothelial deletion of β1 during the hemogenic developmental timeframe.

a) β1 integrin endothelial deletion. Using our tamoxifen inducible VE-cadherin Cre crossed to a β1 integrin floxed line (E3), we will inject animals with tamoxifen, and evaluate the AGM region between E10.5-11.5 as previously described11. We will use the floxed exon 3 (E3, FIG. 3A) line¹¹⁰, as more efficient recombination will be needed, especially in context of an inducible system. We are currently breeding the inducible VE-cadherin Cre line with the floxed β1 integrin (E3) line into both EYFP and LacZ R26R Cre reporter lines to allow for FACS and histological analysis respectively.

b) In vivo induction—Tamoxifen dosage. We may need to change our injection regimen used in the past for fate tracing (1 mg at E9.5) if we do not observe a significant number of cells with Cre excision (as evidenced by the R26R reporter mouse). Thus to begin, we will need to investigate different dosing regimens to maximize Cre excision and tamoxifen kinetics without risking immediate mortality from global β1 integrin loss. We can investigate 1 mg to 2 mg tamoxifen at E9.5 (one time dose), or 1 mg daily from E9.5-10.5, or 2 mg at E9.5/1 mg at E10.5. We will evaluate β-galactosidase (βgal) staining within the AGM endothelium (see below) to measure Cre efficiency with each dose regimen.

c) In vitro induction—β1 integrin deletion will be induced in AGM endothelium (E10.5-11.5) in vitro with 10 μM 4-OHT (for 24 hrs) as described¹¹.

Evaluation of Hematopoiesis by FACS.

By using the EYFP R26R line, we can assess the effect of 1 integrin deletion on the ability of hemogenic endothelium to produce hematopoietic stem cells in vitro. We can compare induced and non-induced AGM organ cultures for hematopoietic cell numbers (EYFP+c-kit+CD41+ and/or CD45+) by FACS.

Gene Expression.

We may also evaluate the phenotype of 1 integrin deleted arterial endothelium by cell sorting EYFP+EphrinB2+ cells and investigating downstream genetic expression. By utilizing differential gene microarray assays, as we have previously employed (FIG. 3E, and above), we can measure genetic expression differences between β1 integrin deleted and non-deleted cells and see if the changes reflect the measured gene effects in mature endothelium (FIG. 3E).

Mitotic Arrest.

After in vitro β1 integrin deletion within AGM organ cultures, mitotic arrest agents can be utilized to allow for visualization of divisional planes²². We plan to add 10 μM nocodazole to AGM organ cultures after 6-48 hrs of tamoxifen induction, and evaluate mitotic arrests 4-5 hrs post nocodazole, as described²². By arresting mitosis at various times after Cre induction and thus β1 integrin deletion, we can delineate the temporal effect of β1 integrin ablation on HSC emergence and the asymmetric divisional plane. However, it may be difficult assessing direction and polarity in vitro as the tissue architecture is not preserved. If we encounter difficulties clearly defining divisional planes within the endothelium and HSCs, we can employ cell culture of AGM vibratome sections within media supplemented agarose, as previously described¹¹¹. As this modified culture system maintains the anatomy of the AGM aortic lumen, we should be able to detect the orientation of divisional planes of hemogenic endothelium.

d) Histological evaluation—We will evaluate the presence or absence of hematopoietic stem cell clusters attached to the dorsal aorta, their cell divisional axis, and location of polarity regulators by immunohistochemical analysis.

β-Galactosidase Staining.

As the inducible system is unlikely to activate Cre expression within the total endothelial population, we will use a R26R Cre LacZ reporter to detect Cre activity (and resultant β1 integrin deletion) on a cellular level. We will stain sections of the AGM for endothelial and hematopoietic β-galactosidase (βgal) labeling as described⁶⁴, and interpret a βgal labeled cell to represent Cre expression in that cell, and likely recombination at the β1 integrin locus. However, Cre expression can occur without the full excision of both β1 integrin loci (if the recombination efficiency of the locus is poor), resulting in a labeled cell that still expresses β1 integrin protein. To determine true loss of protein β1 integrin immunohistochemistry will be conducted.

Immunohistochemistry.

To evaluate protein expression and asymmetric distribution within the vascular niche, we will employ immunohistochemical analysis. Vibratome or paraffin sections of AGM regions will be investigated for β1 integrin (Chemicon), VE-cadherin (ImClone Systems), Par-3 and Par-6 (Santa Cruz), beta-tubulin III (Sigma) and Numb (Upstate). Protocols for all the aforementioned antibodies (except Numb) have already been optimized within our laboratory. For Numb staining we plan on following the previously described protocol¹¹². As we have demonstrated an increase in VE-cadherin, and decrease in Par-3 expression with β1 integrin ablation in mature endothelium (FIGS. 3D and 3E)¹⁸, it will be interesting to evaluate these proteins in the context of hemogenic endothelium. In addition, the recent association between Numb and cadherins in glial cell polarity¹¹² may suggest the existence of a similar association between VEcadherin and Numb in hemogenic endothelium.

### Expected Results

It is unclear if a lack of polarity due to β1 integrin deletion will favor one cell fate over another resulting in overabundance of hematopoietic cells or endothelium. However, we do expect there to be significant effects of β1 integrin ablation on hemogenic endothelium. Furthermore, based on our preliminary findings (FIG. 3F), it is very likely that the AGM functions in a similar manner to other stem cell niches, requiring a defined polarity and asymmetric division to produce blood. We expect that similar proteins involved in other niches—β1 integrin, PAR-3, VE-cadherin and Numb will play a significant role.

### Example 5: Single Cell Resolution of Morphological Changes in Hemogenic Endothelium

### Introduction

Endothelial to hematopoietic transition (EHT) during embryogenesis provides the first long term hematopoietic stem and progenitor cells (HSPC) for the organism. Fate tracing (Zovein et al., 2008), live imaging (Bertrand et al., 2010; Boisset et al., 2010; Eilken et al., 2009), and loss of function studies (Chen et al., 2009) have demonstrated that a subset of endothelial cells, termed hemogenic endothelium, is capable of generating HSPCs, which first appear as rounded cell clusters attached to the endothelium (North et al., 1999). The generation of hematopoietic cells from the endothelium occurs during a narrow window in development (embryonic day (E) 10-12 in mouse (de Bruijn et al., 2000), and ˜4-6 weeks in the human (Tavian et al., 1996)). The most well studied site for HSPC emergence is the developing aorta located in the embryonic aortagonad-mesonephros (AGM) region (de Bruijn et al., 2000; North et al., 1999). Intra-aortic hematopoietic clusters appear transiently in the AGM region, and then are thought to migrate to the fetal liver, and ultimately the bone marrow for long-term adult hematopoiesis. Previous studies have demonstrated a requirement of the transcription factor Runx1 for the transition of endothelial cells to a hematopoietic fate (Chen et al., 2009; North et al., 1999). Runx1 expression is noted within a subset of endothelial cells in hemogenic vascular beds but is then localized to hematopoietic cells as intra-aortic clusters emerge (Tober et al., 2013). The transcription factor Sox17 has also been shown to be important for the generation of hemogenic endothelium (Clarke et al., 2013b), as well as playing a role in HSC survival (Kim et al., 2007). However, while SOX17 promotes hemogenic endothelial specification, continued or overexpression has been noted to inhibit the direct transition to hematopoietic fate (Clarke et al., 2013a; Nobuhisa et al., 2014). The relative protein levels of these two transcription factors (Runx1 and Sox17) during the endothelial to hematopoietic transition (EHT) have not been extensively studied. Herein we present the first report of RUNX1 and SOX17 correlative microscopy analysis in human and murine hemogenic endothelium. The findings illustrate the initiation of hematopoiesis on a single cell level, and elucidate the first events of hematopoietic cell emergence.

### RUNX1 and SOX17 Marks Human Hemogenic Endothelium

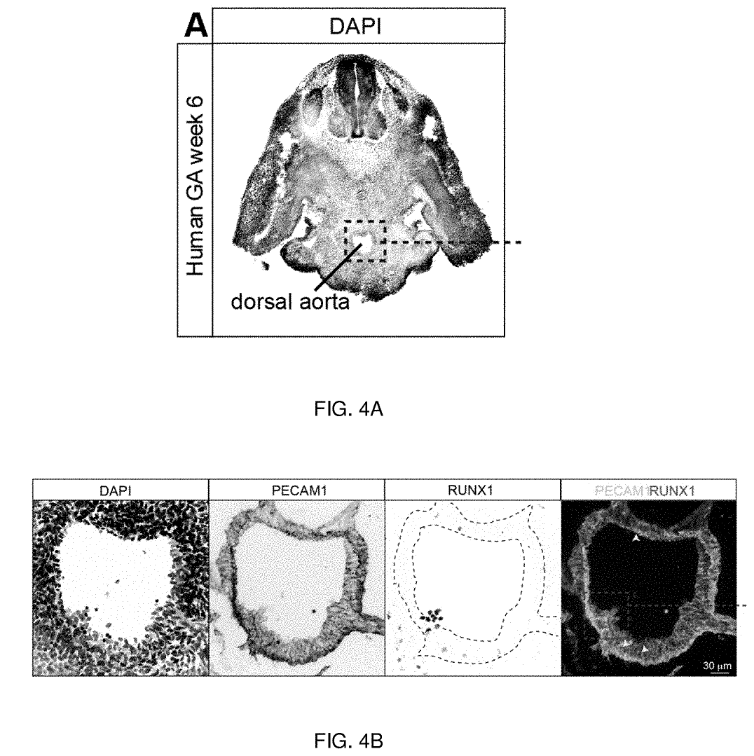

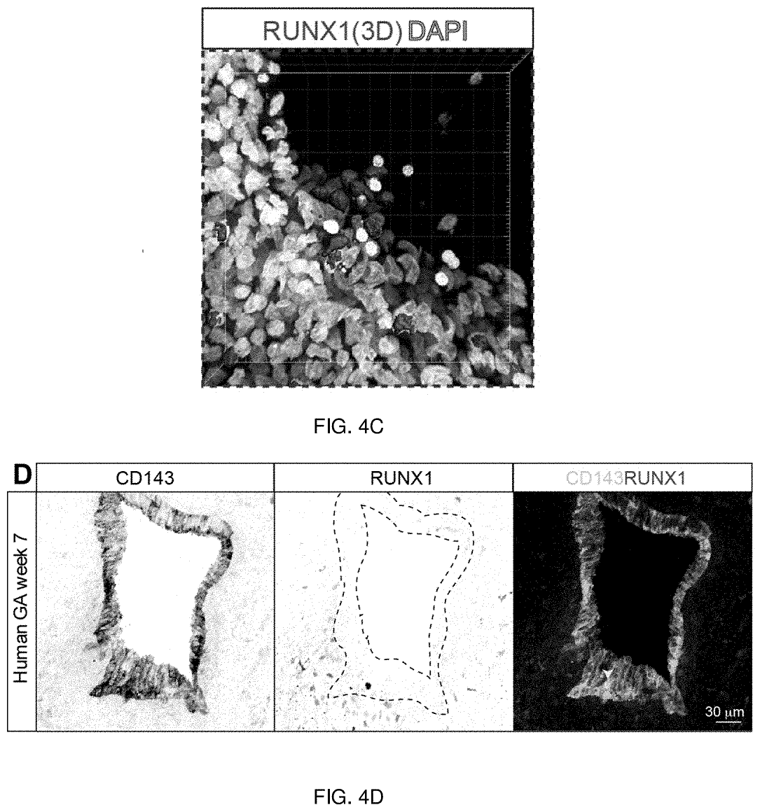

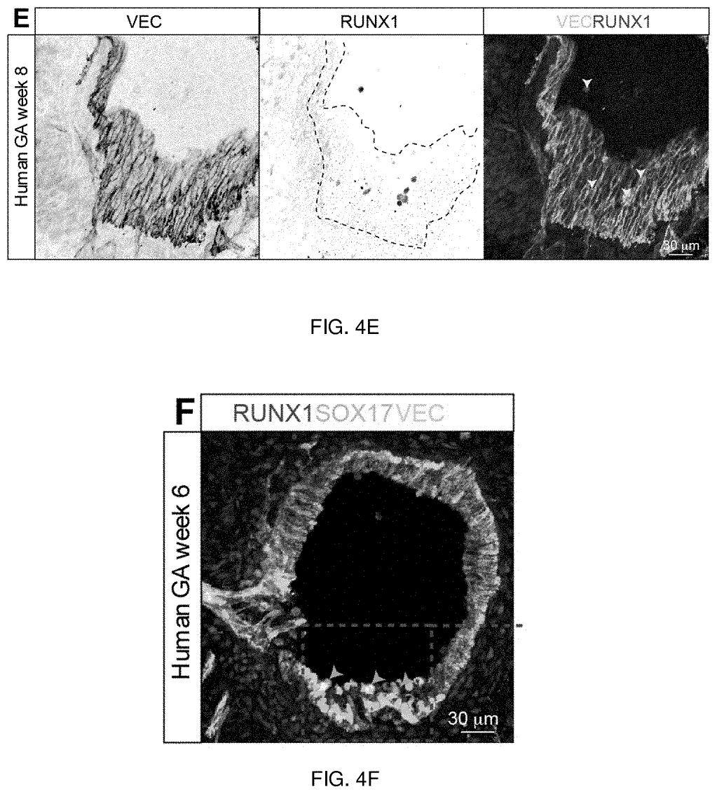

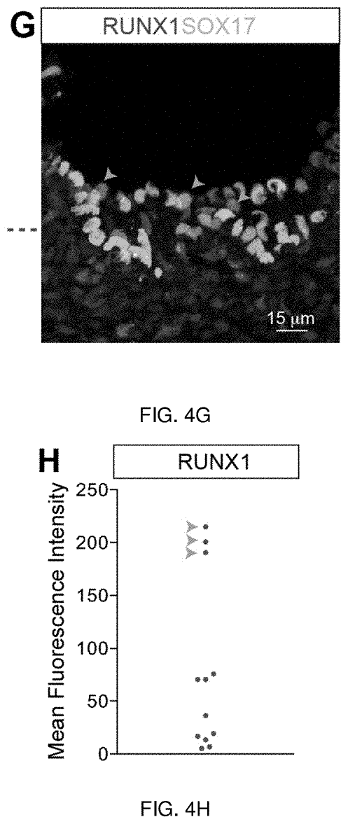

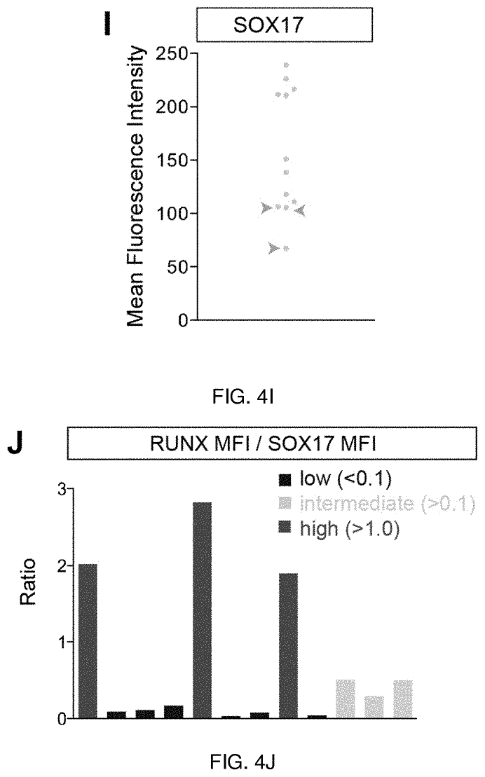

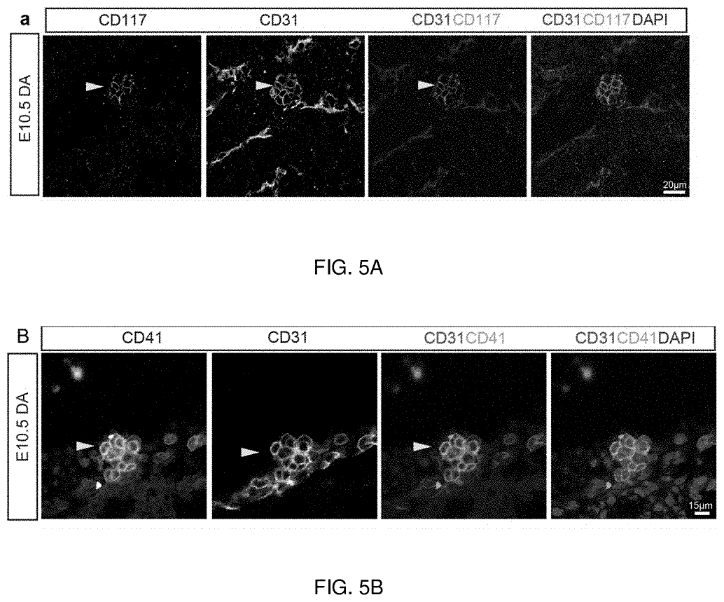

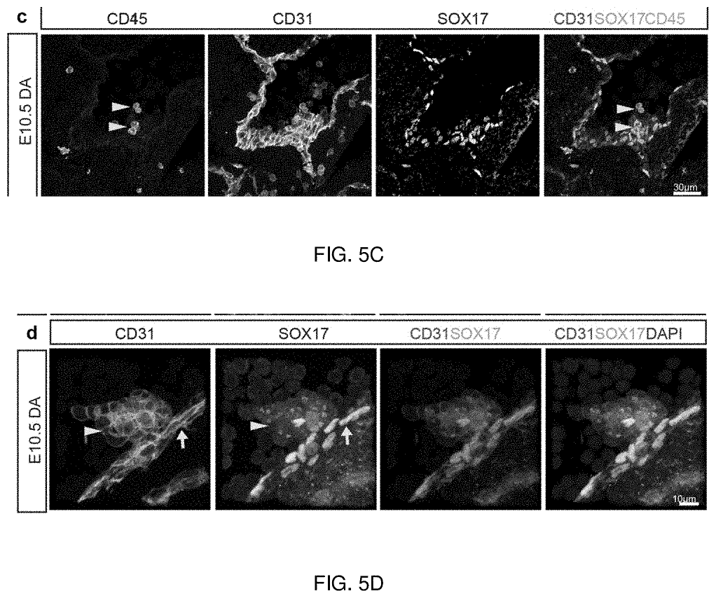

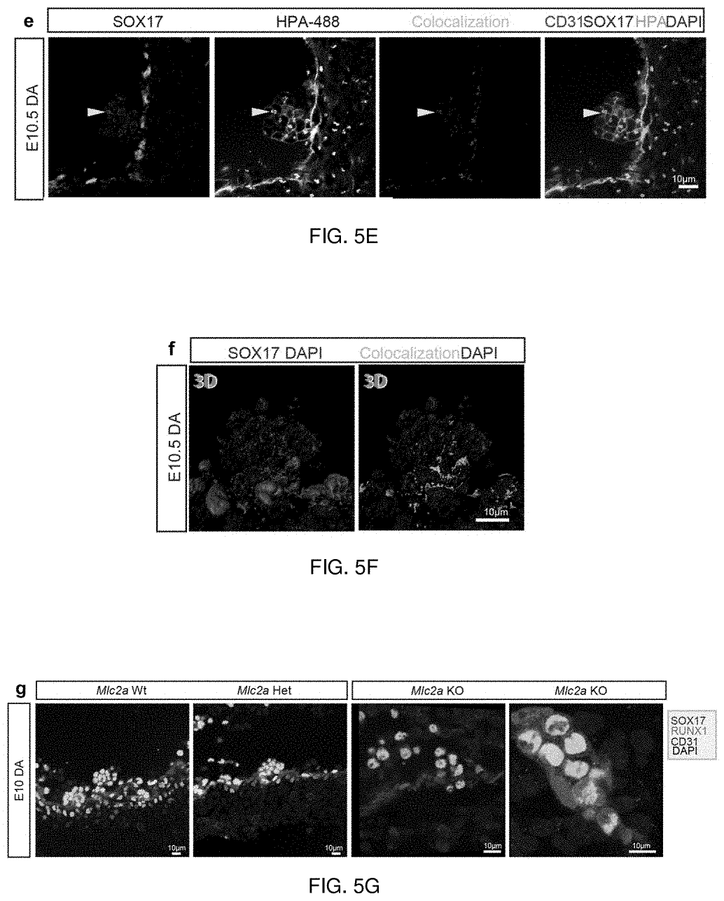

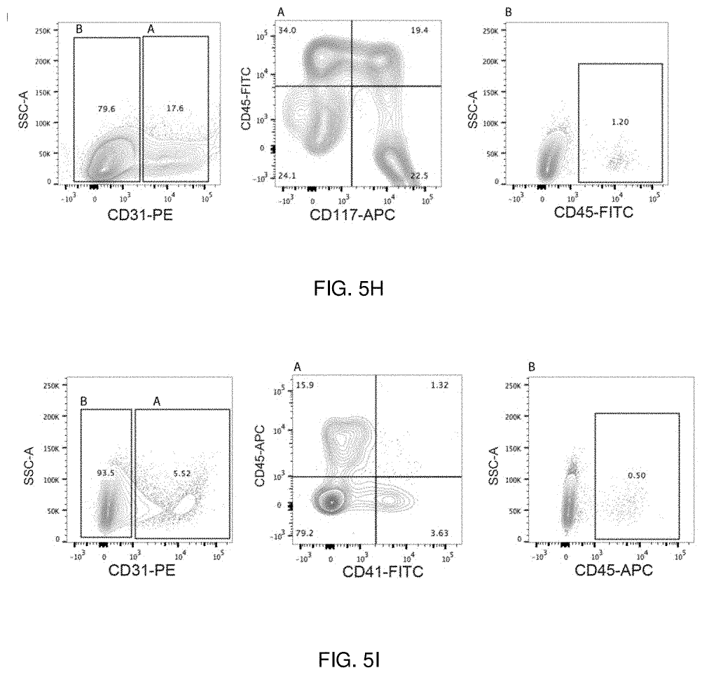

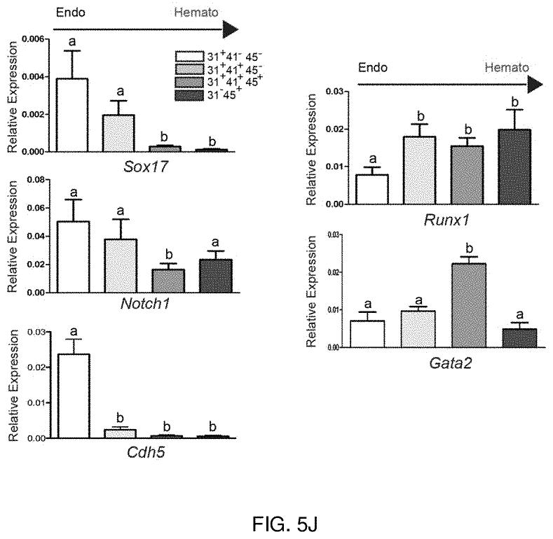

Understanding the basic fundamental aspects of mammalian endothelial to hematopoietic transition (EHT) can provide important information for the generation of hematopoietic cells in vitro. We set out to determine the expression patterns of the putative EHT regulators Sox17 and Runx1 during human embryonic development. First, we evaluated endothelial cells within the human AGM, from 6 to 8 weeks gestational/menstrual age (GA), which corresponds to developmental stages of 4-6 weeks (FIG. 4A and FIG. 5A). Endothelial cells are identified by PECAM-1 and VE-cadherin (VEC), while CD143 (angiotensin-converting enzyme, ACE) has been shown to identify human hemogenic endothelium (Jokubaitis et al., 2008) (FIGS. 4B-4E, FIG. 5B). In addition, since Runx1 has been demonstrated to be critical to EHT in the murine system (Chen et al., 2009), we evaluated levels of the RUNX1 in human hemogenic endothelial cells and hematopoietic cluster cells (FIGS. 4B-4G). RUNX1 in the human system demonstrates high levels within intra-aortic clusters (FIGS. 4B and 4C), but is also present in a small subset of single endothelial cells within the aorta (FIGS. 4D-4G, FIGS. 5C and 5D). In addition, we also observe that SOX17 in the human system is localized to the endothelial cells of the dorsal aorta within the AGM region (FIGS. 4F and 4G; FIGS. 5C and 5D). The localization to arterial endothelium is also observed in another known hemogenic site, the vitelline artery (de Bruijn et al., 2000) (FIGS. 5E and 5F). The single endothelial cells within the aorta that exhibit high RUNX1 immunofluorescence also display minimal detectable levels of SOX17 immunofluorescence (FIGS. 4F and 4G, arrowheads). As SOX17 and RUNX1 appear to have opposing expression domains, we quantified the levels of RUNX1 and SOX17 per individual endothelial cell, and determined the ratio of RUNX1/SOX17 via mean fluorescence intensities (MFI) of 3D rendered nuclear volumes. In our analyses, we were able to specifically visualize and quantify RUNX1/SOX17 ratios per single endothelial cell within the human aorta (FIGS. 4H-4J). Cells with a low ratio (approximately <0.1) are considered mostly endothelial, with undetectable levels of RUNX1 (FIG. 4J, black bars). High RUNX1/SOX17 ratios (>1) suggest either a hemogenic endothelial cell in transition or hematopoietic fate acquisition (FIG. 4J, dark grey bars). Our analysis also reveals a population of endothelial cells that exhibit intermediate ratios, which may present the early stages of EHT (FIG. 4J, light grey bars). Taken together, during human development RUNX1, and separately SOX17, demonstrate high expression in distinct and separate cell populations within the aorta, such that the ratio of RUNX1/SOX17 may predict stages of EHT.

### Single Cell Analysis of Murine Hemogenic Endothelium Reveals Hematopoietic Transition

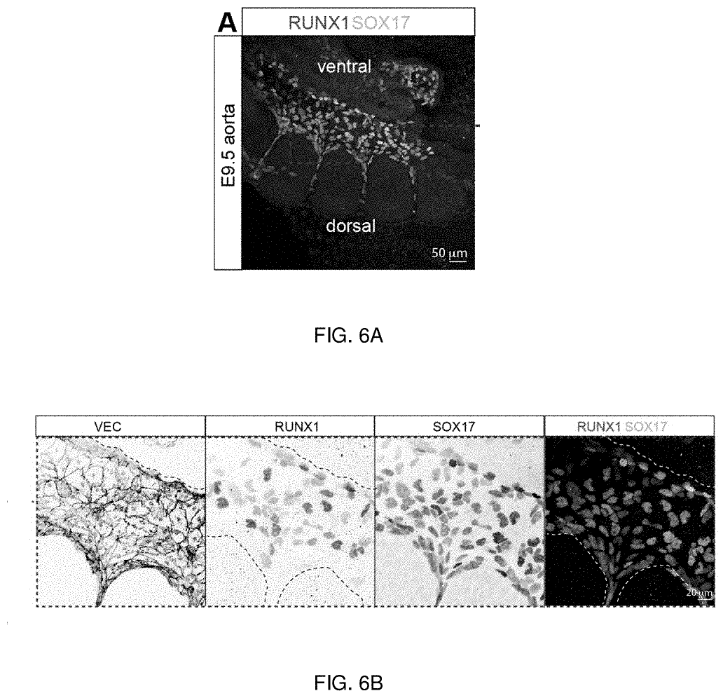

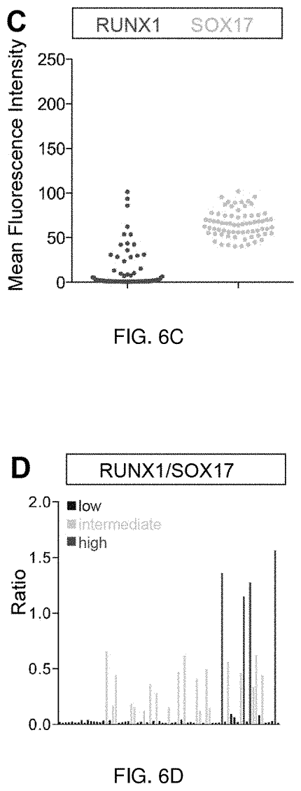

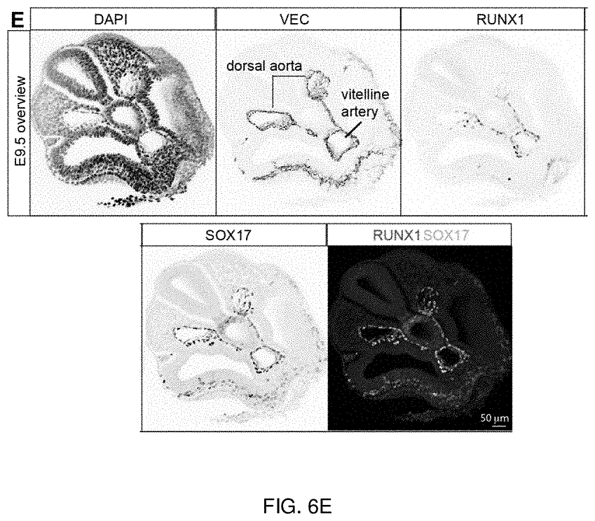

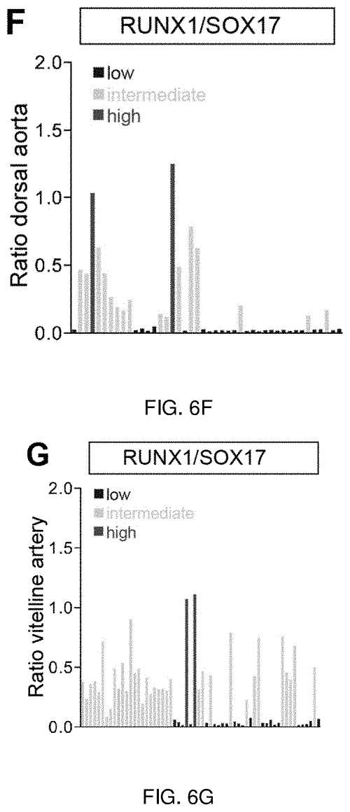

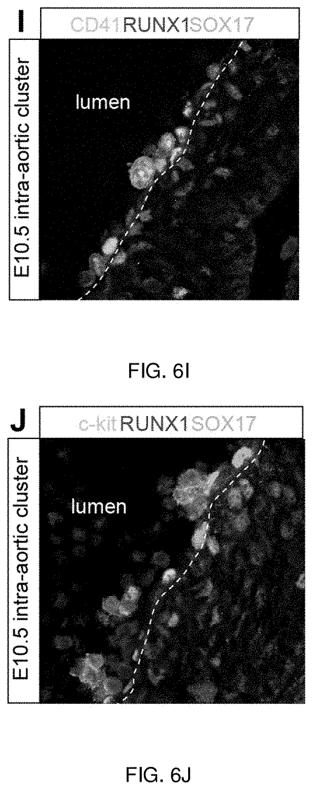

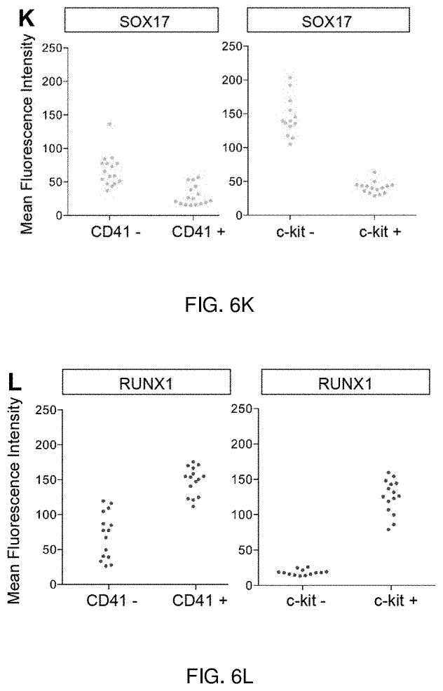

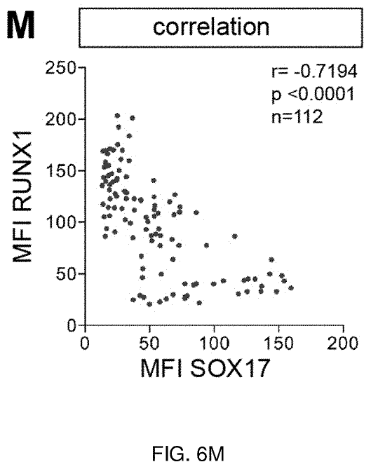

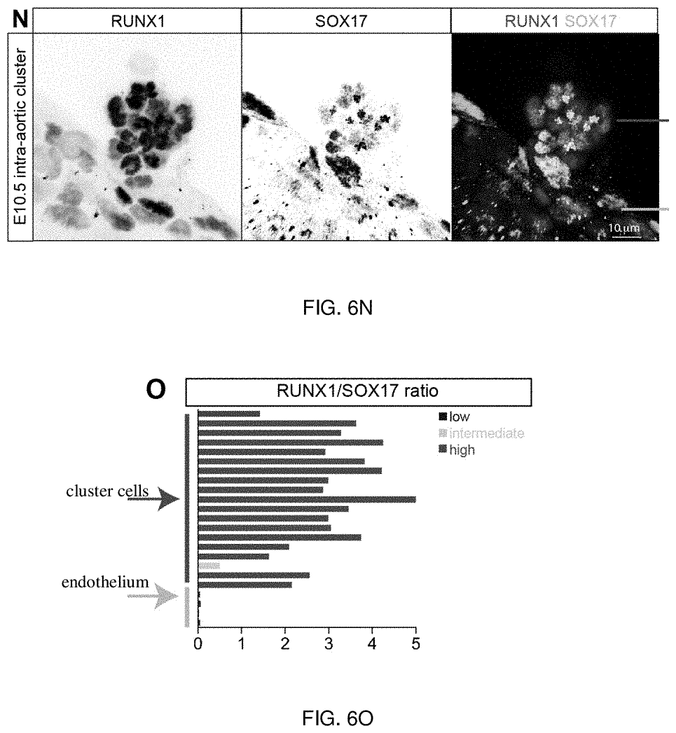

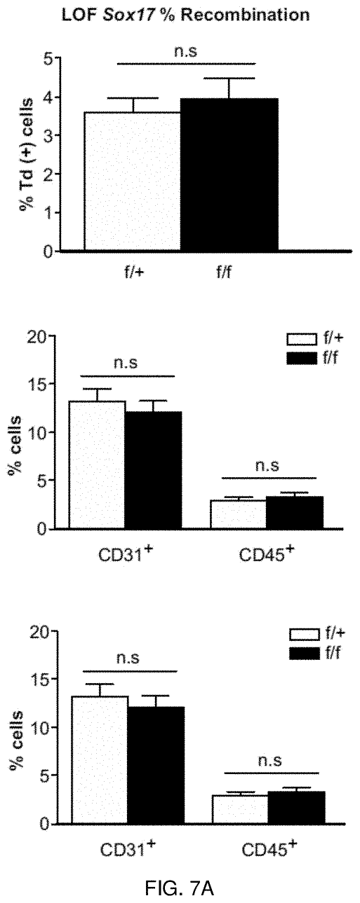

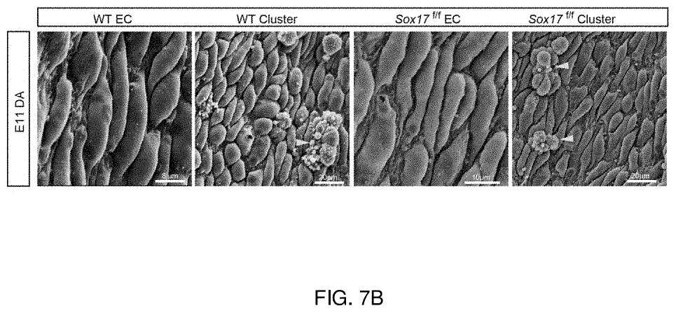

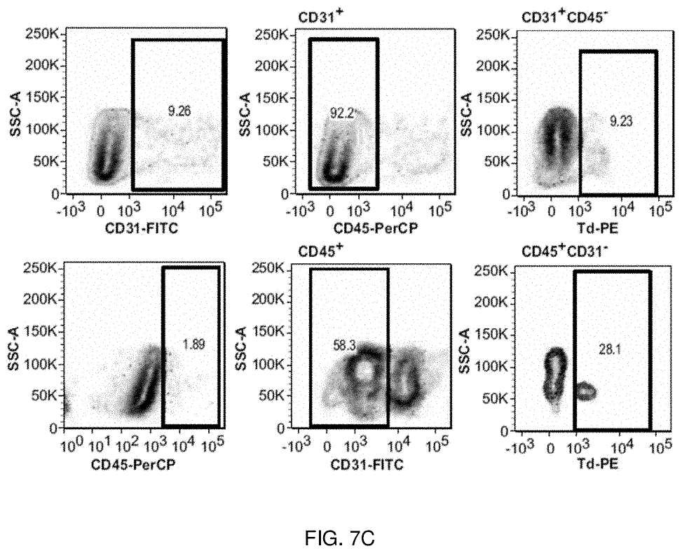

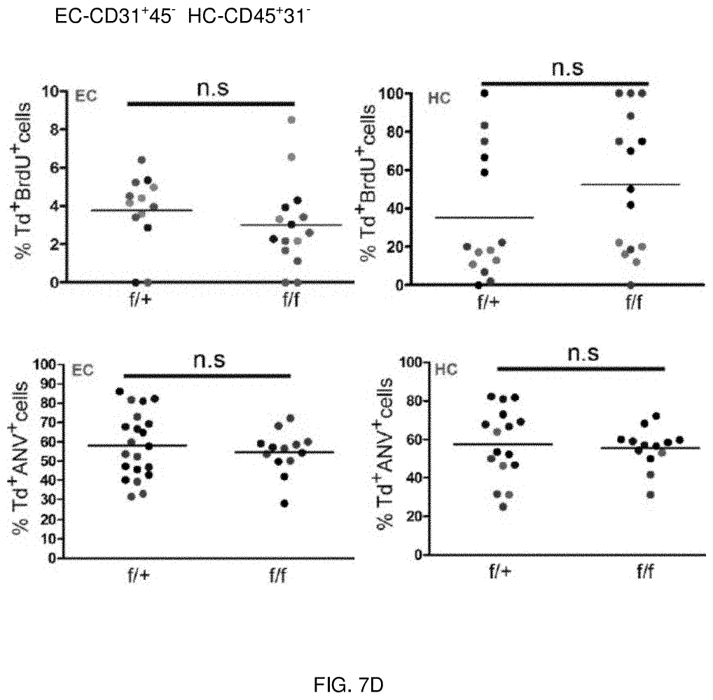

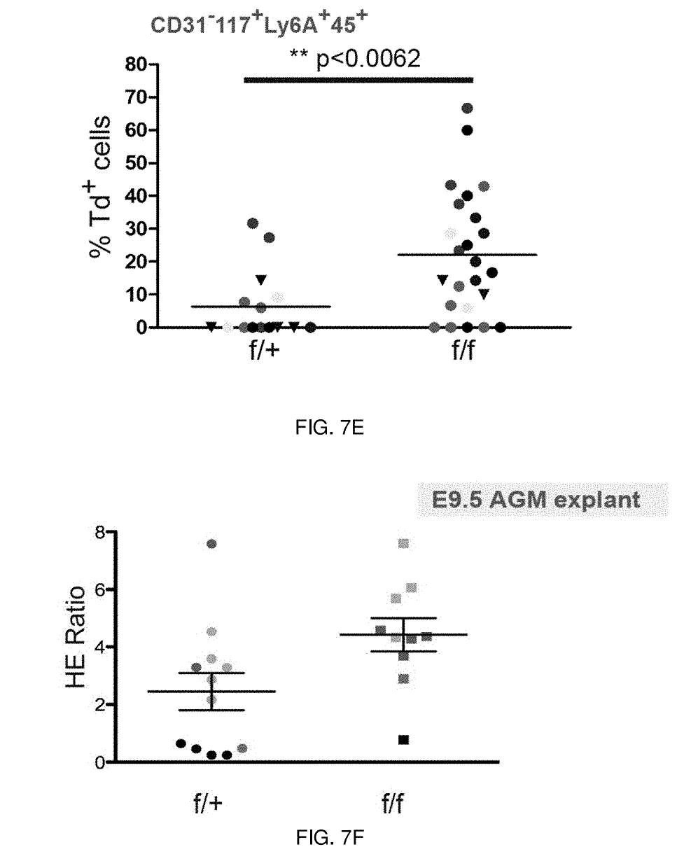

In order to study the developmental stages that span hematopoietic emergence, we expanded our evaluation to include the murine system. To that end, we investigated murine hemogenic endothelial sites prior to the appearance of intra-aortic clusters at E9.5. Endothelial cells exhibited immunofluorescence for VE-cadherin (VEC), SOX17, and RUNX1 (FIG. 6A and FIG. 7A). Immunofluorescence levels of RUNX1 and SOX17 per individual cell were then quantified within the dorsal aorta, and identified a population of endothelial cells with differential SOX17 levels and larger variation in RUNX1 levels (FIG. 6C). Next, we determined the ratio of RUNX1/SOX17 for each individual endothelial cell, and noted a large range of calculated ratios (FIG. 6D), possibly indicating different stages of EHT. We next analyzed two known hemogenic sites, the aorta and vitelline artery, within the same tissue section (FIGS. 6E-6G). Cells with relative high ratios could be identified in the aorta (2 out of 60, FIG. 6F) and vitelline artery (2 out of 44, FIG. 6G), suggesting that in addition to rounded morphology these cells express high nuclear RUNX1 levels with respect to SOX17. The data suggest that early in the hemogenic program, endothelial cells exhibit various levels of RUNX1, which may represent cells early in the hematopoietic transition. To that extent, we also evaluated intra-aortic clusters, a hallmark of EHT, at E10.5 (FIGS. 6I-6O and FIGS. 7F-7I). Cells within intra-aortic clusters can be identified by CD41 (integrin, alpha 2b (Itga2b)) (Robin et al., 2011), and c-kit (Yokomizo and Dzierzak, 2010). Using both CD41 and c-kit as markers of hemogenic endothelium and/or EHT, we evaluated RUNX1 and SOX17 within CD41 positive and negative populations, as well as within c-kit positive and negative populations (FIGS. 6I-6M). Cells identified as c-kit or CD41 positive consistently exhibited high levels of RUNX1 (FIGS. 6I-6L) and low levels of SOX17 (FIGS. 6I-6L). When both positive and negative populations are evaluated, SOX17 and RUNX1 MFIs exhibit a negative correlation (FIG. 6M). Occasionally we observed a primarily non-nuclear localization of SOX17 in subsets of clusters cells (FIG. 6N) suggesting a possible role for this transcription factor in another cellular compartment. The nuclear ratios of RUNX1/SOX17 per individual cell in a cluster exhibited some variability, but a strong tendency towards ratios >1.0 (18 out of 19), consistent with hematopoietic identity (FIG. 6O). The ratios among endothelial cells near the cluster were notably lower (FIG. 6O), possibly signifying non-hemogenic endothelium.

### Correlative Microscopy Reveals Cellular Topography of Hemogenic Endothelium

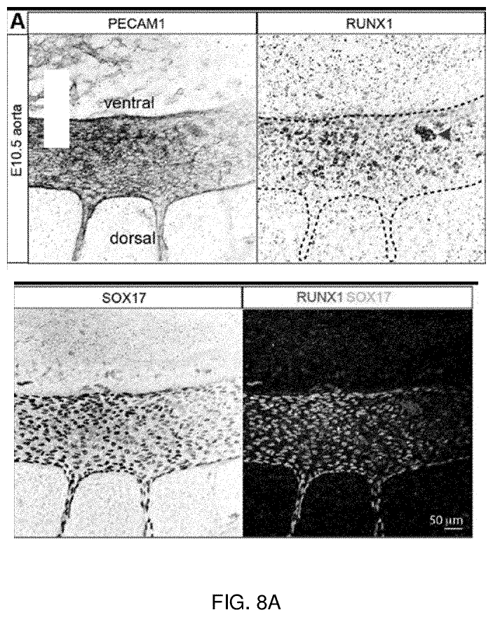

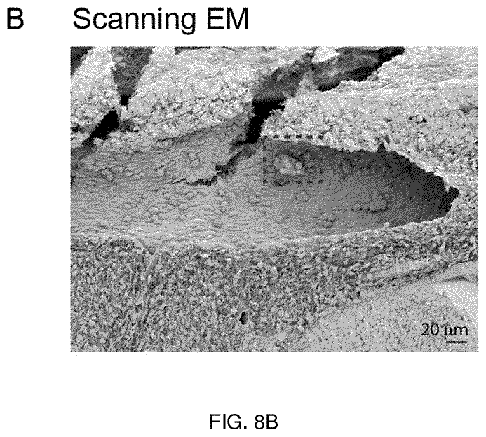

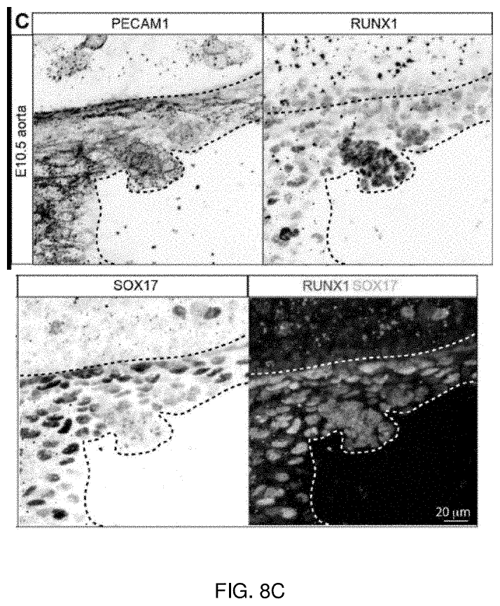

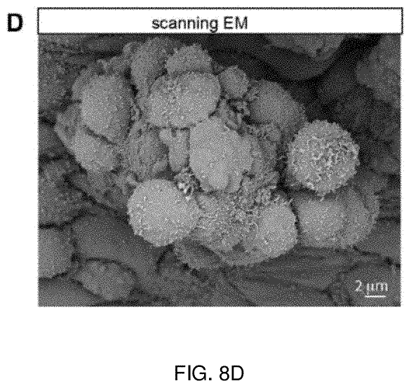

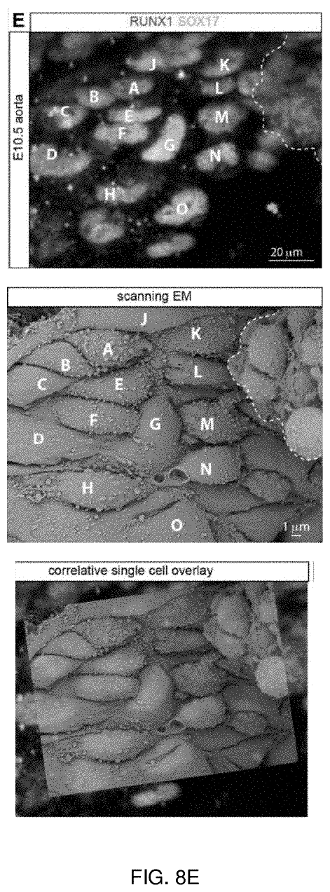

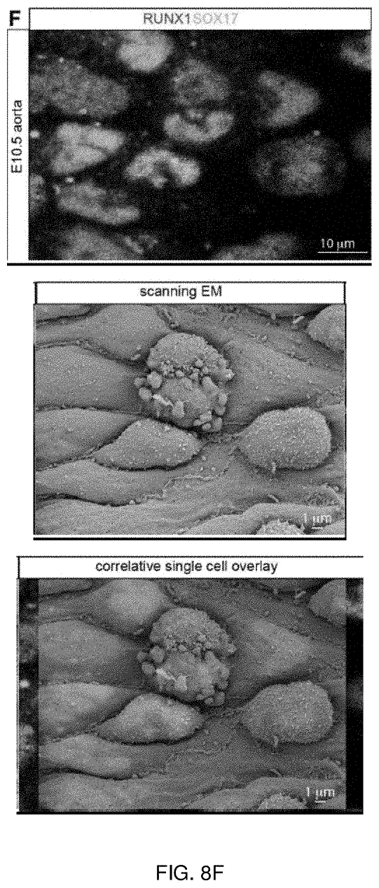

To examine the cellular structure and surface morphology of hemogenic endothelium, immunofluorescence was correlated to scanning electron microscopy (SEM) analysis. Briefly, mouse embryonic vibratome sections were immunostained and imaged, followed processing and evaluation by SEM. The images from both types of microscopic evaluation were correlated via anatomical landmarks, and resulted in a tight overlay of both immunofluorescence and SEM micrographs. Using this strategy, single cells of entire aortas can be evaluated for nuclear levels of RUNX1 and SOX17 with corresponding cellular morphology at ultra-high resolution (FIG. 8A and FIG. 9A). Using this approach, we further investigated the expression of RUNX1 with respect to cellular morphology. Cells within the hemogenic endothelial layer that exhibit nuclear RUNX1 immunofluorescence demonstrate a range of morphological attributes; as compared to endothelial cells that exhibit high SOX17 levels and very low to non-detectable RUNX1 levels (FIGS. 8A-8D, FIGS. 9A-9H). Overall, the correlative approach defines endothelial cells as exhibiting high SOX17 and low RUNX1 levels, with commensurate flat-like cell morphology, and integration into the endothelial layer; with no obvious membrane or cell shape differences (FIGS. 8E and 8F). Strikingly, endothelial cells that exhibit detectable RUNX1 levels demonstrate unique cellular morphology with more oblong and rounded cell bodies and filipodia-like protrusions of the membrane (FIGS. 8E and 8F). Taken together, this correlative microscopy approach allows visualization of aortic endothelium at single cell resolution with corresponding quantification of immunofluorescence levels and high-resolution cell morphology.

### Endothelial to Hematopoietic Transition can be Identified Through Correlative Microscopy

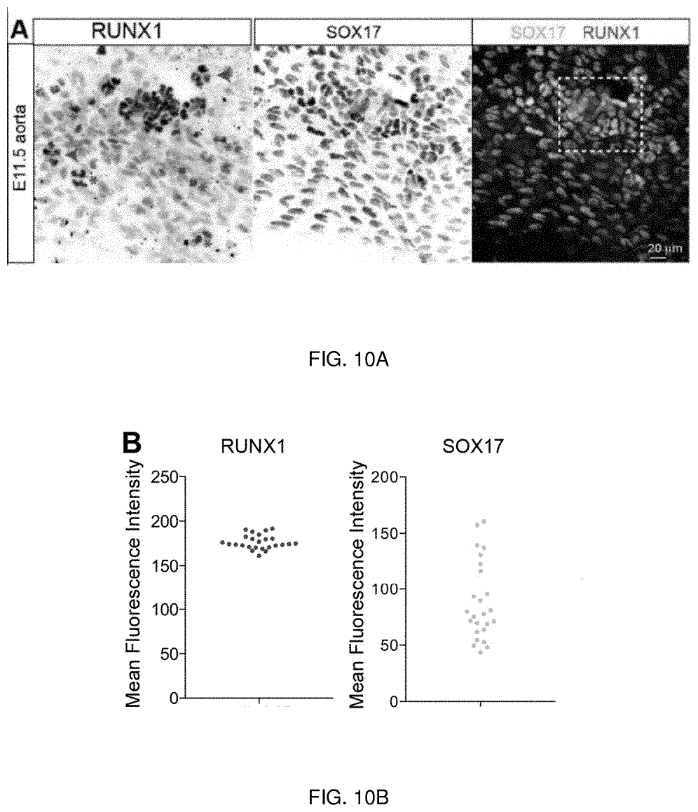

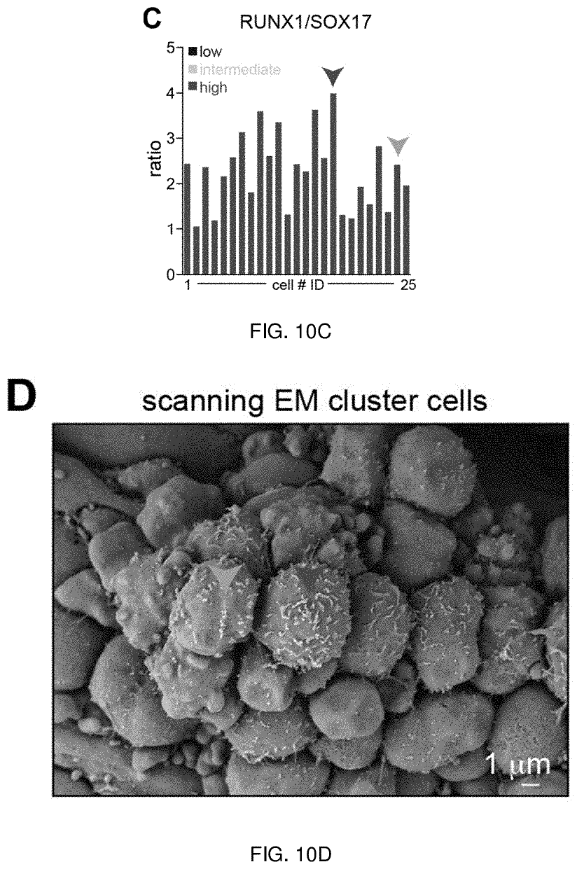

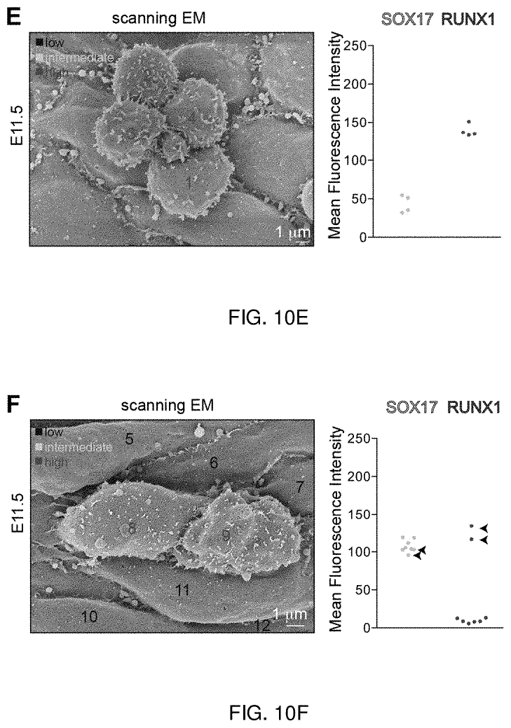

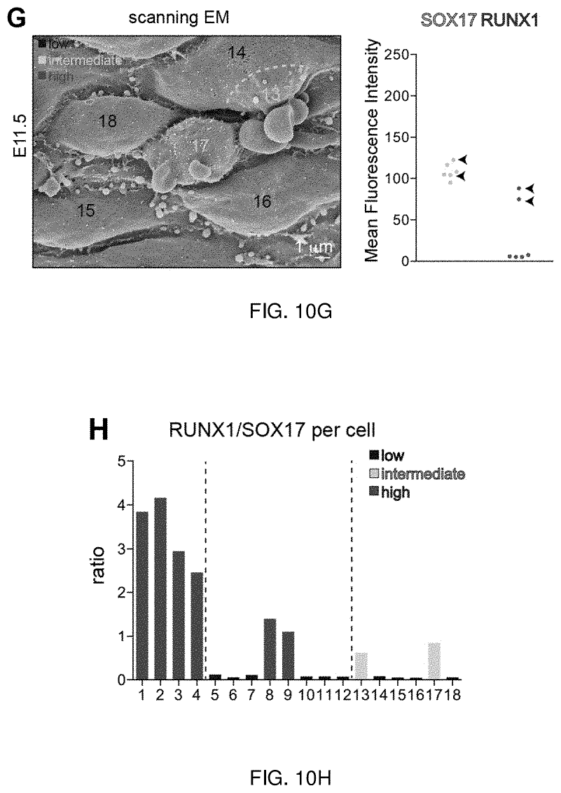

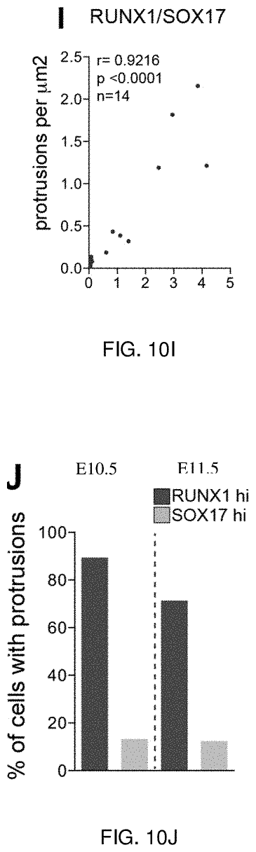

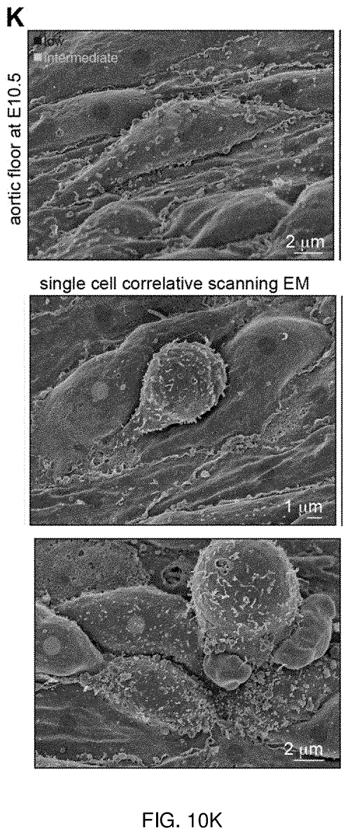

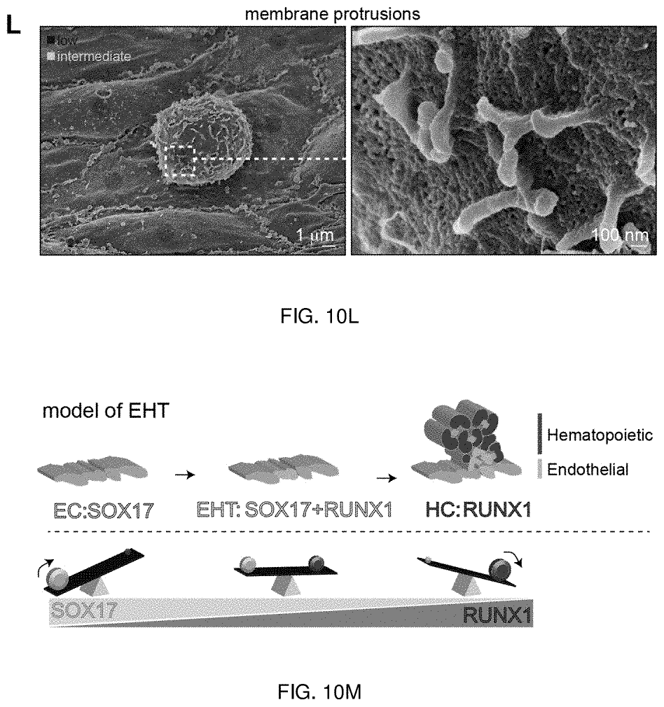

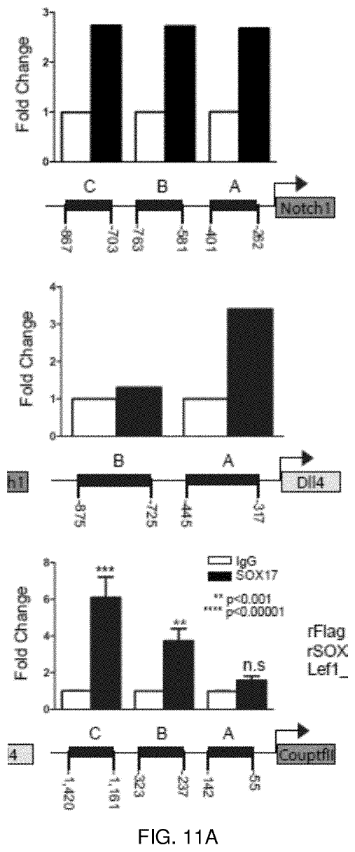

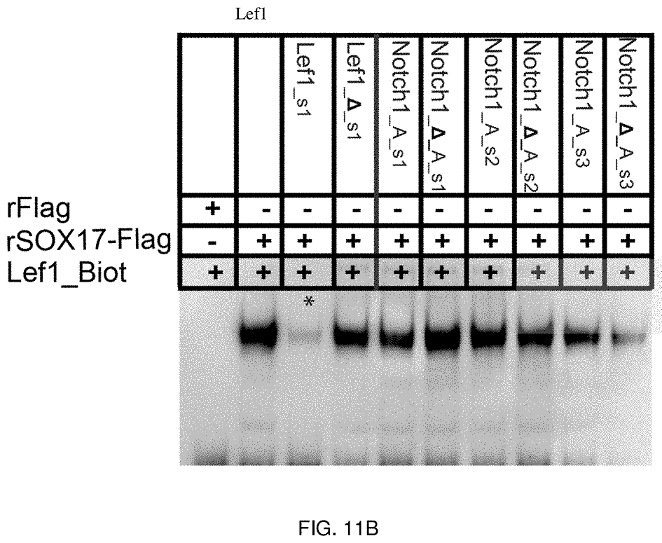

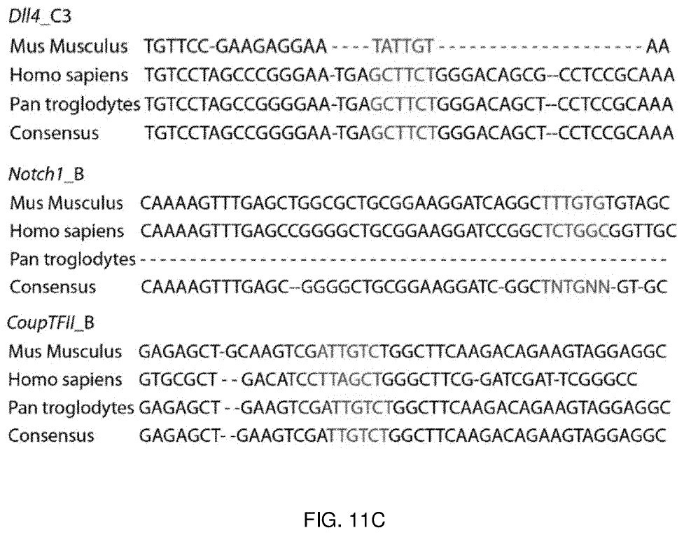

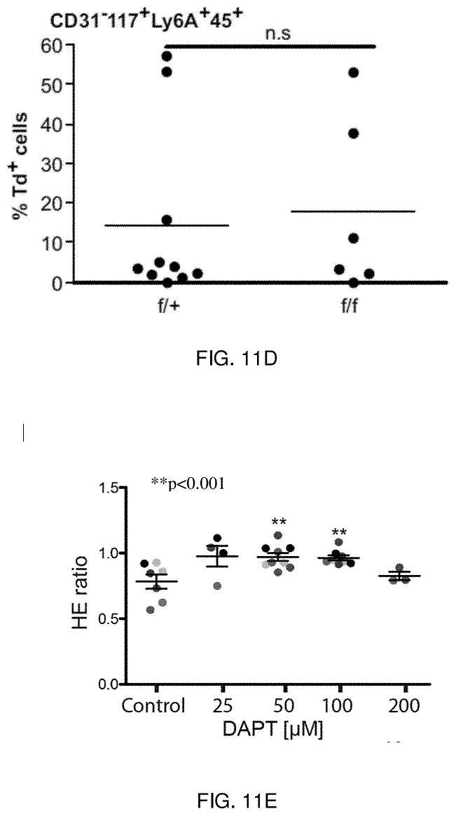

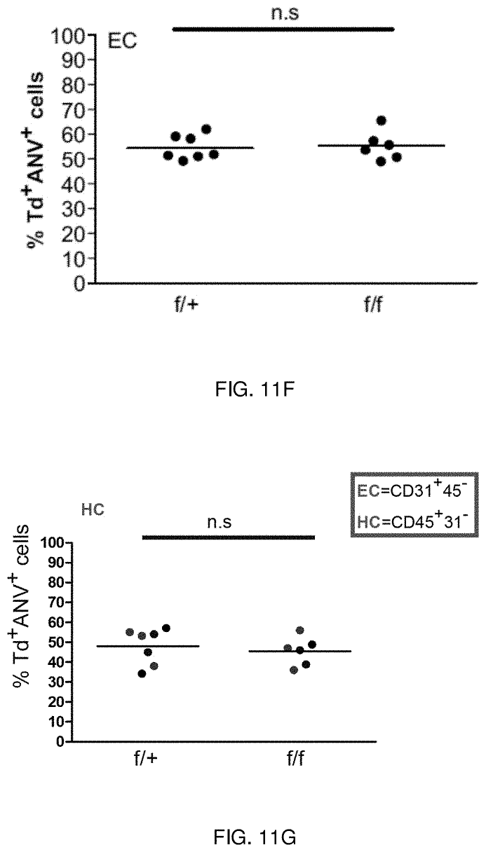

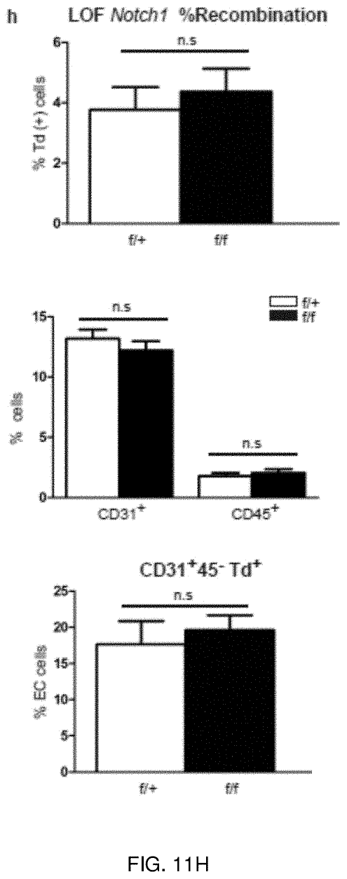

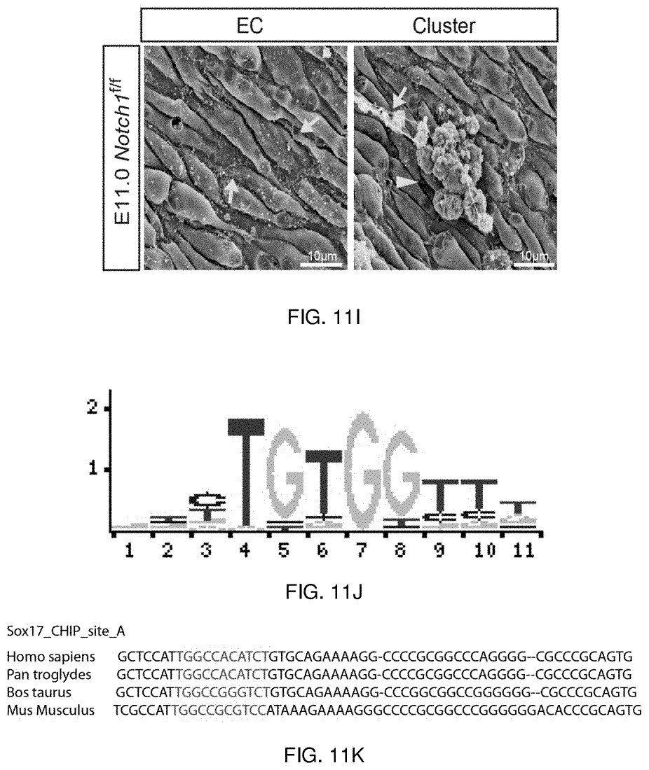

In order to precisely identify single hemogenic cells during all stages of EHT, we analyzed complete aortas on a single cell level via our correlative microscopy approach. Murine aortas at E10.5 and E11.5 were analyzed via correlative microscopy for SOX17 and RUNX1 immunofluorescence (as well as CD41, S4A-E) and cell surface morphology (FIG. 10A and FIG. 11A). Intra-aortic cluster cells were easily identifiable, exhibited high RUNX1/SOX17 ratios and membrane extensions/protrusions (FIGS. 10A-10E, FIG. 11B). Next, we aimed to identify single cells embedded within the endothelium (FIGS. 10F and 10G). These cells exhibit high SOX17 levels, but also detectable levels of RUNX1. Overall, our analysis revealed a heterogeneous population of cells based on RUNX1/SOX17 ratios (FIG. 10H). Cells with little to no RUNX1 exhibit phenotypic endothelial morphology of elongated flattened cell bodies with smoother cell surfaces (FIGS. 10F and 10G). A higher ratio correlated with rounder morphology, and the directly correlated with the presence of membrane protrusions (FIGS. 10F and 10G, FIGS. 10I-10L, FIG. 11H). These protrusions are generally found within the RUNX1+ cell population (FIGS. 10I and 10J). Taken together, this correlative approach allows us to directly study morphological changes with intracellular changes of RUNX1 and SOX17 during endothelial to hematopoietic transition. We also evaluated single cells that exhibited a spectrum of RUNX1/SOX17 levels in order to better elucidate the changes associated with EHT (FIGS. 10K-10L). The data demonstrate that as a cell transitions through EHT (as evidenced by the RUNX1/SOX17 ratio), cell morphological changes occur in association with active changes to the membrane surface.

### Discussion

The prospective identification of endothelial cells with hemogenic capacity remains an important goal towards the ability to generate hematopoietic cells in vitro. In addition, the cellular mechanisms that underlie the process of EHT are still unclear. By employing the novel strategy of correlative scanning electron microscopy, we have defined the early cellular events of EHT at single cell resolution. Our data demonstrate that during the hemogenic window small perturbations in SOX17 levels are accompanied by increased levels of RUNX1 (which appears to precede overt morphological changes) and identify a population of hemogenic endothelium. Once RUNX1 levels are increased with a corresponding decrease in nuclear SOX17 a transition towards hematopoietic fate occurs, as evidenced by rounded cell shape and co-expression of CD41 and/or c-kit. The novel correlative microscopy approach demonstrates previously uncharacterized changes in membrane dynamics. The significance of membrane protrusions throughout the EHT process is unknown. However, similar protrusion-like extensions are observed in immune activation and inflammation (Yamamoto et al., 2015). As inflammation is becoming a more appreciated regulator of HSC emergence (Espin-Palazón et al., 2014; He et al., 2015; Li et al., 2014; Sawamiphak et al., 2014), the observed changes could be due to activation of inflammatory pathways in EHT. Lastly, the observation in the human system that changes in RUNX1 and SOX17 protein levels mirror those seen in the murine system, strongly suggest similar cellular mechanisms take place in human hemogenic endothelium. These findings and novel approach will further help define the changes associated with endothelial to hematopoietic conversion. In addition, we introduce a new method of single cell analysis within tissue/organ and organismal context that is widely applicable to other developmental and cell biological questions.

### Materials and Methods

Tissue collection: Human tissues were collected in accordance with the regulation and approval of Committee on Human Research at the University of California, San Francisco, from elective procedures with informed patient consent in strict compliance with legal and ethical regulations. The Carnegie classification system was used for staging and correlated to gestational/menstrual age (GA).

Animals: Animal protocols were conducted in accordance with University of California at San Francisco Laboratory Animal Research Committee guidelines. Timed pregnancies were dated by vaginal plugs. Wildtype C57Bl/6J animals were evaluated.

Tissue processing for fluorescent microscopy: Embryos were fixed in 2% paraformaldehyde solution overnight and frozen in Tissue-Tek OCT Compound (Sakura Finetek, 4583). 20-30 μm cryosections were obtained (Thermo Scientific Micron, HM550). Slides were dried for 1 hr at room temperature, washed with PBST (0.5% Triton-X100) and incubated in blocking buffer (PBST, 5% donkey serum) for 1 hr. Primary antibodies (for full list of antibodies please see Supplementary table 1) were incubated at room temperature for 6 hrs in blocking buffer. Slides were washed with PBST and incubated with the secondary antibody for 1 hr, washed, stained with 2 ug/ul DAPI and mounted in Vectamount (Vector Laboratories, H-5501). Images were captured on a Leica SPE Confocal Microscope and compiled using ImageJ and Imaris 7.6 (Bitplane; Belfast, UK) software.

Tissue processing and correlative microscopy: Embryos were fixed as above, washed in PBS and embedded in 1% low melting point agarose and then sectioned with a vibratome (Leica VT 100P) at 100-300 μm. Samples were incubated with 1.0% triton in PBS for 1 hr and then immunostained as described above. Images were captured on a Leica SPE Confocal Microscope and/or Zeiss LSM 780 and compiled using ImageJ and Imaris 7.6 (Bitplane; Belfast, UK) software. Following image acquisition, samples were re-fixed in 0.1M sodium cacodylate/1% glutaraldehyde, pH 7.5, for 1 hr, followed by 1 wash of 0.1M sodium cacodylate. Samples were then dehydrated gradually in a series of EtOH (30, 50, 70, 90, 100%). Then, samples were dried using a critical point dryer and sputter coated with 8 nm of Ir labeling, prior to image acquisition on a Zeiss Ultra55 FE-SEM.

Single cell analysis: For single cell analysis images were acquired with optimal z-stack distance ranging from 0.5 to 1.0 μm per stack in 8-bit modus. Image files were analyzed using Imaris 7.6 (Bitplane; Belfast, UK) software. Each individual cell nucleus was volume rendered based on fluorescence signal from DAPI, SOX17 and/or RUNX1 using surface creation algorithm (Imaris 7.6, Bitplane) in order to generate a measurement per channel of fluorescence intensity, and compiled in Excel (Microsoft). Mean fluorescence intensities (MFI) range from 0 to a max of 255. Graphs were generated with Graphpad (Prism). For cells with nuclear SOX17 immunofluorescence, MFIs were measured based on volumes rendered via SOX17. RUNX1 3D nuclear rendering was employed when SOX17 was minimally co-localized with DAPI. Protrusions per cell were measured using FIJI software by determining surface area per cell and total protrusions surface area as percentage of total surface coverage area. Correlation coefficient r was determined by computing X vs Y parameters (ratios vs protrusions and every RUNX1 MFI vs every SOX17 MFI) via non-parametric Spearman correlation in Graphpad (Prism).

Image acquisition and image comparison: Due to inherent variability between microscopes, staining protocols, and developmental stages of the tissue, image files obtained from separate microscopes Zeiss LSM 780 and Leica SP did not undergo cross comparative analyses. Only single cells within a single generated mage file were compared to each other, but not between image files. Comparisons were measured via MFI of RUNX1 and SOX17. The ratio was determined by dividing the MFI of RUNX1 by SOX17

Scanning electron microscopy: Embryos were fixed in 4% glutaraldehyde/4% EM grade formaldehyde then PBS washed and mounted in 4% low melting point agarose and sectioned from 50-200 uM. Tissues were collected and washed in PO4 for 15 min followed by 1% OsO4 (dH2O) for 1 hr room temperature, then washed in dH2O and dehydrated following stepwise increase from 35% to 95% EtOH followed by three washes in 100% EtOH. Slides were then transferred to a critical point dryer and samples mounted on aluminum stubs. Tissues were coated with palladium:gold sputter coat under high vacuum prior to evaluation with a Carl Zeiss Ultra 55 Field Emission Scanning Electron Microscope (Zeiss).

Hematopoietic assays: Td+/CD1117−APC+/CD45−FITC+ DAPI-excluded cells from dissected AGMs of in vivo tamoxifen induced embryos were sorted into IMDM 2% FBS collection medium. For methylcellulose colony formation assay, cells were combined with Methocult medium (Stem Cell Technolgies, M3434) supplemented with 10% IMDM/FBS and plated at 90-100 cells/ML. Colonies were scored at 1 week and picked for excision genotyping. OP9-DL1 T-lyphoid differentiation assay was performed as described. 300 cells from each AGM were sorted onto OP9-DL1s and passaged every 5-7 days for 5 weeks, then analyzed by flow cytometry.

### Example 6: Define the Minimal Number of Factors Required to Manipulate Endothelial Hemogenic Programs for Hematopoietic Production. (Prophetic)

Our overall aim is to reprogram human umbilical arterial cells to become hemogenic. Multiple hemogenic endothelial sites have been implicated in the mouse and human, and include the umbilical arterial endothelium. As human umbilical arterial endothelial cells (HUAECs) are commercially available, and represent endothelium at a developmental time point where patients samples would be readily available (the umbilical cords at newborn deliveries), we plan to investigate whether this endothelium that was once hemogenic earlier in development (at 5 weeks human gestation), can be coaxed into reverting back to a hemogenic phenotype. Multiple candidate genes have been implicated in the hemogenic program, including transcription factors, signaling molecules and growth factors. Using a similar approach of induced pluripotent stem cell (iPS) technology, we can re-introduce a host of factors (separately, or together) into mature arterial endothelium by direct application of molecules and growth factors, and assess their effect This is preferable to deriving hemogenic endothelia directly from embryonic stem cells (ESCs), or iPS cells, as early hemogenicendothelial subsets result in “primitive” hematopoietic cells (HCs) that do not have multi-lineage long term repopulating capacity. Only intra-embryonic endothelium has been associated with “definitive” hematopoiesis.

Initially we must identify the signaling program that are active in hemogenic endothelium, and which of those programs are silenced later in development. In addition, the changes associated with obtaining primary endothelial cultures from ex vivo samples must also be understood. We will apply differential gene expression array and proteomic analyses to murine umbilical arterial endothelium at E10.5 (during its hemogenic activity) and compare it to term (E19.5) umbilical arterial endothelium. As human umbilical arterial endothelium is hemogenic at 5 weeks gestation, a time before most women know they are pregnant, it precludes us from obtaining sufficient samples from this gestational age. However, we will evaluate human term umbilical arterial samples from analysis, and when available, will obtain umbilical cords at earlier gestations from terminations (6-8 weeks).

Patient samples will undergo endothelial isolation by standard protocols, gene array profiling and proteomic analysis will be done using affymetrix gene array and nano-LC MS/MS analyses and run against appropriate databases. In addition, after 2-3 passages, primary cell lines from patient samples will undergo similar analyses, as well as commercial lots of HUAECs. Lastly, murine umbilical arterial endothelium will be pooled and isolated by cell sorting for the same downstream analyses at E10.5 and at term. From the arrays, a list of related genes and proteins that are significantly up-regulated in hemogenic umbilical endothelium, and those alternatively high expressed in the term umbilical endothelium will be investigated.

Candidate factors gleaned from the genomic and proteomic profiling will be chosen based on expression levels, endothelial specificity, and fold change between hemogenic and term endothelium. Of those programs, we will construct lentiviral vectors of plasmids constructed to over-express pro-hemogenic factors, as well as plasmid targeted siRNAs to silence genes that may be actively suppressing the hemogenic program. From the proteomic data, we will also test pathways that can be activated through addition of recombinant proteins and/or growth factors. HUAEC cultures will then be evaluated for HSC production after lentiviral induction, and/or addition of recombinant proteins by FACS analysis of CD45+ (hematopoietic) cells. A combinatorial approach of gene silencing, transcription factor introduction, and recombinant proteins will be achieved through a similar framework that was employed for the identification of the four pluripotent genes from 25 candidates. Each of our chosen candidates will be introduced separately, then together, and with progressive withdrawal of single factors narrow the pool.

We expect to define a subset of specific factors that are active in hemogenic endothelium, and others expressed only in mature endothelium. The studies will determine the minimal combination of factors with reprogramming ability in mature HUAECs to allow for hematopoietic emergence.

### Example 7: Identify New Factors Regulating the Hemogenic Program (Prophetic)

We aim to exploit the unique characteristics of infantile hemangiomas to uncover new regulatory factors that may play a role in hemogenic endothelium. Hemangiomas have on occasion been reported to produce hematopoietic cells in situ. The endothelia that populate hemangiomas have been shown to exhibit placental vascular markers (a known hemogenic vascular bed) leading to one hypothesis that hemangiomas are placental derived. Other curious attributes of hemangiomas include their transitory nature and self-resolution, clonal origins, and endothelial “progenitor” phenotype. The hemangiomas environment is highly secretory, where multiple cytokines and growth factors have been implicated. Support cells have been shown to secrete large amounts of VEGF resulting in high levels VEGFR2 endothelial signaling. Hemangioma endothelial cells have also been shown to exhibit increased Notch1 expression, a pathway downstream of VEGF thought to regulate Runx1 (a critical hematopoietic transcription factors) in the hemogenic program. Hence, Hemangiomas may be primed for hematopoiesis due to their hemogenic endothelial phenotype and environmental exposure to high VEGF levels.

Patient samples from infantile hemangiomas removed at 3-6 months of age will undergo endothelial isolation by standard protocols, and primary endothelial cultures (HemECs) will be derived from genetic analysis. In addition, whole tumor explants will be cultured as described and the conditioned media evaluated from secreted proteins that may enhance hematopoietic emergence. Lastly, augmentation of known pathways up-regulated in hemangiomas, as well as novel factors from out analyses, will be investigated for hemogenic induction.

HenEC cultures will be treated with various concentrations of VEGF and/or augmented Notch activity with soluble Notch ligands: Jagged-1 and/or Dll-4, and undergo downstream analysis of HSC production by FACS of CD45+ (hematopoietic) cells. In addition, conditioned media from tumor explants will also be added to HemEC cultures for HSC production.

Hemangioma endothelium will undergo gene expression array profiling directly following endothelial isolation, after HemEC culture derivation, and after treatment with conditioned tumor media (versus VEGF and/or Notch signaling up-regulation), to delineate the host of genes that permit hemogenic capacity. The hemangioma “secretome” will be evaluated by analyzing tumor conditioned media via iTRAQ labeling and LC Maldi MS/MS as described, to identify a host of pro-hemogenic protein candidates.

HemEC cultures will be treated with candidate secretory proteins from the secretome analysis for hemogenic induction. In addition, candidate novel genes that appear up-regulated during hemogenic induction will be introduced via lentiviral vectors into HUAECs lines, and candidate secretory proteins screened form the ability to induce hematopoietic emergence in a non-hemangioma endothelial line.

We expect that HemEC lines will be induced to produce HSCs by a select subset of secretory proteins and in addition, we will identify novel genes that regulate the hemogenic process. These hemogenic regulatory genes when combined with a pro-hemogenic protein profile will induce hematopoietic emergence in other endothelial subtypes.

### Example 8: EHT Regulation and Through Functional Analyses

Changes in cell fate and identity are essential for endothelial-to-hematopoietic transition (EHT), an embryonic process that generates the first adult populations of hematopoietic stem cells (HSCs) from hemogenic endothelial cells. Dissecting EHT regulation is a critical step towards production of in vitro derived HSCs. Until this proposed experimentation, we did not know how distinct endothelial and hematopoietic fates are parsed during the transition. Herein, temporally regulated genetic loss-of-function studies show that genes required for arterial identity function later to repress hematopoietic fate. Loss of arterial genes (Sox17 and Notch1) during EHT results in increased production of hematopoietic cells due to loss of Sox17-mediated repression of hematopoietic transcription factors (Runx1, Gata2). However, the increase in EHT can be abrogated by increased Notch signaling. These findings demonstrate for the first time that the endothelial-hematopoietic fate switch is actively repressed in a population of endothelial cells, and that de-repression (dedifferentiation) of these programs augments hematopoietic output.

The first hematopoietic stem cells (HSCs) emerge in the embryo from a specialized subset of endothelial cells, collectively termed hemogenic endothelium (HE). The concept of endothelial-derived HSCs has broad clinical implications as it may open new avenues for in vitro blood production. However, the hemogenic capacity of the endothelium is transient and its precise regulation remains unknown. During a narrow developmental time period (approximately embryonic day (E)10-12 in the mouse (1,2) and 4-6 weeks in the human3), hemogenic endothelial cells acquire cell morphology and gene expression consistent with hematopoietic identity, in a process called endothelial to hematopoietic transition (EHT) (4-6). In the mammalian system, the “hemogenic window” is short lived and typified by groups (or clusters) of rounded cells that are observed within the vascular wall. The hematopoietic cell clusters have been demonstrated to contain both hematopoietic stem and progenitor cells (HSPCs) (7,8). Regions known to harbor hemogenic endothelium include the aorta-gonadomesonephros region (AGM)1,9-12, vitelline and umbilical arteries (9,13,14) yolk sac (15,16), placenta (17,18) and others (19,20) but generally encompass arterial vascular beds, as opposed to veins or capillaries (21). Interestingly, regulators of arterial fate including the transcription factor Sox17 and specification is unclear. Here we present data that demonstrates after artery-vein specification, Sox17 actively prevents the transition to hematopoietic fate by repression of key hematopoietic transcription factors, thereby maintaining endothelial identity. The loss of Sox17 promotes hematopoietic conversion, and its dynamic expression imparts a previously unappreciated, but critical step, in endothelial to hematopoietic cell fate transition.

Sox 17 and Notch1 are implicated in hematopoietic emergence from HE, as early loss of either results in hematopoietic defects (24,25). Sox17 positively regulates Notch1 for both arterial fate acquisition and hemogenic endothelial specification (22,26). How these arterial fate specifiers function in endothelial to hematopoietic conversion, separate from their role in artery-vein

### Results

Hematopoietic Clusters and Endothelial Cells Exhibit Contrasting Gene Expression Patterns.

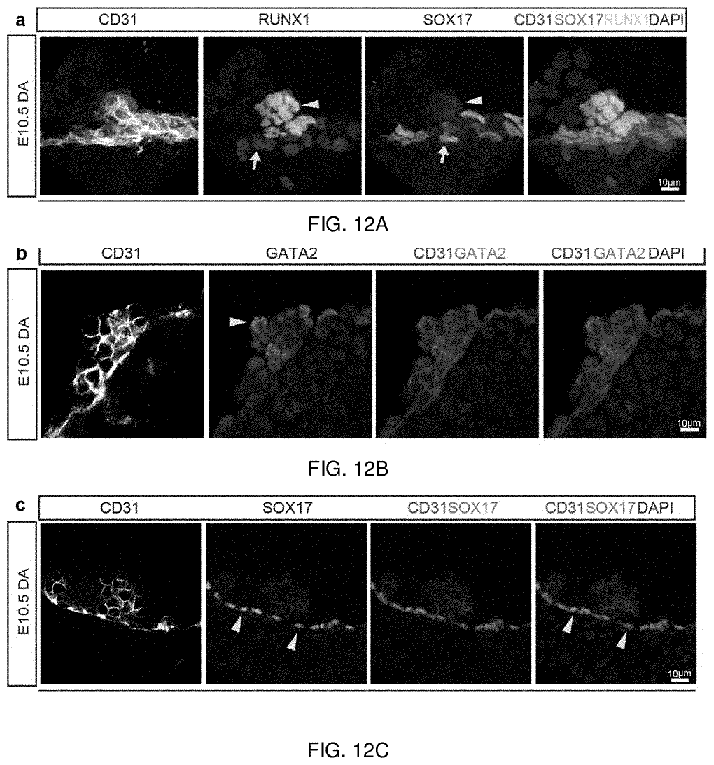

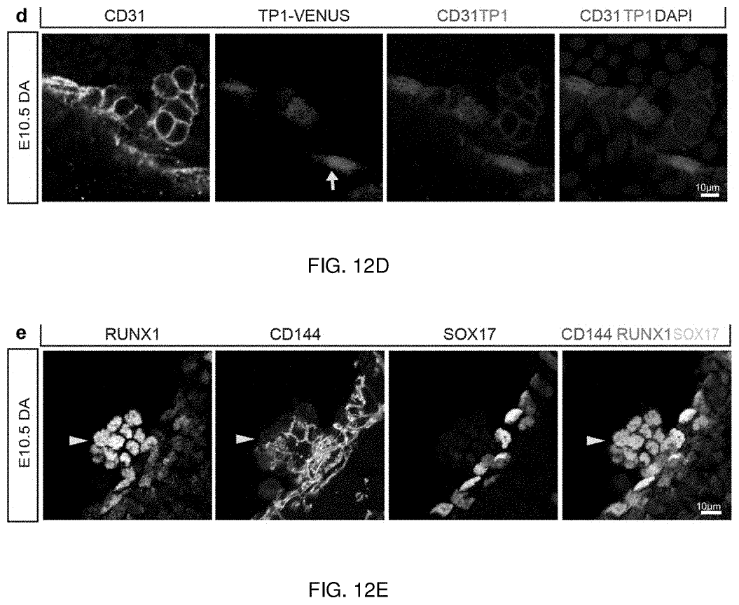

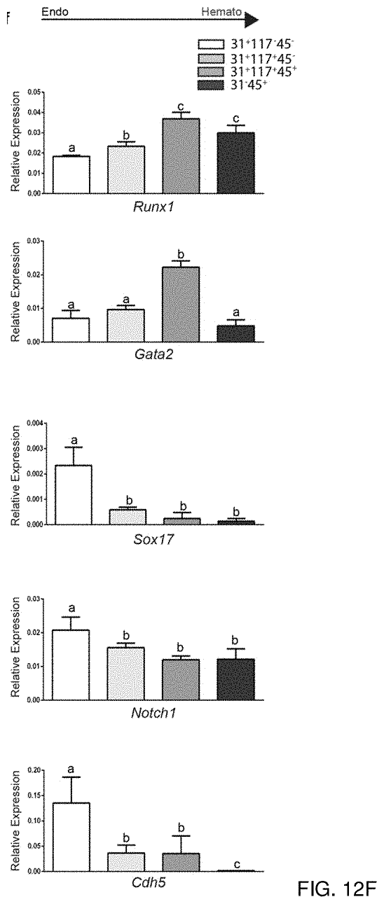

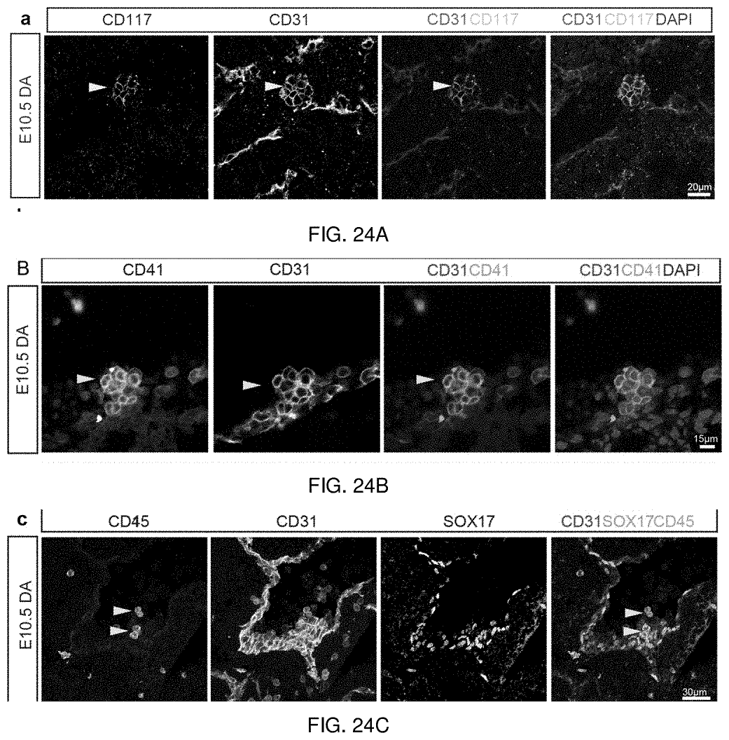

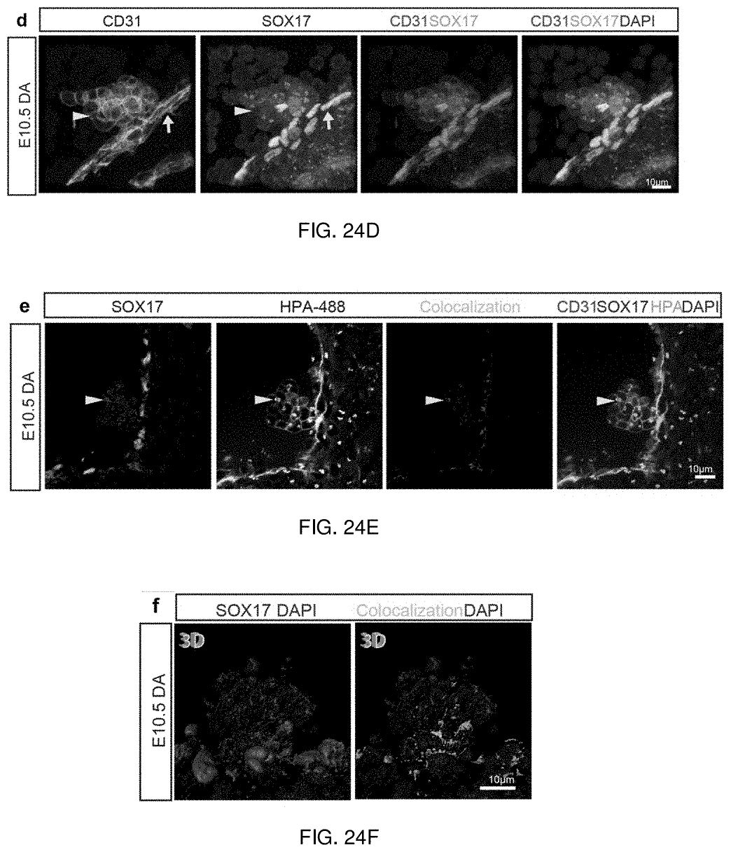

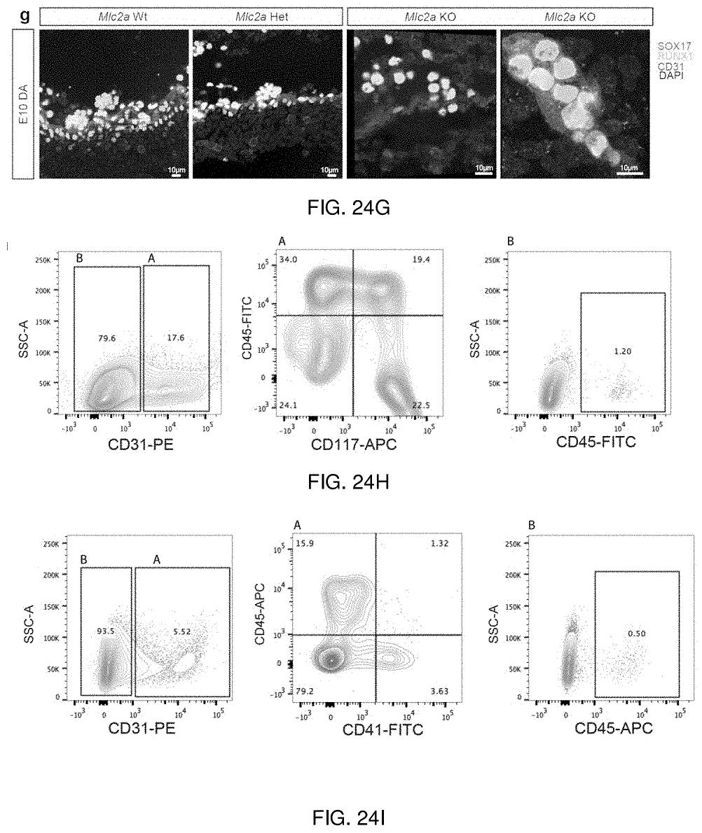

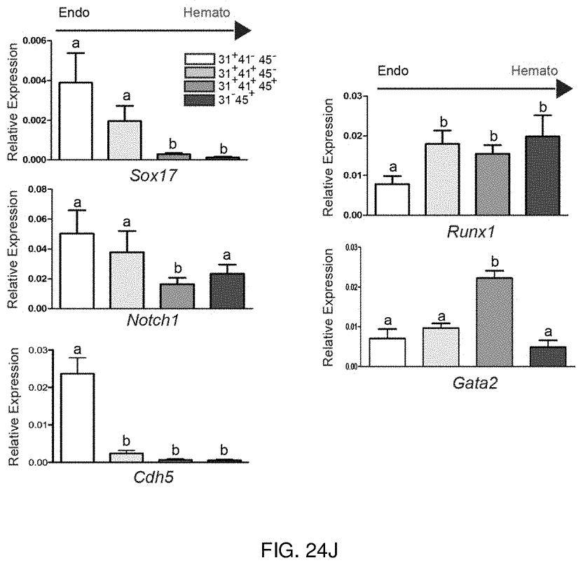

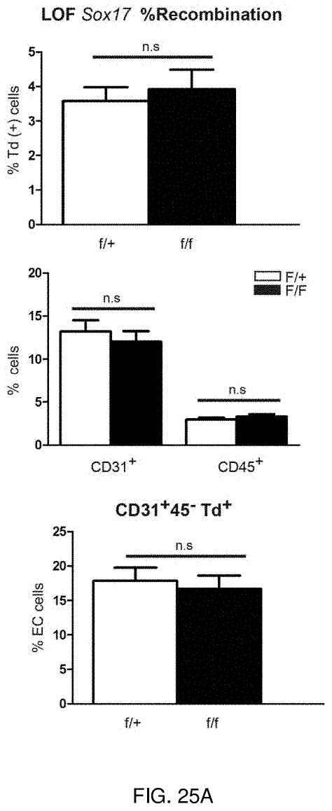

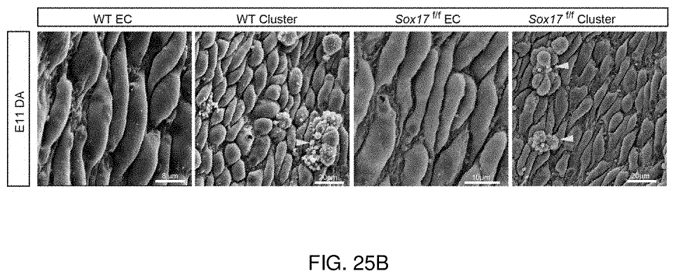

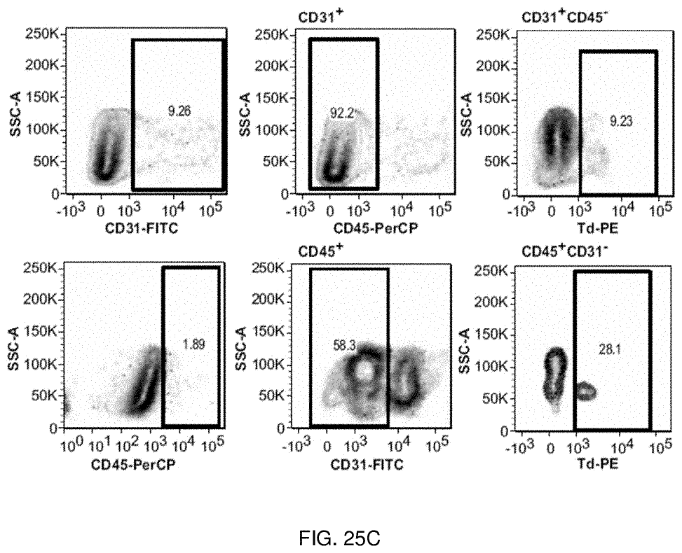

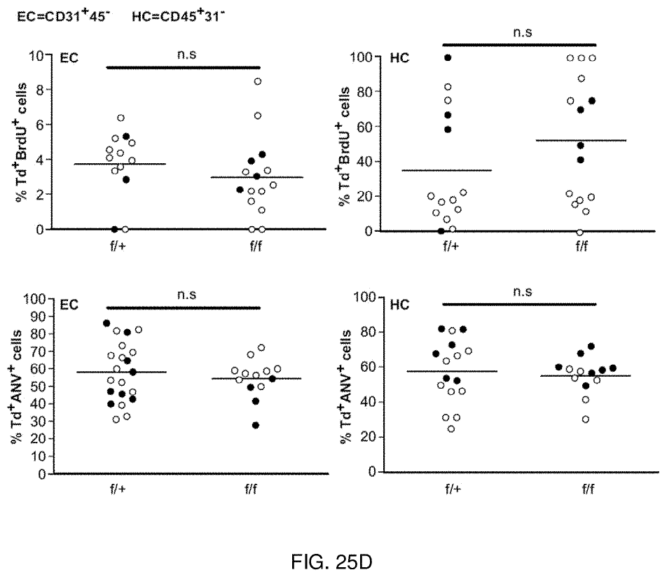

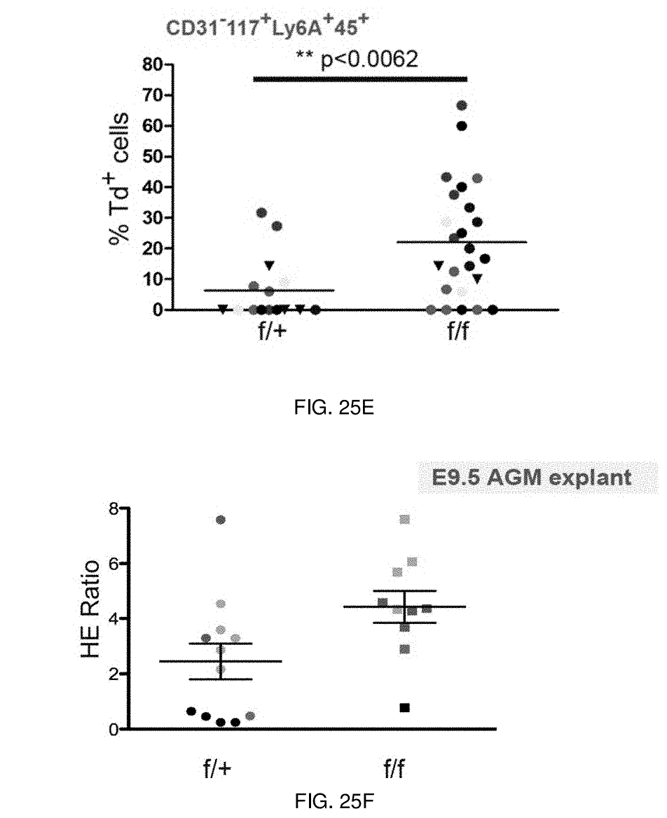

We first evaluated the expression patterns of Sox17, Notch1, Runx1, and Gata2 in the embryonic dorsal aorta (AGM) as all four factors are shown to be required for hematopoietic stem cell emergence. The endothelium of this region can be identified by immunofluorescence of the pan-endothelial cell surface marker PECAM-1 (CD31), and HSPC clusters are easily apparent through their rounded morphology and shared endothelial marker expression (FIG. 12a-d). RUNX127 and GATA228, two transcription factors known to be required for HSPC emergence from hemogenic endothelium, are localized to HSPC clusters, as compared to the adjacent endothelium (FIG. 12a, b). When known regulators of the arterial program including notch signaling (23,29) (visualized by the TP1-Venus reporter mouse line (30,31) and SOX1722 are evaluated, immunofluorescence is localized to the endothelium and not the HSPC clusters (FIG. 12c, d). The appearance of HSPC clusters along the aortic wall is coincident with changes in cell surface marker expression, as cluster cells acquire c-Kit (CD117) (7,32,33) and CD41 (34,35) markers (FIG. 12 a, b), in addition to maintaining endothelial markers CD31 and VE-cadherin (CD144) (36), FIG. 12a-e. Eventually HSPCs also acquire CD45, a pan hematopoietic surface marker (FIG. 24C). Sox17 expression is largely undetectable in cluster cells, but rarely can be seen in a perinuclear pattern with co-expression of golgi markers (FIG. 25E-25F), suggesting it no longer functions as a transcription factor in the cluster cell population. As arterial markers can be flow sensitive (37-39), we also evaluated the expression patterns of SOX17 and RUNX1 in Mcl2a−/− circulation mutants40 (FIG. 24G), and found the segregation of SOX17 immunofluorescence to the endothelium and RUNX1 to hematopoietic cell clusters is preserved. The differential expression of surface markers allows for separation of endothelial and hematopoietic populations, as well as HSPC clusters (CD31+CD117+), by fluorescent activated cell sorting (FACS) (FIG. 24H, 24I). Transcriptional analyses of sorted populations demonstrate that endothelial subsets (CD31+CD117−CD45−) exhibit lower Runx1 and Gata2 transcript levels when compared to HSPC cluster populations (CD31+CD117+CD45−), or as compared to differentiated hematopoietic cells (CD31−CD45+) (FIG. 12f). In contrast, genes associated with arterial identity (Sox17 and Notch1) are decreased in HSPC clusters as compared to the endothelium. Sox17 (26,41) and Notch1 (24,42-45) are known to be important for hemogenic endothelial specification. Thus, the finding that their transcripts and protein levels are actually decreased in HSPC clusters is intriguing. The same trend is also observed when we identify hemogenic cluster cells with the marker CD41 (34,35) (FIG. 24B, 24I, 24J). Together the data suggest that endothelial to hematopoietic fate conversion may require downregulation of critical arterial genes.

Sox17 Negatively Regulates Hematopoietic Fate.

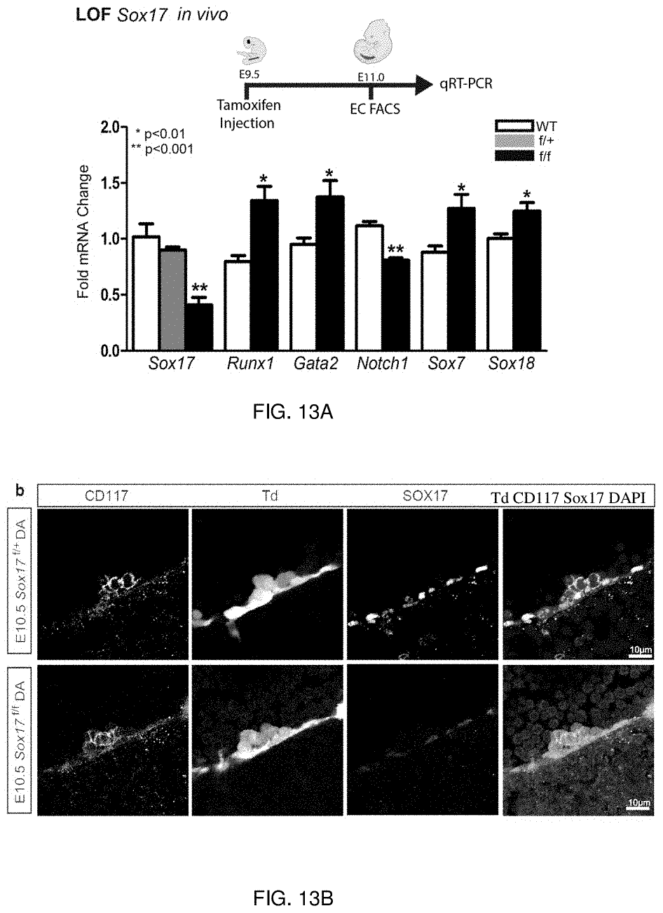

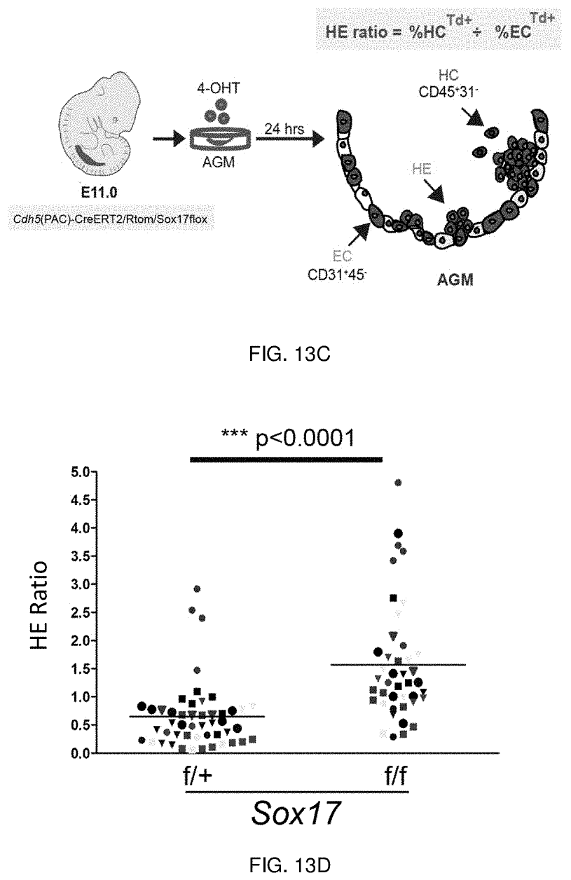

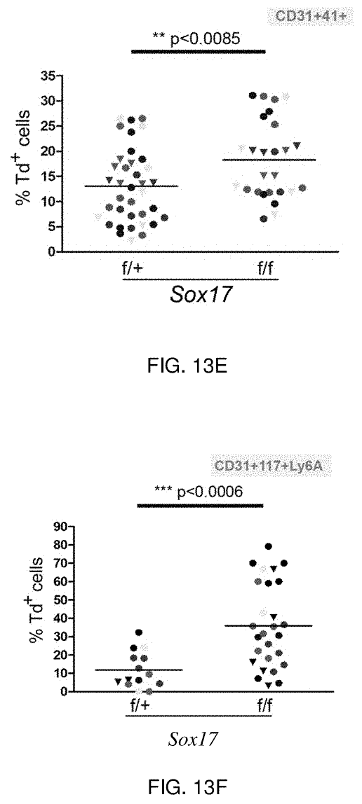

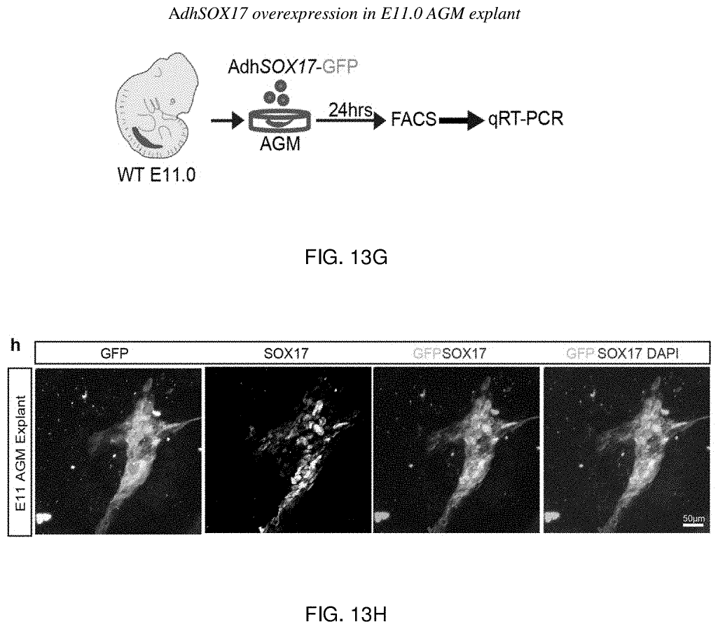

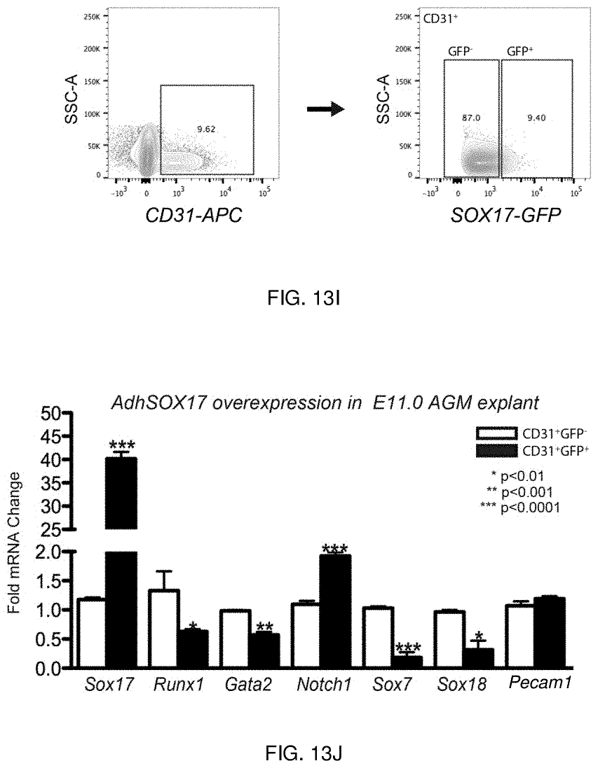

To evaluate the impact of Sox17 on EHT, we undertook both loss and gain-of-function approaches. In vivo endothelial genetic deletion of Sox17 during EHT (induction at E9.5, evaluation at E11, FIG. 13a) was evaluated using a endothelial specific Cre recombinase (Cdh5(PAC)-CreERT246) mouse line crossed to a Sox17 floxed line25 with a ROSA26Cre Reporter (47) (RTom, tdtomato, Td+). The induction strategy is similar to that used in fate tracing studies (48) and allows for timing of Sox17 endothelial recombination early in the hemogenic window and during EHT. Transcript analysis of sorted endothelial cells after in vivo induction uncovered a significant increase in Runx1 and Gata2, two hematopoietic transcription factors known to be critical for HSC development during EHT (27,49,50) (FIG. 13a). Notch1 transcripts are also notably decreased (FIG. 13a), in agreement with previous studies that show Sox17 positively regulates the Notch pathway (22,26). In addition other members of the SoxF family (Sox7 and Sox18) were increased, possibly due to a compensatory response (FIG. 13a). There were no observed differences in endothelial labeling or cell number across homozygous Sox17f/f, heterozygous Sox17f/+, or control animals (FIG. 25A). Immunohistochemical analysis demonstrates the presence of HSPC clusters in the aorta with a marked decrease of endothelial SOX17 in Sox17f/f mutants (FIG. 13b). Also, we did not observe any obvious changes in endothelial morphology as evaluated by scanning electron microscopy (FIG. 25B).

Currently it is not possible to predict which specific endothelial cell within a hemogenic vascular bed will transition to a hematopoietic fate. Also not known is whether endothelial cells comprising the same hemogenic site are all capable of EHT. So whether the actual cell fate conversion is a stochastic event or a predetermined fate change remains to be seen. To circumvent the current obstacles of EHT prediction, we adopted a fate tracing strategy⁴⁸ that allows measurement of traced hematopoietic cell populations from labeled endothelial precursors within a specific hemogenic vascular site. By inducing endothelial recombination of Sox17 in AGM explants using the Cdh5(PAC)-CreERT2/RTom/Sox17flox transgenic mouse line, the number of EHT derived hematopoietic cells can be quantified through fate mapping (FIG. 13c, and FIG. 25C). Tamoxifen induction in vitro with the active metabolite 4-hydroxytamoxifen (4-OHT) at E11.0 allows immediate ablation in AGM explants during EHT, and the calculation of a HE ratio which we define as traced hematopoietic cells (HCs) compared to traced endothelial cells (ECs). Using this assay to temporally and conditionally ablate Sox17, we demonstrate that timed loss of endothelial Sox17 promotes conversion to hematopoietic cell fate in situ (FIG. 13c-f). Sox17f/f mutants exhibit a significant 3-fold increase in HE ratios indicating increased hematopoietic output, in addition to significantly increased labeled HSPC populations (CD31+CD41+Td+) and pre-HSC populations (CD31+CD117+Sca1+Td+), FIG. 13c-f. The observed increase in HE ratios and HSPC number is not due to proliferation effects (as measured by BrdU incorporation, FIG. 25D), nor is the higher HE ratio due to changes in cell death (Annexin-V staining, supplementary FIG. 25D). We also observe increases in maturing hematopoietic populations (CD31−CD117+Sca1+Td+), FIG. 25E. In addition, when a similar strategy is applied to earlier explants (E9.5) prior to hematopoietic cell cluster emergence, we observe similar trends in the HE ratio (FIG. 25F). So while Sox17 has been shown to be critical for HE specification prior to EHT, the loss of Sox17 actually promotes hematopoietic fate over endothelial fate during EHT. To further evaluate the role of Sox17 in this process, we undertook gain-of-function studies in wildtype AGM explants using adenoviral-mediated overexpression of human Sox17 (AdhSox17-GFP), FIG. 13g. GFP expression in explants overlapped with SOX17 co-staining (FIG. 13h), allowing for cell sorting of AGM endothelial cells (CD31+) that were either successfully infected (GFP+) or not infected (GFP−) by AdhSox17-GFP, FIG. 13i. Transcript analysis of endothelial cells with Sox17 overexpression demonstrates significant increases in Sox17 and Notch1 transcripts with significant reduction in Runx1, Gata2, Sox7 and Sox18 transcripts, FIG. 13j. The data altogether suggest that Sox17 negatively regulates hematopoietic fate through repression of Runx1 and Gata2. We also show the known positive regulation of Notch1 by Sox17, and regulation of other SoxF family members, Sox7 and Sox18.

Sox17 Represses Runx1 and Gata2 to Maintain Endothelial Identity.

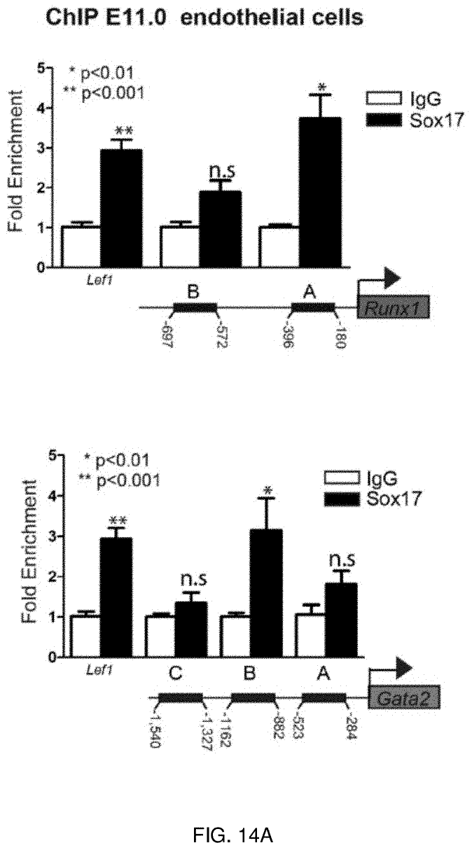

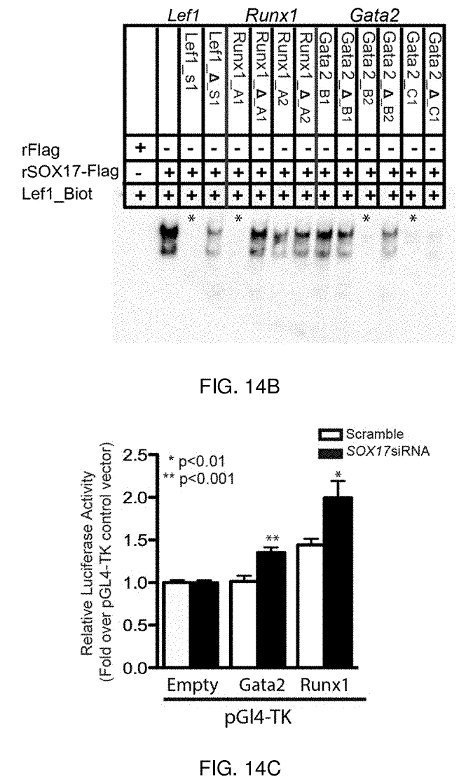

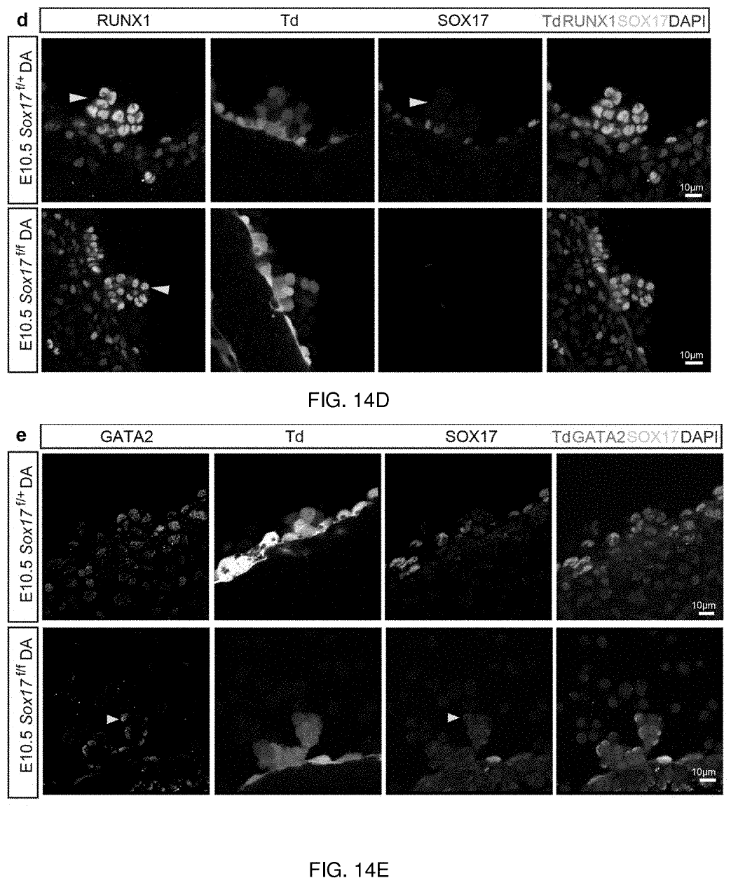

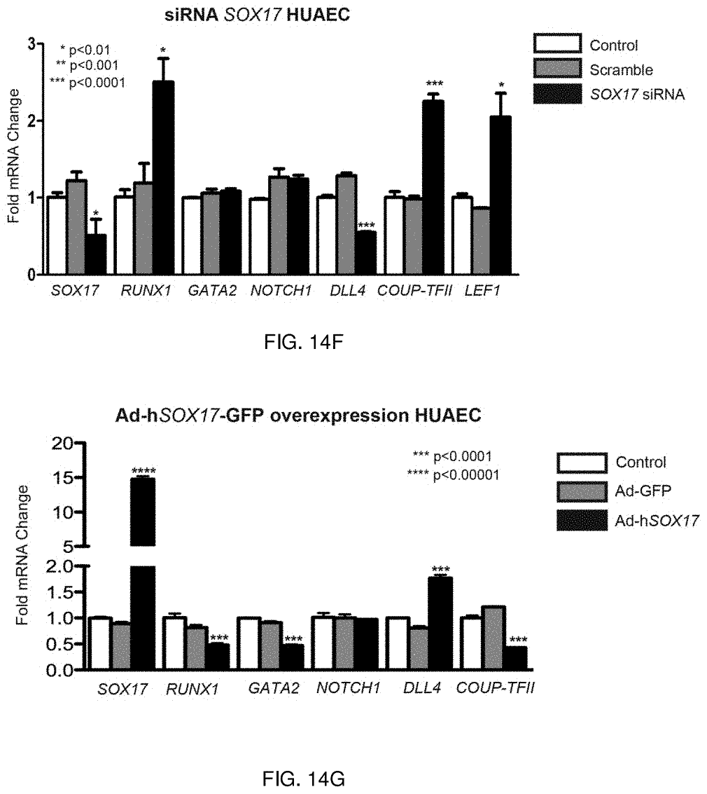

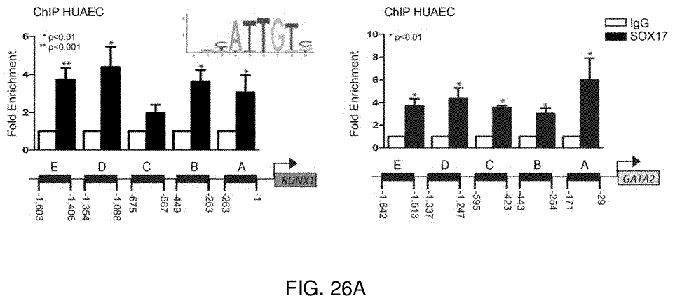

To determine whether the observed changes in Runx1 and Gata2 were due to regulation by SOX17, chromatin immunoprecipitation (ChP) was carried out in sorted endothelial cells at E11 (FIG. 14a), as well as in human umbilical arterial endothelial cell lines, HUAECs (FIG. 26A). Two predicted SOX17 binding sites upstream of Runx1 and Gata2 5′UTRs showed significant enrichment (FIG. 14a). To demonstrate whether SOX17 was capable of direct DNA binding of specific sequences in vitro, electrophoretic mobility shift assays (EMSA) were conducted for sites with high species homology between human and mouse (FIG. 26B). Specific areas within ChIP enriched regions were capable of out-competing the known SOX17 regulatory site in the Lef1 promoter51 (FIG. 14b). We further analyzed the regulation of Runx1 and Gata2 using luciferase reporter assays, which demonstrate de-repression of both Runx1 and Gata2 activity after Sox17 siRNA knockdown (FIG. 14c). In vivo loss of Sox17 demonstrates intact hematopoietic clusters with normal localization of RUNX1 and GATA2 expression (FIG. 14d, e). To investigate how Sox17 may regulate Runx1 and Gata2 in mature endothelium in the human system, we conducted in vitro gain and loss-of-function experiments. SOX17 siRNA inhibition of human umbilical arterial cell lines resulted in significantly elevated RUNX1 transcripts, at similar levels to the control LEF151, a SOX17 repressive target (FIG. 14f). In addition, genes important in arterial and venous identity are altered with decreased arterial gene transcripts (DLL4)52,53 and elevated transcript levels of COUP-TII, an important determinant of venous fate21 (FIG. 14f). In contrast, when SOX17 is overexpressed after adenoviral infection, RUNX1 and GATA2 transcript levels are significantly decreased (FIG. 14g). SOX17 overexpression also altered levels of DLL4 (increased) and COUP-TFII (decreased) (FIG. 14g). The data suggest a novel role of Sox17 as a repressor of hematopoietic fate, while confirming Sox17 as a pro-arterial fate regulator.

Intersecting Roles of Sox17, Runx1, and the Notch Pathway in Hemogenic Endothelium.

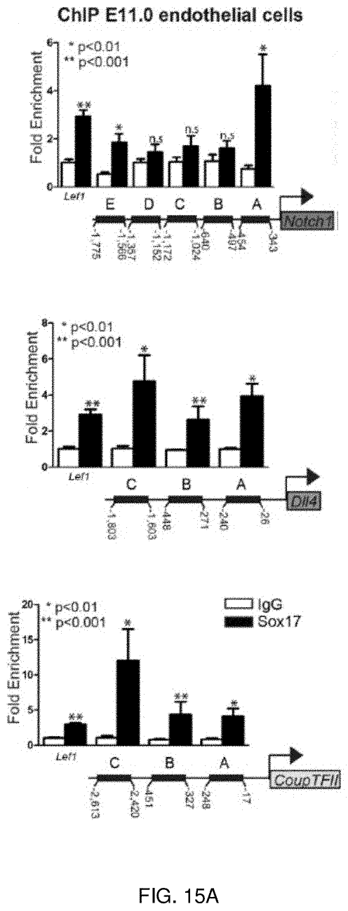

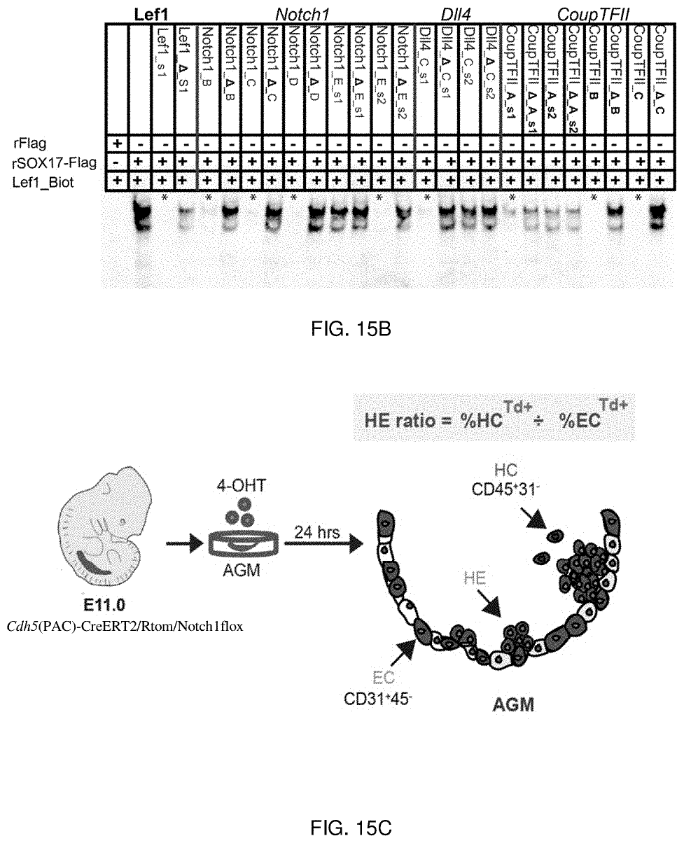

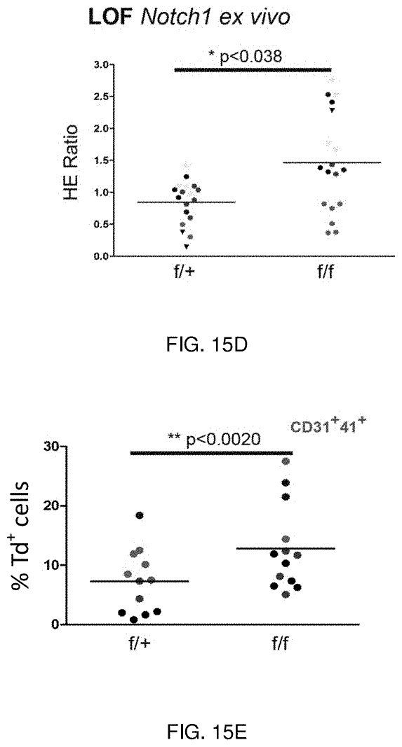

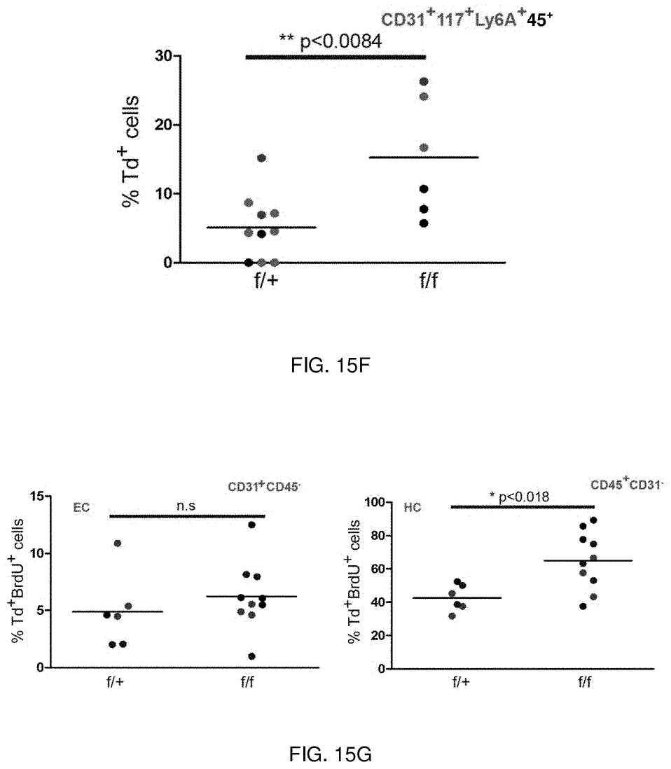

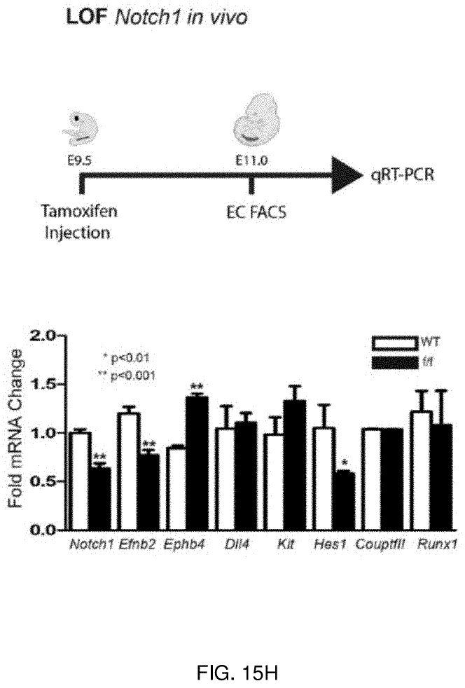

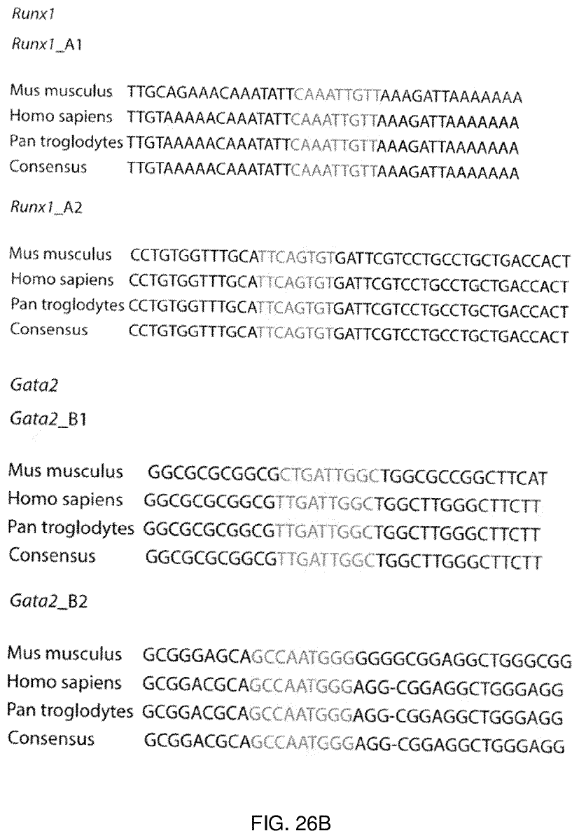

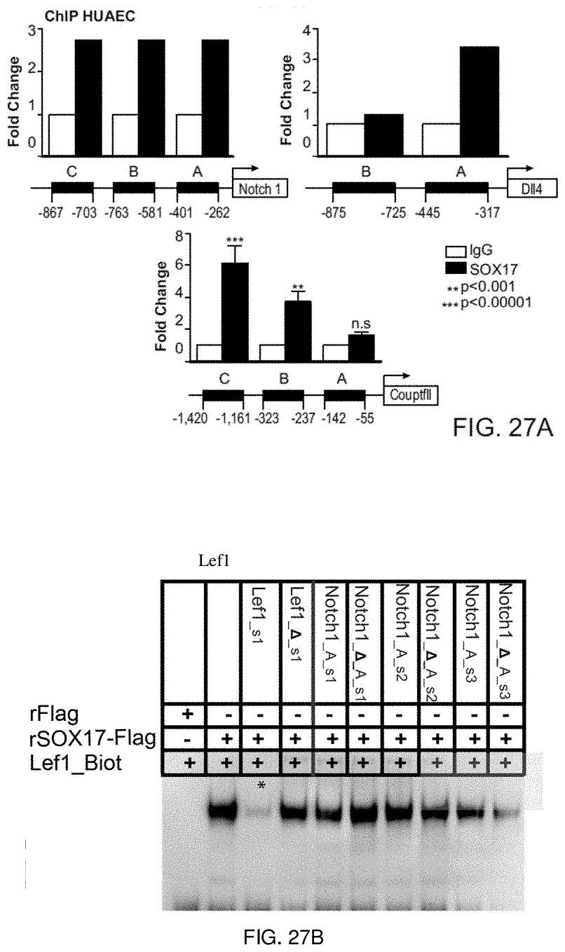

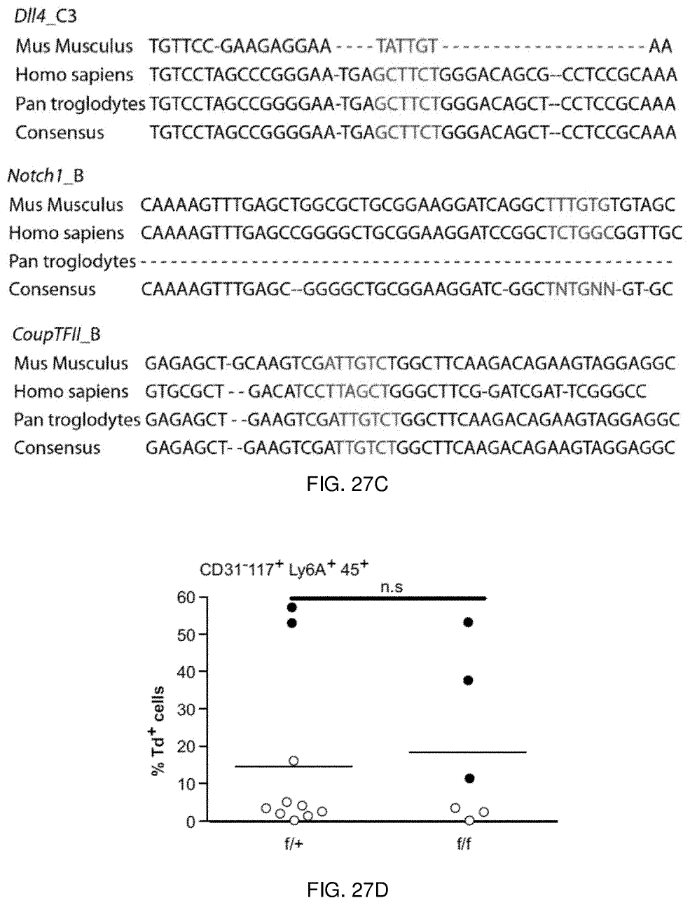

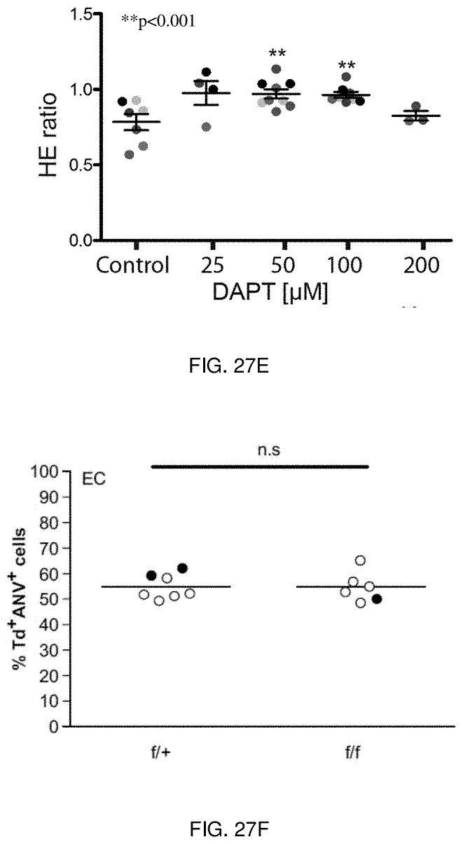

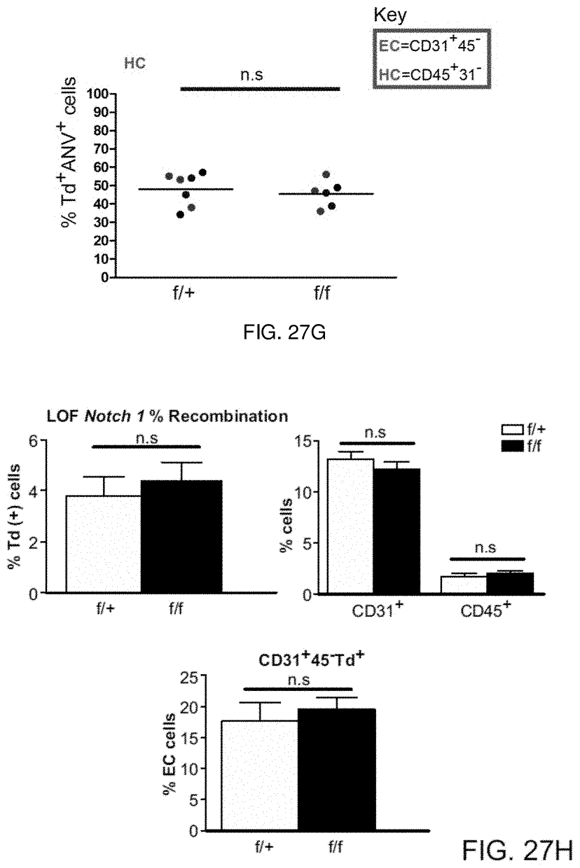

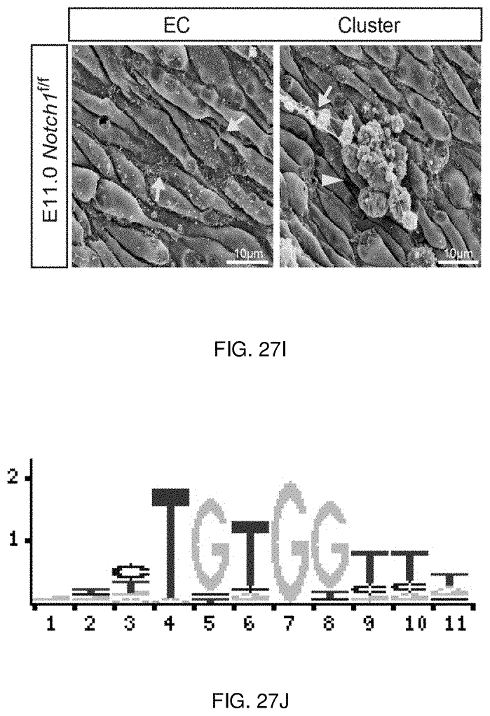

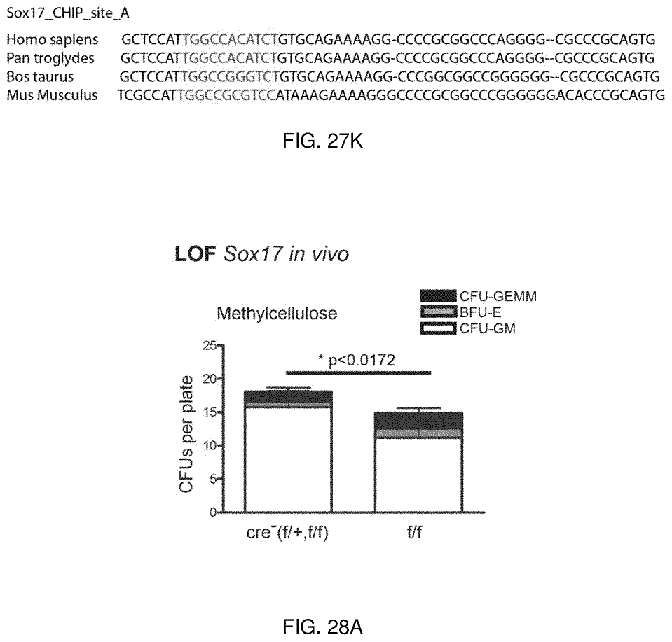

As Sox17 was previously shown to promote arterial identity upstream of the Notch pathway²², we evaluated SOX17 regulation of notch pathway members in our system. SOX17 ChP demonstrates enriched occupancy upstream of the Notch1 5′UTR, and of the Notch ligand D114 (FIG. 15a). In addition, we also observe occupancy upstream of CoupTFII, which has not been previously described (FIG. 15a). Similar enrichment of these sites was observed in HUAECs (FIG. 27A). We further validated direct binding of SOX17 within the enriched ChIP sites via EMSA, and demonstrated multiple SOX17 binding sites are capable of outcompeting Lef1 controls (FIG. 15b, FIG. 27B). To understand whether Notch1, a putative downstream target of SOX17, also plays a repressive role in EHT, we evaluated Notch1 loss-of-function. Similar to Sox17, loss of Notch1 in AGM explants increased the HE ratio, as well as populations of HSPCs, and some pre-HSCs (FIG. 15c-f, FIG. 27D). We also observed increased HE ratios after AGM explants were exposed the γ-secretase inhibitor DAPT (FIG. 27E). However, when BrdU incorporation was evaluated in Notch1 mutant explants, significantly higher levels of incorporation occurred in the hematopoietic compartment (FIG. 15g), suggesting the observed changes may be due to hematopoietic cell proliferation, and not due to an increase in EHT. Annexin-V levels were not notably changed (FIG. 27F-27G). In vivo loss of Notch1 (induction at E9.5), demonstrates expected changes in arterial and venous identity genes23 EfnB2 and EphB4 within sorted endothelial cells (FIG. 15h). No changes in Runx1 transcripts were noted, while expectedly Hes1 transcripts were decreased (FIG. 15h). There were no observed differences in endothelial labeling or cell number across homozygous Notch1f/f, heterozygous Notch1f/+, or control animals (FIG. 27H). Interestingly, we also noted expected changes in endothelial morphology (54) (FIG. 27I).

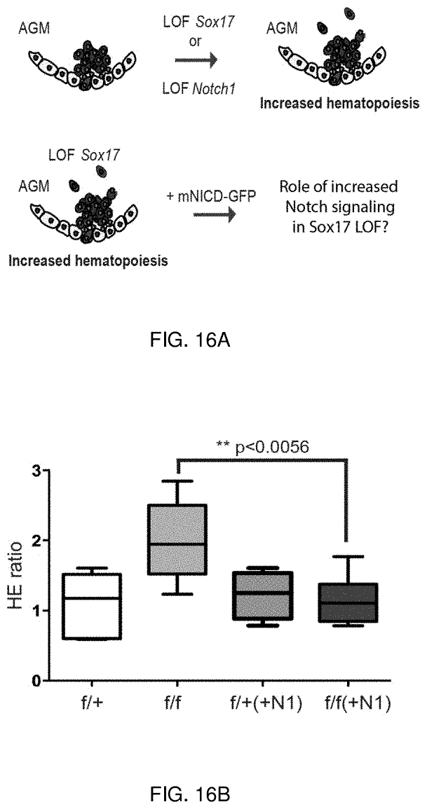

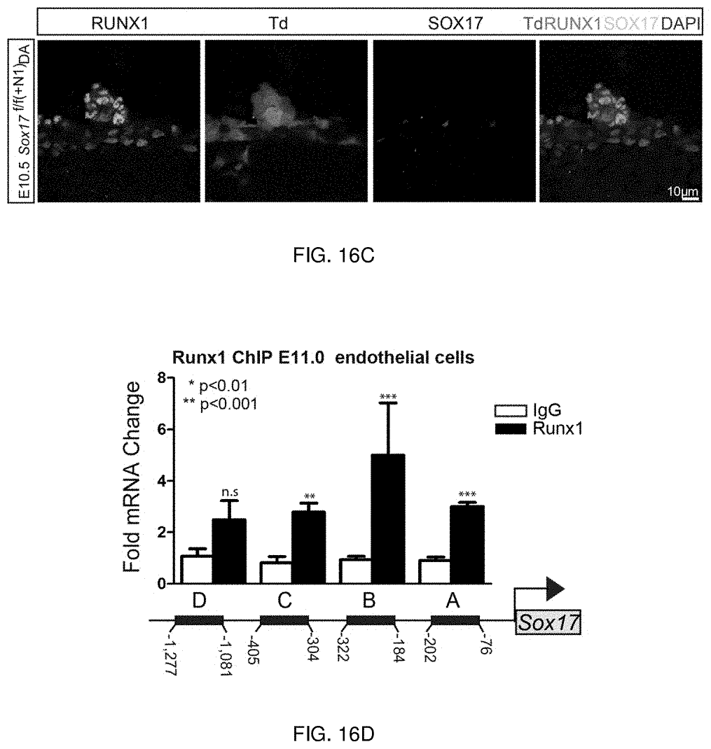

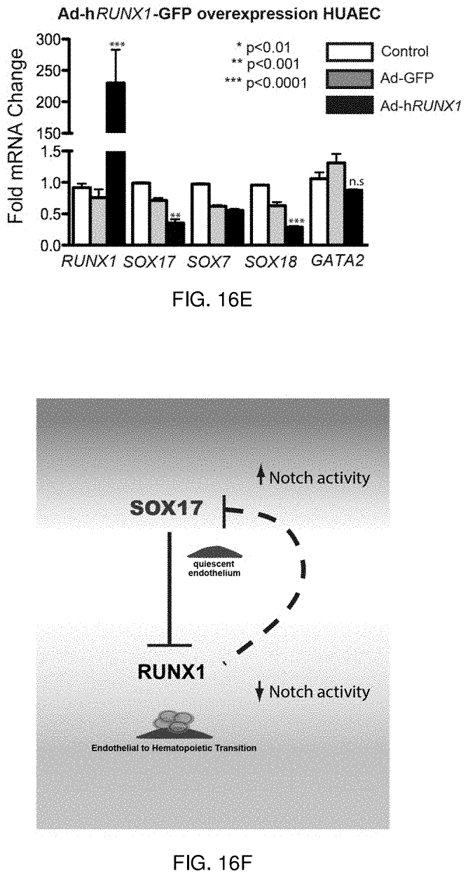

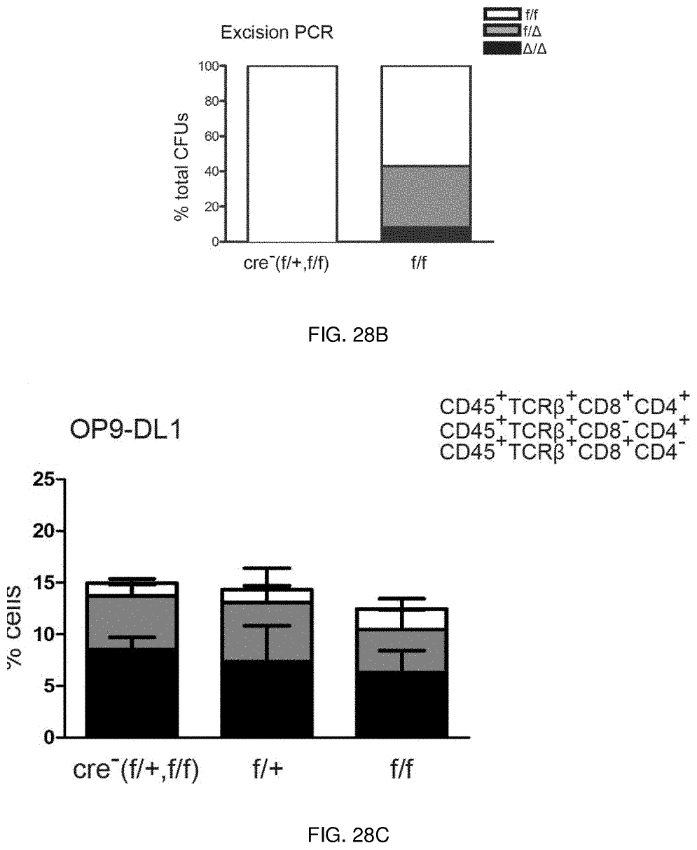

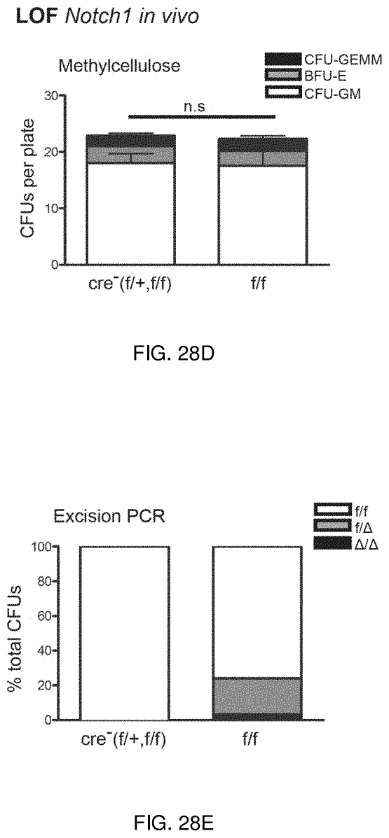

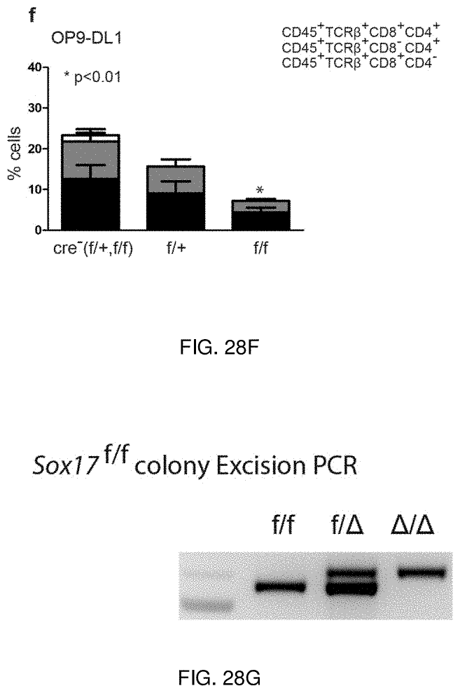

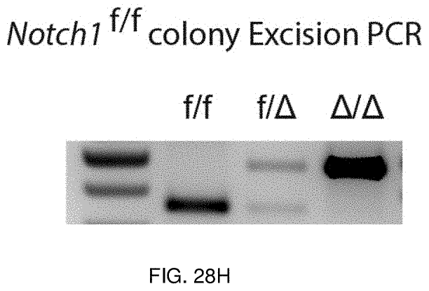

To understand the role of Notch1 signaling in the context of Sox17 loss, we bred R26RNotch1IC-nEGFP lines55 (+mNICD-GFP) that overexpress the Notch1 intracellular domain (NICD) upon Cre activation into our temporal endothelial specific Sox17 loss-of function models (FIG. 16a). Increased Notch activation (induction of Sox17 loss and NICD overexpression at E9.5) was capable of abrogating the observed ET increase in Sox17 mutants (FIG. 16b) with normal appearing HSPC clusters in vivo (FIG. 16c). Thus, the conversion to hematopoietic fate in hemogenic endothelium requires loss of arterial identity programs in addition to de-repression of hematopoietic genes by SOX17. While our data has shown the regulation of Runx1 by SOX17, previous reports suggest RUNX1 may directly bind and repress Sox17⁵⁶. To evaluate whether there may be bi-directional regulation in the endothelium, we performed RUNX1 ChIP of conserved sites upstream of Sox17 transcriptional start sites and found multiple areas of enrichment (FIG. 16d and FIG. 27J-27K). Adenoviral overexpression of RUNX1 (AdhRUNX-GFP) in HUAECs demonstrates decreased SOX17 and SOX18 transcripts (FIG. 16e). Overall, the data presents a complex regulatory network for the maintenance of endothelial cell fate and the conversion to a hematopoietic fate (FIG. 16f). Once hematopoietic fate is achieved, both Sox17 and Notch1 have known roles in hematopoietic cell survival²⁵ and lineage differentiation⁵⁷, as also demonstrated by our hematopoietic colony assay evaluation in mutant hematopoietic cells (FIG. 28).

An important obstacle in recapitulating hemogenic endothelium in culture for in vitro blood production is identification of possible activators and silencers of the hemogenic program. Here we demonstrate important altering requirements for Sox17 and Notch, which highlights the refinements needed for translational models recapitulating EHT. Previous studies have identified Runx1²⁷, Notch1^{24,58}, and Sox17^{25,26} as critical for endothelial to hematopoietic transition. However, dissecting the contributions of these pathways to vascular development versus the process of endothelial to hematopoietic emergence has not been previously reported. Notch1, and more recently Sox17 have demonstrated important roles in arterial specification (22,43,59). As the major vessels that harbor hemogenic endothelium are arterial sites (9,13) it may be that arterial identity is a prerequisite to hemogenic endothelial activity. However, hemogenic activity also occurs in yolk sac and placental vascular beds that are not overtly arterial (9,16,18). In addition, recent evidence in human ESC cultures suggest that while hemogenic endothelial cells incorporate into arterial vascular walls, they have differential surface marker expression profiles than arterial cells (60). There is also evidence that arterial identity can be uncoupled from hemogenic capacity (61,62). So it may be that hemogenic endothelial specification requires the same pathways mobilized in the acquisition of arterial identity, but not arterial identity per se⁴³. However, for the direct transition to hematopoietic fate, the expression levels of arterial/hemogenic specifiers need to be reduced. The complex temporal requirements, elucidated here, explains previous data where continued or overexpression of Sox17 was noted to prevent hematopoiesis in culture (26,63). In addition, the reciprocal repression of Sox17 by RUNX1 introduces another unique aspect of fate determination where once endothelial Sox17 levels decrease, Runx1 levels can rapidly rise during the fate switch, and together they function as a classic bistable system; similar to those described in mesodermal progenitors⁶⁴. Lastly, the data also demonstrate that the EHT program can be manipulated for increased hematopoietic output, suggesting that hemogenic endothelial cell number is not a fixed entity. If EHT is not restricted to a fixed number of endothelial cells within a hemogenic vascular compartment, but instead occurs as a more global transient stochastic process of developing endothelium, it allows for the prospect of endothelial expansion for hematopoietic stem cell production.

### Methods

Animal Care and Use.

Animal protocols were conducted in accordance with University of California at San Francisco Laboratory Animal Research Committee guidelines. Cdh5(PAC)-CreERT2 (Tg(Cdh5-cre/ERT2)1Rha) mice46, Notch1tm2Rko and Sox17tm2Sjm floxed lines (25,65), and R26RNotch1IC-nEGFP (Gt(ROSA)26Sortm1(Notch1)Dam) lines (55) were crossed to R26RTd Cre reporter lines (Gt(ROSA)26Sortm14(CAG-tdTomato)Hze)47. TP1-Venus (Tg(Rbp4*-Venus)#Okn) mice 30,31 were generously provided by RIKEN BioResource Center. Myosin light chain 2 alpha (Mlc2a −/−) mutant lines were provided as described in (40,66). Pregnancies were dated by presence of a vaginal plug (day 0.5 of gestation). Genomic DNA from adult tail tips or conceptus yolk sacs was genotyped using MyTaq Extract PCR Kit (Bioline, BIO21127). Genotype PCR was performed using primers listed in Table 8.

Immunofluorescence and Confocal Microscopy.

E10.5 to E11.5 embryos (in vivo induction with maternal tamoxifen injection at E9.5) were fixed in 2% paraformaldehyde solution overnight and frozen in Tissue-Tek OCT Compound (Sakura Finetek, 4583). 20-30 μm cryosections were obtained (Thermo Scientific Micron, HM550). Slides were dried for 1 hr at room temperature, washed with PBST (0.5% Tween or Triton-X100) and incubated in blocking buffer (PBST, 1% BSA, 5% donkey serum) for 1 hr. Primary antibodies (for full list of antibodies please see Table 7) were incubated at 4° C. overnight or room temperature for 6 hrs in blocking buffer. Slides were washed with PBST and incubated with the secondary antibody for 2 hrs, washed, stained with 2 ug/ul DAPI and mounted in Vectashield (H-1400) or Vectamount (Vector Laboratories, H-5000). Images were captured on a Leica SPE Confocal Microscope and compiled using ImageJ and Imaris 7.6 (Bitplane; Belfast, UK) software.

Flow Cytometric Analyses and Cell Sorting.

Whole embryos or AGMs were dissociated as described67 and stained for 30 min at 4° C. with agitation. Single cell suspensions were sorted in a BD FACS Aria III. Flow cytometric analyses were performed on a FACS Verse or FACS Aria III with FACSDiva 8.0 software (BD Biosciences) and data analyzed using FlowJo v10.0.7 (Tree Star). Gating strategy in FIG. 25C, see Table 7 for a list of antibodies.

Real Time RT-PCR Expression Analysis.

For in vivo transcriptional characterization of the induced endothelium, lineage traced CD31−APC+,CD41−FITC−,CD45−FITC− DAPI-excluded cells were sorted (for full list of antibodies please see Table 7) into MCDB-131 complete medium and RNA was immediately extracted using RNeasy Plus Micro Kit (Qiagen, 74034). 50-300 ng of RNA was reverse transcribed using Superscript III Reverse Transcriptase (Life Technologies, 18080044) according to manufacturer's instructions and cDNA was quantified with Fast SYBR Green Master Mix (Life Technologies, 4385612) in a CFX384 Touch Real-Time PCR Detection System (Bio-Rad). Fluorescence was interpreted relative to GAPDH housekeeping gene expression and quantified using the ΔCt method to obtain relative expression or the ΔΔCt method for fold change values, as indicated. A full list of oligonucleotide sequences is used in Table 4.

AGM Explant Culture and In Vivo Induction.

AGMs from Cdh5(PAC)-CreERT2/R26RTd/Sox17 and Notch1 floxed embryos were dissected and cultured for 24 hrs in 4-hydroxytamoxifen 4-OHT (Sigma H7904) as previously described48, at E11 (and E9.5 for Sox17 mutants). In vivo induction was achieved by intraperitoneal injection of 0.8 mg of tamoxifen of pregnant dams at E9.5. Tamoxifen (MP Biomedical, 156738) prepared as previously described48. DAPT γ-secretase inhibitor (Sigma, D5942) was prepared in DMSO and added directly to explant culture medium at final concentrations of 25 μM, 50 μM, 100 μM, or 200 μM. For overexpression studies, AGMs were incubated with 8×107 adenoviral particles per milliliter at 37 C with agitation for 1 hr prior to explant culture48. Adeno-CMV-hSox17-GFP (AdhSox17-GFP) was produced by Vector Biolabs (ADV-224019, Ref Seq: BC140307).

BrdU.

AGM explants were incubated for 2 hrs with BrdU (10 μM), disaggregated, and stained for extracellular markers CD45-percp and CD31-APC for 30 min. Cells were then fixed and permeabilized with BD Cytofix/Cytoperm™ (BD Biosciences, 554714) according to manufacturer instructions. Cell pellet was washed and incubated in DNase I (300 μg/mL) for 1 hr at 37° C., stained with DAPI and anti-BrdU conjugated with FITC for 30 min, and analyzed by flow cytometry.

Annexin-V.

AGM explants were disaggregated, washed in PBS and resuspended in buffer (10 mM HEPES, 0.9% NaCl, 2.5 mM CaCl2, 0.1% BSA) containing FITC-conjugated Annexin-V (BioLegend, 640906). Cells were incubated at room temperature in the dark for 15 min followed by the addition of buffer containing DAPI, and analyzed by flow cytometry.

siRNA.

Primary human umbilical arterial endothelial cells (HUAEC) (VEC Technologies) were cultured in MCDB-131 Complete medium (VEC Technologies). Sox17 Silencer Select siRNA (Ambion, s34626-8), scramble negative control siRNA (non-targeted sequences), versus water (control) was administered using Lipofectin (Invitrogen, 18292011), and RNA was extracted 48 hrs later using the RNeasy Mini Kit (Qiagen, 74104). Real Time RT PCR was conducted as described above. All cell culture experiments were done between passages from about 4 to about 6. Table 4 lists oligonucleotide sequences of Real Time RT PCR primers.

Recombinant Adenovirus.

Recombinant adenoviral particles were produced by Vector Biolabs (Philadelphia, Pa.). Human SOX17 adenovirus (Ad-hSOX17-GFP) contains Sox17 cDNA (GenBank RefSeq ID BC140307) and enhanced green fluorescent protein (eGFP) driven by CMV promoters. Human RUNX1 adenovirus (Ad-hRUNX1-GFP) contains eGFP-2A preceding RUNX1 cDNA (RefSeq ID BC136381) driven by a single CMV promoter. Ad-GFP control adenovirus (cat #1060) contains CMV driving eGFP only. 1-3×102 viral particles per cell were used to infect subconfluent HUAECs 36 hrs before RNA extraction. All cell culture experiments were done between passages 4-7.

Chromatin Immunoprecipitation (ChIP).

Briefly, HUAEC or E10.5 CD31−APC+ cells were cross-linked with 1% formaldehyde, quenched with 0.125M glycine and re-suspended in lysis buffer (50 mM Hepes-KOH pH7.5, 140 mM NaCl, 1 mM EDTA, 10% glycerol, 0.5% NP-40, 0.25% triton X-100 in ddH20) containing protease inhibitors. The chromatin solution was sonicated, and the supernatant diluted 10-fold. An aliquot of total diluted lysate was used for input gDNA control. Primary antibody or IgG control was incubated with Pierce Protein A/G Magnetic Beads (Thermo Scientific, 88803) at 4° C. overnight to preclear the sample. Sox17 antibody (R&D Systems, AF1924) was used to ChP in both sorted ECs and HUAEC samples, while Runx1 antibody (Cell Signaling, D4A6) was used to perform ChP in HUAECs. The magnetic bead coated by the antibody was washed (PBS, 0.1% Triton X-100) then incubated with the precleared sample at 4° C. overnight. The precipitates were washed, and the chromatin complexes eluted. After reversal of cross-linking (65° C. for 4 hours), the DNA was purified using QIAquick PCR purification kit (Qiagen, 28104) and 100 μg was used as a template in each qPCR reaction for quantitative analysis. Oligonucleotides used in PCR for quantitative ChP are listed in Table 5.

Non-Radioactive Electrophoretic Mobility Shift Assay.

Recombinant SOX17-Flag and Flag alone (pcDNA3 vector (Promega)) were expressed in 293T cells. Plasmids were transfected using Lipofectamine 2000 Transfection Reagent (Life Technologies, 11668019) 36 hrs before cells were lysed in RIPA buffer containing protease inhibitors. Recombinant protein was immunoprecipitated from lysate overnight at 4° C. with Anti-FLAG M2 magnetic beads (Sigma, M8823) and the recombinant protein eluted with excess FLAG peptide. 5-7 ul of the first eluate was used in a binding reaction along with 0.3 pMol of complementary annealed 3′Biotin-labeled oligonucleotides (Integrated DNA 12 Technologies), 300-fold excess competitor probes, 0.02U Poly(dG-dC) (Sigma, P9389), and binding buffer as previously described68. DNA-protein complexes were resolved on 7% native polyacrylamide gel, transferred to neutrally charged nylon membrane, incubated with Streptavidin-POD (Roche, 11089153001) and imaged by chemiluminescence. See Table 6 for probe sequences.

Luciferase reporter assay. Putative regulatory sequences (700-850 bp) including Sox17 ChIP-enriched regions and EMSA-competent Sox17 binding sites were synthesized and cloned (Integrated DNA Technologies) based upon UCSC genome browser murine sequences (see supplementary methods for fragment sequences). The fragments were amplified by PCR (Phusion, New England Biolabs) with appropriate linkers. The pGL4-TK vector (pGL4.54, Promega), containing the gene encoding Firefly luciferase driven by a TK minimal promoter, was digested using kpnI restriction enzyme (NEB) and mung bean nuclease (NEB) followed by ligation using Gibson Assembly mastermix (NEB) and confirmatory sequencing. 30,000 C166 murine yolk sac endothelial cells (ATCC, CRL-2581) were reverse cotransfected with 400 ng of reporter vector along with 10 ng of a Renilla luciferase transfection control plasmid (pRL, Promega) and 30 pMol of a Sox17-targeted or non-targeted “scramble” siRNA pool (ONTARGETplus siRNA SMARTpool, GE Dharmacon) using Lipofectamine 3000 (Life Technologies) according to manufacturer's recommendations. After 48 hours of culture, cells were lysed and luciferase activity assessed using the Dual-Luciferase Reporter Assay System reagents (Promega) in a GloMax 96 Microplate Luminometer with dual injectors. In technical triplicate, relative luciferase activity was calculated by dividing Firefly readings by Renilla readings for each well and then normalized according to baseline values for each treatment condition after transfection of pGL4-TK without a fragment added.

Statistical Analyses.

Student's t-test, one-way and two-way ANOVA analyses were performed as indicated in all experiments where n>3 unless otherwise noted. Mean and standard error were calculated and graphed using GraphPad Prism 6 software. All statistical measurements are listed in Tables 2 and 3.

### REFERENCES

1. Medvinsky, A. & Dzierzak, E. Definitive hematopoiesis is autonomously initiated by the AGM region. Cell 86, 897-906 (1996).
2. Taylor, E., Taoudi, S. & Medvinsky, A. Hematopoietic stem cell activity in the aorta-gonad-mesonephros region enhances after mid-day 11 of mouse development. Int. J. Dev. Biol. 54, 1055-1060 (2010).
3. Tavian, M. & Péault, B. Embryonic development of the human hematopoietic system. Int. J. Dev. Biol. 49, 243-250 (2005).
4. Bertrand, J. Y. et al. Haematopoietic stem cells derive directly from aortic endothelium during development. Nature 464, 108-111 (2010).
5. Kissa, K. & Herbomel, P. Blood stem cells emerge from aortic endothelium by a novel type of cell transition. Nature 464, 112-115 (2010).
6. Eilken, H. M., Nishikawa, S.-I. & Schroeder, T. Continuous single-cell imaging of blood generation from haemogenic endothelium. Nature 457, 896-900 (2009).
7. Boisset, J. C. et al. Progressive maturation towards hematopoietic stem cells in the mouse embryo aorta. Blood (2014). doi:10.1182/blood-2014-07-588954
8. Chen, M. J. et al. Erythroid/Myeloid Progenitors and Hematopoietic Stem Cells Originate from Distinct Populations of Endothelial Cells. Cell Stem cell 9, 541-552 (2011).
9. de Bruijn, M. F., Speck, N. A., Peeters, M. C. & Dzierzak, E. Definitive hematopoietic stem cells first develop within the major arterial regions of the mouse embryo. EMBO J. 19, 2465-2474(2000).
10. North, T. et al. Cbfa2 is required for the formation of intra-aortic hematopoietic clusters. Development 126, 2563-2575 (1999).
11. de Bruijn, M. F. T. R. et al. Hematopoietic stem cells localize to the endothelial cell layer in the midgestation mouse aorta. Immunity 16, 673-683 (2002).
12. Jaffredo, T., Gautier, R., Eichmann, A. & DIETERLEN-LIEVRE, F. Intraaortic hemopoietic cells are derived from endothelial cells during ontogeny. Development 125, 4575-4583 (1998).
13. Gordon-Keylock, S., Sobiesiak, M., Rybtsov, S., Moore, K. & Medvinsky, A. Mouse extraembryonic arterial vessels harbor precursors capable of maturing into definitive HSCs. Blood 122, 2338-2345 (2013).
14. de Bruijn, M. F. et al. CFU-S(11) activity does not localize solely with the aorta in the 18 aorta-gonad-mesonephros region. Blood 96, 2902-2904 (2000).
15. Yoder, M. C., Hiatt, K. & Mukherjee, P. In vivo repopulating hematopoietic stem cells are present in the murine yolk sac at day 9.0 postcoitus. Proc. Natl. Acad. Sci. U.S.A. 94, 6776-6780(1997).
16. Lux, C. T. et al. All primitive and definitive hematopoietic progenitor cells emerging before E10 in the mouse embryo are products of the yolk sac. Blood 111, 3435-3438 (2008).
17. Ottersbach, K. & Dzierzak, E. The murine placenta contains hematopoietic stem cells within the vascular labyrinth region. Dev. Cell 8, 377-387 (2005).
18. Rhodes, K. E. et al. The emergence of hematopoietic stem cells is initiated in the placental vasculature in the absence of circulation. Cell Stem Cell 2, 252-263 (2008).
19. Li, Z. et al. Mouse Embryonic Head as a Site for Hematopoietic Stem Cell Development. Stem Cell 11, 663-675 (2012).
20. Nakano, H. et al. Haemogenic endocardium contributes to transient definitive haematopoiesis. Nature Communications 4, 1564-10 (AD).
21. You, L.-R. et al. Suppression of Notch signalling by the COUP-TFII transcription factor regulates vein identity. Nature 435, 98-104 (2005).
22. Corada, M. et al. Sox17 is indispensable for acquisition and maintenance of arterial identity. Nature Communications 4, 2609 (2013).
23. Lawson, N. D. et al. Notch signaling is required for arterial-venous differentiation during embryonic vascular development. Development 128, 3675-3683 (2001).
24. Kumano, K. et al. Notch1 but not Notch2 is essential for generating hematopoietic stem cells from endothelial cells. Immunity 18, 699-711 (2003).
25. Kim, I., Saunders, T. L. & Morrison, S. J. Sox17 Dependence Distinguishes the Transcriptional Regulation of Fetal from Adult Hematopoietic Stem Cells. Cell 130, 470-483 (2007).
26. Clarke, R. L. et al. The expression of Sox17 identifies and regulates haemogenic endothelium. Nat Cell Biol (2013). doi:10.1038/ncb2724
27. Chen, M. J., Yokomizo, T., Zeigler, B. M., Dzierzak, E. & Speck, N. A. Runx1 is required for the endothelial to haematopoietic cell transition but not thereafter. Nature 457, 887-891 (2009).
28. de Pater, E. et al. Gata2 is required for HSC generation and survival. Journal of Experimental Medicine 464, 116 (2013).
29. Krebs, L. T. et al. Notch signaling is essential for vascular morphogenesis in mice. Genes &amp;amp; Development 14, 1343-1352 (2000).
30. Kohyama, J. et al. Visualization of spatiotemporal activation of Notch signaling: live monitoring and significance in neural development. Developmental Biology 286, 311-325 (2005).
31. Sasaki, N., Kiso, M., Kitagawa, M. & Saga, Y. The repression of Notch signaling occurs via the destabilization of mastermind-like 1 by Mesp2 and is essential for somitogenesis. Development 138, 55-64 (2011).
32. Yokomizo, T. & Dzierzak, E. Three-dimensional cartography of hematopoietic clusters in the vasculature of whole mouse embryos. Development 137, 3651-3661 (2010).
33. Tober, J., Yzaguirre, A. D., Piwarzyk, E. & Speck, N. A. Distinct temporal requirements for Runx1 in hematopoietic progenitors and stem cells. Development (2013). doi:10.1242/dev.094961
34. Robin, C., Ottersbach, K., Boisset, J. C., Oziemlak, A. & Dzierzak, E. CD41 is developmentally regulated and differentially expressed on mouse hematopoietic stem cells. Blood 117, 5088-5091 (2011).
35. Boisset, J. C., Clapes, T., Van Der Linden, R., Dzierzak, E. & Robin, C. Integrin IIb (CD41) plays a role in the maintenance of hematopoietic stem cell activity in the mouse embryonic aorta. Biology Open 2, 525-532 (2013).
36. Taoudi, S. et al. Progressive divergence of definitive haematopoietic stem cells from the endothelial compartment does not depend on contact with the foetal liver. Development 132, 4179-4191(2005).
37. Obi, S. et al. Fluid shear stress induces arterial differentiation of endothelial progenitor cells. J. Appl. Physiol. 106, 203-211 (2009).
38. Jahnsen, E. D. et al. Notch1 is pan-endothelial at the onset of flow and regulated by flow. PLoS ONE 10, e0122622 (2015).
39. Chong, D. C., Koo, Y., Xu, K., Fu, S. & Cleaver, O. Stepwise arteriovenous fate acquisition during mammalian vasculogenesis. Dev. Dyn. 240, 2153-2165 (2011).
40. Huang, C. et al. Embryonic atrial function is essential for mouse embryogenesis, cardiac morphogenesis and angiogenesis. Development 130, 6111-6119 (2003).
41. Nakajima-Takagi, Y. et al. Role of SOX17 in hematopoietic development from human embryonic stem cells. Blood 121, 447-458 (2013).
42. Yoon, M.-J. et al. Mind bomb-1 is essential for intraembryonic hematopoiesis in the aortic endothelium and the subaortic patches. Molecular and Cellular Biology 28, 4794-4804 (2008).
43. Marcelo, K. L. et al. Hemogenic Endothelial Cell Specification Requires c-Kit, Notch Signaling, and p27-Mediated Cell-Cycle Control. DEVCEL 27, 504-515 (2013).
44. Kim, A. D. et al. Discrete Notch signaling requirements in the specification of hematopoietic stem cells. EMBO J. (2014). doi:10.15252/embj.201488784
45. Richard, C. et al. Endothelio-Mesenchymal Interaction Controls runx1 Expression and Modulates the notch Pathway to Initiate Aortic Hematopoiesis. DEVCEL 24, 600-611 (2013).
46. Wang, Y. et al. Ephrin-B2 controls VEGF-induced angiogenesis and lymphangiogenesis. Nature 465, 483-486 (2010).
47. Madisen, L. et al. A robust and high-throughput Cre reporting and characterization system for the whole mouse brain. Nat Neurosci 13, 133-140 (2009).
48. Zovein, A. C. et al. Fate tracing reveals the endothelial origin of hematopoietic stem cells. Cell Stem Cell 3, 625-636 (2008).
49. de Pater, E. et al. Gata2 is required for HSC generation and survival. Journal of Experimental Medicine 210, 2843-2850 (2013).
50. North, T. E. et al. Runx expression marks long-term repopulating hematopoietic stem cells in the midgestation mouse embryo. Immunity 16, 661-672 (2002).
51. Liu, X. et al. Sox17 modulates Wnt3A/-catenin-mediated transcriptional activation of the Lef-1 promoter. AJP: Lung Cellular and Molecular Physiology 299, L694-L710 (2010).
52. Shutter, J. R. et al. D114, a novel Notch ligand expressed in arterial endothelium. Genes &amp;amp; Development 14, 1313-1318 (2000).
53. Duarte, A. et al. Dosage-sensitive requirement for mouse D114 in artery development. Genes &amp;amp; Development 18, 2474-2478 (2004).
54. Limbourg, F. P. et al. Essential role of endothelial Notch1 in angiogenesis. Circulation 111, 1826-1832 (2005).
55. Murtaugh, L. C., Stanger, B. Z., Kwan, K. M. & Melton, D. A. Notch signaling controls multiple steps of pancreatic differentiation. Proc. Natl. Acad. Sci. U.S.A. 100, 14920-14925 (2003).
56. Lichtinger, M. et al. RUNX1 reshapes the epigenetic landscape at the onset of haematopoiesis. EMBO J. 1-16 (2012). doi:10.1038/emboj.2012.275
57. Radtke, F. et al. Deficient T cell fate specification in mice with an induced inactivation of Notch1. Immunity 10, 547-558 (1999).
58. Burns, C. E., Traver, D., Mayhall, E., Shepard, J. L. & Zon, L. I. Hematopoietic stem cell fate is established by the Notch-Runx pathway. Genes &amp; Development 19, 2331-2342 (2005).
59. Lawson, N. D. et al. Notch signaling is required for arterial-venous differentiation during embryonic vascular development. Development 128, 3675-3683 (2001).
60. Ditadi, A. et al. Human definitive haemogenic endothelium and arterial vascular endothelium represent distinct lineages. Nat Cell Biol 17, 580-591 (2015).
61. Burns, C. E. et al. A genetic screen in zebrafish defines a hierarchical network of pathways required for hematopoietic stem cell emergence. Blood 113, 5776-5782 (2009).
62. Robert-Moreno, A. et al. Impaired embryonic haematopoiesis yet normal arterial development in the absence of the Notch ligand Jagged1. EMBO J. 27, 1886-1895 (2008).
63. Nobuhisa, I. et al. Sox17-mediated maintenance of fetal intra-aortic hematopoietic cell clusters. Mol. Cell. Biol. 34, 1976-1990 (2014).
64. Lagha, M. et al. Pax3:Foxc2 Reciprocal Repression in the Somite Modulates Muscular versus Vascular Cell Fate Choice in Multipotent Progenitors. DEVCEL 17, 892-899 (2009).
65. Radtke, F. et al. Deficient T cell fate specification in mice with an induced inactivation of Notch1. Immunity 10, 547-558 (1999).
66. Lucitti, J. L. et al. Vascular remodeling of the mouse yolk sac requires hemodynamic force. Development 134, 3317-3326 (2007).
67. Zovein, A. C. et al. Beta1 integrin establishes endothelial cell polarity and arteriolar lumen formation via a Par3-dependent mechanism. Dev. Cell 18, 39-51 (2010).
68. Donohoe, M. E., Zhang, L.-F., Xu, N., Shi, Y. & Lee, J. T. Identification of a Ctcf cofactor, Yy1, for the X chromosome binary switch. Molecular Cell 25, 43-56 (2007).

### Example 9: Alterations to Current Methodology for the Differentiation of an Endothelial Cell (Prophetic)

Overall the plan is to use our method of non-integrative feeder-free minimal factor (Sox17/Runx1) based reprogramming to allow for large scale banking of human cord endothelial cells that can then, when needed, be converted to produce hematopoietic cells for transplantation. The goals of this method include the production of hematopoietic cells that are of higher number and quality than that currently used for cord blood transplantation, and even perhaps bone marrow transplantation. This would then allow for long term banking of possible donor sources for bone marrow transplantation obviating the need to find live donors or short term availability of cord blood banks. Our long term goal would be to evaluate conversion of endothelial cells stored for >2-5 years. Additionally, we would evaluate whether entire process can be reproduced in a vectorless system, or by entirely chemical means.

Task 1 Rationale:

(1) To increase production of putative blood cells we will test other endothelial cell types (arterial, venous, capillaries), increase cell confluence in culture to increase conversion and test whether low oxygen enhances conversion. (2) To test whether converted cells appear and behave like true hematopoietic cells—initial morphology, surface marker analysis and culture functional analysis.

Task 1 Methodology:

(1) Evaluate HUVECs and HUAECs in parallel to evaluate whether arterial cells convert more easily. (2) Step 6 of protocol—mix transfected cells back to 2:1 to increase cell confluence during hematopoietic conversion. (3) Evaluate low oxygen conditions on process, including keep 02 culture conditions low (<7% throughout process); decrease 02 conditions on day 3 of protocol after initial transfection recovery. (4) Evaluate “hematopoietic” conversion of all Day 8 and Day 9 budded cells: FACS cultured cells for cord blood markers CD34/CD45 and for other markers of new hematopoietic cells CD45/CD144 and Runx1; Giemsa stain of cells in culture for hematopoietic morphological attributes; Functional assay of hematopoiesis—test cultured cells for colony formation in methylcellulose assays; and Analyze cells for DNA content and ploidy.

Task 2 Rationale:

(1) To increase production of putative blood cells by increasing numbers of starting material, and evaluating adjuvants. (2) To test whether converted cells behave like true hematopoietic cells—functional transplantation assays, comparison to cord blood and BM HSCs.

Task 2 Methodology:

(1) Increase number of cells taken through the protocol from 5×105 to 5×106, and calculate number of conversion events per initial cell. (2) Evaluate whether BMP/TGF-beta signaling pathways enhance or inhibit the pathway, as well as other possible enhancers/silencers (see Table 1). (3) Evaluate putative hematopoietic cells in xenograft transplantation assays for full lineage engraftment, e.g. true HSC potential. If so, compare transcriptional signatures of commercially available cord blood and BM HSCs and progenitors to those we obtain in culture.

Task 3 Rationale:

Optimize and/or troubleshoot process to produce large numbers of hematopoietic stem cells for transplantation.

Task 3 Methodology:

(1) Continue testing adjuvants to enhance the process. (2) Screen small molecules that may replace Sox17 and Runx1 episomals so that entire process can be chemical based. (3) Begin HLA subtyping primary endothelial cells and evaluate HLA subtype as well as comparisons of gene expression changes between starting material (endothelial cells) and the produced hematopoietic cells. (4) Evaluate the conversion rates of endothelial cells stored at −80 C for short (weeks/months) or long periods (>6 months-2 years) of time as well as low (2-6) versus high passages of endothelial cells (>6).

### Example 10: Modified Transfection and Culture Protocol

### Sox/Runx Transfection and Culture Protocol

Culture dish-collagen coating:
Make sterile filtered 0.02 M acetic acid
(11.5 μl glacial acetic acid in 10 mL sterile ddH20)
Coat dishes in 1:100 dilution of Bovine Collagen-I (Trevigen)
1 mL per 35 mm well.
37° C. for >1 hour to polymerize.
Wash three times with 1×PBS. Let air dry in hood. Use within two days.

Recovery Media:
Medium 200 (Gibco)+1×LVES (Gibco, 50×), sterile filter

General HUVEC Transfection Protocol:
Life technologies Neon 100 ul Transfection kit. Use standard protocol for adherent cells.
In brief: 5×10⁵ HUVECs (passage 5 or less)(VEC technologies) and 2 μg plasmid per each 100 ul transfection reaction, using R buffer.
Pulse Voltage: 1350v, Pulse Width: 30 ms, Pulse Number: 1
After transfection, suspend cells into 2 mL of recovery media in a collagen-coated 35 mm dish. Let recover overnight.

### Workflow:

1) Prior to experiment, passage HUVECs 1:3 every two days at 37° C., 5% CO₂.
2) Day 0. Transfect HUVECs with 2 μg pCXLE-CAG:Sox17+CMV:eGFP (SEQ ID NO:1) maxi-prepped episomal plasmid (endotoxin-free) onto collagen-coated plates with 2 mL recovery media.
3) Day 1. Switch from recovery media to MCDB-131 media (VEC technologies)
4) Day 3. Confluent cells should be trypsinized (0.25%), quenched with HEK media (DMEM+10% FBS+1% pen/strep) and passage cells 1:2 onto collagen-coated dishes.
5) Day 6 Transfect HUVECs (same protocol as above) with 2 μg pCXLE-CAG:Runx1+CMV:E2 (SEQ ID NO:2)—Crimson maxi-prepped episomal plasmid (endotoxin-free) onto collagen-coated plates in recovery media.
6) Day 7 morning: switch to MCDB-131 complete media.
7) Day 7 afternoon: Add MCDB-131 complete with DAPT (Sigma) (1×, 25 uM) from 1000× stock in DMSO.
8) Day 7 will be recovery. Budding is observed on days 8 and 9.
9) Media is replenished on day 9 for extended observation.

To establish vascular niche platform, endothelial cells were purified and transduced with a lentiviral vector expressing the adenoviral E4ORF1 gene (E4ECs, VeraVecs, Angiocrine Bioscience, New York, N.Y.). Purified CD45− CD133− c-Kit− CD31+ and clonal populations of CD45− CD144+ CD31+ CD62E+ full-term human umbilical vein endothelial cells (HUVECs) and adult primary human dermal microvascular endothelial cells (hDMEC) were cultured in endothelial cell growth medium. Then, HUVECs or hDMECs were transduced with lentiviral vectors expressing GFP and a combination of transcription factors: FOSB, GFI1, RUNX1 and SPI1 (FGRS). After 3 days, GFP+ FGRS-transduced endothelial cells were plated in co-culture with 30-50% subconfluent E4EC monolayers supplemented with serum-free haematopoietic media composed of Stem-Span SFEM, 10% KnockOut serum replacement, 5 ng ml-1 FGF-2, 10 ng ml-1 EGF, 20 ng ml-1 SCF, 20 ng ml-1 FLT3, 20 ng ml-1 TPO, 20 ng ml-1 IGF-1, 10 ng ml-1 IGF-2, 10 ng ml-1 IL-3 and 10 ng ml-1 IL-6. After 3-4 weeks of co-culture, outgrown GFP1 reprogrammed endothelial cells into human multipotent progenitor cells (rEC-hMPPs) formed typical grape-like hematopoietic colonies. After 4 weeks, human CD45+ rEC-hMPPs were FACS sorted for: (1) immunophenotypic analyses; (2) methylcellulose-CFC assay; (3) molecular profiling; (4) comparative genomic hybridization; and (5) transplanted retro-orbitally into primary sublethally irradiated (275 rad) 6-week-old NSG mice or sublethally irradiated (100 rad) 2-weekold mice neonates. After 3 months, sorted, bone-marrow-derived human CD45+ cells (hCD45+ cells) or whole bone marrow of the primary engrafted mice were transplanted into secondary recipients. After 3 months of primary and 6 months of the secondary transplantation, engrafted hCD45+ cells in bone marrow, spleen and peripheral blood of mice were FACS sorted and processed for: (1) multivariate immunophenotypic analyses; (2) clonal and oligo-clonal CFC assay; and (3) molecular profiling. Tissues of the engrafted mice were processed for histological examination to rule out malignant transformation.

A. Cultures.

Adult and neonatal dermal fibroblasts were cultured in F12-DMEM media supplemented with (1) IGFII and bFGF, or (2) IGFII, bFGF, Flt3 and SCF, on Matrigel-coated plates. Lentiviral vectors (pSIN) containing cDNAs of OCT4. NANOG, SOX2 and LIN28 were obtained from Addgene and were transfected into 293-FT cells using the virapower packaging kit (Invitrogen). Fibroblast transductions were performed at 24 h post 104 seeding on Matrigel. For derivation of CD45+ cells, fibroblasts were transduced with OCT4 expressing lentivirus and cultured in media (1) or (2), and iPSCs were derived as previously described15. Further haematopoietic differentiation was carried out using EB media supplemented with haematopoietic cytokines.

B. Functional/Phenotype Analysis.

Flow cytometry analysis of hematopoietic and pluripotency markers was performed using FACSCalibur (Beckman Coulter), and analysis was performed using the FlowJo 8.8.6 software. Cell sorting was performed using FACSAria II (Becton-Dickinson); Histological profiling of hematopoietic cells was performed using Cytospin and Giemsa-Wright staining and confirmed by the McMaster Pathology and Hematology Group; CFU formation was assayed using Methocult and Megacult kits from Stem Cell Technologies; Macrophage phagocytosis assay was performed using Fluorescein conjugated latex beads (Sigma) as particle tracers to analyse uptake bymonocytes derived from CD45+Fibs^{OCT4} cells; in vivo engraftment capacity was evaluated by intrafemoral injection of CD45^{+ve} cells into NSG mice. Ten weeks later bone marrow from injected femur, contralateral bones and spleen was analysed for the presence of human cells by flow cytometry; teratoma formation was evaluated by intratesticular injection into NOD/SCID mice. Resulting teratomas were evaluated for the presence of mesoderm, endoderm and ectoderm through histological examination.

C. Molecular Analysis.

For qPCR and microarray analysis, RNA was extracted using a total RNA purification kit (Norgen). Microarray analysis was done using Human Gene 1.0 ST arrays (Affymetrix) and dChP software. OCT4 DNA occupancy (OCT4 ChIP) was done as previously described⁴⁵.

D. Preparation of Cells.

The autologous fibroblasts are derived by outgrowth from a tissue biopsy followed by expansion in culture using standard cell culture techniques. The starting material is composed of three 3-mm punch biopsies collected using standard aseptic practices. The biopsies are collected by the treating physician, placed into a vial containing sterile phosphate buffered saline (PBS). The biopsies are shipped in a 2-8° C. refrigerated shipper back to the manufacturing facility.

After arrival at the manufacturing facility, the biopsy is inspected and, upon acceptance, transferred directly to the manufacturing area. Upon initiation of the process, the biopsy tissue is then washed prior to enzymatic digestion. After washing, a Liberase Digestive Enzyme Solution is added without mincing, and the biopsy tissue is incubated at 37.0±2° C. for one hour. Time of biopsy tissue digestion is a critical process parameter that can affect the viability and growth rate of cells in culture. Liberase is a collagenase/neutral protease enzyme cocktail obtained formulated from Lonza Walkersville, Inc. (Walkersville, Md.) and unformulated from Roche Diagnostics Corp. (Indianapolis, Ind.). Alternatively, other commercially available collagenases may be used, such as Serva Collagenase NB6.

After digestion, Initiation Growth Media (IMDM, GA, 10% Fetal Bovine Serum (FBS)) is added to neutralize the enzyme, cells are pelleted by centrifugation and resuspended in 5.0 mL Initiation Growth Media. Alternatively, centrifugation is not performed, with full inactivation of the enzyme occurring by the addition of Initiation Growth Media only. Initiation Growth Media is added prior to seeding of the cell suspension into a T-175 cell culture flask for initiation of cell growth and expansion. A T-75, T-150, T-185 or T-225 flask can be used in place of the T-75 flask.

Cells are incubated at 37±2.0° C. with 5.0±1.0% C02 and fed with fresh Complete Growth Media every three to five days. All feeds in the process are performed by removing half of the Complete Growth Media and replacing the same volume with fresh media. Alternatively, full feeds can be performed. Cells should not remain in the T-175 flask greater than 30 days prior to passaging. Confluence is monitored throughout the process to ensure adequate seeding densities during culture splitting. When cell confluence is greater than or equal to 40% in the T-175 flask, they are passaged by removing the spent media, washing the cells, and treating with Trypsin-EDTA to release adherent cells in the flask into the solution. Cells are then trypsinized and seeded into a T-500 flask for continued cell expansion. Alternately, one or two T-300 flasks, One Layer Cell Stack (1 CS), One Layer Cell Factory (1 CF) or a Two Layer Cell Stack (2 CS) can be used in place of the T-500 Flask.

Morphology is evaluated at each passage and prior to harvest to monitor the culture purity throughout the culture purity throughout the process. Morphology is evaluated by comparing the observed sample with visual standards for morphology examination of cell cultures. The cells display typical fibroblast morphologies when growing in cultured monolayers. Cells may display either an elongated, fusiform or spindle appearance with slender extensions, or appear as larger, flattened stellate cells which may have cytoplasmic leading edges. A mixture of these morphologies may also be observed. Fibroblasts in less confluent areas can be similarly shaped, but randomly oriented. The presence of keratinocytes in cell cultures is also evaluated. Keratinocytes appear round and irregularly shaped and, at higher confluence, they appear organized in a cobblestone formation. At lower confluence, keratinocytes are observable in small colonies.

Cells are incubated at 37±2.0° C. with 5.0±1.0% C02 and fed every three to five days in the T-500 flask and every five to seven days in the ten layer cell stack (IOCS). Cells should not remain in the T-500 flask for more than 10 days prior to passaging. Quality Control (QC) release testing for safety of the Bulk Pharmaceutical composition includes sterility and endotoxin testing. When cell confluence in the T-500 flask is >95%, cells are passaged to a 10 CS culture vessel. Alternately, two Five Layer Cell Stacks (5 CS) or a 10 Layer Cell Factory (10 CF) can be used in place of the 10 CS. IOCS.

Passage to the 10 CS is performed by removing the spent media, washing the cells, and treating with Trypsin-EDTA to release adherent cells in the flask into the solution. Cells are then transferred to the 10 CS. Additional Complete Growth Media is added to neutralize the trypsin and the cells from the T-500 flask are pipetted into a 2 L bottle containing fresh Complete Growth Media. The contents of the 2 L bottle are transferred into the 10 CS and seeded across all layers. Cells are then incubated at 37±2.0° C. with 5.0±1.0% C02 and fed with fresh Complete Growth Media every five to seven days. Cells should not remain in the IOCS for more than 20 days prior to passaging. The passaged fibroblasts are rendered substantially free of immunogenic proteins present in the culture medium by incubating the expanded fibroblasts for a period of time in protein free medium.

Primary Harvest: When cell confluence in the 10 CS is 95% or more, cells are harvested. Harvesting is performed by removing the spent media, washing the cells, treating with Trypsin-EDTA to release adherent cells into the solution, and adding additional Complete Growth Media to neutralize the trypsin. Cells are collected by centrifugation, resuspended, and in-process QC testing performed to determine total viable cell count and cell viability.

For treatment of nasolabial folds, the total cell count must be 3.4×10⁸ cells and viability 85% or higher. Alternatively, total cell yields for other indications can range from about 3.4×10⁸ to 1×10⁹ cells. Cell count and viability at harvest are critical parameters to ensure adequate quantities of viable cells for formulation of the Pharmaceutical composition. If total viable cell count is sufficient for the intended treatment, an aliquot of cells and spent media are tested for mycoplasma contamination. Mycoplasma testing is performed. Harvested cells are formulated and cryopreserved. If additional cells are required after receiving cell count results from the primary 10 CS harvest, an additional passage into multiple cell stacks (up to four 10 CS) is performed (Step 5a in FIG. 1). For additional passaging, cells from the primary harvest are added to a 2 L media bottle containing fresh Complete Growth Media. Resuspended cells are added to multiple cell stacks and incubated at 37±2.0° C. with 5.0±1.0% CO₂. The cell stacks are fed and harvested as described above, except cell confluence must be 80% or higher prior to cell harvest. The harvest procedure is the same as described for the primary harvest above. A mycoplasma sample from cells and spent media is collected, and cell count and viability performed as described for the primary harvest above.

The method decreases or eliminates immunogenic proteins be avoiding their introduction from animal-sourced reagents. To reduce process residuals, cells are cryopreserved in protein-free freeze media, then thawed and washed prior to prepping the final injection to further reduce remaining residuals.

E. Preparation of Cell Suspension.

At the completion of culture expansion, the cells are harvested and washed, then formulated to contain from about 1.0 to about 2.7×10⁷ cells/mL, with a target of 2.2×10⁷ cells/mL. Alternatively, the target can be adjusted within the formulation range to accommodate different indication doses. The pharmaceutical composition consists of a population of viable, autologous human fibroblast cells suspended in a cryopreservation medium consisting of Iscove's Modified Dulbecco's Medium (IMDM) and Profreeze-CDM™ (Lonza, Walkerville, Md.) plus 7.5% dimethyl sulfoxide (DMSO). Alternatively, a lower DMSO concentration may be used in place of 7.5% or CryoStor™ CS5 or CryoStor™ CS10 (BioLife Solutions, Bothell, Wash.) may be used in place of IMDM/Profreeze/DMSO. The freezing process consists of a control rate freezing step to the following ramp program:

STEP 1: Wait at 4.0° C.

STEP 2: 1.0° C./minC/m to −4.0° C. (sample probe)

STEP 3: 25.0° C./minC/m to −40° C. (chamber probe)

STEP 4: 10.0° C./minC/m to −12.0° C. (chamber probe)

STEP 5: 1.0° C./minC/m to −40° C. (chamber probe)

STEP 6: 10.0° C./minC/m to −90° C. (chamber probe)

STEP 7: End

After completion of the controlled rate freezing step, Bulk Pharmaceutical composition vials are transferred to a cryogenic freezer for storage in the vapor phase. After cryogenic freezing, the Pharmaceutical composition is submitted for Quality Control testing. Pharmaceutical composition specifications also include cell count and cell viability testing performed prior to cryopreservation and performed again for Pharmaceutical composition—Cryovial. Viability of the cells must be 85%> or higher for product release. Cell count and viability are conducted using an automated cell counting system (Guava Technologies), which utilizes a combination of permeable and impermeable fluorescent, DNA-intercalating dyes for the detection and differentiation of live and dead cells.

Alternatively, a manual cell counting assay employing the trypan blue exclusion method may be used in place of the automated cell method above. Alternatively, other automated cell counting systems may be used to perform the cell count and viability method, including Cedex (Roche Innovatis AG, Bielefield, Germany), ViaCell™ (Beckman Coulter, Brea, Calif.),

NuceloCounter™ (New Brunswick Scientific, Edison, N.J.), Countless® (Invitrogen, division of Life Technologies, Carlsbad, Calif.), or Cellometer® (Nexcelom Biosciences, Lawrence, Mass.). Pharmaceutical composition—Cryovial samples must meet a cell count specification of 1.0-2.7×107 cells/mL prior to release. Sterility and endotoxin testing are also conducted during release testing. In addition to cell count and viability, purity/identity of the Pharmaceutical composition is performed and must confirm the suspension contains 98% or more fibroblasts. The usual cell contaminants include keratinocytes. The purity/identify assay employs fluorescent-tagged antibodies against CD90 and CD 104 (cell surface markers for fibroblast and keratinocyte cells, respectively) to quantify the percent purity of a fibroblast cell population. CD90 (Thy-1) is a 35 kDa cell-surface glycoprotein. Antibodies against CD90 protein have been shown to exhibit high specificity to human fibroblast cells. CD 104, integrin β4 chain, is a 205 kDa transmembrane glycoprotein which associates with integrin a6 chain (CD49f) to form the α6/β4 complex. This complex has been shown to act as a molecular marker for keratinocyte cells (Adams and Watt 1991).

Antibodies to CD 104 protein bind to 100% of human keratinocyte cells. Cell count and viability is determined by incubating the samples with Viacount Dye Reagent and analyzing samples using the Guava PCA system. The reagent is composed of two dyes, a membrane—permeable dye which stains all nucleated cells, and a membrane-impermeable dye which stains only damaged or dying cells. The use of this dye combination enables the Guava PCA system to estimate the total number of cells present in the sample, and to determine which cells are viable, apoptotic, or dead.

Alternatively, cells can be passaged from either the T-175 flask (or alternatives) or the T-500 flask (or alternatives) into a spinner flask containing microcamers as the cell growth surface. Microcamers are small bead-like structures that are used as a growth surface for anchorage dependent cells in suspension culture. They are designed to produce large cell yields in small volumes.

In this apparatus, a volume of Complete Growth Media ranging from 50 mL-300 mL is added to a 500 mL, IL or 2 L sterile disposable spinner flask. Sterile microcarriers are added to the spinner flask. The culture is allowed to remain static or is placed on a stir plate at a low RPM (15-30 RRM) for a short period of time (1-24 hours) in a 37±2.0° C. with 5.0±1.0% C02 incubator to allow for adherence of cells to the carriers. After the attachment period, the speed of the spin plate is increased (30-120 RPM). Cells are fed with fresh Complete Growth Media every one to five days, or when media appears spent by color change.

Cells are collected at regular intervals by sampling the microcarriers, isolating the cells and performing cell count and viability analysis. The concentration of cells per carrier is used to determine when to scale-up the culture. When enough cells are produced, cells are washed with PBS and harvested from the microcarriers using trypsin-EDTA and seeded back into the spinner flask in a larger amount of microcarriers and higher volume of Complete Growth Media (300 mL-2 L). Alternatively, additional microcarriers and Complete Growth Media can be added directly to the spinner flask containing the existing microcarrier culture, allowing for direct bead-to-bead transfer of cells without the use of trypsinization and reseeding. Alternatively, if enough cells are produced from the initial T-175 or T-500 flask, the cells can be directly seeded into the scale-up amount of microcarriers.

After the attachment period, the speed of the spin plate is increased (30-120 RPM). Cells are fed with fresh Complete Growth Media every one to five days, or when media appears spent by color change. When the concentration reaches the desired cell count for the intended indication, the cells are washed with PBS and harvested using trypsin-EDTA. All release testing, cryopreservation and preparation of Drug Product—Injection would follow the process described in Sections C and D. Microcarriers used within the disposable spinner flask may be made from poly blend such as BioNOC II® (Cesco Bioengineering, distributed by Bellco Biotechnology, Vineland, N.J.) and FibraCel® (New Brunswick Scientific, Edison, N.J.), gelatin, such as Cultispher-G (Percell Biolytica, Astrop, Sweden), cellulose, such as Cytopore™ (GE Healthcare, Piscataway, N.J.) or coated/uncoated polystyrene, such as 2D MicroHex™ (Nunc, Weisbaden, Germany), Cytodex® (GE Healthcare, Piscataway, N.J.) or Hy-Q Sphere™ (Thermo Scientific Hyclone, Logan, Utah).

Alternatively, cells can be processed on poly blend 2D microcarriers such as BioNOC II® and FibraCel® using an automatic bellow system, such as FibraStage™ (New Brunswick Scientific, Edison, N.J.) or BelloCell® (Cesco Bioengineering, distributed by Bellco Biotechnology, Vineland, N.J.) in place of the spinner flask apparatus. Cells from the T-175 (or alternatives) or T-500 flask (or alternatives) are passaged into a bellow bottle containing microcarriers with the appropriate amount of Complete Growth Media, and placed into the system. The system pumps media over the microcarriers to feed cells, and draws away media to allow for oxygenation in a repeating fixed cycle. Cells are monitored, fed, washed and harvested in the same sequence as described above.

Alternatively, cells can be processed using automated systems. After digestion of the biopsy tissue or after the first passage is complete (T-175 flask or alternative), cells may be seeded into an automated device. One method is an Automated Cellular Expansion (ACE) system, which is a series of commercially available or custom fabricated components linked together to form a cell growth platform in which cells can be expanded without human intervention. Cells are expanded in a cell tower, consisting of a stack of disks capable of supporting anchorage-dependent cell attachment. The system automatically circulates media and performs trypsinization for harvest upon completion of the cell expansion stage.

Alternatively, the ACE system can be a scaled down, single lot unit version comprised of a disposable component that consists of cell growth surface, delivery tubing, media and reagents, and a permanent base that houses mechanics and computer processing capabilities for heating/cooling, media transfer and execution of the automated programming cycle. Upon receipt, each sterile irradiated ACE disposable unit will be unwrapped from its packaging and loaded with media and reagents by hanging pre-filled bags and connecting the bags to the existing tubing via aseptic connectors. The process continues as follows:

Inside a biological safety cabinet (BSC), a suspension of cells from a biopsy that has been enzymatically digested is introduced into the “pre-growth chamber” (small unit on top of the cell tower), which is already filled with Initiation Growth Media containing antibiotics. From the BSC, the disposable would be transferred to the permanent ACE unit already in place.

After approximately three days, the cells within the pre-growth chamber are trypsinized and introduced into the cell tower itself, which is pre-filled with Complete Growth Media. Here, the “bubbling action” caused by CO₂ injection force the media to circulate at such a rate that the cells spiral downward and settle on the surface of the discs in an evenly distributed manner.

For approximately seven days, the cells are allowed to multiply. At this time, confluence will be checked (method unknown at time of writing) to verify that culture is growing. Also at this time, the Complete Growth Media will be replaced with fresh Complete Growth Media. CGM will be replaced every seven days for three to four weeks. At the end of the culture period, the confluence is checked once more to verify that there is sufficient growth to possibly yield the desired quantity of cells for the intended treatment.

If the culture is sufficiently confluent, it is harvested. The spent media (supernatant) is drained from the vessel. PBS will then is pumped into the vessel (to wash the media, FBS from the cells) and drained almost immediately. Trypsin-EDTA is pumped into the vessel to detach the cells from the growth surface. The trypsin/cell mixture is drained from the vessel and enter the spin separator. Cryopreservative is pumped into the vessel to rinse any residual cells from the surface of the discs, and be sent to the spin separator as well. The spin separator collects the cells and then evenly resuspend the cells in the shipping/injection medium. From the spin separator, the cells will be sent through an inline automated cell counting device or a sample collected for cell count and viability testing via laboratory analyses. Once a specific number of cells has been counted and the proper cell concentration has been reached, the harvested cells are delivered to a collection vial that can be removed to aliquot the samples for cryogenic freezing.

Alternatively, automated robotic systems may be used to perform cell feeding, passaging, and harvesting for the entire length or a portion of the process. Cells can be introduced into the robotic device directly after digest and seed into the T-175 flask (or alternative). The device may have the capacity to incubate cells, perform cell count and viability analysis and perform feeds and transfers to larger culture vessels. The system may also have a computerized cataloging function to track individual lots. Existing technologies or customized systems may be used for the robotic option.

### Example 11: Directed Reprogramming of Endothelium to Hemogenic Endothelium Using Sox17 and Runx1 Episomals in Addition to DAPT

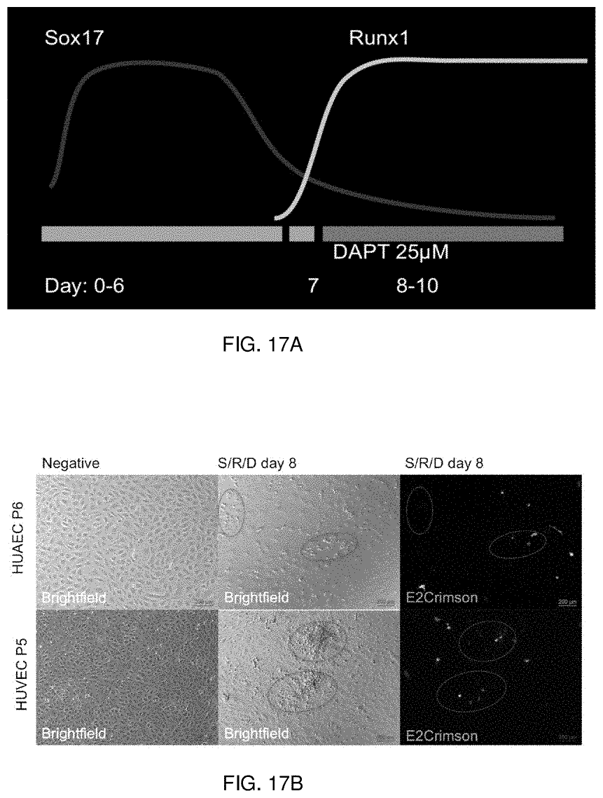

Sox17 and Runx1 expression levels were increased in endothelial cells in combination with DAPT treatment for directed reprogramming of endothelium to hemogenic endothelium. Sox17 and Runx1 expression levels were increased in the endothelial cells by sequentially transfecting the cells with Sox17 and Runx1 episomals as described below. See FIG. 17A.

### Materials List:

| | | |
|---|---|---|
| Bovine Collagen I | Trevigen | 3442-005-01 |
| DAPT | Sigma | D5942-5MG |
| DMEM H21 | UCSF CCF | CCFAA005 |
| EndoFree Maxi-prep kit | Qiagen | CCFAA005 |
| FBS JRS | UCSF CCF | CCFAQ008 |
| hCD45-V450 Antibody | BD | 560368 |
| IgG isotype control | BD | 560373 |
| LVES (50X) | Gibco | A146081 |
| MCDB-131-complete | VEC | MCDB-131C/500 mL |
| Medium 200 | Gibco | M200500 |
| Neon Transfection Kit 100 ul | Thermo Fischer | MPK10096 |
| Penn/Strep | UCSF CCF | CCFGK004 |
| Trypsin 0.25% | UCSF CCF | ccfgp003-15am01 |

### List of Plasmids:

pCXLE-CAG:Sox17+CMV:eGFP
pCXLE-CAG:Runx1+CMV:E2-Crimson
pCXLE-CAG:Sox17
pCXLE-CAG:Runx1
eGFP Dummy (eGFP under CMV, CAG empty)
Crimson Dummy (Crimson under CMV, CAG empty)
   pCXLE-CAG:Runx1+shTP53+CMV:mCherry-2A-puro

### Culture Dish-Collagen Coating:

Make sterile filtered 0.02 M acetic acid
(11.5 μl glacial acetic acid in 10 mL sterile ddH20)
Coat dishes in 1:100 dilution of Bovine Collagen-I (Trevigen)
1 mL per 35 mm well.
37° C. for >1 hour to polymerize.
Wash three times with 1×PBS. Let air dry in hood. Use within two days.

### Recovery Media:

Medium 200 (Gibco)+1×LVES (Gibco, 50×), sterile filter

### General HUVEC Transfection Protocol:

Life technologies Neon 100 ul Transfection kit. Use standard protocol for adherent cells. In brief: 5×10⁵ HUVECs (passage 5 or less)(VEC technologies) and 2 μg plasmid per each 100 ul transfection reaction, using R buffer.

### Pulse Voltage: 1350v, Pulse Width: 30 ms, Pulse Number: 1

After transfection, suspend cells into 2 mL of recovery media in a collagen-coated 35 mm dish.
Let recover overnight. Recommended to change recovery media after 4 hours.

### Workflow:

1) Prior to experiment, passage HUVECs 1:3 every two days at 37° C., 5% CO2.
a. MCDB-131 Complete media, always pre-warmed and properly gassed.
2) Day 0 Transfect 5×10⁵ HUVECs with 2 μg pCXLE-CAG:Sox17+CMV:eGFP maxi-prepped episomal plasmid (endotoxin-free) onto collagen-coated plates with 2 mL recovery media.
3) Day 1 Switch from recovery media to MCDB-131 media (VEC technologies)
4) Day 3 Confluent cells should be trypsinized (0.25%), quenched with HEK media (DMEM+10% FBS+1% pen/strep) and passage cells 1:2 onto collagen-coated dishes.
a. Note: Never use accutase
5) Day 6 Transfect 5×10⁵ HUVECs (same protocol as above) with 2 μg pCXLE-CAG:Runx1+CMV:E2-Crimson maxi-prepped episomal plasmid (endotoxin-free) onto collagen-coated plates in recovery media. Use two transfections onto one well.
6) Day 7 morning: switch to MCDB-131 complete media.
7) Day 7 afternoon: Add MCDB-131 complete with DAPT (Sigma) (1×, 25 uM) from 1000× stock in DMSO.
8) Day 7 will be recovery. Budding is observed on days 8 and 9.
9) Media is replenished on day 9 for extended observation.

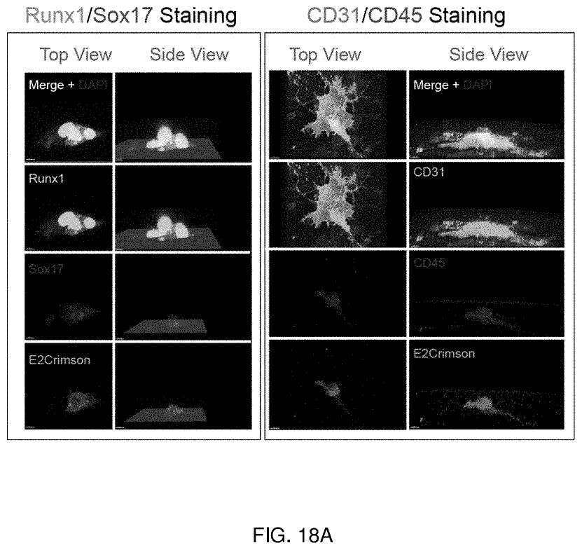

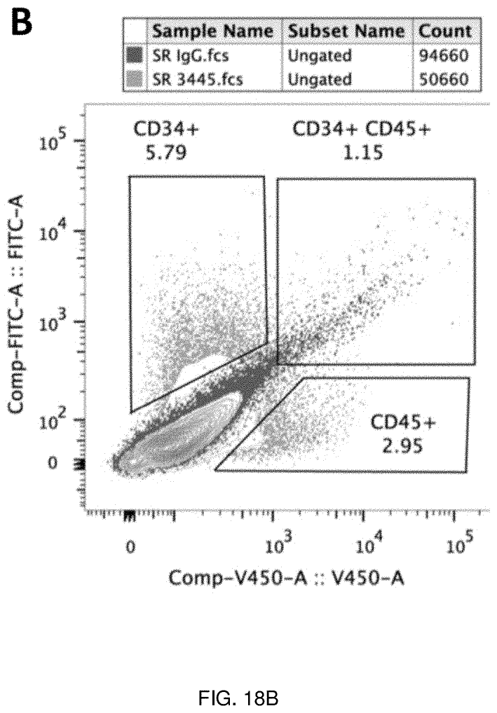

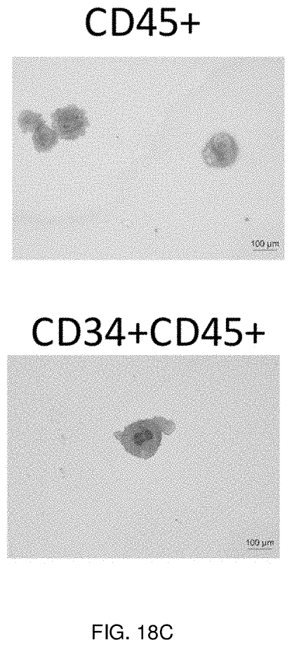

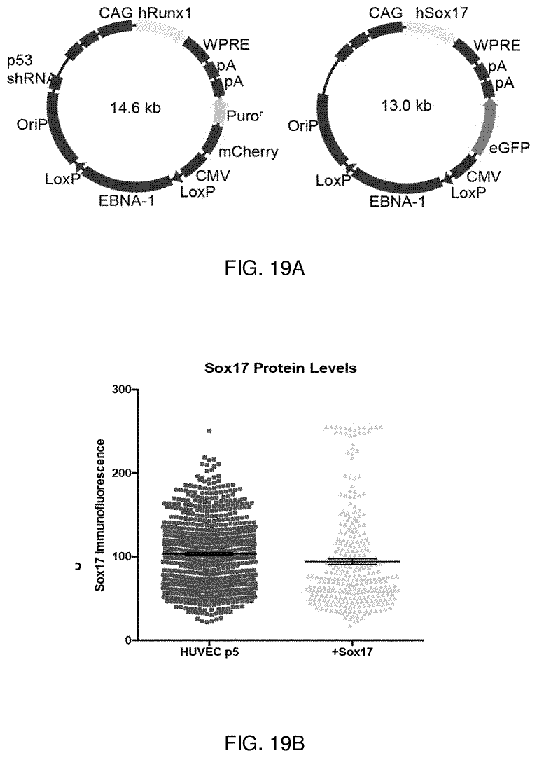

Sox17 and Runx1 expression in combination with DAPT resulted in hematopoietic like cells emerging from mature endothelial populations. Runx1 episomal plasmid has an E2Crimson tracer which can track cells still retaining the vector. See FIG. 17B. Cells emerging from culture were round in morphology and expressed hematopoietic markers Runx1 and CD45 after losing Runx1-Crimson labeled episomal and Sox17, although early budding of hematopoietic cells appeared to retain Runx1 episomals (CD31/CD45 right panel) initially. See FIG. 18A. FACS analysis of cultures after reprogramming demonstrated new populations of CD45+ and CD34+CD45+ hematopoietic cells. See FIG. 18B. A Giemsa stain of sorted hematopoietic cell subsets that emerged from the endothelium is shown in FIG. 18C. The episomals engineered to deliver direct reprogramming factors Sox17 and Runx1 to the endothelial cells are shown in FIG. 19A.

Endothelial cell subsets exhibited Sox17 protein levels of high, mid and low after the first step of the protocol. As shown in FIG. 19B, when stained for endogenous Sox17 protein after introduction of Sox17 episomal (right) versus endogenous expression in passage 5 (p5) human umbilical venous endothelial cells (left), there is an increase in endogenous Sox17 levels, but in a very heterogeneous pattern. Thus future iterations of the protocol will be to sort Sox17high, Sox17mid and Sox17low cells to test which may be best for reprogramming.

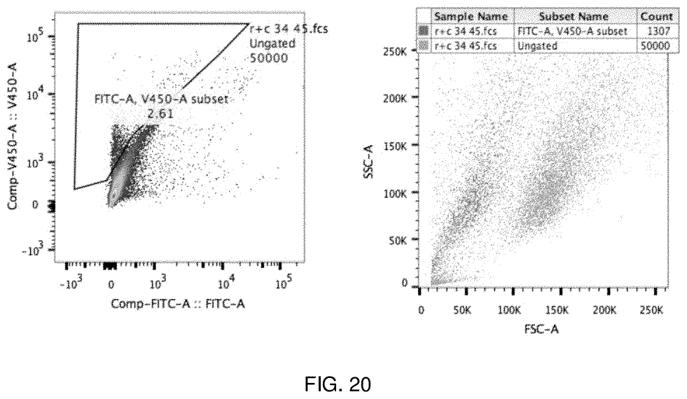

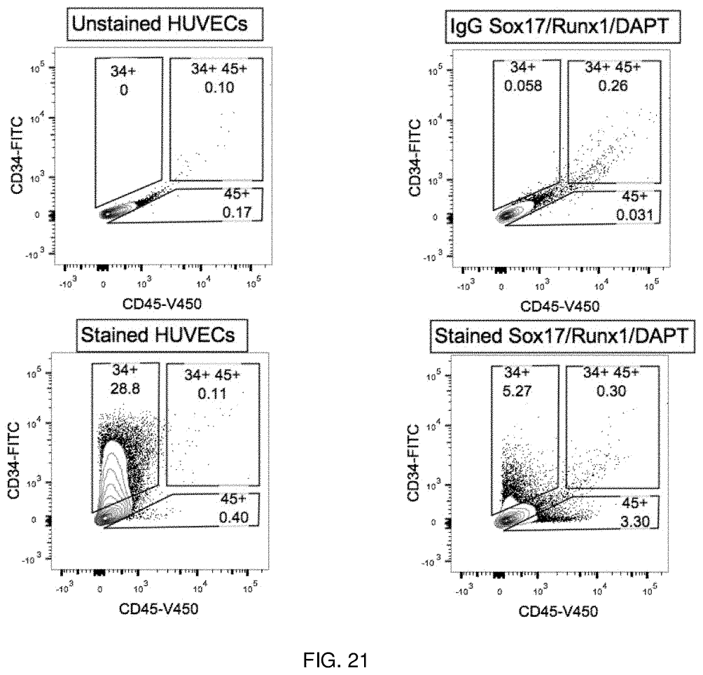

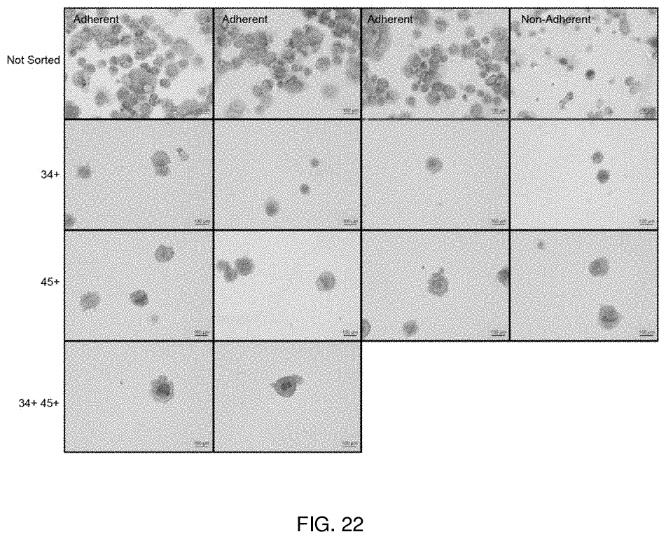

FACS analysis of cultures after reprogramming demonstrated that CD45+ CD34− hematopoietic cells are of smaller size (FSC), which may be indicative of their hematopoietic fate/potential. See FIG. 20. FACS analysis of hematopoietic output suggested a replacement of CD34+ only endothelial cells to CD45+ only hematopoietic cells. See FIG. 21. Giemsa stains of sorted hematopoietic cell subsets (from different replicates) and cells that are maintained in the culture dish suggested that there are various hematopoietic morphology types that are not captured by sorting on current markers. See FIG. 22.

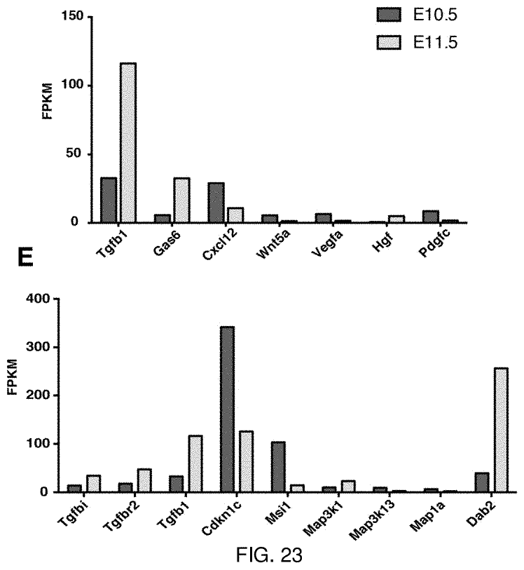

The change in gene transcript levels for Tgfβ1, Gas6, Cxcl12, Wnt5a, VEGFa, Hgf, Pdgfc, Tgfbr2, Tgfb1, Cdkn1c, Mal1, Map3k1, Map3k13 and Dab2 in hematopoietic stem/progenitor cells (HSPCs) during cell maturation was determined. See FIG. 23. Tgfβ1 and cyclin genes increase as hematopoietic stem and progenitor cells (HSPCs) mature in the mouse, and become transplantable in adults. Preliminary mouse studies suggested that adding Tgfβ1 to newly formed murine HSPCs may accelerate their maturation. Hence adding Tgfβ1 to the final stages of the human reprogramming cultures may enhance “transplantability” of cells generated from reprogramming.

### Example 12: Evaluation of Reprogrammed HSPCs for Human Bone Marrow Transplantation (Prophetic)

The reprogrammed HSPCs described above will be evaluated in human clinical trials in comparison with available umbilical cord blood (UBC) for human bone marrow transplantation. Methods for evaluating hematopoietic cells for human bone marrow transplantation are known in the art and are described, for example, in Cutler et al., 2013, Blood 122(17): 3074-3081, which in incorporated by reference herein in its entirety.

Patients with hematologic malignancies will be evaluated in the trial. For example, patients enrolled in the trial may be afflicted with acute lymphoblastic leukemia (ALL), acute myeloblastic leukemia (AML), myelodysplastic syndrome (MDS), or non-Hodgkin lymphoma/chronic lymphocytic leukemia (NHL/CLL). Participants in the trial with hematologic malignancies for whom no HLA-matched donor was available will be conditioned with fludarabine (180 mg/m²), melphalan (100 mg/m²), and antithymocyte globulin (4 mg/kg) and receive graft-versus-host disease (GVHD) prophylaxis with sirolimus (target trough concentration, 3-12 ng/mL) and tacrolimus (target trough concentration 5-10 ng/mL), as described in Cutler et al., 2011, Bone Marrow Transplant. 46(5):659-667.

Umbilicial cord blood (UCB) units will be required to be >4/6 HLA-allele matched with the recipient and each other. Each UCB unit will be required to be >1.5 3 107 total nucleated cells (TNCs)/kg before cryopreservation, and the combined cell dose will be required to be >3.7 3 107 TNC/kg. UCB units will be hierarchically selected from international cord blood banks based on TNC count, HLA match, and unit age. Units against which participants had preformed anti-HLA antibodies will be excluded. On the day of transplantation, cryopreserved UCB units will be thawed and resuspended in a saline solution (0.9% NaCl) containing 5% human serum albumin (Baxter or Talecris) and 8% Dextran 40 (Hospira) (LMD/HSA).

A total of 3 cohorts of patients will be enrolled. In cohort 1, patients will receive UCB. In cohort 2, patients will receive the hematopoietic stem and progenitor cells (HSPC) described above. In cohort 3, patients will receive HSPCs treated with transforming growth factor β1 (Tgfβ1) during the final stages of the human reprogramming cultures. Standard posttransplantation care will be delivered to all participants.

Patient baseline characteristics will be measured and reported descriptively. Patients will be evaluated for neutrophil engraftment, platelet engraftment, donor chimerism, overall survival, and progression free survival. Neutrophil engraftment will be defined as the first of 3 consecutive days with neutrophil recovery to at least 0.5 3109 cells/L. Platelet engraftment will be defined as the first day of a platelet count of at least 20 3 109 cells/L, without supporting transfusion in the prior 3 days. Donor chimerism will be determined from peripheral blood mononuclear cells by analyses of informative short tandem repeat loci using the ABI Profiler-Plus Kit (Applied Biosystems) and the ABI 310 GeneticAnalyzer. Overall survival (OS) will be defined as the time from transplant to death from any cause, whereas progression-free survival (PFS) will be defined as the time from transplant to malignant disease progression or death from any cause. Surviving patients will be censored at their date of last known follow-up.

It is expected that patients treated with the HSPCs will exhibit decreased time to engraftment, lower graft failure rates, and reduced mortality compared to the patients treated with UBCs. In addition, it is expected that the Tgfβ1 treatment of the HSPCs will enhance “transplantability” of the cells generated from reprogramming.

SEQ ID NO:3=the coding sequence for SOX17 which appears within SEQ ID NO:1 beginning at bp #1736 and continues to between bp #2970 and bp #2980

SEQ ID NO:4=the coding sequence for RUNX1 which appears within SEQ ID NO:2 beginning between bp #1736 and bp #1750 and continuing to between bp #3180 and bp #3190.

TABLE 2
Chd5(PAC) - CreERT2/R26RTd/Sox17^{flox} explant statistics.

| Sox17 | | | | | 2-way ANOVA p-value¹ Student's t-test | | | |
|---|---|---|---|---|---|---|---|---|
| Experiment | Genotype | Mean | SEM | N | p-value | Column factor | Row Factor | FIG. |
| HE Ratio | f/⁺ | 0.5187 | 0.04758 | 45 | ***p < 0.0001 | ***p < 0.0001 | ***p < 0.0001 | 2C |
| | f/f | 1.701 | 0.1645 | 38 | | | | |
| BrdU EC | f/⁺ | 3.755 | 0.5285 | 14 | n.s | n.s. | n.s. | S2B |
| | f/f | 2.999 | 0.5819 | 15 | | | | |
| BrdU HC | f/⁺ | 35.28 | 9.048 | 14 | n.s | n.s. | n.s. | S2B |
| | f/f | 52.58 | 9.344 | 15 | | | | |
| Annexin-V | f/⁺ | 54.54 | 3.958 | 18 | n.s | n.s. | n.s. | S2B |
| EC | f/f | 54.62 | 3.072 | 13 | | | | |
| Annexin-V | f/⁺ | 55.67 | 4.624 | 16 | n.s | n.s. | n.s. | S2B |
| HC | f/f | 55.53 | 2.915 | 13 | | | | |
| CD41⁺ % Td⁺ | f/⁺ | 13.03 | 1.209 | 37 | ** p < 0.0085 | ***p < 0.0001 | ***p < 0.0001 | 2D |
| | f/f | 18.28 | 1.532 | 26 | | | | |
| (CD31⁺) | f/⁺ | 11.8 | 2.678 | 14 | *** p < 0.0006 | n.s. | ***p < 0.0030 | 2E |
| 117⁺Ly6a⁺ | f/f | 35.91 | 4.423 | 27 | | | | |
| % Td⁺ | | | | | | | | |
| (CD31⁻) | f/⁺ | 6.399 | 2.683 | 16 | ** p < 0.0062 | n.s. | * p < 0.0189 | S2C |
| 117⁺Ly6a⁺ | f/f | 22.07 | 3.906 | 24 | | | | |
| % Td⁺ | | | | | | | | |
| HE Ratio | f/⁺(−NICD) | 1.079 | 0.1638 | 7 | | * p < 0.205 | ** p < 0.0023 | 5B |
| (+NICD) | f/⁺(+NICD) | 1.370 | 0.1263 | 3 | | | | |
| | f/f(−NICD) | 1.998 | 0.2614 | 5 | ** p < 0.0010 | | | |
| | f/f(+NICD) | 1.131 | 0.1192 | 8 | | | | |
| Total cells | f/⁺ | 13.23 | 1.287 | 13 | n.s | n.s. | n.s. | S2E |
| % CD31⁺ | f/f | 12.04 | 1.214 | 16 | | | | |
| Total cells | f/⁺ | 2,962 | 0.2206 | 15 | n.s | n,s. | n.s. | S2E |
| % CD45+ | f/f | 3.306 | 0.2622 | 21 | | | | |
| % ECs Td⁺ | f/⁺ | 17.84 | 1.909 | 10 | n.s | n.s. | n.s. | S2E |
| | f/f | 16.7 | 1.913 | 10 | | | | |
| Total cells | f/⁺ | 3.577 | 0.3987 | 22 | n.s | n.s. | n.s. | S2E |
| % Td⁺ | f/f | 3.92 | 0.5669 | 22 | | | | |
| E9.5 HE Ratio | f/⁺ | 2.448 | 0.6427 | 12 | n.s. | u.s. | ** p < 0.0018 | S2D |
| | f/f | 4.426 | 0.5841 | 10 | | | | |

¹For 2-way ANOVA, each litter is treated as a different group.

TABLE 3
Cdh5(PAC)-CreERT2/R26RTd/Notchl^{flox} and WT explant statistics.

| Notch 1 | | | | | 2-way ANOVA p-value¹ Student's t-test | | | |
|---|---|---|---|---|---|---|---|---|
| Experiment | Genotype | Mean | SEM | N | p-value | Column factor | Row Factor | FIG. |
| HE Ratio | f/⁺ | 0.8455 | 0.08111 | 18 | * p < 0.038 | * p < 0.0107, | *** p < 0.0001 | 4C |
| | f/f | 1.893 | 0.2834 | 21 | | | | |
| BrdU EC | f/⁺ | 4.897 | 1.33 | 6 | n.s | n.s. | n.s. | 4F |
| | f/f | 5.527 | 0.7001 | 10 | | | | |
| BrdU HC | f/⁺ | 42.57 | 3.258 | 6 | * p < 0.018 | n.s. | * p < 0.0494 | 4F |
| | f/f | 64.9 | 5.497 | 10 | | | | |
| Annexin-V | f/⁺ | 54.60 | 1.852 | 7 | n.s | n.s. | n.s. | S4D |
| EC | f/f | 55.17 | 2.40 | 6 | | | | |
| Annexin-V | f/⁺ | 48.09 | 3.435 | 7 | n.s | n.s. | n.s. | S4E |
| HC | f/f | 45.20 | 2.936 | 6 | | | | |
| CD41⁺ % Td⁺ | f/⁺ | 7.26 | 1.552 | 12 | ** p < 0.0020 | *** p < 0.0001 | *** p < 0.0007 | 4D |
| | f/f | 12.83 | 1.994 | 13 | | | | |
| (CD31⁺) | f/⁺ | 5.101 | 1.499 | 10 | ** p < 0.0084 | ** p < 0.0027 | *** p < 0.0002 | 4E |
| 117⁺Ly6a⁺ | f/f | 15.21 | 3.513 | 6 | | | | |
| % Td⁺ | | | | | | | | |
| (CD31⁻) | f/⁺ | 14.33 | 6.925 | 10 | n.s. | n.s. | n.s. | S4F |
| 117⁺Ly6a⁺ | f/f | 17.81 | 9.02 | 6 | | | | |
| % Td⁺ | | | | | | | | |
| Total cells | f/⁺ | 13.18 | 0.758 | 14 | n.s | n.s. | n.s. | S4H |
| % CD31⁺ | f/f | 12.22 | 0.7345 | 8 | | | | |
| Total cells | f/⁺ | 1.78 | 0.2435 | 15 | n.s | n.s. | n.s. | S4H |
| % CD45⁺ | f/f | 2.027 | 0.2379 | 7 | | | | |
| % ECs Td⁺ | f/⁺ | 17.67 | 3.196 | 6 | n.s | n.s. | n.s. | S4H |
| | f/f | 19.58 | 2.075 | 13 | | | | |
| Total cells | f/⁺ | 2.761 | 0.7531 | 9 | n.s | n.s. | n.s. | S4H |
| % Td⁺ | f/f | 4.366 | 0.761 | 9 | | | | |
| Wildtype HE | WT + DMSO | 0.7825 | 0.05456 | 7 | control | 1-way ANOVA p-value | | S4G |
| ratio (DAPT) | WT + 25 μM | 0.9771 | 0.07913 | 4 | n.s. | ** p < 0.058 | | |
| | WT + 50 μM | 0.9702 | 0.02999 | 9 | ** p < 0.001 | | | |
| | WT + 100 μM | 0.9649 | 0.01957 | 8 | ** p < 0.001 | | | |
| | WT + 200 μM | 0.8264 | 0.0321 | 3 | n.s. | | | |

¹For 2-way ANOVA, each litter is treated as a different group.

TABLE 4
Oligonucleotides used for RT-PCR expression analysis experiments.

| Target | Species | Forward primer | Reverse primer |
|---|---|---|---|
| CD117 | Mouse | 5′-AAATCCAGGCCCACACTCTG | 5′-TAAGGAAGTTGCGTCGGGTC |
| | | (SEQ ID NO: 5) | (SEQ ID NO: 6) |
| CD31 | Mouse | 5′-GAGCCCAATCACGTTTCAGT | 5′-TAAGGAAGTTGCGTCGGGTC |
| | | (SEQ ID NO: 7) | (SEQ ID NO 8) |
| COUP-TFII | H/M | 5′-CGGAGGAACCTGAGCTAC | 5′-CCACTTTGAGGCACTTTTTGA |
| | | (SEQ ID NO 9) | (SEQ ID NO 10) |
| DLL4 | Human | 5′-GCCTATCTGTCTTTCGGGCT | 5′-ATTGTGGGGGATGCATTCGT |
| | | (SEQ ID NO 11) | (SEQ ID NO 12) |
| | Mouse | 5′-CCGGACTTTCTTCCGCATCT | 5′-TGCCGCTATTCTTGTCCCTG |
| | | (SEQ ID NO 13) | (SEQ ID NO 14) |
| EFNB2 | Human | 5′-CCATGGTAACCAGCCACAGT | 5′-CCCCTCTCCCCATCCTAAA |
| | | (SEQ ID NO 15) | (SEQ ID NO 16) |
| | Mouse | 5′-CGAGGTGGCAACAACAATGG | 5′-ATAGTCCCCGCTGACCTTCT |
| | | (SEQ ID NO 17) | (SEQ ID NO 18) |
| EPHB4 | Human | 5′-GCGGAGTATCGGCGTCC | 5′-AGCAGGGTCTCTTCCAAAGC |
| | | (SEQ ID NO 19) | (SEQ ID NO 20) |
| | Mouse | 5′-GCACTTTGAACAGAGGGGGT | 5′-GAGAGAGCCCTCTGGGAAGA |
| | | (SEQ ID NO 21) | (SEQ ID NO 22) |
| GAPDH | Human | 5′-CCACTCCTCCACCTTTGA | 5′-ACCCTGTTGCTGTAGCCA |
| | | (SEQ ID NO 23) | (SEQ ID NO 24) |
| | Mouse | 5′-TGTGTCCGTCGTGGATCTGA | 5′-CCTGCTTCACCACCTTCTTGA |
| | | (SEQ ID NO 25) | (SEQ ID NO 26) |
| GATA2 | Mouse | 5′-TCCAGCTTCACCCCTAAGCA | 5′-ACAGGCATTGCACAGGTAGT |
| | | (SEQ ID NO 27) | (SEQ ID NO 28) |
| HES1 | Mouse | 5′-CATGGAGAAGAGGCGAAGGG | 5′-GGAATGCCGGGAGCTATCTTT |
| | | (SEQ ID NO 29) | (SEQ ID NO 30) |
| LEF1 | HIM | 5′-ATCTTCGCCGAGATCAGTCA | 5′-GTTCTCTGGCCTTGTCGTGG |
| | | (SEQ ID NO 31) | (SEQ ID NO 32) |
| NOTCH1 | Human | 5′-GTTCTTGCAGGGGGTGC | 5′- GGTGAGACCTGCCTGAATG |
| | | (SEQ ID NO 33) | (SEQ ID NO 34) |
| | Mouse | 5′-AACAGTGCCGAATGTGAGTGG | 5′-AAGTGACGCAAGAGCACCTAG |
| | | (SEQ ID NO 35) | (SEQ ID NO 36) |
| RUNX1 | Human | 5′-TTGGGGAGTCCCAGAGGTATC | 5′-CGGAGCGAAAACCAAGACAG |
| | | (SEQ ID NO 37) | (SEQ ID NO 38) |
| | Mouse | 5′-GACCGCAGCATGGTGGAGGT | 5′-GTCTTGTTGCAGCGCCAGTG |
| | | (SEQ ID NO 39) | (SEQ ID NO 40) |
| SOX7 | Human | 5′-CTCTCCTGGGACAGCGTCA | 5′-GCCAAGGACGAGAGGAAA |
| | | (SEQ ID NO 41) | (SEQ ID NO 42) |
| | Mouse | 5′-GGGTCTCTTCTGGGACAGTG | 5′-GGATGAGAGGAAACGTCTGG |
| | | (SEQ ID NO 43) | (SEQ ID NO 44) |
| SOX17 | Human | 5′-AGTGACGACCAGAGCCAGAC | 5′-CCTTAGCCCACACCATGAAA |
| | | (SEQ ID NO 45) | (SEQ ID NO 46) |
| Sox/17 flox | Mouse | 5′- CAGTAAGCCAGATTTGGTCTCTGA | 5′-CCAAGACCTCTTGGGGAAATAGG |
| | | (SEQ ID NO 47) | (SEQ ID NO 48) |
| Sox17 ORF | Mouse | 5′-AAAGACGAACGCAAGCGGTT | 5′-GTCAACGCCTTCCAAGACTT |
| | | (SEQ ID NO 49) | (SEQ ID NO 50) |
| Sox18 | Mouse | 5′-TTGTAGTTGGGATGGTCGC | 5′-CGCAGTACTGAGCAAGATGC |
| | | (SEQ ID NO 51) | (SEQ ID NO 52) |

Sequences shown in Table 4 are CD117 forward primer (SEQ ID NO: 5); CD117 reverse primer (SEQ ID NO: 6); CD31 forward primer (SEQ ID NO: 7); CD31 reverse primer (SEQ ID NO: 8); COUP-TFII forward primer (SEQ ID NO: 9); COUP-TFII reverse primer (SEQ ID NO: 10); DLL4 human forward primer (SEQ ID NO: 11); DLL4 human reverse primer (SEQ ID NO: 12); DLL4 mouse forward primer (SEQ ID NO: 13); DLL4 mouse reverse primer (SEQ ID NO: 14); EFNB2 human forward primer (SEQ ID NO: 15); EFNB2 human reverse primer (SEQ ID NO: 16); EFNB2 mouse forward primer (SEQ ID NO: 17); EFNB2 mouse reverse primer (SEQ ID NO: 18); EPHB4 human forward primer (SEQ ID NO: 19); EPHB4 human reverse primer (SEQ ID NO: 20); EPHB4 mouse forward primer (SEQ ID NO: 21); EPHB4 mouse reverse primer (SEQ ID NO: 22); GAPDH human forward primer (SEQ ID NO: 23); GAPDH human reverse primer (SEQ ID NO: 24); GAPDH mouse forward primer (SEQ ID NO: 25); GAPDH mouse reverse primer (SEQ ID NO: 26); GATA2 mouse forward primer (SEQ ID NO: 27); GATA2 mouse reverse primer (SEQ ID NO: 28); HES1 forward primer (SEQ ID NO: 29); HES1 reverse primer (SEQ ID NO: 30); LEF1 forward primer (SEQ ID NO: 31); LEF1 reverse primer (SEQ ID NO: 32); NOTCH1 human forward primer (SEQ ID NO: 33); NOTCH1 human reverse primer (SEQ ID NO: 34); NOTCH1 mouse forward primer (SEQ ID NO: 35); NOTCH1 mouse reverse primer (SEQ ID NO: 36); RUNX1 human forward primer (SEQ ID NO: 37); RUNX1 human reverse primer (SEQ ID NO: 38); RUNX1 mouse forward primer (SEQ ID NO: 39); RUNX1 mouse reverse primer (SEQ ID NO: 40); SOX7 human forward primer (SEQ ID NO: 41); SOX7 human reverse primer (SEQ ID NO: 42); SOX7 mouse forward primer (SEQ ID NO: 43); SOX7 mouse reverse primer (SEQ ID NO: 44); SOX17 forward primer (SEQ ID NO: 45); SOX17 reverse primer (SEQ ID NO: 46); Sox17flox forward primer (SEQ ID NO: 47); Sox17flox reverse primer (SEQ ID NO: 48); Sox17ORF forward primer (SEQ ID NO: 49); SOX17ORF reverse primer (SEQ ID NO: 50); Sox18 forward primer (SEQ ID NO: 51); and Sox18 reverse primer (SEQ ID NO: 52).

TABLE 5
Oligonucleotides used in RT-PCR for quantitative Sox17 and Runx1 ChIP.

| Species (IP) 5′UTR | | | ATG | | |
|---|---|---|---|---|---|
| Region | | Site | Position | Forward primer | Reverse primer |
| Mouse | Lama1 | | | 5′- CCTCAGCTCCAAGAAAGGAG | 5′- |
| (Sox17) | | | | (SEQ ID NO 53) | AGGATGCTTCCCTGAAATCC |
| | | | | | (SEQ ID NO 54) |
| | CoupTFII | A | −17 bp to | 5′- TCCGGACTTCTGCTCCCCT | 5′- |
| | | | −284 bp | (SEQ ID NO 55) | ACAAACACACCGGGCCAGACA |
| | | | | | (SEQ ID NO 56) |
| | | B | −327 bp | 5′- AGAGAGTGGGAGCAGAACGT | 5′- |
| | | | to −451 | (SEQ ID NO 57) | CGAGCGAGATCTTTAGAGAG |
| | | | bp | | (SEQ ID NO 58) |
| | | C | −2420 bp | 5′- CACCCTCTGTACACACATGT | 5′- CTCTTATGAGTTATGCTGGT |
| | | | to −2613 | (SEQ ID NO 59) | (SEQ ID NO 60) |
| | | | bp | | |
| | Dll4 | A | −26 bp to | 5′- CGCTCGAGACCCTAGGATTT | 5′- GGACTCCGAATCTGCTTGTT |
| | | | −240 bp | (SEQ ID NO 61) | (SEQ ID NO 62) |
| | | B | −271 bp | 5′- TGCTGGGACTGTAGCCACTA | 5′- ACTTTGGCTGCAGCTCTTGG |
| | | | to −448 | (SEQ ID NO 63) | (SEQ ID NO 64) |
| | | | bp | | |
| | | C | −1603 bp | 5′- AATTCTCCATCACCACCACC | 5′- CTGTGGCTTCAGCTGTCA |
| | | | to −1803 | (SEQ ID NO 65) | (SEQ ID NO 66) |
| | | | bp | | |
| | Gata2 | A | −284 bp | 5′- AGGAACTGCGGGTGCGTTTT | 5′- |
| | | | to −523 | (SEQ ID NO 67) | TAGGTCCTGACATCGGTGAC |
| | | | bp | | (SEQ ID NO 68) |
| | | B | −882 bp | 5′- AGAGGTTGGAAGACCTGAGC | 5′- |
| | | | to −1162 | (SEQ ID NO 69) | ACTCCTGCACAGACGTGAAG |
| | | | bp | | (SEQ ID NO 70) |
| | | C | −1327 bp | 5′- TTCAGCCTGGTGGTCTACTA | 5′-CTCTCTGTCCTTCTATCAGG |
| | | | to −1540 | (SEQ ID NO 71) | (SEQ ID NO 72) |
| | Notch1 | A | −343 bp | 5′- CTGGTTCCTGCGAACCCTT | 5′- GATCCTTAGATCCTGGCTC |
| | | | to −454 | (SEQ ID NO 73) | (SEQ ID NO 74) |
| | | | bp | | |
| | | B | −497 bp | 5′- GGGCATCTAGGAACTACTTC | 5′- CCGTACCTCCTCTACTATTG |
| | | | to −640 | (SEQ ID NO 75) | (SEQ ID NO 76) |
| | | | bp | | |
| | | C | −1024 bp | 5′- TTCCACGGTCACCCTTCTCA | 5- GCTTAGCACAGGATGTCCA |
| | | | to −1172 | (SEQ ID NO 77) | (SEQ ID NO 78) |
| | | | bp | | |
| | | D | −1152 bp | 5′- TTGGACATCCTGTGCTAAGC | 5′- |
| | | | to −1357 | (SEQ ID NO 79) | TGCCTTTCAGGAACAGGTGT |
| | | | bp | | (SEQ ID NO 80) |
| | | E | −1566 bp | 5′- CTGAAGGCCTCTAACTGCTT | 5′- TCTGCTGTGCAGCCATACTC |
| | | | to −1775 | (SEQ ID NO 81) | (SEQ ID NO 82) |
| | | | bp | | |
| | Runx1 | A | −180 bp | 5′- GTGGGGGAAAGAATTATTGC | 5′- |
| | | | to −396 | (SEQ ID NO 83) | AGAACCACAAGTTGGGTAGC |
| | | | bp | | (SEQ ID NO 84) |
| | | B | −572 bp | 5′- CCAGGCTGTGTAAGGAAACA | 5′- |
| | | | to −697 | (SEQ ID NO 85) | ACAGGACAGAGAGAGCAAGA |
| | | | bp | | (SEQ ID NO 86) |
| Mouse | Sox17 | A | −76 bp to | 5′- AGTGTCACTAGGCCGGCT | 5′- |
| (Runx1) | | | −202 bp | (SEQ ID NO 87) | GGAGTGAGGCACTGAGATGC |
| | | | | | (SEQ ID NO 88) |
| | | B | −184 bp | 5′- TGGGACGTGGGACTCGGA | 5′- AGCCGGCCTAGTGACACT |
| | | | to −322 | (SEQ ID NO 89) | (SEQ ID NO 90) |
| | | | bp | | |
| | | C | −304 bp | 5′- AGCTCCGGCTAGTTTTCCCG | 5′- TCCGAGTCCCACGTCCCA |
| | | | to −405 | (SEQ ID NO 91) | (SEQ ID NO 92) |
| | | | bp | | |
| | | D | −1081 bp | 5′- TTTGCTATTGCTGGAGGGCG | 5′- GCGGTTATTCTGGCAGAT |
| | | | to −1277 | (SEQ ID NO 93) | (SEQ ID NO 94) |
| | | | bp | | |
| Human | LAMA1 | | | 5′- AAAGTGCAGGGCTGGCTTGT | 5′- TGCAGAGTTTCACAATTC |
| (So17) | | | | (SEQ ID NO 95) | (SEQ ID NO 96) |
| | COUP- | A | −55 bp to | 5′- CTGCAGGCTAGTGCCTACTT | 5′- |
| | TFII | | −142 bp | (SEQ ID NO 97) | TGTTGGCCCCCTGAAAAGAT |
| | | | | | (SEQ ID NO 98) |
| | | B | −237 bp | 5′- AAGGCGAGGCTTGCATTCCT | 5′- GTATTAGGCTCTCTCAGC |
| | | | to −323 | (SEQ ID NO 99) | (SEQ ID NO 100) |
| | | | bp | | |
| | | C | −1161 bp | 5′- GGAAAAACTTCTGTAGCCC | 5′- |
| | | | to −1420 | (SEQ ID NO 101) | ATGGAACTAACGCTCTTCGG |
| | | | bp | | (SEQ ID NO 102) |
| | DLL4 | A | −317 bp | 5′- | 5′- AGCGCCGCTACTGAAACC |
| | | | to −445 | TGGGACTGTAGCAGCTAGAGG | (SEQ ID NO 104) |
| | | | bp | (SEQ ID NO 103) | |
| | | B | −727 bp | 5′- TGGGCACTCATAGGTTGG | 5′- |
| | | | to −875 | (SEQ ID NO 105) | AGGCGCTAGTTACCTAGTGT |
| | | | bp | | (SEQ ID NO 106) |
| | GATA2 | A | −29 bp to | 5′- CTTCTCCAGTCCTCAGAGAA | 5′- |
| | | | −171 bp | (SEQ ID NO 107) | CCCAAAACACCTTTAGAGGG |
| | | | | | (SEQ ID NO 108) |
| | | B | −254 bp | 5′- | 5′- |
| | | | to −443 | AGATTCTGGGGGCTGCGTTGA | AAATGCGACGCCAAGTAGCA |
| | | | bp | (SEQ ID NO 109) | (SEQ ID NO 110) |
| | | C | −423 bp | 5′- TGCTACTTGGCGTCGCATTT | 5′- AGTTCTGCCAGGTCCTTTCA |
| | | | to −595 | (SEQ ID NO 111) | (SEQ ID NO 112) |
| | | | hp | | |
| | | D | −1247 bp | 5′- CGTAAGCTAAAGGATGGGA | 5′- TTGGCGTTCCCCTCAACGC |
| | | | to −1337 | (SEQ ID NO 113) | (SEQ ID NO 114) |
| | | | bp | | |
| | | E | −1513 bp | 5′- GGGAGGCTTAGCAGGCGGCT | 5′- TTTGTCTGTCCGAGGCCTCA |
| | | | to −1642 | (SEQ ID NO 115) | (SEQ ID NO 116) |
| | | | bp | | |
| | NOTCH1 | A | −262 bp | 5′- TGTTGCAGGCCTCGTCCTTT | 5′- |
| | | | to −401 | (SEQ ID NO 117) | CTGCCTCACACACAGAGAGT |
| | | | bp | | (SEQ ID NO 118) |
| | | B | −581 bp | 5′- TACCCTCCCTGGACCCAGTT | 5′- GACCCTATCCCATGCCTCAT |
| | | | to −763 | (SEQ ID NO 119) | (SEQ ID NO 120) |
| | | | bp | | |
| | | C | −703 bp | 5′- TGTAGGCCTCGAGAGCTGCA | 5′- CTGAGCCACGTGGAAAGG |
| | | | to −867 | (SEQ ID NO 121) | (SEQ ID NO 122) |
| | | | bp | | |
| | RUNX1 | A | −1 bp to | 5′- CGCTTCCTCCTGAAAATGCA | 5′- |
| | | | −263 bp | (SEQ ID NO 123) | CATCACCAACCCACAGCCAA |
| | | | | | (SEQ ID NO 124) |
| | | B | −263 bp | 5′- TTGGTGTGGGTTGGTGATG | 5′- |
| | | | to −449 | (SEQ ID NO 125) | CTGTGGAAAGGGGAACAGTT |
| | | | bp | | (SEQ ID NO 126) |
| | | C | −567 bp | 5′- ATAGCCGAGTAGACTTTGC | 5′- |
| | | | to −675 | (SEQ ID NO 127) | TAACAACAGGAGCCGAGTTG |
| | | | bp | | (SEQ ID NO 128) |
| | | D | −1088 bp | 5′- CACACACACACACACACACA | 5′- AAGTGTCTCCTCCTGGTTC |
| | | | to −1354 | (SEQ ID NO 129) | (SEQ ID NO 130) |
| | | | bp | | |
| | | E | −1406 bp | 5′-GACATGCCTGTTTGAAGATG | 5′- |
| | | | to −1603 | (SEQ ID NO 131) | TAGGCAGAGCAGAGCCAAAT |
| | | | bp | | (SEQ ID NO 132) |

Sequences shown in Table 5 are LAMA1 mouse forward primer (SEQ ID NO: 53); LAMA1 mouse reverse primer (SEQ ID NO: 54); COUP TFII A mouse forward primer (SEQ ID NO: 55); COUP TFII A mouse reverse primer (SEQ ID NO: 56); COUP TFII B mouse forward primer (SEQ ID No: 57); COUP TFII B mouse reverse primer (SEQ ID NO: 58); COUP TFII C mouse forward primer (SEQ ID NO: 59); COUP TFII C mouse reverse primer (SEQ ID NO: 60); DII4 A mouse forward primer (SEQ ID NO: 61); DII4 A mouse reverse primer (SEQ ID NO: 62); DII4 B mouse forward primer (SEQ ID NO: 63); DII4 B mouse reverse primer (SEQ ID NO: 64); DII4 C mouse forward primer (SEQ ID NO: 65); DII4 C mouse reverse primer (SEQ ID NO: 66); GATA2 A mouse forward primer (SEQ ID NO: 67); GATA2 A mouse reverse primer (SEQ ID NO: 68); GATA2 B mouse forward primer (SEQ ID NO: 69); GATA2 B mouse reverse primer (SEQ ID NO: 70); GATA2 C mouse forward primer (SEQ ID NO: 71); GATA2 C mouse reverse primer (SEQ ID NO: 72); NOTCH1 A mouse forward primer (SEQ ID NO: 73); NOTCH1 A mouse reverse primer (SEQ ID NO: 74); NOTCH1 B mouse forward primer (SEQ ID NO: 75); NOTCH1 B mouse reverse primer (SEQ ID NO: 76); NOTCH1 C mouse forward primer (SEQ ID NO: 77); NOTCH1 C mouse reverse primer (SEQ ID NO: 78); NOTCH1 D mouse forward primer (SEQ ID NO: 79); NOTCH 1 D mouse reverse primer (SEQ ID NO: 80); NOTCH1 E mouse forward primer (SEQ ID NO: 81); NOTCH 1 E mouse reverse primer (SEQ ID NO: 82); RUNX1 A mouse forward primer (SEQ ID NO: 83); RUNX1 A mouse reverse primer (SEQ ID NO: 84); RUNX1 B mouse forward primer (SEQ ID NO: 85); RUNX1 B mouse reverse primer (SEQ ID NO: 86); SOX17 A mouse forward primer (SEQ ID NO: 87); SOX17 A mouse reverse primer (SEQ ID NO: 88); SOX17 B mouse forward primer (SEQ ID NO: 89); SOX17 B mouse reverse primer (SEQ ID NO: 90); SOX17 C mouse forward primer (SEQ ID NO: 91); SOX17 C mouse reverse primer (SEQ ID NO: 92); SOX17 D mouse forward primer (SEQ ID NO: 93); SOX17 D mouse reverse primer (SEQ ID NO: 94); LAMA1 human forward primer (SEQ ID NO: 95); LAMA1 human reverse primer (SEQ ID NO: 96); COUP-TFII A human forward primer (SEQ ID NO: 97); COUP-TFII A human reverse primer (SEQ ID NO: 98); COUP-TFII B human forward primer (SEQ ID NO: 99); COUP-TFII B human reverse primer (SEQ ID NO: 100); COUP-TFII C human forward primer (SEQ ID NO: 101); COUP-TFII C human reverse primer (SEQ ID NO: 102); DLL4 A human forward primer (SEQ ID NO: 103); DLL4 A human reverse primer (SEQ ID NO: 104); DLL4 B human forward primer (SEQ ID NO: 105): DLL4 B human reverse primer (SEQ ID NO: 106); GATA2 A human forward primer (SEQ ID NO: 107); GATA2 A human reverse primer (SEQ ID NO: 108); GATA2 B human forward primer (SEQ ID NO: 109); GATA2 B human reverse forward (SEQ ID NO: 110); GATA2 C human forward primer (SEQ ID NO: 111); GATA2 C human reverse primer (SEQ ID NO: 112); GATA2 D human forward primer (SEQ ID NO: 113); GATA2 D human reverse primer (SEQ ID NO: 114); GATA2 E human forward primer (SEQ ID NO: 115); GATA2 E human reverse primer (SEQ ID NO: 116); NOTCH1 A human forward primer (SEQ ID NO: 117); NOTCH1 A human reverse primer (SEQ ID NO: 118); NOTCH1 B human forward primer (SEQ ID NO: 119); NOTCH1 B human reverse primer (SEQ ID NO: 120); NOTCH1 C human forward primer (SEQ ID NO: 121); NOTCH1 C human reverse primer (SEQ ID NO: 122); RUNX1 A human forward primer (SEQ ID NO: 123); RUNX1 A human reverse primer (SEQ ID NO: 124); RUNX1 B human forward primer (SEQ ID NO: 125); RUNX1 B human reverse primer (SEQ ID NO: 126); RUNX1 C human forward primer (SEQ ID NO: 127); RUNX1 C human reverse primer (SEQ ID NO: 128); RUNX1 D human forward primer (SEQ ID NO: 129); RUNX1 D human reverse primer (SEQ ID NO: 130); RUNX1 E human forward primer (SEQ ID NO: 131); RUNX1 E human reverse primer (SEQ ID NO: 132);

TABLE 6
Duplex oligonucleotides used in EMSA.

| | Sox17 binding | |
|---|---|---|
| ChIP site | seq | EMSA probe sequence (duplex) |
| Lef1 | | WT 5′-CATTTCTTTATGTCCTTTGTTTACTGTTCTG (-3′BIOTIN) |
| | | (SEQ ID NO 133) |
| | | MT 5′-CATTTCTTTTATGTCAGGGTGGTACTGTTCTG (SEQ ID |
| | | NO 134) |
| Coup TFII site A | COUPTF2_A1 | WT 5′-CGCGCCGCCTTTTGTGTGTGC (SEQ ID NO 135) |
| | | MT 5′-CGCGCCGGGGTGGGTGTGTGC (SEQ ID NO 136) |
| | COUPTF2_A2 | WT 5′-TTTTGCAAAGTTTTGTCGATTG (SEQ ID NO 137) |
| | | MT 5′-TTTTGCAAAGGGGTGGCGATTG (SEQ ID NO 138) |
| Coup TFII site B | COUPTF2_B | WT 5′-GCTGCAAGTCGATTGTCTGGC (SEQ ID NO 139) |
| | | MT 5′-GCTGCAAGTCGGGTGGCTGGC (SEQ ID NO 140) |
| Coup TFII site C | COUPTF2_C | WT 5′-ACTTCACCTCATTGTTATGATG (SEQ ID NO 141) |
| | | MT 5′-ACTTCACCTGGGTGGTATGATG (SEQ ID NO 142) |
| Dll4 site C | Dll4_C1 | WT 5′-AGAGGAATATTGTAATAGGT (SEQ ID NO 143) |
| | | MT 5′-TGGGGGACCCAGAGAGAAGG (SEQ ID NO 144) |
| | Dll4_C2 | WT 5′-GCACTGATCTTATCGTCCGACCAT (SEQ ID NO 145) |
| | | MT 5′-GCACTGATAGGGTGGTCCGACCAT (SEQ ID NO 146) |
| Gata2 site B | Gata2 B1 | WT 5′-GCGCGGCGCTGATTGGCTGG (SEQ ID NO 147) |
| | | MT 5′-GCGCGGCGCTGGGTGGCTGG (SEQ ID NO 148) |
| | Gata2_B2 | WT 5′-CGGGAGCAGCCAATGGGGGG (SEQ ID NO 149) |
| | | MT 5′-CGGGAGCAGGGGTGGGGGGG (SEQ ID NO 150) |
| Gata2 site C | Gata2_C | WT 5′-CGCGACCATTATTGGTCTAGC (SEQ ID NO 151) |
| | | MT 5′-CGCGACCGGGTGGGGTCTAGC (SEQ ID NO 152) |
| Notch1 site A | Notch1_A1 | WT 5′-CCCTTACCCCCTTGTGGACCC (SEQ ID NO 153) |
| | | MT 5′-CCCTTACCCAGGGTGGGACCC (SEQ ID NO 154) |
| | Notch1_A2 | WT 5′-TCGCAAGACAAGGAGGAATGG (SEQ ID NO 155) |
| | | MT 5′-TCGCAAGAAGGGTGGGAATGG (SEQ ID NO 156) |
| | Notch1_A3 | WT 5′-GTCGACTATATTCCAGCTTTGTCAGCA (SEQ ID NO |
| | | 157) |
| | | MT 5′-GTCAAAGGGCCCCCACCCGGGAAAGCA (SEQ ID NO 158) |
| Notch1 site B | Notch1_B | WT 5′-GGATCAGGCTTTGTGTGTAGCCGC (SEQ ID NO 159) |
| | | MT 5′-GGATCAGGAGGGTGGTGTAGCCGC (SEQ ID NO 160) |
| Notch1 site C | Notch1_C | WT 5′-GTTCCAGTACAATGACTGCTAGCG (SEQ ID NO 161) |
| | | MT 5′-GTTCCAGTAAGGGTGGTGCTAGCG (SEQ ID NO 162) |
| Notch1 site D | Notch1_D | WT 5′-CCAGATTATATTGTCCTAGGACCC (SEQ ID NO 163) |
| | | MT 5′-GGGTCCTAGGACAATATAATCTGG (SEQ ID NO 164) |
| Notch1 site E | Notch1_E1 | WT 5′-TCCACTGTCTTTGTCTAGCAATGA (SEQ ID NO 165) |
| | | MT 5′-TCCACTGTAGGGTGGTAGCAATGA (SEQ ID NO 166) |
| | Notch1_E2 | WT 5′-TGTGTCGACTATATTCCAGCT (SEQ ID NO 167) |
| | | MT 5′-TGTGTCGAAGGGTGGCCAGCT (SEQ ID NO 168) |
| Runx1 site A | Runx1_A1 | WT 5′-AAATATTCAAATTGTTAAAGATTA (SEQ ID NO 169) |
| | | MT 5′-AAATATTCAAAGGGGAAAAGATTA (SEQ ID NO 170) |
| Runx1_A2 | | WT 5′-GTTTGCATTCAGTGTGATTCGTCC (SEQ ID NO 171) |
| | | MT 5′-GTTTGCATAGGGTGGGATTCGTCC (SEQ ID NO 172) |

Sequences shown in Table 6 are LEF1 WT (SEQ ID NO: 133); LEF MT (SEQ ID NO: 134); COUPTF2_A1 WT (SEQ ID NO: 135): COUPTF2_A1 MT (SEQ ID NO: 136); COUPTF2_A2 WT (SEQ ID NO: 137); COUPTF2_A2 MT (SEQ ID NO: 138); COUPTF2_B WT (SEQ ID NO: 139); COUPTF2_B MT (SEQ ID NO: 140); COUPTF2_C WT (SEQ ID NO: 141); COUPTF2_C MT (SEQ ID NO: 142); DLL4_C1 WT (SEQ ID NO: 143); DLL4_C1 MT (SEQ ID NO: 144); DLL4_C2 WT (SEQ ID NO: 145); DLL4_C2 MT (SEQ ID NO: 146); GATA2_B1 WT (SEQ ID NO: 147); GATA2_B1 MT (SEQ ID NO: 148); GATA2_B2 WT (SEQ ID NO: 149); GATA2_B2 MT (SEQ ID NO: 150); GATA2_C WT (SEQ ID NO: 151); GATA2_C MT (SEQ ID NO: 152); NOTCH1_A1 WT (SEQ ID NO: 153); NOTCH1_A1 MT (SEQ ID NO: 154); NOTCH1_A2 WT (SEQ ID NO: 155); NOTCH1_A2 MT (SEQ ID NO: 156); NOTCH1_A3 WT (SEQ ID NO: 157); NOTCH1_A3 MT (SEQ ID NO: 158); NOTCH1_B WT (SEQ ID NO: 159); NOTCH1_B MT (SEQ ID NO: 160); NOTCH1_C WT (SEQ ID NO: 161); NOTCH1_C MT (SEQ ID NO: 162); NOTCH1_D WT (SEQ ID NO: 163); NOTCH1_D MT (SEQ ID NO: 164); NOTCH1_E1 WT (SEQ ID NO: 165); NOTCH1_E1 MT (SEQ ID NO: 166); NOTCH1_E2 WT (SEQ ID NO: 167); NOTCH1_E2 MT (SEQ ID NO: 168); RUNX1_A1 WT (SEQ ID NO: 169); RUNX1_A1 MT (SEQ ID NO: 170); RUNX1_A2 WT (SEQ ID NO: 171); and RUNX1_A2 MT (SEQ ID NO: 172).

TABLE 7
Antibodies used for ChIP, IF and Flow Cytometry.

| Antibody | Target | Manufacturer | Clone/Catalog # | Source/Isotype |
|---|---|---|---|---|
| 1° Ab | CD117 | BD Pharmingen | 553352 | Rat |
| | CD31 | BD Pharmingen | 553370 | Rat |
| | CD41 | Abeam | Ab11024 | Rat |
| | Dll4 | R&D Systems | AF1389 | Goat |
| | Gata2 | Pierce | PA1-100 | Rabbit |
| | Notch1 | Cell Signaling | D6F11 | Rabbit |
| | Runx1 | Abcam | Ab92336 | Rabbit (monoclonal) |
| | Runx1* | Abcam | Ab35962 | Rabbit (polyclonal) |
| | Runx1 (AML1 ChIP) | Cell signaling | D4A6 | Rabbit (monoclonal) |
| | Sox17 | R&D Systems | AF1924 | Goat |
| 2° Ab | Alexa 488 α-goat | Invitrogen | A11055 | Donkey |
| | Alexa 488 α-rabbit | Invitrogen | A21206 | Donkey |
| | Alexa 488 α-rat | Invitrogen | A21208 | Donkey |
| | Alexa 594 α-mouse | Invitrogen | A21203 | Donkey |
| | Alexa 594 α-rabbit | Invitrogen | A21207 | Donkey |
| | Alexa 594 α-rat | Invitrogen | A21209 | Donkey |
| | Alexa 647 α-goat | Invitrogen | A21447 | Donkey |
| | Alexa 647 α-rabbit | Invitrogen | A31573 | Donkey |
| Conjugated | CD117-APC | BD Biosciences | 553356 | Rat IgG2b, κ |
| | CD31-APC | BD Biosciences | 551262 | Rat IgG2a, κ |
| | CD31-PE | BD Biosciences | 553373 | Rat IgG2a, κ |
| | CD4-PB | Invitrogen | MCD0428 | Rat IgG2a, κ |
| | CD41-FITC | BD Biosciences | 553848 | Rat IgG1, κ |
| | CD45-FITC | BD Biosciences | 553080 | Rat IgG2b, κ |
| | CD45-PerCP | Biolegend | 103130 | Rat IgG2b, κ |
| | CD8-FITC | BD Biosciences | 553031 | Rat IgG2a, κ |
| | Ly6A-PE-Cy7 | BD Biosciences | 558162 | Rat IgG2a, κ |
| | TCRβ-APC | Biolegend | 109212 | Armenian Hamster IgG |
| Isotype controls | IgG-APC | Biolegend | 400912 | Armenian Hamster IgG |
| | IgG-APC | BD Biosciences | 553991 | Rat IgG2b, κ |
| | IgG-APC | BD Biosciences | 554690 | Rat IgG2a, κ |
| | IgG-FITC | BD Biosciences | 554684 | Rat IgG1, κ |
| | IgG-FITC | BD Biosciences | 554688 | Rat IgG2a, κ |
| | IgG-FITC | BD Biosciences | 556923 | Rat IgG2b, κ |
| | IgG-PE | BD Biosciences | 554689 | Rat IgG2a, κ |
| | IgG-PE-Cy7 | BD Biosciences | 552784 | Rat IgG2a, κ |
| | IgG-PerCP | Biolegend | 400336 | Rat IgG2a, κ |
| Other | AnnexinV-FITC | BD Pharmingen | 556570 | |
| | BrdU Cell Cycle | BD Pharmingen | 558662 | |
| | Lectin HPA Alexa-488 | Life Technologies | 111271 | Helix pomatia snail |

TABLE 8
Oligonucleotides used for mouse genotyping and excision PCR.

| Line | | Forward primer | Reverse primer |
|---|---|---|---|
| Cdh5(PAC)- | +/− | 5′-ATCCAGGTTACGGATATAGT | 5′-CCAAAATTTGCCTGCATTACCGGTCGA |
| CreERT2 | | (SEQ ID NO: 173) | (SEQ ID NO: 174) |
| R26R- | WT/Td | 5′-CTCTGCTGCCTCCTGGCTTCT | 5′-CCAGGCGGATCACAAGCAATA |
| tdTomato | | (SEQ ID NO: 175) | (SEQ ID NO: 176) |
| | | | 5′-TCAATGGGCGGGGGTCGTT |
| | | | (SEQ ID NO: 177) |
| Notch1^{f/f} | WT/Flox | 5′-CTGACTTAGTAGGGGGAAAAC | 5′-AGTGGTCCAGGGTGTGAGTGT |
| | | (SEQ ID NO: 178) | (SEQ ID NO: 179) |
| | Δ | | 5′-TAAAAAGCGACAGCTGCGGAG |
| | | | (SEQ ID NO: 180) |
| R26R- | +/− | 5′-ACAGATCTGGATGCCCGAAT | 5′-TTGTTGGCTCCGTTCTTCAG |
| NICD-GFP | | (SEQ ID NO: 181) | (SEQ ID NO: 182) |
| Sox17^{f/f} | WT/Flox | 5′-TTGCCGAACACACAAAAGGAG | 5′-TGGAGGTGCTGCTCACTGTAA |
| | | (SEQ ID NO: 183) | (SEQ ID NO: 184) |
| Sox17^{f/f} | WT/Flox | 5′-TCTTGATCCCACTTCCCACA | 5′-GGACTGGAAAATGAGAGAATA |
| excision | | (SEQ ID NO: 185) | (SEQ ID NO: 186) |
| | Δ | 5′-TTGCCGAACACACAAAAGGAG | 5′-GGACTGGAAAATGAGAGAATA |
| | | (SEQ ID NO: 252) | (SEQ ID NO: 253) |
| TP1-Venus | +/− | 5′-GGCAGATCACTTCAGCTTCTGC | 5′-CGTTCTTCTGCTTGTCGGCGG |
| (ICR) | | (SEQ ID NO: 187) | (SEQ ID NO: 188) |
| Mlc2α^{f/f} | Cre | 5′-GGCACGATCACTCAGTCAGA | 5′-CCTGTTTTGCACGTTCACCG |
| | | (SEQ ID NO: 189) | (SEQ ID NO: 190) |
| | WT/Flox | | 5′-ATCCCTGTTCTGGTCAATGC |
| | | | (SEQ ID NO: 191) |

Sequences shown in Table 8 are CDH5(PAC)-CREERT2+/−forward primer (SEQ ID NO: 173); CDH5(PAC) CREERT2+/−reverse primer (SEQ ID NO: 174); R26R-TDTOMATO WT/TD forward primer (SEQ ID NO: 175); R26R-TDTOMATO WT/TD reverse primer (top) (SEQ ID NO: 176); R26R-TDTOMATO reverse primer (bottom) (SEQ ID NO: 177); NOTCH1F/F WT/FLOX forward primer (SEQ ID NO: 178); NOTCH1F/F WT/FLOX reverse primer (SEQ ID NO: 179); NOTCH1F/F Δ reverse primer (SEQ ID NO: 180); R26R-NICD-GFP+/−forward primer (SEQ ID NO: 181); R26R-NICD-GFP+/−reverse primer (SEQ ID NO: 182); SOX17F/F WT/FLOX forward primer (SEQ ID NO: 183); SOX17F/F WT/FLOX reverse primer (SEQ ID NO: 184); SOX17F/F EXCISION WT/FLOX forward primer (SEQ ID NO: 185); SOX17F/F EXCISION WT/FLOX reverse primer (SEQ ID NO: 186); TP1-Venus (ICR)+/−forward primer (SEQ ID NO: 187); TP1-Venus (ICR)+/−reverse primer (SEQ ID NO: 188); MLC2AF/F CRE forward primer (SEQ ID NO: 189); MLC2AF/F CRE reverse primer (SEQ ID NO: 190); and MLC2AF/F WT/FLOX reverse primer (SEQ ID NO: 191).

Gata2 nucleic acid sequence (SEQ ID NO: 248):
Gata2 (724 bp)
5′AGGCCCCGCCCGGAGCCCTTCCCCCTCCCTGGGCCACTGGCTTGACCG
CGACCATTATTGGTCTAGCACAGCCTCAAGTGTCTTAGTGCTCAAAGTTC
GGGTGCCCTAGAGAAGTCCACAATCCCTAGACTCATGTTGTCCAGCGGAT
CCTACCAGCCTCTTGCACAGCTATCCCCTGATAGAAGGACAGAGAGTTTG
GGGAGTCAGTTGGATTTGGGCTGGCCGTCGTCCGTAGCAGTGGAGGTGGG
GCTCCGCCCGAGAGTAGAAAGCTGTGGTCCCAGCAGAGAGATACCCAGAA
GGTGCACGTCTCGGCTCCTGGGAAGTCAGGGACCCTATTCGTGCCTAGTT
GCTGGGAGGGCAGAGGTTGGAAGACCTGAGCGTCTGCCGGAGGGGTGCAG
GGTCTGCCCACGGCGAAGGTCCCCTGGGGGGGGGGGCGTTGGCATCAGAG
GCCGCAGAGAGGGCGCTGGTAGGGGGCCAGGCAGCCTAGGAGGCCAGCTT
GCGGGTCATTCCCGAAGTCCAGCGGCCAAAGCGGCGGGAGCAGCCAATGG
GGGGGCGGAGGCTGGGCGGC
GCGCGGCGCTGATTGGCTGGCGCCGGCTTCATAGGCGTGCGCGGCCCCCG
CTTCACGTCTGTGCAGGAGTCGGCAGCTGGCGCCAGGGCGGCCCGGAGGA
TGCAGAGGGGCCGGAGCCGGGCGGGCCGGAAGCCGAGACGCGCGCTGTCC
CCCACCC
Runx1 nucleic acid sequence (SEQ ID NO: 249):
Runx1 (851 bp)
5′GCATCCGGGCTCAGCAGCAAGTTGGTGCCAACGTTGAATTGCTGTTGA
ATAAACAGCAAGGCAATCTTTATCTAAATAATCAGTTGTTCCTCAAACCA
CAAATAACAACAGGATCTGAAAGCCACCAAATCCGCACAGGACAGAGAGA
GCAAGAAAAGACTGAGGCAGGGGATTTCTGTTTGCTTGTTTGTTGTGCTT
TTTTTTCTCTTACAGCCCCTCTCTGCTAAGCTCTGCTCAACTGTTTCCTT
ACACAGCCTGGGGGAGGGCAGGTGGAGGGCAGGAAGGGCATAGCTCAGAA
AGTTTAAAAAAAAAAAATTGACATCACTTAAGTCACGTGATTGGCAAGAG
CCAATGGCGGTGGGCTGTGGAAAGGGGAACAGTTAAATTTGTAATTTGGG
TTGTGTGAAAACTTCTTTGGACCTCATAAACAACCACAGAACCACAAGTT
GGGTGCCTGGCAGTGTCAGAAGTGTAAGCCCAGCACAGTGGTCAGCAGGC
AGGACGAATCACACTGAATGCAAACCACAGGCTTTCGCAGAGCGGTGAGC
AGTTCAACCCACAGCATAGGCGGTGCTTTCGTCTTTTTTTTTTCCTTTTT
TTAATCTTTAACAATTTGAATATTTGTTTCTGCAATAATTCTTTCCCCCA
CCCCCACCCCATAGGACCCATGGAGTACCAGAAGTGTTAGGGTTGGGGGT
AGAAAGAGACGTGGGGAGCCATGGTGGGAGGTGAGGTCAGAGTAAGTGAC
ATTTCTTGGTTTTTGCTCTGAAGGTGAAAGAAATTATAGAATCCCCCGCC
TTCAGGAGAGGTGCGTTTTCGAAAGGAAACGATGGCTTCAGACAGCATTT

Human (Homo sapiens) transforming growth factor β1 (TGFβ1) is described, for example, in Uniprot database entry accession number P01137, which is incorporated by reference herein in its entirety.

```
Human (Homo sapiens) transforming growth factor (β1 (TGFβ1) amino
acid sequence (SEQ ID NO: 250):
                10 20 30 40 50
MPPSGLRLLL LLLPLLWLLV LTPGRPAAGL STCKTIDMEL VKRKRIEAIR
                60 70 80 90 100
GQILSKLRLA SPPSQGEVPP GPLPEAVLAL YNSTRDRVAG ESAEPEPEPE
              110 120 130 140 150
ADYYAKEVTR VLMVETHNEI YDKFKQSTHS IYMFFNTSEL REAVPEPVLL
              160 170 180 190 200
SRAELRLLRL KLKVEQHVEL YQKYSNNSWR YLSNRLLAPS DSPEWLSFDV
              210 220 230 240 250
TGVVRQWLSR GGEIEGFRLS AHCSCDSRDN TLQVDINGFT TGRRGDLATI
              260 270 280 290 300
HGMNRPFLLL MATPLERAQH LQSSRHRRAL DTNYCFSSTE KNCCVRQLYI
              310 320 330 340 350
DFRKDLGWKW IHEPKGYHAN FCLGPCPYIW SLDTQYSKVL ALYNQHNPGA
              360 370 380 390
SAAPCCVPQA LEPLPIVYYV GRKPKVEQLS NMIVRSCKCS
Human (Homo sapiens) transforming growth factor (β1 (TGFβ1) nucleic
acid sequence (SEQ ID NO: 251):
ATGCCGCCCTCCGGGCTGCGGCTGCTGCTGCTGCTGCTACCGCTGCTGTGGCTACTGGTG
CTGACGCCTGGCCGGCCGGCCGCGGGACTATCCACCTGCAAGACTATCGACATGGAGCTG
GTGAAGCGGAAGCGCATCGAGGCCATCCGCGGCCAGATCCTGTCCAAGCTGCGGCTCGCC
AGCCCCCCGAGCCAGGGGGAGGTGCCGCCCGGCCCGCTGCCCGAGGCCGTGCTCGCCCTG
TACAACAGCACCCGCGACCGGGTGGCCGGGGAGAGTGCAGAACCGGAGCCCGAGCCTGAG
GCCGACTACTACGCCAAGGAGGICACCCGCGTGCTAATGGIGGAAACCCACAACGAAATC
TATGACAAGTTCAAGCAGAGTACACACAGCATATATATGTTCTTCAACACATCAGAGCTC
CGAGAAGCGGTACCTGAACCCGTGTTGCTCTCCCGGGCAGAGCTGCGTCTGCTGAGGCTC
AAGTTAAAAGIGGAGCAGCACGTGGAGCTGTACCAGAAATACAGCAACAATTCCTGGCGA
TACCTCAGCAACCGGCTGCTGGCACCCAGCGACTCGCCAGAGTGGTTATCTTTTGATGTC
ACCGGAGTIGTGCGGCAGIGGTIGAGCCGTGGAGGGGAAATTGAGGGCTITCGCCTTAGC
GCCCACTGCTCCTGTGACAGCAGGGATAACACACTGCAAGTGGACATCAACGGGTTCACT
ACCGGCCGCCGAGGTGACCTGGCCACCATTCATGGCATGAACCGGCCTTTCCTGCTTCTC
ATGGCCACCCCGCTGGAGAGGGCCCAGCATCTGCAAAGCTCCCGGCACCGCCGAGCCCIG
GACACCAACTATTGCTTCAGCTCCACGGAGAAGAACTGCTGCGTGCGGCAGCTGTACATT
GACTTCCGCAAGGACCTCGGCTGGAAGTGGATCCACGAGCCCAAGGGCTACCATGCCAAC
TTCTGCCTCGGGCCCTGCCCCTACATTTGGAGCCTGGACA
```

1. A method of differentiating an endothelial cell into a hematopoietic stem cell comprising:
exposing the endothelial cell to an effective amount of at least one hematopoietic effector for a time period sufficient to induce increased expression of, activation of or differentiation into a hematopoietic pathway as compared to an endothelial cell unexposed to the hematopoietic effector; and
exposing the endothelial cell to an effective amount of an hematopoietic effector for a time period sufficient to inhibit, deactivate the hematopoietic pathway or differentiation of the endothelial cell as compared to an endothelial cell unexposed to the hematopoietic effector.

2. The method of claim 1, wherein the step of exposing the endothelial cell to an effective amount of an hematopoietic effector for a time period sufficient to induce expression of a hematopoietic pathway is preceded by a step of isolating one or a plurality of endothelial cells.

3.-4. (canceled)

5. The method of of claim 1, wherein the time period sufficient to induce expression of a hematopoietic pathway is from about 1 day to about 6 days.

6. (canceled)

7. The method of of claim 1, wherein the step of exposing the endothelial cell to an effective amount of an hematopoietic effector comprises culturing the endothelial cell in the presence of a nucleic acid sequence encoding: (i) a hematopoietic activator or a functional fragment thereof; or (ii) a hematopoietic silencer or a functional fragment thereof.

8. (canceled)

9. The method of claim 7, wherein the nucleic acid sequence encoding the hematopoietic activator is an episome or plasmid.

10.-12. (canceled)

13. The method of claim 1, wherein the step of exposing the endothelial cell to an effective amount of an hematopoietic effector comprises exposing the endothelial cell with one or a plurality of small chemical compounds at a pharmacologically effective concentration and for a time period sufficient to silence the hematopoietic pathway.

14. The method of claim 1, wherein the at least one hematopoietic effector comprises Sox17 or a functional fragment thereof.

15. The method of claim 1, wherein the at least one hematopoietic effector comprises Runx1 or a functional fragment thereof.

16. The method of claim 1 further comprising exposing the endothelial cell to one or a plurality of cellular transcription factors chosen from one or a combination of: OCT4, SOX2, KLF4, cMYC, LIN28, NANOG, or any functional fragment thereof.

17. The method of claim 1 further comprising culturing the endothelial cell for a period of time and under conditions sufficient to cause expression of CD41 and/or c-kit.

18.-35. (canceled)

36. The method of claim 1, further comprising:
(a) exposing the endothelial cell to a pharmacologically effective amount of transforming growth factor β1 (TGFβ1) or a functional fragment thereof; or a pharmacologically effective amount of a nucleic acid sequence encoding the TGFβ1 or a functional fragment thereof; or
(b) culturing the endothelial cell in the presence of a nucleic acid sequence encoding a TGFβ1 or a functional fragment thereof.

37. A hematopoietic stem cell produced by the method of claim 1.

38. (canceled)

41. A method of generating a library of hematopoietic cells comprising:
exposing an endothelial cell to an effective amount of an hematopoietic effector for a time period sufficient to induce activation of a hematopoietic pathway; and
exposing the endothelial cell to an effective amount of an hematopoietic effector for a time period sufficient to inhibit the hematopoietic pathway.

42. The method of claim 41 further comprising isolating an endothelial cell from a subject with a predetermined genetic background before exposing the endothelial cell to an effective amount of an hematopoietic effector for a time period sufficient to induce activation or expression of a hematopoietic pathway.

43. (canceled)

44. The method of claim 41 further comprising analyzing an endothelial cell to identify a predetermined genetic background of the endothelial cell before exposing the endothelial cell to an effective amount of an hematopoietic effector for a time period sufficient to induce activation of a hematopoietic pathway.

45. The method of claim 41 further comprising storing the endothelial cell at or below −80 degrees Celsius.

46. (canceled)

47. The method of claim 41, wherein the steps of (a) exposing an endothelial cell to an effective amount of an hematopoietic effector for a time period sufficient to induce expression of or activate a hematopoietic pathway; and (b) exposing the endothelial cell to an effective amount of an hematopoietic effector for a time period sufficient to inhibit the hematopoietic pathway are repeated in respect to a plurality of endothelial cells; and wherein each endothelial cell exposed to a hematopoietic effector is stored at or below −80 degrees Celsius.

48.-63. (canceled)

64. A method of decreasing rejection of transplanted hematopoietic cells in a subject comprising transplanting one or a plurality of hematopoietic cells derived from an endothelial cell known to contain a Human Leukocyte Antigen (HLA) class I, HLC class II, and/or endothelial cell antigens that are compatible with the subject.

65.-68. (canceled)

69. A cell comprising a heterologous nucleic acid sequence encoding one or a plurality of hematopoietic silencers and/or one or a plurality of hematopoetic activators.

70. (canceled)

71. The cell of claim 69, wherein the nucleic acid sequence encoding one or a plurality of hematopoietic silencers comprises a nucleic acid sequence with at least 70%, 75%, 80%, 85%, 90%, 95%, 96%, 97%, 98%, 99% or 100% sequence identity to SEQ ID NO:2; and wherein the a nucleic acid sequence encoding one or a plurality of hematopoietic activators comprises a nucleic acid sequence with at least 70%, 75%, 80%, 85%, 90%, 95%, 96%, 97%, 98%, 99% or 100% sequence identity to SEQ ID NO:1.

72.-78. (canceled)

79. A method of performing a cellular transplant in a subject in need of bone marrow cells comprising: administering to the subject a therapeutically effective amount of one or a plurality of hematopoietic stem cells derived from one or a plurality of endothelial cells.

80.-81. (canceled)

82. A library of cells comprising any one or plurality of cells of claim 69.

83. (canceled)

84. A pharmaceutical composition comprising the cell of of claim.


SEQUENCE LISTING (253 sequences)

SEQ ID NO: 1 - DNA, 13044 bp, Artificial Sequence
  sox17 Plasmid
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcatgagcagcccggatgcgggatacgccagtgacgaccagagccagacccagagcgcgctgcccgcggtgatggccgggctgggcccctgcccctgggccgagtcgctgagccccatcggggacatgaaggtgaagggcgaggcgccggcgaacagcggagcaccggccggggccgcgggccgagccaagggcgagtcccgtatccggcggccgatgaacgctttcatggtgtgggctaaggacgagcgcaagcggctggcgcagcagaatccagacctgcacaacgccgagttgagcaagatgctgggcaagtcgtggaaggcgctgacgctggcggagaagcggcccttcgtggaggaggcagagcggctgcgcgtgcagcacatgcaggaccaccccaactacaagtaccggccgcggcggcgcaagcaggtgaagcggctgaagcgggtggagggcggcttcctgcacggcctggctgagccgcaggcggccgcgctgggccccgagggcggccgcgtggccatggacggcctgggcctccagttccccgagcagggcttccccgccggcccgccgctgctgcctccgcacatgggcggccactaccgcgactgccagagtctgggcgcgcctccgctcgacggctacccgttgcccacgcccgacacgtccccgctggacggcgtggaccccgacccggctttcttcgccgccccgatgcccggggactgcccggcggccggcacctacagctacgcgcaggtctcggactacgctggccccccggagcctcccgccggtcccatgcacccccgactcggcccagagcccgcgggtccctcgattccgggcctcctggcgccacccagcgcccttcacgtgtactacggcgcgatgggctcgcccggggcgggcggcgggcgcggcttccagatgcagccgcaacaccagcaccagcaccagcaccagcaccaccccccgggccccggacagccgtcgccccctccggaggcactgccctgccgggacggcacggaccccagtcagcccgccgagctcctcggggaggtggaccgcacggaatttgaacagtatctgcacttcgtgtgcaagcctgagatgggcctcccctaccaggggcatgactccggtgtgaatctccccgacagccacggggccatttcctcggtggtgtccgacgccagctccgcggtatattactgcaactatcctgacgtgtgagaattcaagcttcggggactagtcatatgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggtaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagcctaactcacattaattgcgttgcgctcactgcccgctttccagtcgggaaacctgtcgtgccagcggatctcaattccgatcatattcaataacccttaatataacttcgtataatgtatgctatacgaagttattaggtctgaagaggagtttacgtccagccaagcttaggatcaattctcatgtttgacagcttatcatcgatggccggccgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacaaccggtcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggctcgagatctgagtccggtagcgctagcgatcctctagtagagtcggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactcccaagcttgcgatcgcatcgataagctgatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaataaagatgtctattgatctcttttagtgtgaatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgccggaacccttaatataacttcgtataatgtatgctatacgaagttattaggtccctcgaagaggttcactagcggatctcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgcgaagaaggggcagagatgtcgtagtcaggtttagttcgtccggggcggggcatcgatcctctagagtcgacgctagcggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 2 - DNA, 13282 bp, Artificial Sequence
  Runx1 and Crimson plasmid
gtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcactagtgatgcgatggcttcagacagcatatttgagtcatttccttcgtacccacagtgcttcatgagagaatgcatacttggaatgaatccttctagagacgtccacgatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactgaatcgaattcaagcttcggggactagtcatatgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcggtaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagcctaactcacattaattgcgttgcgctcactgcccgctttccagtcgggaaacctgtcgtgccagcggatctcaattccgatcatattcaataacccttaatataacttcgtataatgtatgctatacgaagttattaggtctgaagaggagtttacgtccagccaagcttaggatcaattctcatgtttgacagcttatcatcgatggccggccgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacaaccggtttacagctcgtccttctcgcttgcggccccattcagatcctcttctgagatgagtttttgttctgcggccgcctggaacaggtggtggcgggcctcggcgcgctcgtactgctccaccacggtgtagtcctcgttgtgggaggtgatgtcgagcttgtagtccacgtagtggtagccgggcagcttcacgggcttcttggccatgtagatggacttgaactcacacaggtagtggccgccgcccttcagcttcagcgccatgtggttctcgcccttcagcacgccgtcgcgggggtagttgcgctcagtggagggctcccagcccagagtcttcttctgcattacggggccgtcggaggggaagttcacgccgatgaacttcacgtggtagatgagggtgccgtcctgcagggaggagtcctgggtcacggtcaccacgccgccgtcctcgaagttcatcacgcgctcccacttgaagccctcggggaaggactgcttgaggtagtcggggatgtcggcggggtgcttgatgtacgccttggagccgtagaagaactggggggacaggatgtcccaggcgaagggcagggggccgcccttggtcacttgcagcttggcggtctgggtgccctcgtagggcttgccctcgcccacgccctcgatctcgaactcgtggccgttcacggagccctccatgtgcaccttgaagcgcatgaagggcttgatgacgttctcagtgctatccatgctcgagatctgagtccggtagcgctagcgatcctctagtagagtcggtgtcttctatggaggtcaaaacagcgtggatggcgtctccaggcgatctgacggttcactaaacgagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactcccaagcttgcgatcgcatcgataagctgatcctcacaggccgcacccagcttttcttccgttgccccagtagcatctctgtctggtgaccttgaagaggaagaggaggggtcccgagaatccccatccctaccgtccagcaaaaagggggacgaggaatttgaggcctggcttgaggctcaggacgcaaatcttgaggatgttcagcgggagttttccgggctgcgagtaattggtgatgaggacgaggatggttcggaggatggggaattttcagacctggatctgtctgacagcgaccatgaaggggatgagggtgggggggctgttggagggggcaggagtctgcactccctgtattcactgagcgtcgtctaataaagatgtctattgatctcttttagtgtgaatcatgtctgacgaggggccaggtacaggacctggaaatggcctaggagagaagggagacacatctggaccagaaggctccggcggcagtggacctcaaagaagagggggtgataaccatggacgaggacggggaagaggacgaggacgaggaggcggaagaccaggagccccgggcggctcaggatcagggccaagacatagagatggtgtccggagaccccaaaaacgtccaagttgcattggctgcaaagggacccacggtggaacaggagcaggagcaggagcgggaggggcaggagcaggaggggcaggagcaggaggaggggcaggagcaggaggaggggcaggaggggcaggaggggcaggaggggcaggagcaggaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggaggggcaggaggggcaggagcaggaggaggggcaggaggggcaggaggggcaggagcaggaggaggggcaggagcaggaggggcaggaggggcaggaggggcaggagcaggaggggcaggagcaggaggaggggcaggaggggcaggaggggcaggagcaggaggggcaggagcaggaggggcaggagcaggaggggcaggagcaggaggggcaggaggggcaggagcaggaggggcaggaggggcaggagcaggaggggcaggaggggcaggagcaggaggaggggcaggaggggcaggagcaggaggaggggcaggaggggcaggagcaggaggggcaggaggggcaggagcaggaggggcaggaggggcaggagcaggaggggcaggaggggcaggagcaggaggaggggcaggagcaggaggggcaggagcaggaggtggaggccggggtcgaggaggcagtggaggccggggtcgaggaggtagtggaggccggggtcgaggaggtagtggaggccgccggggtagaggacgtgaaagagccagggggggaagtcgtgaaagagccagggggagaggtcgtggacgtggagaaaagaggcccaggagtcccagtagtcagtcatcatcatccgggtctccaccgcgcaggccccctccaggtagaaggccatttttccaccctgtaggggaagccgattattttgaataccaccaagaaggtggcccagatggtgagcctgacgtgcccccgggagcgatagagcagggccccgcagatgacccaggagaaggcccaagcactggaccccggggtcagggtgatggaggcaggcgcaaaaaaggagggtggtttggaaagcatcgtggtcaaggaggttccaacccgaaatttgagaacattgcagaaggtttaagagctctcctggctaggagtcacgtagaaaggactaccgacgaaggaacttgggtcgccggtgtgttcgtatatggaggtagtaagacctccctttacaacctaaggcgaggaactgcccttgctattccacaatgtcgtcttacaccattgagtcgtctcccctttggaatggcccctggacccggcccacaacctggcccgctaagggagtccattgtctgttatttcatggtctttttacaaactcatatatttgctgaggttttgaaggatgcgattaaggaccttgttatgacaaagcccgctcctacctgcaatatcagggtgactgtgtgcagctttgacgatggagtagatttgcctccctggtttccacctatggtggaaggggctgccgcggagggtgatgacggagatgacggagatgaaggaggtgatggagatgagggtgaggaagggcaggagtgatgtaacttgttaggagacgccctcaatcgtattaaaagccgtgtattcccccgcactaaagaataaatccccagtagacatcatgcgtgctgttggtgtatttctggccatctgtcttgtcaccattttcgtcctcccaacatggggcaattgccggaacccttaatataacttcgtataatgtatgctatacgaagttattaggtccctcgaagaggttcactagcggatctcaattgggcatacccatgttgtcacgtcactcagctccgcgctcaacaccttctcgcgttggaaaacattagcgacatttacctggtgagcaatcagacatgcgacggctttagcctggcctccttaaattcacctaagaatgggagcaaccagcaggaaaaggacaagcagcgaaaattcacgcccccttgggaggtggcggcatatgcaaaggatagcactcccactctactactgggtatcatatgctgactgtatatgcatgaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctacccagatatagattaggatagcatatgctatccagatatttgggtagtatatgctacccagatataaattaggatagcatatactaccctaatctctattaggatagcatatgctacccggatacagattaggatagcatatactacccagatatagattaggatagcatatgctacccagatatagattaggatagcctatgctacccagatataaattaggatagcatatactacccagatatagattaggatagcatatgctacccagatatagattaggatagcctatgctacccagatatagattaggatagcatatgctatccagatatttgggtagtatatgctacccatggcaacattagcccaccgtgctctcagcgacctcgtgaatatgaggaccaacaaccctgtgcttggcgctcaggcgcaagtgtgtgtaatttgtcctccagatcgcagcaatcgcgcccctatcttggcccgcccacctacttatgcaggtattccccggggtgccattagtggttttgtgggcaagtggtttgaccgcagtggttagcggggttacaatcagccaagttattacacccttattttacagtccaaaaccgcagggcggcgtgtgggggctgacgcgtgcccccactccacaatttcaaaaaaaagagtggccacttgtctttgtttatgggccccattggcgtggagccccgtttaattttcgggggtgttagagacaaccagtggagtccgctgctgtcggcgtccactctctttccccttgttacaaatagagtgtaacaacatggttcacctgtcttggtccctgcctgggacacatcttaataaccccagtatcatattgcactaggattatgtgttgcccatagccataaattcgtgtgagatggacatccagtctttacggcttgtccccaccccatggatttctattgttaaagatattcagaatgtttcattcctacactagtatttattgcccaaggggtttgtgagggttatattggtgtcatagcacaatgccaccactgaaccccccgtccaaattttattctgggggcgtcacctgaaaccttgttttcgagcacctcacatacaccttactgttcacaactcagcagttattctattagctaaacgaaggagaatgaagaagcaggcgaagattcaggagagttcactgcccgctccttgatcttcagccactgcccttgtgactaaaatggttcactaccctcgtggaatcctgaccccatgtaaataaaaccgtgacagctcatggggtgggagatatcgctgttccttaggacccttttactaaccctaattcgatagcatatgcttcccgttgggtaacatatgctattgaattagggttagtctggatagtatatactactacccgggaagcatatgctacccgtttagggttaacaagggggccttataaacactattgctaatgccctcttgagggtccgcttatcggtagctacacaggcccctctgattgacgttggtgtagcctcccgtagtcttcctgggcccctgggaggtacatgtcccccagcattggtgtaagagcttcagccaagagttacacataaaggcaatgttgtgttgcagtccacagactgcaaagtctgctccaggatgaaagccactcagtgttggcaaatgtgcacatccatttataaggatgtcaactacagtcagagaacccctttgtgtttggtccccccccgtgtcacatgtggaacagggcccagttggcaagttgtaccaaccaactgaagggattacatgcactgccccgcgaagaaggggcagagatgtcgtagtcaggtttagttcgtccggggcggggcatcgatcctctagagtcgacgctagcggatccgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgg

SEQ ID NO: 3 - DNA, 1245 bp, Artificial Sequence
  Sox17 + egfp plasmid
atgagcagcccggatgcgggatacgccagtgacgaccagagccagacccagagcgcgctgcccgcggtgatggccgggctgggcccctgcccctgggccgagtcgctgagccccatcggggacatgaaggtgaagggcgaggcgccggcgaacagcggagcaccggccggggccgcgggccgagccaagggcgagtcccgtatccggcggccgatgaacgctttcatggtgtgggctaaggacgagcgcaagcggctggcgcagcagaatccagacctgcacaacgccgagttgagcaagatgctgggcaagtcgtggaaggcgctgacgctggcggagaagcggcccttcgtggaggaggcagagcggctgcgcgtgcagcacatgcaggaccaccccaactacaagtaccggccgcggcggcgcaagcaggtgaagcggctgaagcgggtggagggcggcttcctgcacggcctggctgagccgcaggcggccgcgctgggccccgagggcggccgcgtggccatggacggcctgggcctccagttccccgagcagggcttccccgccggcccgccgctgctgcctccgcacatgggcggccactaccgcgactgccagagtctgggcgcgcctccgctcgacggctacccgttgcccacgcccgacacgtccccgctggacggcgtggaccccgacccggctttcttcgccgccccgatgcccggggactgcccggcggccggcacctacagctacgcgcaggtctcggactacgctggccccccggagcctcccgccggtcccatgcacccccgactcggcccagagcccgcgggtccctcgattccgggcctcctggcgccacccagcgcccttcacgtgtactacggcgcgatgggctcgcccggggcgggcggcgggcgcggcttccagatgcagccgcaacaccagcaccagcaccagcaccagcaccaccccccgggccccggacagccgtcgccccctccggaggcactgccctgccgggacggcacggaccccagtcagcccgccgagctcctcggggaggtggaccgcacggaatttgaacagtatctgcacttcgtgtgcaagcctgagatgggcctcccctaccaggggcatgactccggtgtgaatctccccgacagccacggggccatttcctcggtggtgtccgacgccagctccgcggtatattactgcaactatcctgacgtgtga

SEQ ID NO: 4 - DNA, 1443 bp, Artificial Sequence
  Runx1 and Crimson plasmid
atggcttcagacagcatatttgagtcatttccttcgtacccacagtgcttcatgagagaatgcatacttggaatgaatccttctagagacgtccacgatgccagcacgagccgccgcttcacgccgccttccaccgcgctgagcccaggcaagatgagcgaggcgttgccgctgggcgccccggacgccggcgctgccctggccggcaagctgaggagcggcgaccgcagcatggtggaggtgctggccgaccacccgggcgagctggtgcgcaccgacagccccaacttcctctgctccgtgctgcctacgcactggcgctgcaacaagaccctgcccatcgctttcaaggtggtggccctaggggatgttccagatggcactctggtcactgtgatggctggcaatgatgaaaactactcggctgagctgagaaatgctaccgcagccatgaagaaccaggttgcaagatttaatgacctcaggtttgtcggtcgaagtggaagagggaaaagcttcactctgaccatcactgtcttcacaaacccaccgcaagtcgccacctaccacagagccatcaaaatcacagtggatgggccccgagaacctcgaagacatcggcagaaactagatgatcagaccaagcccgggagcttgtccttttccgagcggctcagtgaactggagcagctgcggcgcacagccatgagggtcagcccacaccacccagcccccacgcccaaccctcgtgcctccctgaaccactccactgcctttaaccctcagcctcagagtcagatgcaggatacaaggcagatccaaccatccccaccgtggtcctacgatcagtcctaccaatacctgggatccattgcctctccttctgtgcacccagcaacgcccatttcacctggacgtgccagcggcatgacaaccctctctgcagaactttccagtcgactctcaacggcacccgacctgacagcgttcagcgacccgcgccagttccccgcgctgccctccatctccgacccccgcatgcactatccaggcgccttcacctactccccgacgccggtcacctcgggcatcggcatcggcatgtcggccatgggctcggccacgcgctaccacacctacctgccgccgccctaccccggctcgtcgcaagcgcagggaggcccgttccaagccagctcgccctcctaccacctgtactacggcgcctcggccggctcctaccagttctccatggtgggcggcgagcgctcgccgccgcgcatcctgccgccctgcaccaacgcctccaccggctccgcgctgctcaaccccagcctcccgaaccagagcgacgtggtggaggccgagggcagccacagcaactcccccaccaacatggcgccctccgcgcgcctggaggaggccgtgtggaggccctactga

SEQ ID NO: 5 - DNA, 20 bp, Artificial Sequence
  CD117 forward primer
aaatccaggcccacactctg

SEQ ID NO: 6 - DNA, 20 bp, Artificial Sequence
  CD117 reverse primer
taaggaagttgcgtcgggtc

SEQ ID NO: 7 - DNA, 20 bp, Artificial Sequence
  CD31 forward primer
gagcccaatcacgtttcagt

SEQ ID NO: 8 - DNA, 20 bp, Artificial Sequence
  CD31 reverse primer
taaggaagttgcgtcgggtc

SEQ ID NO: 9 - DNA, 18 bp, Artificial Sequence
  COUP-TFII forward primer
cggaggaacctgagctac

SEQ ID NO: 10 - DNA, 21 bp, Artificial Sequence
  COUP-TFII reverse primer
ccactttgaggcactttttga

SEQ ID NO: 11 - DNA, 20 bp, Artificial Sequence
  DLL4 human forward primer
gcctatctgtctttcgggct

SEQ ID NO: 12 - DNA, 20 bp, Artificial Sequence
  DLL4 human reverse primer
attgtgggggatgcattcgt

SEQ ID NO: 13 - DNA, 20 bp, Artificial Sequence
  DLL4 mouse forward primer
ccggactttcttccgcatct

SEQ ID NO: 14 - DNA, 20 bp, Artificial Sequence
  DLL4 mouse reverse primer
tgccgctattcttgtccctg

SEQ ID NO: 15 - DNA, 20 bp, Artificial Sequence
  EFNB2 human forward primer
ccatggtaaccagccacagt

SEQ ID NO: 16 - DNA, 20 bp, Artificial Sequence
  EFNB2 human reverse primer
cccctctcccccatcctaaa

SEQ ID NO: 17 - DNA, 20 bp, Artificial Sequence
  EFNB2 mouse forward primer
cgaggtggcaacaacaatgg

SEQ ID NO: 18 - DNA, 20 bp, Artificial Sequence
  EFNB2 mouse reverse primer
atagtccccgctgaccttct

SEQ ID NO: 19 - DNA, 18 bp, Artificial Sequence
  EPHB4 human forward primer
gcgtgagtatcggcgtcc

SEQ ID NO: 20 - DNA, 20 bp, Artificial Sequence
  EPHB4 human reverse primer
agcagggtctcttccaaagc

SEQ ID NO: 21 - DNA, 20 bp, Artificial Sequence
  EPHB4 mouse forward primer
gcactttgaacagagggggt

SEQ ID NO: 22 - DNA, 20 bp, Artificial Sequence
  EPHB4 mouse reverse primer
gagagagccctctgggaaga

SEQ ID NO: 23 - DNA, 17 bp, Artificial Sequence
  GAPDH human forward primer
ccactcctccaccttga

SEQ ID NO: 24 - DNA, 18 bp, Artificial Sequence
  GAPDH human reverse primer
accctgttgctgtagcca

SEQ ID NO: 25 - DNA, 20 bp, Artificial Sequence
  GAPDH mouse forward primer
tgtgtccgtcgtggatctga

SEQ ID NO: 26 - DNA, 21 bp, Artificial Sequence
  GAPDH mouse reverse primer
cctgcttcaccaccttcttga

SEQ ID NO: 27 - DNA, 20 bp, Artificial Sequence
  GATA2 mouse forward primer
tccagcttcacccctaagca

SEQ ID NO: 28 - DNA, 20 bp, Artificial Sequence
  GATA2 mouse reverse primer
acaggcattgcacaggtagt

SEQ ID NO: 29 - DNA, 20 bp, Artificial Sequence
  HES1forward primer
catggagaagaggcgaaggg

SEQ ID NO: 30 - DNA, 21 bp, Artificial Sequence
  HES1 reverse primer
ggaatgccgggagctatcttt

SEQ ID NO: 31 - DNA, 20 bp, Artificial Sequence
  LEF1 forward primer
atcttcgccgagatcagtca

SEQ ID NO: 32 - DNA, 20 bp, Artificial Sequence
  LEF1 reverse primer
gttctctggccttgtcgtgg

SEQ ID NO: 33 - DNA, 17 bp, Artificial Sequence
  NOTCH1 human forward primer
gttcttgcagggggtgc

SEQ ID NO: 34 - DNA, 19 bp, Artificial Sequence
  NOTCH1 human reverse primer
ggtgagacctgcctgaatg

SEQ ID NO: 35 - DNA, 21 bp, Artificial Sequence
  NOTCH1 mouse forward primer
aacagtgccgaatgtgagtgg

SEQ ID NO: 36 - DNA, 21 bp, Artificial Sequence
  NOTCH1 mouse reverse primer
aagtgacgcaagagcacctag

SEQ ID NO: 37 - DNA, 21 bp, Artificial Sequence
  RUNX1 human forward primer
ttggggagtcccagaggtatc

SEQ ID NO: 38 - DNA, 20 bp, Artificial Sequence
  RUNX1 human reverse primer
cggagcgaaaaccaagacag

SEQ ID NO: 39 - DNA, 20 bp, Artificial Sequence
  RUNX1 mouse forward primer
gaccgcagcatggtggaggt

SEQ ID NO: 40 - DNA, 20 bp, Artificial Sequence
  RUNX1 mouse reverse primer
gtcttgttgcagcgccagtg

SEQ ID NO: 41 - DNA, 19 bp, Artificial Sequence
  SOX7 human forward primer
ctctcctgggacagcgtca

SEQ ID NO: 42 - DNA, 18 bp, Artificial Sequence
  SOX7 human reverse primer
gccaaggacgagaggaaa

SEQ ID NO: 43 - DNA, 20 bp, Artificial Sequence
  SOX7 mouse forward primer
gggtctcttctgggacagtg

SEQ ID NO: 44 - DNA, 20 bp, Artificial Sequence
  SOX7 mouse reverse primer
ggatgagaggaaacgtctgg

SEQ ID NO: 45 - DNA, 20 bp, Artificial Sequence
  SOX17 forward primer
agtgacgaccagagccagac

SEQ ID NO: 46 - DNA, 20 bp, Artificial Sequence
  SOX17 reverse primer
ccttagcccacaccatgaaa

SEQ ID NO: 47 - DNA, 24 bp, Artificial Sequence
  Sox17flox forward primer
cagtaagccagatttggtctctga

SEQ ID NO: 48 - DNA, 23 bp, Artificial Sequence
  Sox17flox reverse primer
ccaagacctcttggggaaatagg

SEQ ID NO: 49 - DNA, 20 bp, Artificial Sequence
  Sox17ORF forward primer
aaagacgaacgcaagcggtt

SEQ ID NO: 50 - DNA, 20 bp, Artificial Sequence
  SOX17ORF reverse primer
gtcaacgccttccaagactt

SEQ ID NO: 51 - DNA, 19 bp, Artificial Sequence
  Sox18 forward primer
ttgtagttgggatggtcgc

SEQ ID NO: 52 - DNA, 20 bp, Artificial Sequence
  Sox18 reverse primer
cgcagtactgagcaagatgc

SEQ ID NO: 53 - DNA, 20 bp, Artificial Sequence
  LAMA1 mouse forward primer
cctcagctccaagaaaggag

SEQ ID NO: 54 - DNA, 20 bp, Artificial Sequence
  LAMA1 mouse reverse primer
aggatgcttccctgaaatcc

SEQ ID NO: 55 - DNA, 19 bp, Artificial Sequence
  COUP TFII A mouse forward primer
tccggacttctgctcccct

SEQ ID NO: 56 - DNA, 21 bp, Artificial Sequence
  COUP TFII A mouse reverse primer
acaaacacaccgggccagaca

SEQ ID NO: 57 - DNA, 20 bp, Artificial Sequence
  COUP TFII B mouse forward primer
agagagtgggagcagaacgt

SEQ ID NO: 58 - DNA, 20 bp, Artificial Sequence
  COUP TFII B mouse reverse primer
cgagcgagatctttagagag

SEQ ID NO: 59 - DNA, 20 bp, Artificial Sequence
  COUP TFII C mouse forward primer
caccctctgtacacacatgt

SEQ ID NO: 60 - DNA, 20 bp, Artificial Sequence
  COUP TFII C mouse reverse primer
ctcttatgagttatgctggt

SEQ ID NO: 61 - DNA, 20 bp, Artificial Sequence
  DII4 A mouse forward primer
cgctcgagaccctaggattt

SEQ ID NO: 62 - DNA, 20 bp, Artificial Sequence
  DII4 A mouse reverse primer
ggactccgaatctgcttgtt

SEQ ID NO: 63 - DNA, 20 bp, Artificial Sequence
  DII4 B mouse forward primer
tgctgggactgtagccacta

SEQ ID NO: 64 - DNA, 20 bp, Artificial Sequence
  DII4 B mouse reverse primer
actttggctgcagctcttgg

SEQ ID NO: 65 - DNA, 20 bp, Artificial Sequence
  DII4 C mouse forward primer
aattctccatcaccaccacc

SEQ ID NO: 66 - DNA, 18 bp, Artificial Sequence
  DII4 C mouse reverse primer
ctgtggcttcagctgtca

SEQ ID NO: 67 - DNA, 20 bp, Artificial Sequence
  GATA2 A mouse forward primer
aggaactgcgggtgcgtttt

SEQ ID NO: 68 - DNA, 20 bp, Artificial Sequence
  GATA2 A mouse reverse primer
taggtcctgacatcggtgac

SEQ ID NO: 69 - DNA, 20 bp, Artificial Sequence
  GATA2 B mouse forward primer
agaggttggaagacctgagc

SEQ ID NO: 70 - DNA, 20 bp, Artificial Sequence
  GATA2 B mouse reverse primer
actcctgcacagacgtgaag

SEQ ID NO: 71 - DNA, 20 bp, Artificial Sequence
  GATA2 C mouse forward primer
ttcagcctggtggtctacta

SEQ ID NO: 72 - DNA, 20 bp, Artificial Sequence
  GATA2 C mouse reverse primer
ctctctgtccttctatcagg

SEQ ID NO: 73 - DNA, 19 bp, Artificial Sequence
  NOTCH1 A mouse forward primer
ctggttcctgcgaaccctt

SEQ ID NO: 74 - DNA, 19 bp, Artificial Sequence
  NOTCH1 A mouse reverse primer
gatccttagatcctggctc

SEQ ID NO: 75 - DNA, 20 bp, Artificial Sequence
  NOTCH1 B mouse forward primer
gggcatctaggaactacttc

SEQ ID NO: 76 - DNA, 20 bp, Artificial Sequence
  NOTCH1 B mouse reverse primer
ccgtacctcctctactattg

SEQ ID NO: 77 - DNA, 20 bp, Artificial Sequence
  NOTCH1 C mouse forward primer
ttccacggtcacccttctca

SEQ ID NO: 78 - DNA, 19 bp, Artificial Sequence
  NOTCH1 C mouse reverse primer
gcttagcacaggatgtcca

SEQ ID NO: 79 - DNA, 20 bp, Artificial Sequence
  NOTCH1 D mouse forward primer
ttggacatcctgtgctaagc

SEQ ID NO: 80 - DNA, 20 bp, Artificial Sequence
  NOTCH 1 D mouse reverse primer
tgcctttcaggaacaggtgt

SEQ ID NO: 81 - DNA, 20 bp, Artificial Sequence
  NOTCH1 E mouse forward primer
ctgaaggcctctaactgctt

SEQ ID NO: 82 - DNA, 20 bp, Artificial Sequence
  NOTCH 1 E mouse reverse primer
tctgctgtgcagccatactc

SEQ ID NO: 83 - DNA, 20 bp, Artificial Sequence
  RUNX1 A mouse forward primer
gtgggggaaagaattattgc

SEQ ID NO: 84 - DNA, 20 bp, Artificial Sequence
  RUNX1 A mouse reverse primer
agaaccacaagttgggtagc

SEQ ID NO: 85 - DNA, 20 bp, Artificial Sequence
  RUNX1 B mouse forward primer
ccaggctgtgtaaggaaaca

SEQ ID NO: 86 - DNA, 20 bp, Artificial Sequence
  RUNX1 B mouse reverse primer
acaggacagagagagcaaga

SEQ ID NO: 87 - DNA, 18 bp, Artificial Sequence
  SOX17 A mouse forward primer
agtgtcactaggccggct

SEQ ID NO: 88 - DNA, 20 bp, Artificial Sequence
  SOX17 A mouse reverse primer
ggagtgaggcactgagatgc

SEQ ID NO: 89 - DNA, 18 bp, Artificial Sequence
  SOX17 B mouse forward primer
tgggacgtgggactcgga

SEQ ID NO: 90 - DNA, 18 bp, Artificial Sequence
  SOX17 B mouse reverse primer
agccggcctagtgacact

SEQ ID NO: 91 - DNA, 20 bp, Artificial Sequence
  SOX17 C mouse forward primer
agctccggctagttttcccg

SEQ ID NO: 92 - DNA, 18 bp, Artificial Sequence
  SOX17 C mouse reverse primer
tccgagtcccacgtccca

SEQ ID NO: 93 - DNA, 20 bp, Artificial Sequence
  SOX17 D mouse forward primer
tttgctattgctggagggcg

SEQ ID NO: 94 - DNA, 18 bp, Artificial Sequence
  SOX17 D mouse reverse primer
gcggttattctggcagat

SEQ ID NO: 95 - DNA, 20 bp, Artificial Sequence
  LAMA1 human forward primer
aaagtgcagggctggcttgt

SEQ ID NO: 96 - DNA, 18 bp, Artificial Sequence
  LAMA1 human reverse primer
tgcagagtttcacaattc

SEQ ID NO: 97 - DNA, 20 bp, Artificial Sequence
  COUP-TFII A human forward primer
ctgcaggctagtgcctactt

SEQ ID NO: 98 - DNA, 20 bp, Artificial Sequence
  COUP-TFII A human reverse primer
tgttggccccgtgaaaagat

SEQ ID NO: 99 - DNA, 20 bp, Artificial Sequence
  COUP-TFII B human forward primer
aaggcgaggcttgcattcct

SEQ ID NO: 100 - DNA, 18 bp, Artificial Sequence
  COUP-TFII B human reverse primer
gtattaggctctctcagc

SEQ ID NO: 101 - DNA, 19 bp, Artificial Sequence
  COUP-TFII C human forward primer
ggaaaaacttctgtagccc

SEQ ID NO: 102 - DNA, 20 bp, Artificial Sequence
  COUP-TFII C human reverse primer
atggaactaacgctcttcgg

SEQ ID NO: 103 - DNA, 21 bp, Artificial Sequence
  DLL4 A human forward primer
tgggactgtagcagctagagg

SEQ ID NO: 104 - DNA, 18 bp, Artificial Sequence
  DLL4 A human reverse primer
agcgccgctactgaaacc

SEQ ID NO: 105 - DNA, 18 bp, Artificial Sequence
  DLL4 B human forward primer
tgggcactcataggttgg

SEQ ID NO: 106 - DNA, 20 bp, Artificial Sequence
  DLL4 B human reverse primer
aggcgctagttacctagtgt

SEQ ID NO: 107 - DNA, 20 bp, Artificial Sequence
  GATA2 A human forward primer
cttctccagtcctcagagaa

SEQ ID NO: 108 - DNA, 20 bp, Artificial Sequence
  GATA2 A human reverse primer
cccaaaacacctttagaggg

SEQ ID NO: 109 - DNA, 21 bp, Artificial Sequence
  GATA2 B human forward primer
agattctgggggctgcgttga

SEQ ID NO: 110 - DNA, 20 bp, Artificial Sequence
  GATA2 B human reverse primer
aaatgcgacgccaagtagca

SEQ ID NO: 111 - DNA, 20 bp, Artificial Sequence
  GATA2 C human forward primer
tgctacttggcgtcgcattt

SEQ ID NO: 112 - DNA, 20 bp, Artificial Sequence
  GATA2 C human reverse primer
agttctgccaggtcctttca

SEQ ID NO: 113 - DNA, 19 bp, Artificial Sequence
  GATA2 D human forward primer
cgtaagctaaaggatggga

SEQ ID NO: 114 - DNA, 19 bp, Artificial Sequence
  GATA2 D human reverse primer
ttggccttcccctcaacgc

SEQ ID NO: 115 - DNA, 20 bp, Artificial Sequence
  GATA2 E human forward primer
gggaggcttagcaggcggct

SEQ ID NO: 116 - DNA, 20 bp, Artificial Sequence
  GATA2 E human reverse primer
tttgtctgtccgaggcctca

SEQ ID NO: 117 - DNA, 20 bp, Artificial Sequence
  NOTCH1 A human forward primer
tgttgcaggcctcgtccttt

SEQ ID NO: 118 - DNA, 20 bp, Artificial Sequence
  NOTCH1 A human reverse primer
ctgcctcacacacagagagt

SEQ ID NO: 119 - DNA, 20 bp, Artificial Sequence
  NOTCH1 B human forward primer
taccctccctggacccagtt

SEQ ID NO: 120 - DNA, 20 bp, Artificial Sequence
  NOTCH1 B human reverse primer
gaccctatcccatgcctcat

SEQ ID NO: 121 - DNA, 20 bp, Artificial Sequence
  NOTCH1 C human forward primer
tgtaggcctcgagagctgca

SEQ ID NO: 122 - DNA, 18 bp, Artificial Sequence
  NOTCH1 C human reverse primer
ctgagccacgtggaaagg

SEQ ID NO: 123 - DNA, 20 bp, Artificial Sequence
  RUNX1 A human forward primer
cgcttcctcctgaaaatgca

SEQ ID NO: 124 - DNA, 20 bp, Artificial Sequence
  RUNX1 A human reverse primer
catcaccaacccacagccaa

SEQ ID NO: 125 - DNA, 19 bp, Artificial Sequence
  RUNX1 B human forward primer
ttggtgtgggttggtgatg

SEQ ID NO: 126 - DNA, 20 bp, Artificial Sequence
  RUNX1 B human reverse primer
ctgtggaaaggggaacagtt

SEQ ID NO: 127 - DNA, 19 bp, Artificial Sequence
  RUNX1 C human forward primer
atagccgagtagactttgc

SEQ ID NO: 128 - DNA, 20 bp, Artificial Sequence
  RUNX1 C human reverse primer
taacaacaggagccgagttg

SEQ ID NO: 129 - DNA, 20 bp, Artificial Sequence
  RUNX1 D human forward primer
cacacacacacacacacaca

SEQ ID NO: 130 - DNA, 19 bp, Artificial Sequence
  RUNX1 D human reverse primer
aagtgtctcctcctggttc

SEQ ID NO: 131 - DNA, 20 bp, Artificial Sequence
  RUNX1 E human forward primer
gacatgcctgtttgaagatc

SEQ ID NO: 132 - DNA, 20 bp, Artificial Sequence
  RUNX1 E human reverse primer
taggcagagcagagccaaat

SEQ ID NO: 133 - DNA, 31 bp, Artificial Sequence
  LEF1 WT
catttctttatgtcctttgtttactgttctg

SEQ ID NO: 134 - DNA, 31 bp, Artificial Sequence
  LEF1 MT
catttctttatgtcagggtggtactgttctg

SEQ ID NO: 135 - DNA, 21 bp, Artificial Sequence
  COUPTF2_A1 WT
cgcgccgccttttgtgtgtgc

SEQ ID NO: 136 - DNA, 21 bp, Artificial Sequence
  COUPTF2_A1 MT
cgcgccggggtgggtgtgtgc

SEQ ID NO: 137 - DNA, 22 bp, Artificial Sequence
  COUPTF2_A2 WT
ttttgcaaagttttgtcgattg

SEQ ID NO: 138 - DNA, 22 bp, Artificial Sequence
  COUPTF2_A2 MT
ttttgcaaaggggtggcgattg

SEQ ID NO: 139 - DNA, 21 bp, Artificial Sequence
  COUPTF2_B WT
gctgcaagtcgattgtctggc

SEQ ID NO: 140 - DNA, 22 bp, Artificial Sequence
  COUPTF2_B MT
gctgcaagtcggggtggctggc

SEQ ID NO: 141 - DNA, 22 bp, Artificial Sequence
  COUPTF2_C WT
acttcacctcattgttatgatg

SEQ ID NO: 142 - DNA, 22 bp, Artificial Sequence
  COUPTF2_C MT
acttcacctgggtggtatgatg

SEQ ID NO: 143 - DNA, 20 bp, Artificial Sequence
  DLL4_C1 WT
agaggaatattgtaataggt

SEQ ID NO: 144 - DNA, 20 bp, Artificial Sequence
  DLL4_C1 MT
tgggggacccagagagaagg

SEQ ID NO: 145 - DNA, 24 bp, Artificial Sequence
  DLL4_C2 WT
gcactgatcttatcgtccgaccat

SEQ ID NO: 146 - DNA, 24 bp, Artificial Sequence
  DLL4_C2 MT
gcactgatagggtggtccgaccat

SEQ ID NO: 147 - DNA, 20 bp, Artificial Sequence
  GATA2_B1 WT
gcgcggcgctgattggctgg

SEQ ID NO: 148 - DNA, 20 bp, Artificial Sequence
  GATA2_B1 MT
gcgcggcgctgggtggctgg

SEQ ID NO: 149 - DNA, 20 bp, Artificial Sequence
  GATA2_B2 WT
cgggagcagccaatgggggg

SEQ ID NO: 150 - DNA, 20 bp, Artificial Sequence
  GATA2_B2 MT
cgggagcaggggtggggggg

SEQ ID NO: 151 - DNA, 21 bp, Artificial Sequence
  GATA2_C WT
cgcgaccattattggtctagc

SEQ ID NO: 152 - DNA, 21 bp, Artificial Sequence
  GATA2_C MT
cgcgaccgggtggggtctagc

SEQ ID NO: 153 - DNA, 21 bp, Artificial Sequence
  NOTCH1_A1 WT
cccttacccccttgtggaccc

SEQ ID NO: 154 - DNA, 21 bp, Artificial Sequence
  NOTCH1_A1 MT
cccttacccagggtgggaccc

SEQ ID NO: 155 - DNA, 21 bp, Artificial Sequence
  NOTCH1_A2 WT
tcgcaagacaaggaggaatgg

SEQ ID NO: 156 - DNA, 21 bp, Artificial Sequence
  NOTCH1_A2 MT
tcgcaagaagggtgggaatgg

SEQ ID NO: 157 - DNA, 27 bp, Artificial Sequence
  NOTCH1_A3 WT
gtcgactatattccagctttgtcagca

SEQ ID NO: 158 - DNA, 27 bp, Artificial Sequence
  NOTCH1_A3 MT
gtcaaagggcccccacccgggaaagca

SEQ ID NO: 159 - DNA, 24 bp, Artificial Sequence
  NOTCH1_B WT
ggatcaggctttgtgtgtagccgc

SEQ ID NO: 160 - DNA, 24 bp, Artificial Sequence
  NOTCH1_B MT
ggatcaggagggtggtgtagccgc

SEQ ID NO: 161 - DNA, 24 bp, Artificial Sequence
  NOTCH1_C WT
gttccagtacaatgactgctagcg

SEQ ID NO: 162 - DNA, 24 bp, Artificial Sequence
  NOTCH1_C MT
gttccagtaagggtggtgctagcg

SEQ ID NO: 163 - DNA, 24 bp, Artificial Sequence
  NOTCH1_D WT
ccagattatattgtcctaggaccc

SEQ ID NO: 164 - DNA, 24 bp, Artificial Sequence
  NOTCH1_D MT
gggtcctaggacaatataatctgg

SEQ ID NO: 165 - DNA, 24 bp, Artificial Sequence
  NOTCH1_E1 WT
tccactgtctttgtctagcaatga

SEQ ID NO: 166 - DNA, 24 bp, Artificial Sequence
  NOTCH1_E1 MT
tccactgtagggtggtagcaatga

SEQ ID NO: 167 - DNA, 21 bp, Artificial Sequence
  NOTCH1_E2 WT
tgtgtcgactatattccagct

SEQ ID NO: 168 - DNA, 21 bp, Artificial Sequence
  NOTCH1_E2 MT
tgtgtcgaagggtggccagct

SEQ ID NO: 169 - DNA, 24 bp, Artificial Sequence
  RUNX1_A1 WT
aaatattcaaattgttaaagatta

SEQ ID NO: 170 - DNA, 24 bp, Artificial Sequence
  RUNX1_A1 MT
aaatattcaaaggggaaaagatta

SEQ ID NO: 171 - DNA, 24 bp, Artificial Sequence
  RUNX1_A2 WT
gtttgcattcactgtgattcgtcc

SEQ ID NO: 172 - DNA, 24 bp, Artificial Sequence
  RUNX1_A2 MT
gtttgcatagggtgggattcgtcc

SEQ ID NO: 173 - DNA, 20 bp, Artificial Sequence
  CDH5(PAC)-CREERT2 +/- forward primer
atccaggttacggatatagt

SEQ ID NO: 174 - DNA, 27 bp, Artificial Sequence
  CDH5(PAC) CREERT2 +/- reverse primer
ccaaaatttgcctgcattaccggtcga

SEQ ID NO: 175 - DNA, 21 bp, Artificial Sequence
  R26R-TDTOMATO WT/TD forward primer
ctctgctgcctcctggcttct

SEQ ID NO: 176 - DNA, 21 bp, Artificial Sequence
  R26R-TDTOMATO WT/TD reverse primer
ccaggcggatcacaagcaata

SEQ ID NO: 177 - DNA, 19 bp, Artificial Sequence
  R26R-TDTOMATO reverse primer
tcaatgggcgggggtcgtt

SEQ ID NO: 178 - DNA, 21 bp, Artificial Sequence
  NOTCH1F/F WT/FLOX forward primer
ctgacttagtagggggaaaac

SEQ ID NO: 179 - DNA, 21 bp, Artificial Sequence
  NOTCH1F/F WT/FLOX reverse primer
agtggtccagggtgtgagtgt

SEQ ID NO: 180 - DNA, 21 bp, Artificial Sequence
  NOTCH1F/F delta reverse primer
taaaaagcgacagctgcggag

SEQ ID NO: 181 - DNA, 20 bp, Artificial Sequence
  R26R-NICD-GFP +/- forward primer
acagatctggatgcccgaat

SEQ ID NO: 182 - DNA, 20 bp, Artificial Sequence
  R26R-NICD-GFP +/- reverse primer
ttgttggctccgttcttcag

SEQ ID NO: 183 - DNA, 20 bp, Artificial Sequence
  SOX17F/F WT/FLOX forward primer
ttgccgaacacacaaaagag

SEQ ID NO: 184 - DNA, 21 bp, Artificial Sequence
  SOX17F/F WT/FLOX reverse primer
tggaggtgctgctcactgtaa

SEQ ID NO: 185 - DNA, 20 bp, Artificial Sequence
  SOX17F/F EXCISION WT/FLOX forward primer
tcttgatcccacttcccaca

SEQ ID NO: 186 - DNA, 21 bp, Artificial Sequence
  SOX17F/F EXCISION WT/FLOX reverse primer
ggactggaaaatgagagaata

SEQ ID NO: 187 - DNA, 22 bp, Artificial Sequence
  TP1-Venus (ICR) +/- forward primer
ggcagatcacttcagcttctgc

SEQ ID NO: 188 - DNA, 21 bp, Artificial Sequence
  TP1-Venus (ICR) +/- reverse primer
cgttcttctgcttgtcggcgg

SEQ ID NO: 189 - DNA, 20 bp, Artificial Sequence
  MLC2AF/F CRE forward primer
ggcacgatcactcagtcaga

SEQ ID NO: 190 - DNA, 20 bp, Artificial Sequence
  MLC2AF/F CRE reverse primer
cctgttttgcacgttcaccg

SEQ ID NO: 191 - DNA, 20 bp, Artificial Sequence
  MLC2AF/F WT/FLOX reverse primer
atccctgttctggtcaatgc

SEQ ID NO: 192 - DNA, 40 bp, Mus musculus
ttgcagaaacaaatattcaaattgttaaagattaaaaaaa

SEQ ID NO: 193 - DNA, 40 bp, Homo sapiens
ttgtaaaaacaaatattcaaattgttaaagattaaaaaaa

SEQ ID NO: 194 - DNA, 40 bp, Pan troglodytes; identical to SEQ ID NO: 193

SEQ ID NO: 195 - DNA, 40 bp, Artificial Sequence; identical to SEQ ID NO: 193
  Runx1_A1 Consensus

SEQ ID NO: 196 - DNA, 45 bp, Mus musculus
cctgtggtttgcattcagtgtgattcgtcctgcctgctgaccact

SEQ ID NO: 197 - DNA, 45 bp, Homo sapiens; identical to SEQ ID NO: 196

SEQ ID NO: 198 - DNA, 45 bp, Pan troglodytes; identical to SEQ ID NO: 196

SEQ ID NO: 199 - DNA, 45 bp, Artificial Sequence; identical to SEQ ID NO: 196
  Runx1_A2 Consensus

SEQ ID NO: 200 - DNA, 23 bp, Mus Musculus
tgttccgaagaggaatattgtaa

SEQ ID NO: 201 - DNA, 44 bp, Homo sapiens
tgtcctagcccgggaatgagcttctgggacagcgcctccgcaaa

SEQ ID NO: 202 - DNA, 44 bp, Pan troglodytes
tgtcctagccggggaatgagcttctgggacagctcctccgcaaa

SEQ ID NO: 203 - DNA, 47 bp, Artificial Sequence
  DII4_C3 Consensus
  misc_feature (17)..(17): n is a, c, g, or t
  misc_feature (36)..(37): n is a, c, g, or t
tgtcctagccggggaantgagcttctgggacagctnncctccgcaaa

SEQ ID NO: 204 - DNA, 47 bp, Mus Musculus
caaaagtttgagctggcgctgcggaaggatcaggctttgtgtgtagc

SEQ ID NO: 205 - DNA, 47 bp, Homo sapiens
caaaagtttgagccggggctgcggaaggatccggctctggcggttgc

SEQ ID NO: 206 - DNA, 53 bp, Pan troglodytes
  misc_feature (1)..(53): n is a, c, g, or t
nnnnnnnnnnnnnnnnnnnnnnnnnnnnnnnnnnnnnnnnnnnnnnnnnnnnn

SEQ ID NO: 207 - DNA, 47 bp, Artificial Sequence
  DII4_C3 Consensus
  misc_feature (14)..(15): n is a, c, g, or t
  misc_feature (33)..(33): n is a, c, g, or t
  misc_feature (38)..(38): n is a, c, g, or t
  misc_feature (41)..(43): n is a, c, g, or t
caaaagtttgagcnnggggctgcggaaggatcnggctntgnnngtgc

SEQ ID NO: 208 - DNA, 46 bp, Mus Musculus
gagagctgcaagtcgattgtctggcttcaagacagaagtaggaggc

SEQ ID NO: 209 - DNA, 42 bp, Homo sapiens
gtgcgctgacatccttagctgggcttcggatcgattcgggcc

SEQ ID NO: 210 - DNA, 45 bp, Pan troglodytes
gagagctgaagtcgattgtctggcttcaagacagaagtaggaggc

SEQ ID NO: 211 - DNA, 47 bp, Artificial Sequence
  Couptfll_B Consensus
  misc_feature (8)..(9): n is a, c, g, or t
gagagctnngaagtcgattgtctggcttcaagacagaagtaggaggc

SEQ ID NO: 212 - DNA, 57 bp, Homo sapiens
gctccattggccacatctgtgcagaaaaggccccgcggcccaggggcgcccgcagtg

SEQ ID NO: 213 - DNA, 57 bp, Pan troglodytes; identical to SEQ ID NO: 212

SEQ ID NO: 214 - DNA, 57 bp, Bos taurus
gctccattggccgggtctgtgcagaaaaggcccggcggccggggggcgcccgcagtg

SEQ ID NO: 215 - DNA, 60 bp, Mus Musculus
tcgccattggccgcgtccataaagaaaagggccccgcggcccggggggacacccgcagtg

SEQ ID NO: 216 - DNA, 40 bp, Mus musculus; identical to SEQ ID NO: 192

SEQ ID NO: 217 - DNA, 40 bp, Homo sapiens; identical to SEQ ID NO: 193

SEQ ID NO: 218 - DNA, 40 bp, Pan troglodytes; identical to SEQ ID NO: 193

SEQ ID NO: 219 - DNA, 40 bp, Artificial Sequence; identical to SEQ ID NO: 193
  Runx1_A1 Consensus

SEQ ID NO: 220 - DNA, 45 bp, Mus musculus; identical to SEQ ID NO: 196

SEQ ID NO: 221 - DNA, 45 bp, Homo sapiens; identical to SEQ ID NO: 196

SEQ ID NO: 222 - DNA, 45 bp, Pan troglodytes; identical to SEQ ID NO: 196

SEQ ID NO: 223 - DNA, 45 bp, Artificial Sequence; identical to SEQ ID NO: 196
  Consensus

SEQ ID NO: 224 - DNA, 35 bp, Mus musculus
ggcgcgcggcgctgattggctggcgccggcttcat

SEQ ID NO: 225 - DNA, 35 bp, Homo sapiens
ggcgcgcggcgttgattggctggcttgggcttctt

SEQ ID NO: 226 - DNA, 35 bp, Pan troglodytes; identical to SEQ ID NO: 225

SEQ ID NO: 227 - DNA, 35 bp, Artificial Sequence; identical to SEQ ID NO: 225
  Consensus

SEQ ID NO: 228 - DNA, 35 bp, Mus musculus
gcgggagcagccaatgggggggggaggctgggcgg

SEQ ID NO: 229 - DNA, 35 bp, Homo sapiens
gcggacgcagccaatgggaggcggaggctgggagg

SEQ ID NO: 230 - DNA, 35 bp, Pan troglodytes; identical to SEQ ID NO: 229

SEQ ID NO: 231 - DNA, 35 bp, Artificial Sequence; identical to SEQ ID NO: 229
  Consensus

SEQ ID NO: 232 - DNA, 23 bp, Mus musculus
tgttccgaagaggaatattgtaa

SEQ ID NO: 233 - DNA, 44 bp, Homo sapiens; identical to SEQ ID NO: 201

SEQ ID NO: 234 - DNA, 44 bp, Pan troglodytes; identical to SEQ ID NO: 202

SEQ ID NO: 235 - DNA, 47 bp, Artificial Sequence; identical to SEQ ID NO: 203
  Consensus
  misc_feature (17)..(17): n is a, c, g, or t
  misc_feature (36)..(37): n is a, c, g, or t

SEQ ID NO: 236 - DNA, 46 bp, Mus musculus
caaagtttgagctggcgctgcggaaggatcaggctttgtgtgtagc

SEQ ID NO: 237 - DNA, 47 bp, Homo sapiens; identical to SEQ ID NO: 205

SEQ ID NO: 238 - DNA, 44 bp, Pan troglodytes
  misc_feature (1)..(44): n is a, c, g, or t
nnnnnnnnnnnnnnnnnnnnnnnnnnnnnnnnnnnnnnnnnnnn

SEQ ID NO: 239 - DNA, 47 bp, Artificial Sequence
  Consensus
  misc_feature (13)..(14): n is a, c, g, or t
  misc_feature (32)..(32): n is a, c, g, or t
  misc_feature (37)..(37): n is a, c, g, or t
  misc_feature (40)..(42): n is a, c, g, or t
  misc_feature (45)..(45): n is a, c, g, or t
caaagtttgagcnnggggctgcggaaggatcnggctntgnnngtngc

SEQ ID NO: 240 - DNA, 46 bp, Mus musculus; identical to SEQ ID NO: 208

SEQ ID NO: 241 - DNA, 42 bp, Homo sapiens; identical to SEQ ID NO: 209

SEQ ID NO: 242 - DNA, 45 bp, Pan troglodytes; identical to SEQ ID NO: 210

SEQ ID NO: 243 - DNA, 47 bp, Artificial Sequence
  Consensus
  misc_feature (8)..(9): n is a, c, g, or t
gagagctnngaggtcgattgtctggcttcaggacagaagtaggaggc

SEQ ID NO: 244 - DNA, 57 bp, Homo sapiens
gctccattggccacatctgtgcagaaaaggcccgcgggcccaggggcgcccgcagtg

SEQ ID NO: 245 - DNA, 57 bp, Pan troglodytes; identical to SEQ ID NO: 212

SEQ ID NO: 246 - DNA, 57 bp, Bos taurus; identical to SEQ ID NO: 214

SEQ ID NO: 247 - DNA, 60 bp, Mus musculus; identical to SEQ ID NO: 215

SEQ ID NO: 248 - DNA, 724 bp, Homo sapiens
aggccccgcccggagcccttccccctccctgggccactggcttgaccgcgaccattattggtctagcacagcctcaagtgtcttagtgctcaaagttcgggtgccctagagaagtccacaatccctagactcatgttgtccagcggatcctaccagcctcttgcacagctatccctgatagaaggacagagagtttggggagtcagttggatttgggctggccgtcgtccgtagcagtggaggtggggctccgcccgagagtagaaagctgtggtcccagcagagagatacccagaaggtgcacgtctcggctcctgggaagtcagggaccctattcgtgcctagttgctgggagggcagaggttggaagacctgagcgtctgccggaggggtgcagggtctgcccacggcgaaggtcccctgggggggggggcgttggcatcagaggccgcagagagggcgctggtagggggccaggcagcctaggaggccagcttgcgggtcattcccgaagtccagcggccaaagcggcgggagcagccaatgggggggcggaggctgggcggcgcgcggcgctgattggctggcgccggcttcataggcgtgcgcggcccccgcttcacgtctgtgcaggagtcggcagctggcgccagggcggcccggaggatgcagaggggccggagccgggcgggccggaagccgagacgcgcgctgtcccccaccc

SEQ ID NO: 249 - DNA, 849 bp, Homo sapiens
gcatccgggctcagcagcaagttggtgccaacgttgaattgctgttgaataaacagcaaggcaatctttatctaaataatcagttgttcctcaaaccacaaataacaacaggatctgaaagccaccaaatccgcacaggacagagagagcaagaaaagactgaggcaggggatttctgtttgcttgtttgttgtgcttttttttctcttacagcccctctctgctaagctctgctcaactgtttccttacacagcctgggggagggcaggtggagggcaggaagggcatagctcagaaagtttaaaaaaaaaaaattgacatcacttaagtcacgtgattggcaagagccaatggcggtgggctgtggaaaggggaacagttaaatttgtaatttgggttgtgtgaaaacttctttggacctcataaacaaccacagaaccacaagttgggtagcctggcagtgtcagaagtgtaagcccagcacagtggtcagcaggcaggacgaatcacactgaatgcaaaccacaggctttcgcagagcggtgagcagttcaacccacagcataggcggtgctttcgtcttttttttttcctttttttaatctttaacaatttgaatatttgtttctgcaataattctttcccccacccccaccccataggacccatggagtaccagaagtgttagggttgggggtagaaagagacgtggggagccatggtgggaggtgaggtcagagtaagtgacatttcttggtttttgctctgaaggtgaaagaaattatagaatcccccgccttcaggagaggtgcgttttcgaaaggaaacgatggcttcagacagcattt

SEQ ID NO: 250 - protein, 390 aa, Homo sapiens
MPPSGLRLLLLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 251 - DNA, 1000 bp, Homo sapiens
atgccgccctccgggctgcggctgctgctgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggaca

SEQ ID NO: 252 - DNA, 21 bp, Artificial Sequence
  Sox 17ff excision WT Flox delta forward primer
ttgccgaacacacaaaaggag

SEQ ID NO: 253 - DNA, 21 bp, Artificial Sequence
  Sox 17ff excision WT/Flox delta reverse primer
ggactggaaaatgagagaata